(19) United States
(12) Patent Application Publication
(10) Pub. No.: US 2024/0324599 A1
(43) Pub. Date: Oct. 3, 2024

(54) PESTICIDAL ENZYMES FOR USE ON NEMATODES, INSECTS, AND MOLLUSKS

(71) Applicant: Spogen Biotech Inc., St. Louis, MO (US)

(72) Inventors: Brian M. Thompson, Creve Coeur, MO (US); Andrew M. Mutka, St. Louis, MO (US); Alejandro Tovar-Mendez, Columbia, MO (US); Erica D. Mueller, Wentzville, MO (US)

(21) Appl. No.: 18/615,771

(22) Filed: Mar. 25, 2024

Related U.S. Application Data
(60) Provisional application No. 63/492,672, filed on Mar. 28, 2023.

(51) Int. Cl. A01N 63/22; A01N 63/50; A01P 5/00; A01P 7/04; A01P 9/00; A01P 21/00; C12N 1/00; C12N 9/20; C12N 9/42; C12N 9/50

(52) U.S. Cl. CPC A01N 63/22; A01N 63/50; A01P 5/00; A01P 7/04; A01P 9/00; A01P 21/00; C12N 1/00; C12N 9/20; C12N 9/2442; C12N 9/50

(57) ABSTRACT

Isolated enzymes exhibiting insecticidal, nematicidal, or pesticidal activity, and recombinant microorganisms expressing said enzymes are provided as well as methods of using the same to protect a plant from a pathogen or pest, improve plant health, or improve plant growth are provided.

### CROSS-REFERENCE TO RELATED APPLICATIONS

This application claims the priority of U.S. Appl. Ser. No. 63/492,672, filed Mar. 28, 2023, the entire disclosure of which is incorporated herein by reference.

### INCORPORATION OF SEQUENCE LISTING

A sequence listing containing the file named “LMNE133US_ST26.xml” which is 457 kilobytes (measured in MS-WINDOWS®) created on Mar. 20, 2024, and comprising 328 sequences, is incorporated herein by reference in its entirety.

### TECHNICAL FIELD

The present invention relates to isolated enzymes as well as recombinant microorganisms comprising enzymes exhibiting insecticidal, nematicidal, or pesticidal activity. Methods of using the isolated enzymes and recombinant microorganisms to improve plant health or growth are further provided.

### BACKGROUND

The invention generally relates to protecting plants from plant pathogens and pests as well as the prevention and management of plant disease caused by plant pathogens and pests. Novel compositions and methods are disclosed exhibiting insecticidal, nematicidal, and pesticidal activity against agriculturally relevant pests of plants and seeds. In particular, provided are enzymes as well as recombinant microorganisms expressing enzymes for protecting a plant from a plant pathogen or pest. Plant parasites and pathogens, including insects, mollusks, arachnids, and nematodes, cause major economic losses annually and affect most agricultural crops worldwide. Treatment of plants with isolated enzymes and recombinant microorganisms via seed, foliar, or soil treatments can improve plant health. The activity of certain enzymes can protect plants from pathogens or pests by acting directly on the pathogen or pest, or indirectly in the environment of the pathogen or pest. A continuing need exists in the art for the development of novel compositions and methods that can be used to further improve crop protection in a variety of agricultural field environments.

### SUMMARY

In one aspect, the provided herein is an isolated enzyme selected from an esterase, a chitinase, a protease, a lipase, a polyurethanase, a collagenase, and combinations of any thereof; wherein the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328; and wherein the enzyme exhibits insecticidal, nematicidal, or pesticidal activity. In some embodiments, the esterase comprises a sequence having at least 85% sequence identity to a sequence selected from SEQ ID NOs: 83-100, 248, 252-255, and 287-289. In other embodiments, a composition for protecting a plant from a pest or pathogen comprising at least one isolated enzyme is provided, wherein the composition exhibits insecticidal, nematicidal, or pesticidal activity. In certain embodiments, the pest or pathogen is defined as an insect, a mollusk, an arachnid, or a nematode. In other embodiments, a plant seed coated with the composition is provided. In still further embodiments, the composition further comprises at least one agriculturally acceptable carrier; or agrochemical. In some embodiments, the agrochemical is an insecticide, or the agriculturally acceptable carrier is a surfactant. In certain embodiments, the agrochemical is a nematicide. In specific embodiments, the nematicide is fluopyram or pydiflumetofen.

In another aspect, provided herein is a method for protecting a plant from a pest or pathogen comprising applying at least one isolated enzyme to a plant growth medium, a plant, a plant seed, or an area surrounding a plant, or a plant seed, wherein the enzyme is selected from an esterase, a chitinase, a protease, a lipase, a polyurethanase, a collagenase, and combinations of any thereof; wherein the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328. In some embodiments, the esterase comprises a sequence having at least 85% sequence identity to a sequence selected from SEQ ID NOs: 83-100, 248, 252-255, and 287-289. In other embodiments, the method further comprises applying at least one agrochemical or agriculturally acceptable carrier. In specific embodiments, the agriculturally acceptable carrier comprises a surfactant or a preservative. In still other embodiments, the method comprises a foliar application to the plant; or applying the enzyme to the area surrounding the plant or plant seed. In certain embodiments, the enzyme is applied to soil surrounding the plant or plant seed. In still further embodiments, the method comprises (a) applying the enzyme to the plant or plant area; (b) applying the enzyme to the plant seed at the time of planting; or (c) coating the plant seed with the enzyme.

Also provided is a recombinant microorganism that expresses an enzyme, wherein expression of the enzyme is increased as compared to the expression level of the enzyme in a wild-type microorganism of the same kind under the same conditions; and the enzyme is selected from an esterase, a chitinase, a protease, a lipase, a polyurethanase, a collagenase, and combinations of any thereof; wherein the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328; and wherein the recombinant microorganism exhibits insecticidal, nematicidal, or pesticidal activity. In some embodiments, the esterase comprises a sequence having at least 85% sequence identity to a sequence selected from SEQ ID NOs: 83-100, 248, 252-255, and 287-289. In other embodiments, a composition for protecting a plant from a pest or pathogen comprising the recombinant microorganism is provided. In certain embodiments, the pest or pathogen is defined as an insect, a mollusk, an arachnid, or a nematode. In other embodiments, a plant seed coated with the composition is provided. In still further embodiments, the composition further comprises at least one agriculturally acceptable carrier; or agrochemical. In specific embodiments, the agriculturally acceptable carrier comprises a surfactant or a preservative. A fermentation product of the recombinant microorganism is also provided. In still further embodiments, a formulation comprising the fermentation product and at least one agriculturally acceptable carrier is provided.

In another aspect, provided herein is a formulation for protecting a plant from a pest or pathogen comprising an esterase. In some embodiments, the formulation comprises a fertilizer and an esterase.

In yet another aspect, provided herein is a formulation for promoting plant growth or plant nutrient uptake comprising an esterase. In some embodiments, the formulation comprises an agrochemical. In other embodiments, a plant seed coated with the formulation is provided. In yet other embodiments, the formulation comprises a fertilizer or a nitrogen stabilizer. In still further embodiments, the fertilizer comprises monoammonium phosphate, di-ammonium phosphate, urea, or a combination of any thereof. In specific embodiments, the nitrogen stabilizer comprises N-(n-butyl) thiophosphoric triamide (NBPT).

In yet another aspect, provided herein is method for promoting plant growth or plant nutrient uptake comprising applying at least one isolated esterase to a plant growth medium, a plant, a plant seed, or an area surrounding a plant or a plant seed. Additionally, provided herein is a method for promoting plant growth or plant nutrient uptake comprising treating a fertilizer with at least one isolated esterase, and applying the treated fertilizer to a plant, a plant seed, or an area surrounding a plant or a plant seed. In some embodiments, the fertilizer comprises monoammonium phosphate, di-ammonium phosphate, urea, or a combination of any thereof. In other embodiments, the fertilizer is further treated with a nitrogen stabilizer. In particular embodiments, the esterase comprises a sequence having at least 80% sequence identity to SEQ ID NO: 254; or the nitrogen stabilizer comprises N-(n-butyl) thiophosphoric triamide.

In another aspect, provided herein is a formulation for protecting a plant from a pest or pathogen comprising a fertilizer and a recombinant microorganism that expresses an esterase, wherein expression of the esterase is increased as compared to the expression level of the enzyme in a wild-type microorganism of the same kind under the same conditions. In some embodiments, a plant seed coated with the formulation is provided. Also provided is a formulation for promoting plant growth or plant nutrient uptake comprising a fertilizer and a recombinant microorganism that expresses an esterase, wherein expression of the esterase is increased as compared to the expression level of the enzyme in a wild-type microorganism of the same kind under the same conditions. In some embodiments, said formulation protects against a plant from a pest or pathogen and promotes plant growth or plant nutrient uptake.

Further provided herein is a method for protecting a plant from a pest or pathogen comprising applying at least one recombinant microorganism that expresses an enzyme to a plant growth medium, a plant, a plant seed, or an area surrounding a plant or a plant seed, wherein expression of the enzyme is increased as compared to the expression level of the enzyme in a wild-type microorganism of the same kind under the same conditions; wherein the enzyme is selected from an esterase, a chitinase, a protease, a lipase, a polyurethanase, a collagenase, and combinations of any thereof; wherein the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328. In some embodiments, the esterase comprises a sequence having at least 80% sequence identity to a sequence selected from SEQ ID NOs: 83-100, 248, 252-255, and 287-289. In other embodiments, the method comprises a foliar application to the plant. In other embodiments, the method comprises applying the recombinant microorganism to the area surrounding the plant or plant seed. In certain embodiments, the recombinant microorganism is applied to soil surrounding the plant or plant seed. In still further embodiments, the method comprises (a) applying the recombinant microorganism to the plant or plant area; (b) applying the recombinant microorganism to the plant seed at the time of planting; or (c) coating the plant seed with the recombinant microorganism.

In another aspect, a formulation for protecting a plant from a pest or pathogen comprising a fertilizer and an esterase is provided. Also provided is a formulation for promoting plant growth or plant nutrient uptake comprising a fertilizer and an esterase. In some embodiments, the compositions, methods, or formulations provided herein may comprise an esterase comprising a sequence having at least 80% sequence identity to a sequence selected from SEQ ID NOs: 83-100, 248, 252-255, and 287-289. In other embodiments, the compositions, methods, or formulations provided herein may further comprise a glucanase. In still other embodiments, the compositions, methods, or formulations provided herein may comprise at least two isolated enzymes. In certain embodiments, at least two enzymes are present in synergistically effective amounts.

In another aspect, provided herein is a formulation for protecting a plant from a pest or pathogen comprising a fertilizer and a recombinant microorganism that expresses an esterase, wherein expression of the esterase is increased as compared to the expression level of the enzyme in a wild-type microorganism of the same kind under the same conditions.

In yet another aspect, provided herein is a formulation for promoting plant growth or plant nutrient uptake comprising a fertilizer and a recombinant microorganism that expresses an esterase, wherein expression of the esterase is increased as compared to the expression level of the enzyme in a wild-type microorganism of the same kind under the same conditions. In some embodiments, the formulation comprises an agrochemical. In other embodiments, the formulation comprises a fertilizer or a nitrogen stabilizer. In still further embodiments, the fertilizer comprises monoammonium phosphate, di-ammonium phosphate, urea, or a combination of any thereof. In specific embodiments, the nitrogen stabilizer comprises N-(n-butyl) thiophosphoric triamide (NBPT).

In further aspect, provided herein is a method for promoting plant growth or plant nutrient uptake comprising treating a fertilizer with at least one recombinant microorganism that expresses an esterase, wherein expression of the esterase is increased as compared to the expression level of the enzyme in a wild-type microorganism of the same kind under the same conditions, and applying the treated fertilizer to a plant, a plant seed, or an area surrounding a plant or a plant seed. In some embodiments, the fertilizer comprises monoammonium phosphate, di-ammonium phosphate, urea, or a combination of any thereof. In other embodiments, the fertilizer is further treated with a nitrogen stabilizer. In further embodiments, the esterase comprises a sequence having at least 80% sequence identity to SEQ ID NO:254 and the nitrogen stabilizer comprises N-(n-butyl) thiophosphoric triamide.

In one aspect, provided herein is a method of producing a formulation for protecting a plant from a pest or pathogen, comprising mixing a recombinant microorganism disclosed herein with at least one agrochemical or agriculturally acceptable carrier. In some embodiments, the method comprises mixing the recombinant microorganism with an agrochemical and an agriculturally acceptable carrier.

Also provided herein is a method of producing a formulation for protecting a plant from a pest or pathogen, comprising mixing a recombinant microorganism described herein with at least one agrochemical or agriculturally acceptable carrier. In specific embodiments, the method comprises mixing the recombinant microorganism with an agrochemical and an agriculturally acceptable carrier.

In yet another aspect, provided herein is a method of producing a composition for protecting a plant from a pest or pathogen, comprising obtaining a recombinant microorganism that expresses an enzyme; wherein the enzyme is selected from an esterase, a chitinase, a protease, a lipase, a polyurethanase, a collagenase, and combinations of any thereof; wherein the esterase comprises a sequence having at least 80% sequence identity to a sequence selected from SEQ ID NOs: 83-100, 248, 252-255, and 287-289, the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328; purifying the enzyme from the recombinant microorganism; and combining the purified enzyme with an agrochemical or agriculturally acceptable carrier. In some embodiments, purifying the enzyme may comprises centrifugation, filtration, concentration, or removal of cell material and debris, or a combination any thereof. In other embodiments, purifying the enzyme comprises lyophilizing, spray drying, or freeze drying the enzyme. In other embodiments, wherein the purified enzyme is combined with a liquid agrochemical.

In still yet another aspect, provided herein is a method of producing a composition for protecting a plant from a pest or pathogen, comprising obtaining a recombinant microorganism that expresses an enzyme, wherein expression of the enzyme is increased as compared to the expression level of the enzyme in a wild-type microorganism of the same kind under the same conditions; and the enzyme is selected from an esterase, a chitinase, a protease, a lipase, a polyurethanase, a collagenase, and combinations of any thereof; wherein the esterase comprises a sequence having at least 80% sequence identity to a sequence selected from SEQ ID NOs: 83-100, 248, 252-255, and 287-289, the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328, wherein the recombinant microorganism exhibits insecticidal, nematicidal, or pesticidal activity; purifying the recombinant microorganism; and combining the purified recombinant microorganism with an agrochemical or agriculturally acceptable carrier. In some embodiments, purifying the recombinant microorganism may comprises centrifugation, filtration, concentration, or a combination any thereof. In some embodiments, purifying the recombinant microorganism comprises lyophilizing, spray drying, or freeze drying the enzyme. In other embodiments, wherein the purified recombinant microorganism is combined with a liquid agrochemical.

In another aspect, provided is a method for controlling a plant pest or plant pest infestation, said method comprising contacting the pest with an effective amount of an isolated enzyme selected from an esterase, a chitinase, a protease, a lipase, a polyurethanase, a collagenase, and combinations of any thereof; wherein the chitinase comprises a sequence having at least 85% sequence identity to a sequence selected from SEQ ID NOs: 179-205, and 249, the protease comprises a sequence having at least 85% sequence identity to a sequence selected from SEQ ID NOs: 122-178, 247, and 310-327, the lipase comprises a sequence having at least 85% sequence identity to a sequence selected from SEQ ID NOs: 1-82, 250, 251, 256-260, and 290-307, the polyurethanase comprises a sequence having at least 85% sequence identity to a sequence selected from SEQ ID NOs: 101-121, 308, and 309, or the collagenase comprises a sequence having at least 85% sequence identity to a sequence selected from SEQ ID NOs: 206-208, and 328; and wherein the enzyme exhibits insecticidal, nematicidal, or pesticidal activity. In some embodiments, the esterase comprises a sequence having at least 80% sequence identity to a sequence selected from SEQ ID NOs: 83-100, 248, 252-255, and 287-289. In other embodiments, the plant pest comprises Black armyworm (Spodoptera cosmioides), Black cutworm (Agrotis ipsilon), Corn carworm (Helicoverpa zea), Cotton leaf worm (Alabama argillacea), Diamondback moth (Plutella xylostella), European corn borer (Ostrinia nubilalis), Fall armyworm (Spodoptera frugiperda), Cry1Fa1 resistant Fall armyworm (Spodoptera frugiperda), Old World bollworm (OWB, Helicoverpa armigera), Southern armyworm (Spodoptera eridania), Soybean looper (Chrysodeixis includens), Spotted bollworm (Earias vittella), Southwestern corn borer (Diatraea grandiosella), Sugarcane borer (Diatraea saccharalis), Sunflower looper (Rachiplusia nu), Tobacco budworm (Heliothis virescens), Tobacco cutworm (Spodoptera litura, also known as cluster caterpillar), Western bean cutworm (Striacosta albicosta), and Velvet bean caterpillar (Anticarsia gemmatalis) Garden snails (Cornu aspersum) or slugs (Deroceras reticulatum). In other embodiments, the plant pest comprises a nematode species from the genera Heterodera and Meloidogynes. In certain embodiments, the plant pest comprises a nematode species selected from the group consisting of: Aglenchus spp., Anguina spp., Aphelenchoides spp., Belonolaimus spp., Bursaphelenchus spp., Cacopaurus spp., Criconemella spp., Criconemoides spp., Ditylenchus spp., Dolichodorus spp., Globodera spp., Helicotylenchus spp., Hemicriconemoides spp., Hemicycliophora spp., Heterodera spp., Hoplolaimus spp., Longidorus spp., Lygus spp., Meloidogyne spp., Meloinema spp., Nacobbus spp., Neotylenchus spp., Paralongidorus spp., Paraphelenchus spp., Paratrichodorus spp., Pratylenchus spp., Pseudohalenchus spp., Psilenchus spp., Punctodera spp., Quinisulcius spp., Radopholus spp., Rotylenchulus spp., Rotylenchus spp., Scutellonema spp., Subanguina spp., Trichodorus spp., Tylenchulus spp., Tylenchorhynchus spp., and Xiphinema spp.

### BRIEF DESCRIPTION OF THE SEQUENCES

SEQ ID NOs: 1-82, 250, 251, 256-260, and 290-307 are lipase polypeptide sequences.

SEQ ID NOs: 83-100, 248, 252-255, and 287-289 are esterase polypeptide sequences.

SEQ ID NOs: 101-121, 308, and 309 are polyurethanase polypeptide sequences.

SEQ ID NOs: 122-178, 247, and 310-327 are protease polypeptide sequences.

SEQ ID NOs: 179-205, and 249 are chitinase polypeptide sequences.

SEQ ID NOs: 206-208, and 328 are collagenase polypeptide sequences.

SEQ ID NOs: 209-246, and 267-286 are amino acid sequences of signal peptides.

SEQ ID NOs: 261-266 are chaperone polypeptide sequences.

### DETAILED DESCRIPTION

Plant pathogens and pests including fungi, fungal-like organisms, bacteria, phytoplasmas, viruses, viroids, insects, and nematodes, can cause significant damage to crop plants, leading to substantial economic loss. Such plant pathogens and pests may infect all types of plant tissue and may be transmitted from plant to plant by a vector or through direct exposure to the pathogen. A number of strategies are currently available and have been employed to control and limit damage caused by plant pathogens and pests. For example, organophosphorus and carbamate nematicides, endophytic fungi, and transgenic plants producing Cry-proteins are currently available to protect against damage caused by nematodes. However, recent estimates place the economic cost of plant parasitic nematodes at USD 157 billion annually (e.g., Mendoza-de Gives, Pathogens. 2022 Jun. 1; 11 (6): 640). Moreover, the global use of these and similar methods to control plant pathogens and pests has created selection pressure for existing alleles that impart resistance to the plant pathogen or pest. Therefore, a continuing need exists in the art to develop novel compositions and methods to protect plants from pathogens and pests, and thus improve plant health and yield.

The present disclosure overcomes the limitations of the prior art by providing enzymes and recombinant microorganisms expressing said enzymes as well as compositions and methods using the same. These enzymes exhibit insecticidal, nematicidal, or pesticidal activity; and yield significantly increased protection against plant pathogens and pests.

In order for a plant pathogen or pest to cause plant damage and disease, a susceptible host plant, a virulent pathogen or pest, and a suitable environment are all required. Accordingly, provided herein are methods and compositions for altering one or more of these requirements to protect a plant from a pathogen or pest. The ability to produce these desirable effects using the enzymes described herein offers unique benefits not otherwise available in the art. To produce such benefits, the present disclosure provides, in certain embodiments, methods and compositions for protecting a plant from a pathogen or pest comprising the enzymes described herein. For example, these enzymes may act directly on the plant pathogen or pest, e.g., contacting the pest or being provided in the diet of the target pest; or indirectly, e.g., by acting on the environment of the plant pathogen or pest.

The following definitions and methods are provided to better define the present invention and to guide those of ordinary skill in the art in the practice of the present invention. Unless otherwise noted, terms are to be understood according to conventional usage by those of ordinary skill in the relevant art.

Reference in this application to the terms “active” or “activity”, “insecticidal” or “insecticidal activity”, “nematicidal” or “nematicidal activity”, “pesticidal” or “pesticidal activity”, and similar terms, refer to efficacy of a polypeptide, such as an enzyme disclosed herein, in inhibiting (inhibiting growth, feeding, fecundity, or viability of a plant pathogen or pest), suppressing (suppressing growth, feeding, fecundity, or viability of a plant pathogen or pest), controlling (controlling the pathogen or pest infestation, controlling the pest feeding activities on a particular crop) or killing (causing the morbidity, mortality, or reduced fecundity of a plant pathogen or pest) a pathogen or pest. These terms are intended to include the result of providing an effective amount of a polypeptide to a pathogen or pest where the exposure of the pathogen or pest to the enzyme results in inhibiting, suppressing, controlling, or killing. These terms also include repulsion of the pathogen or pest from the plant, a tissue of the plant, a plant part, seed, plant cells, or from the particular geographic location where the plant may be growing, as a result of providing an effective amount of the polypeptide on the plant. In some embodiments, the polypeptide can be applied to the plant or to the environment within the location where the plant is located. The terms “bioactivity”, “effective”, “efficacious” or variations thereof are also terms interchangeably utilized in this application to describe the effects of polypeptides of the present invention on plant pathogens and pests. In certain embodiments, such terms describe, for example, a decrease in the growth of a nematode plant pest, a decrease in the ability of the nematode to survive, grow, move, feed, and/or reproduce, a decrease in the infectivity of a nematode plant pest, a decrease in the infestation of a plant by a nematode plant pest, and/or a decrease in nematode cyst development by a nematode plant pest on roots of a plant as compared to an appropriate control. In other embodiments, such terms describe, for example, a decrease in the growth of a snail pest, a decrease in the ability of the snail to survive, gain weight, grow, move, feed, and/or reproduce.

An effective amount of a polypeptide provided herein, when provided in the vicinity of a target pest or in the diet of a target pest, or when in contact with a target pest, exhibits pesticidal activity when the polypeptide contacts the pest. A polypeptide can be an enzyme. Pesticidal or insecticidal chemical agents can be used alone or in combinations with one or more polypeptides of the present disclosure. Chemical agents include but are not limited to dsRNA molecules targeting specific genes for suppression in a target pest, organochlorides, organophosphates, carbamates, pyrethroids, neonicotinoids, and ryanoids.

The phrases “present together” and “co-located” are intended to include any instance of which a target pest has been contacted by the polypeptide as well as any other agent also present in an effective amount relative to the target pest. “Contacted” is intended to refer to being present in the vicinity of the target pest, or the delivery of a pesticidally effective amount of the polypeptide and/or agent to the target pest through exterior contact with the pest or through ingestion by the pest.

It is intended that reference to a pest, means pests of a crop or ornamental plant, including Lepidoptera, Coleopteran, Hemipteran and Homopteran insect pests of plants, as well as nematodes, snails, and pathogenic fungi of plants.

The insect pests of the order Lepidoptera include, but are not limited to, armyworms, cutworms, loopers, and heliothines in the family Noctuidae, e.g., Fall armyworm (Spodoptera frugiperda), Beet armyworm (Spodoptera exigua), Black armyworm (Spodoptera cosmioides), Southern armyworm (Spodoptera eridania), bertha armyworm (Mamestra configurata), black cutworm (Agrotis ipsilon), cabbage looper (Trichoplusia ni), soybean looper (Pseudoplusia includens), Sugarcane borer (Diatraea saccharalis), Sunflower looper (Rachiplusia nu), velvetbean caterpillar (Anticarsia gemmatalis), green cloverworm (Hypena scabra), tobacco budworm (Heliothis virescens), granulate cutworm (Agrotis subterranea), armyworm (Pseudaletia unipuncta), Sunflower looper (Rachiplusia nu), South American podworm (Helicoverpa gelotopoeon), western cutworm (Agrotis orthogonia); borers, casebearers, webworms, coneworms, cabbageworms and skeletonizers from the Family Pyralidae, e.g., European corn borer (Ostrinia nubilalis), navel orange worm (Amyelois transitella), corn root webworm (Crambus caliginosellus), sod webworm (Herpetogramma licarsisalis), sunflower moth (Homoeosoma electellum), lesser cornstalk borer (Elasmopalpus lignosellus); leafrollers, budworms, seed worms, and fruit worms in the Family Tortricidae, e.g., codling moth (Cydia pomonella), grape berry moth (Endopiza viteana), oriental fruit moth (Grapholita molesta), sunflower bud moth (Suleima helianthana); and many other economically important Lepidoptera, e.g., diamondback moth (Plutella xylostella), pink bollworm (Pectinophora gossypiella), and gypsy moth (Lymantria dispar). Other insect pests of order Lepidoptera include, e.g., cotton leaf worm (Alabama argillacea), fruit tree leaf roller (Archips argyrospila), European leafroller (Archips rosana) and other Archips species, (Chilo suppressalis, Asiatic rice borer, or rice stem borer), rice leaf roller (Cnaphalocrocis medinalis), corn root webworm (Crambus caliginosellus), bluegrass webworm (Crambus teterrellus), southwestern corn borer (Diatraea grandiosella), surgarcane borer (Diatraea saccharalis), spiny bollworm (Earias insulana), spotted bollworm (Earias vittella), American bollworm (Helicoverpa armigera), corn carworm (Helicoverpa zea, also known as soybean pod worm and cotton bollworm), tobacco budworm (Heliothis virescens), sod webworm (Herpetogramma licarsisalis), Western bean cutworm (Striacosta albicosta), European grape vine moth (Lobesia botrana), citrus leaf miner (Phyllocnistis citrella), large white butterfly (Pieris brassicae), small white butterfly (Pieris rapae, also known as imported cabbageworm), beet armyworm (Spodoptera exigua), tobacco cutworm (Spodoptera litura, also known as cluster caterpillar), and tomato leaf miner (Tuta absoluta).

The pests may also include phytoparasitic pests from the phylum Nematoda, for example, Aglenchus spp., Anguina spp., Aphelenchoides spp., Belonolaimus spp., Bursaphelenchus spp., Cacopaurus spp., Criconemella spp., Criconemoides spp., Ditylenchus spp., Dolichodorus spp., Globodera spp., Helicotylenchus spp., Hemicriconemoides spp., Hemicycliophora spp., Heterodera spp., Hoplolaimus spp., Longidorus spp., Lygus spp., Meloidogyne spp., Meloinema spp., Nacobbus spp., Neotylenchus spp., Paralongidorus spp., Paraphelenchus spp., Paratrichodorus spp., Pratylenchus spp., Pseudohalenchus spp., Psilenchus spp., Punctodera spp., Quinisulcius spp., Radopholus spp., Rotylenchulus spp., Rotylenchus spp., Scutellonema spp., Subanguina spp., Trichodorus spp., Tylenchulus spp., Tylenchorhynchus spp., Xiphinema spp.

Reference in this application to an “isolated enzyme”, or an equivalent term or phrase, is intended to mean that the enzyme is one that is present alone or in combination with other compositions, but not within its natural environment. For example, a polypeptide or enzyme, would be “isolated” within the scope of this disclosure so long as it is produced in a space in which it is not normally found to be produced in nature, i.e., in a transgenic or recombinant cell, in a transgenic or recombinant bacterium or microorganism, or in a DNA vacant cell or minicell produced from a transgenic or recombinant bacterium or microorganism. Polypeptides and polynucleotide sequences/coding sequences can be isolated from the organism in which these are produced, i.e., any number of means known in the art, such as filtration, precipitation, capture (using various molecules which exhibit affinity specifically to the protein or nucleic acid structure), and the like, further “isolating” the molecules from constituents that create an impurity and the like. A recombinant cell (e.g. recombinant microorganism), whether plant or bacterium, by its very nature is not naturally occurring, is isolated, and so is not a product of nature, and so is patentable in every territory in the world on this basis.

The term “free enzyme” as used herein refers to an enzyme preparation that is substantially free of intact cells. The term “free enzyme” includes, but is not limited to, crude cell extracts containing an enzyme, partially purified, substantially purified, or purified enzyme.

The term “partially purified” as used herein in reference to the enzymes means that a crude preparation of the enzyme (e.g., a cell lysate) has been subjected to procedures that remove at least some non-enzyme components (e.g., waste proteins, dead cell material, excess water, and/or unwanted cell debris). In a partially purified enzyme preparation, the enzyme preferably comprises at least 1% of the total protein content in the preparation, more preferably at least 3% of the total protein content in the preparation, and even more preferably greater than 5% of the total protein content in the preparation.

The term “substantially purified” as used herein in reference to the enzymes means that the enzyme preparation has been subjected to procedures that remove a substantial amount of non-enzyme components (e.g., waste proteins, dead cell material, excess water, and/or unwanted cell debris). In a substantially purified enzyme preparation, the enzyme preferably comprises greater than 30% of the total protein content in the preparation, more preferably greater than about 40% of the total protein content in the preparation, and even more preferably greater than 50% of the total protein content in the preparation.

The term “synergistically effective amount” as used herein refers an amount of a first substance (e.g., a first enzyme) that when used in combination with a second substance (e.g., a second enzyme) that produces a biological effect that is greater than the sum of the biological effects of each of the respective first and second substances when used alone.

The term “segment” or “fragment” is used in this application to describe consecutive amino acid or nucleic acid sequences that are shorter than the complete amino acid or nucleic acid sequence describing the enzyme or an enzyme variant or the respective nucleotide sequences encoding such amino acid sequences. A segment or fragment exhibiting activity is also disclosed in this application if alignment of such segment or fragment, with the corresponding section of the polypeptide disclosed herein, results in amino acid sequence identity of any fraction percentage from about 85 to about 100 percent between the segment or fragment and the corresponding segment of amino acids within disclosed polypeptide. A fragment as described herein may comprise at least 50, at least 100, at least 250, at least 400, or at least 500, contiguous amino acid residues of a polypeptide sequence disclosed herein. Embodiments disclosed herein further include any segment or fragment of a sequence having at least 85% sequence identity to a sequence selected from SEQ ID NOs: 1-208, 247-260, and 287-328 having the described activity, e.g. a segment or fragment comprising a catalytic domain of an enzyme provided herein.

The term “nitrogen stabilizer” as used herein can comprising a nitrification inhibitor, a urease inhibitor, or a nitrogen leaching preventative agent. The nitrogen stabilizer can further comprise N (n-butyl) thiophosphoric acid triamide (NBPT), N (n-propyl) thiophosphoric acid triamide (NPPT), nitropyrin, dicyandiamide (DCD), ammonium thiosulfate (ATS), calcium heteropolysaccharide, or poly coated ureas.

One skilled in the art will recognize that, due to the redundancy of the genetic code, many sequences are capable of encoding the polypeptides disclosed herein, and those sequences, to the extent that they function to express as enzyme exhibiting insecticidal, nematicidal, or pesticidal activity either when expressed in a recombinant microorganism or in a cell-free environment, are embodiments of the present invention. Embodiments disclosed herein further include any polynucleotides encoding any of SEQ ID NOs: 1-328 or any fragment of SEQ ID NOs: 1-328 having the described activity.

As described further in this application, the present disclosure includes methods comprising applying isolated enzymes or recombinant microorganisms that express and/or overexpress enzymes to a plant growth medium, a plant, a plant seed, or an area surrounding a plant seed. The present invention is also directed to seeds treated or coated with isolated enzymes or recombinant microorganism that overexpress enzymes. The present invention is also directed to compositions and formulations comprising at least one isolated enzyme or recombinant microorganism that overexpress an enzyme. The use of isolated enzymes or recombinant microorganism that overexpress enzymes allows for bursts of insecticidal, nematicidal, or pesticidal activity, yielded by the enzymes disclosed herein.

In certain embodiments, an isolated enzyme is the active ingredient of composition prepared by culturing recombinant Bacillus or any other recombinant microorganism transformed to express the enzyme under conditions suitable to express active enzyme. Such a composition can be prepared by desiccation, lyophilization, homogenization, extraction, filtration, centrifugation, sedimentation, or concentration of a culture of such recombinant cells expressing/producing said recombinant polypeptide. Such a process can result in a Bacillus or other recombinant microorganism cell extract, cell suspension, cell homogenate, cell lysate, cell supernatant, cell filtrate, or cell pellet. By obtaining the recombinant polypeptides so produced, a composition that includes the recombinant polypeptides can be formulated for various uses, including as agricultural pest inhibitory spray products, as pest inhibitory seed coatings or as pest inhibitory formulations in diet bioassays.

### I. Esterases

The present disclosure provides esterase polypeptides. Esterases (EC 3.1) are a broad class of enzymes that catalyze the hydrolysis of an ester bond resulting in the production of an acid and an alcohol, e.g., a carboxylic acid and a primary alcohol. Examples of esterases within the broader enzyme family include, but are not limited to carboxylic ester hydrolases (EC 3.1.1; e.g. a carboxylesterase or a feruloyl esterase); thioester hydrolases (EC 3.1.2); phosphoric monoester hydrolases (EC 3.1.3); phosphoric diester hydrolases (EC 3.1.4); triphosphoric monoester hydrolases (EC 3.1.4); sulfuric ester hydrolases (EC 3.1.4); diphosphoric monoester hydrolases (EC 3.1.4); and phosphoric triester hydrolases (EC 3.1.4). The three-dimensional structure of esterase enzymes show the characteristic α/β-hydrolase fold-a definite order of α-helices and β-sheets. See, e.g. Bornscheuer, FEMS Microbiology Reviews 26 (2002) 73-81. The catalytic triad is composed of Ser-Asp-His or Ser-Glu-His and a consensus sequence (Gly-×-Ser-×-Gly) is typically found around the active site serine.

Esterases may exhibit broad substrate specificity or may be specific to a single substrate or set of substrates. These enzymes have known applications in the food industry, the paper industry, in the degradation of plastics and pesticides, and in the synthesis of optically pure compounds. However, esterases having insecticidal, nematicidal, or pesticidal activity are not believed to be known in the art. Therefore, the present disclosure provides for the first-time methods and compositions for protecting a plant from a pathogen or pest comprising an esterase. In another embodiment, the esterase may comprise a sequence having at least 85% sequence identity to a sequence identifier (SEQ ID NO) provided in the Table 1 below. In addition, the esterase can comprise an amino acid sequence having at least 70%, 75%, 80%, 90%, 95%, 96%, 97% 98%, or 99% sequence identity to any one of SEQ ID NOs: 83-100, 248, 252-255, and 287-289 and exhibiting insecticidal, nematicidal, or pesticidal activity.

TABLE 1
Exemplary Polypeptide Sequences for Esterases

| | SEQ |
|---|---|
| Enzyme | ID NO: |
| Bacillus cereus SGNH/GDSL Esterase | 83 |
| Bacillus sp. Carboxylesterase | 84 |
| Priestia megaterium GDSL-type Esterase | 85 |
| Priestia megaterium GDSL-type Esterase | 86 |
| Cytobacillus firmus GDSL-type Esterase | 87 |
| Bacillus subtilis Esterase (EstB) | 88 |
| Bacillus subtilis Esterase (EstB without signal sequence) | 89 |
| Beauveria bassiana Feruloyl Esterase A | 90 |
| Beauveria bassiana Feruloyl Esterase A2 | 91 |
| Rhizomucor miehei Esterase | 92 |
| Rhizomucor miehei Esterase | 93 |
| Bacillus subtilis Esterase (pnbA) | 94 |
| Sphingobium wenxiniae Esterase (pytH) | 95 |
| Methylobacterium sp. Esterase (msE1) | 96 |
| Bacillus pumilus Esterase_2_aa | 97 |
| Bacillus pumilus Esterase_3_aa | 98 |
| Pseudomonas fluorescens Esterase | 99 |
| Pseudomonas fluorescens Esterase A | 100 |
| Pseudomonas sp. Esterase | 248 |
| Geobacillus stearothermophilus (a.k.a B. Stearothermophilus) | 252 |
| Esterase | |
| Rhizopus oryzae Esterase | 253 |
| Bacillus subtilis EstA Esterase | 254 |
| Sphingobium wenxiniae (PytH) Esterase | 255 |
| Beauveria bassiana Acetyl esterase | 287 |
| Pseudomonas sp Esterase 2 | 288 |
| Purpureocillium lilacinum Esterase B | 289 |

### II. Chitinases

The present disclosure provides chitinase polypeptides. Chitinases (E.C 3.2.2.14) are enzymes that can hydrolytically cleave β-1,4-glycosidic bonds between individual N-acetylglucosamine moieties in the backbone of chitin molecules. Chitin is an unbranched structural polysaccharide consisting of β-1,4-glycosidic linked N-acetylglucosamine moieties. Chitin is the second most abundant polysaccharide in nature after cellulose, is found in the exoskeleton of insects, fungi, yeast, and algae, and in the internal structures of other vertebrates.

Chitinases are a diverse group of enzymes that show differences in their molecular structure, substrate specificity, and catalytic mechanism. Chitinases may be divided into two main groups designated as endochitinases (E.C 3.2.1.14) and exo-chitinases. The endochitinases randomly split chitin at internal sites, thereby forming the dimer di-acetylchitobiose and soluble low molecular mass multimers of GlcNAc such as chitotriose, and chitotetraose. The exo-chitinases may be further divided into two subcategories designated as chitobiosidases (E.C. 3.2.1.29), which are involved in catalyzing the progressive release of di-acetylchitobiose starting at the non-reducing end of the chitin microfibril, and 1-4-β-glucosaminidases (E.C. 3.2.1.30), which cleave the oligomeric products of endochitinases and chitobiosidases, thereby generating monomers of GlcNAc.

Chitinases may also be classified in two glycoside hydrolase families, GH18 and GH19, with different structures and catalytic mechanisms. Family GH18 includes the chitinases from viruses, bacteria, fungi and animals as well as classes III and V from plants; and GH19 chitinases are identified mostly in plants (classes I, II and IV), nematodes, and some bacteria. For example, Chi19F (SEQ ID NO: 182) comprises a GH19 domain (amino acid residues 65-266) and a carbohydrate binding domain (amino acid residues 8-50). ChiC (SEQ ID NO: 249) is comprises a GH18 chitinase domain (amino acid residues 9-418) and a carbohydrate binding domain (amino acid residues 547-630).

Chitinases have wide-ranging applications including the preparation of pharmaceutically important chitooligosaccharides and N-acetyl D glucosamine, preparation of single-cell protein, isolation of protoplasts from fungi and yeast, treatment of chitinous waste, mosquito control and morphogenesis. The present disclosure provides chitinases having insecticidal, nematicidal, or pesticidal activity. In particular, the present disclosure provides methods and compositions for protecting a plant from a pathogen or pest comprising a chitinase described herein. In certain embodiments, the chitinase may comprise a sequence having at least 85% sequence identity to a sequence identifier (SEQ ID NO) provided in the Table 2 below. In addition, the chitinase can comprise an amino acid sequence having at least 70%, 75%, 80%, 90%, 95%, 96%, 97% 98%, or 99% sequence identity to any one of SEQ ID NOs: 179-205, and 249 and exhibiting insecticidal, nematicidal, or pesticidal activity.

TABLE 2
Exemplary Polypeptide Sequences for Chitinases

| | SEQ |
|---|---|
| Enzyme | ID NO: |
| Serratia marcescens Chitinase (SmChiA) | 179 |
| Photorhabdus luminescens Chitinase Q84F70-PhoLum-MetP+ | 180 |
| Aspergillus niger Chitinase-AspNig | 181 |
| Streptomyces coelicolor Chitinase F (Chi19F) | 182 |
| Lecanicillium psalliotae Chitinase 1 (Lpchi1) | 183 |
| Lecanicillium lecanii Acidic Chitinase 1 (Chit1) | 184 |
| Akanthomyces lecanii Acidic Chitinase 1 (Chit1) | 185 |
| Helicoverpa zea Chitinase_Chi10a | 186 |
| Spodoptera frugiperda Chitinase_Chi10a | 187 |
| Plutella xylostella Chitinase_Chi10a | 188 |
| Manduca sexta Chitinase_Chi10a | 189 |
| Spodoptera frugiperda Chitinase_Chi10b | 190 |
| Plutella xylostella Chitinase_Chi10b | 191 |
| Manduca sexta Chitinase_Chi10b | 192 |
| Bombyx mori Chitinase_Chi10b | 193 |
| Spodoptera exigua Chitinase_Chi10b | 194 |
| Helicoverpa zea Chitinase_Chi2a | 195 |
| Spodoptera frugiperda Chitinase_Chi2a | 196 |
| Manduca sexta Chitinase_Chi2a | 197 |
| Plutella xylostella Chitinase_Chi2a | 198 |
| Plutella xylostella Chitinase_Chi2a | 199 |
| Plutella xylostella Chitinase_Chi2b | 200 |
| Spodoptera frugiperda Chitinase_Chi2b | 201 |
| Manduca sexta Chitinase_Chi2b | 202 |
| Helicoverpa zea Chitinase_Chi2b | 203 |
| Bombyx mori Chitinase_Chi2b | 204 |
| Spodoptera exigua Chitinase Chi2b | 205 |
| Chitinase C | 249 |

### III. Proteases

The present disclosure also provides protease polypeptides. Proteases, also known as proteinases or proteolytic enzymes, are a large group of enzymes that catalyze the hydrolysis of peptide bonds in proteins and polypeptides. Proteases differ with respect to substrate specificity, active site and catalytic mechanism, pH and temperature optima, and stability profile. In general, proteases can be broadly divided into seven classifications including, serine proteases, cysteine proteases, threonine proteases, aspartic proteases, glutamic proteases, metalloproteases, and asparagine peptide lyases. The present disclosure provides proteases having insecticidal, nematicidal, or pesticidal activity. In particular, the present disclosure provides methods and compositions for protecting a plant from a pathogen or pest comprising a protease described herein. In certain embodiments, the protease may comprise a sequence having at least 85% sequence identity to a sequence identifier (SEQ ID NO) provided in the Table 3 below. In addition, the protease can comprise an amino acid sequence having at least 70%, 75%, 80%, 90%, 95%, 96%, 97% 98%, or 99% sequence identity to any one of SEQ ID NOs: 122-178, 247, and 310-327 and exhibiting insecticidal, nematicidal, or pesticidal activity.

TABLE 3
Exemplary Polypeptide Sequences for Proteases

| Enzyme | SEQ ID NO: |
|---|---|
| Bacillus thuringiensis e0Collag | 122 |
| Orbilia oigospora Aoz1-Cutinase | 123 |
| Trichoderma koningiopsis prb-Trich | 124 |
| Trichoderma lixii QYS92649-Trich | 125 |
| Trichoderma lixii trypsin-like protease | 126 |
| Bacillus sp. B16 neutral protease | 127 |
| Hirsutella rhossiliensis protease | 128 |
| Bacillus sp. B16 protease apr (Alkaline serine | 129 |
| protease) | |
| Bacillus subtilis protease NprE | 130 |
| Bacillus mycoides protease | 131 |
| Bacillus tequilensis protease NprE | 132 |
| Bacillus subtilis protease NprB | 133 |
| Bacillus thuringiensis protease | 134 |
| Geobacillus sp. protease | 135 |
| Bacillus sp. Thermolysin | 136 |
| Geobacillus thermoleovorans protease | 137 |
| Bacillus paralicheniformis Protease_Mpr | 138 |
| Bacillus sonorensis Protease_Mpr | 139 |
| Bacillus amyloliquefaciens Protease 1 | 140 |
| Bacillus velezensis Protease 1 | 141 |
| Bacillus mojavensis Protease | 142 |
| Bacillus halotolerans Protease | 143 |
| Bacillus subtilis Protease_Mpr | 144 |
| Bacillus tequilensis Protease | 145 |
| Bacillus mycoides Protease_Vpr | 146 |
| Gottfriedia luciferensis Protease | 147 |
| Bacillus amyloliquefaciens Protease Vpr-like | 148 |
| Bacillus velezensis Protease Vpr | 149 |
| Bacillus tequilensis Protease Vpr | 150 |
| Gottfriedia luciferensis Protease_Vpr-like | 151 |
| Gottfriedia acidiceleris Protease_Vpr-like | 152 |
| Bacillus safensis Protease_AprE | 153 |
| Bacillus subtilis Protease_AprE | 154 |
| Bacillus subtilis Protease_AprE 2 | 155 |
| Bacillus licheniformis Protease_keratinase | 156 |
| Bacillus glycinifermentans Protease | 157 |
| Bacillus subtilis Protease Epr | 158 |
| Bacillus siamensis Protease | 159 |
| Bacillus velezensis Protease 2 | 160 |
| Bacillus amyloliquefaciens Protease 2 | 161 |
| Bacillus velezensis Protease 3 | 162 |
| Bacillus paralicheniformis Protease 1 | 163 |
| Bacillus sonorensis Protease 1 | 164 |
| Bacillus sonorensis Protease 2 | 165 |
| Bacillus licheniformis Protease | 166 |
| Bacillus paralicheniformis Protease 2 | 167 |
| Bacillus atrophaeus Protease | 168 |
| Bacillus amyloliquefaciens Protease 3 | 169 |
| Bacillus velezensis Protease 4 | 170 |
| Bacillus subtilis Protease | 171 |
| Gottfriedia luciferensis Protease_inhA | 172 |
| Gottfriedia luciferensis Protease_inhA 2 | 173 |
| Bacillus mycoides Protease_inhA | 174 |
| Bacillus salacetis Protease_inhA | 175 |
| Bacillus haikouensis Protease_inhA | 176 |
| Bacillus thuringiensis Protease_inhA | 177 |
| Photorhabdus luminescens Protease_Serralysin | 178 |
| Bacillus subtilis SP2 catalytic domain | 247 |
| Bacillus sp Protease_NprB 2 | 310 |
| Bacillus subtilis Protease_V8-like | 311 |
| Bacillus subtilis Protease_Apr 2 | 312 |
| Bacillus subtilis Protease_Apr 3 | 313 |
| Bacillus thuringiensis Protease 2 | 314 |
| Bacillus thuringiensis Protease_inhA 2 | 315 |
| Bacillus thuringiensis Protease_ColA | 316 |
| Bacillus velezensis Protease_Peptidase | 317 |
| Bacillus velezensis Protease_Apr | 318 |
| Metarhizium robertsii Protease_Apr | 319 |
| Mythimna unipuncta granulovirus | 320 |
| Protease_inhA | |
| Paenibacillus larvae Protease_inhA | 321 |
| Serratia marcescens Protease_inhA | 322 |
| Serratia sp Protease_Serralysin | 323 |
| Trichoderma atroviride Protease_Apr | 324 |
| Trichoplusia ni granulovirus Protease_inhA 1 | 325 |
| Trichoplusia ni granulovirus Protease_inhA 2 | 326 |
| Trichoplusia ni granulovirus Protease_inhA 3 | 327 |

### IV. Collagenases

In some embodiments, the present disclosure also provides collagenase polypeptides. Collagenases are a specific class of proteases that catalyze the hydrolysis of the peptide bonds in collagen. Specifically, collagenases possess the unique ability to degrade native collagen which is otherwise resistant to breakdown by other known proteases. The present disclosure provides collagenases having insecticidal, nematicidal, or pesticidal activity. In particular, the present disclosure provides methods and compositions for protecting a plant from a pathogen or pest comprising a collagenase described herein. In certain embodiments, the collagenase may comprise a sequence having at least 85% sequence identity to a sequence identifier (SEQ ID NO) provided in the Table 4 below. In addition, the collagenase can comprise an amino acid sequence having at least 70%, 75%, 80%, 90%, 95%, 96%, 97% 98%, or 99% sequence identity to any one of SEQ ID NOs: 206-208, and 328 and exhibiting insecticidal, nematicidal, or pesticidal activity.

TABLE 4
Amino acid sequences for Collagenases

| Enzyme | SEQ ID NO: |
|---|---|
| Bacillus thuringiensis e0Collag | 122 |
| Clostridium histolyticum Collagenase A | 206 |
| (ColA) | |
| Bacillus pumulisCollagenaseB (ColB) | 207 |
| Bacillus pumulis Collagenase B (ColB) | 208 |
| Clostridium histolyticum Collagenase A | 328 |
| (ColA) | |

### V. Lipases

The present disclosure provides lipase polypeptides. Lipase polypeptides catalyze the hydrolysis of fats. Some lipases display broad substrate scope including esters of cholesterol, phospholipids, and of lipid-soluble vitamins and sphingomyelinases. Unlike esterases, which function in water, lipases are usually activated only when adsorbed to an oil-water interface. Lipases perform essential roles in digestion, transport and processing of dietary lipids in most, if not all, organisms. The lipases exhibit insecticidal, nematicidal, or pesticidal activity. In particular, the present disclosure provides methods and compositions for protecting a plant from a pathogen or pest comprising a lipase described herein. In certain embodiments, the lipase may comprise a sequence having at least 85% sequence identity to a sequence identifier (SEQ ID NO) provided in the Table 5 below. In addition, the lipase can comprise an amino acid sequence having at least 70%, 75%, 80%, 90%, 95%, 96%, 97% 98%, or 99% sequence identity to any one of SEQ ID NOs: 1-82, 250, 251, 256-260, and 290-307 and exhibiting insecticidal, nematicidal, or pesticidal activity. Similar to esterases, the three-dimensional structure of lipase enzymes show the characteristic α/β-hydrolase fold. See, e.g. Bornscheuer, FEMS Microbiology Reviews 26 (2002) 73-81. The catalytic triad is composed of Ser-Asp-His or Ser-Glu-His and a consensus sequence (Gly-×-Ser-×-Gly) is typically found around the active site serine.

TABLE 5
Exemplary Polypeptide Sequences for Lipases

| Enzyme | SEQ ID NO: |
|---|---|
| Bacillus cereus Lipase | 1 |
| Bacillus sp. Lipase | 2 |
| Bacillus sp. SGNH/GDSL Hydrolase /Lipase | 3 |
| Bacillus sp. Alpha/Beta Hydrolase/Lipase | 4 |
| Bacillus sp. Triacylglycerol Lipase | 5 |
| Priestia megaterium Lipase | 6 |
| Priestia megaterium Triacylglycerol Lipase | 7 |
| Cytobacillus firmus Lipase 1 | 8 |
| Cytobacillus firmus Lipase 2 | 9 |
| Bacillus subtilis Lipase (LipC) | 10 |
| Bacillus cereus Lipase (Lip 1) | 11 |
| Bacillus licheniformis Lipase | 12 |
| Beauveria bassiana Triacylglycerol Lipase | 13 |
| Beauveria bassiana Lipase 1 | 14 |
| Beauveria bassiana Lipase 2 | 15 |
| Beauveria bassiana Lipase 3 | 16 |
| Beauveria bassiana Lipase 4 | 17 |
| Beauveria bassiana Lipase-like 1 | 18 |
| Beauveria bassiana Lipase-like 2 | 19 |
| Beauveria bassiana Lipase-like 3 | 20 |
| Beauveria bassiana Lipase 5 | 21 |
| Beauveria bassiana Lipase class 2a | 22 |
| Beauveria bassiana Lipase 6 | 23 |
| Beauveria bassiana Triacylglycerol Lipase | 24 |
| Beauveria bassiana Lipase class 2b | 25 |
| Beauveria bassiana Lipase 7 | 26 |
| Beauveria bassiana Lipase 8 | 27 |
| Beauveria bassiana Lipase 9 | 28 |
| Metarhizium anisopliae Lipase 1 | 29 |
| Metarhizium anisopliae Lipase 2 | 30 |
| Metarhizium anisopliae Lipase 3 | 31 |
| Burkholderia cepacia Triacylglycerol Lipase 1 | 32 |
| Pseudomonas sp. Lipase 1 | 33 |
| Pseudomonas sp. Lipase 2 | 34 |
| Pseudomonas fluorescens Lipase 1 | 35 |
| Pseudomonas fluorescens Lipase 2 | 36 |
| Pseudomonas fluorescens Lipase 3 | 37 |
| Pseudomonas fluorescens Lipase 4 | 38 |
| Pseudomonas sp. Triacylglycerol lipase 1 | 39 |
| Pseudomonas fluorescens Lipase 5 | 40 |
| Pseudomonas fluorescens Triacylglycerol Lipase 1 | 41 |
| Pseudomonas fluorescens Lipase class 3 | 42 |
| Pseudomonas fluorescens Lipase 6 | 43 |
| Pseudomonas fluorescens Monoacylglycerol Lipase | 44 |
| Pseudomonas fluorescens Lipase 7 | 45 |
| Pseudomonas fluorescens Lipase 8 | 46 |
| Pseudomonas fluorescens Lipase 9 | 47 |
| Pseudomonas fluorescens Lipase 10 | 48 |
| Pseudomonas fluorescens Triacylglycerol Lipase 2 | 49 |
| Pseudomonas fluorescens Lipase 11 | 50 |
| Pseudomonas fluorescens Lipase 12 | 51 |
| Burkholderia cepacia Triacylglycerol Lipase 2 | 52 |
| Burkholderia cepacia Lipase 1 | 53 |
| Burkholderia cepacia Lipase 2 | 54 |
| Burkholderia cepacia Lipase 3 | 55 |
| Burkholderia cepacia Lipase 4 | 56 |
| Burkholderia cepacia Lipase 5 | 57 |
| Burkholderia cepacia Lipase 6 | 58 |
| Burkholderia cepacia Triacylglycerol Lipase 3 | 59 |
| Burkholderia cepacia Triacylglycerol Lipase 4 | 60 |
| Burkholderia cepacia Lipase 7 | 61 |
| Burkholderia cepacia Lipase 8 | 62 |
| Burkholderia cepacia Lipase 9 | 63 |
| Burkholderia cepacia Triacylglycerol Lipase 5 | 64 |
| Burkholderia territorii Lipase | 65 |
| Paraburkholderia acidiphila Lipase | 66 |
| Paraburkholderia atlantica Lipase 1 | 67 |
| Paraburkholderia atlantica Lipase 2 | 68 |
| Paraburkholderia atlantica Lipase 3 | 69 |
| Burkholderia plantarii Triacylglycerol lipase | 70 |
| Pseudomonas sp. Triacylglycerol Lipase 2 | 71 |
| Beauveria bassiana Lipase 10 | 72 |
| Pseudomonas sp. Lipase LipC | 73 |
| Pseudomonas fragi Lipase-like | 74 |
| Serratia marcescens Lipase | 75 |
| Bacillus thuringiensis Lipase (Lip1) | 76 |
| Bacillus tropicus Lipase (Lip2) | 77 |
| Rhizomucor miehei Lipase 1 | 78 |
| Rhizomucor miehei Triacylglycerol Lipase 2 | 79 |
| Rhizomucor miehei Triacylglycerol Lipase 1 | 80 |
| Rhizomucor miehei Lipase 2 | 81 |
| Rhizomucor miehei Triacylglycerol Lipase 3 | 82 |
| Bacillus thuringiensis Lipase 1 | 250 |
| Bacillus thuringiensis Lipase 2 | 251 |
| Burkholderia sp. Bp8992 Triacylglycerol lipase | 256 |
| Burkholderia cepacian Triacylglycerol lipase | 257 |
| Burkholderia sp. Triacylglycerol lipase | 258 |
| Burkholderia ambifaria Triacylglycerol lipase | 259 |
| Burkholderia oklahomensis Triacylglycerol lipase | 260 |
| Archaeoglobus fulgidus Triacylglycerol Lipase | 290 |
| Bacillus paralicheniformis Triacylglycerol Lipase | 291 |
| Bacillus sp Lipase 2 | 292 |
| Bacillus thuringiensis Lipase 3 | 293 |
| Beauveria bassiana Lipase 11 | 294 |
| Beauveria bassiana Triacylglycerol Lipase 3 | 295 |
| Beauveria bassiana Triacylglycerol Lipase 4 | 296 |
| Beauveria bassiana Triacylglycerol Lipase 5 | 297 |
| Beauveria bassiana Lipase 12 | 298 |
| Beauveria bassiana Lipase 13 | 299 |
| Beauveria bassiana Lipase 14 | 300 |
| Neobacillus niacin GDSL-type Lipase | 301 |
| Neobacillus niacin Triacylglycerol Lipase | 302 |
| Pseudomonas aeruginosa Triacylglycerol Lipase | 303 |
| Pseudomonas sp Triacylglycerol Lipase 3 | 304 |
| Pseudomonas sp Triacylglycerol Lipase 4 | 305 |
| Pseudomonas sp Triacylglycerol Lipase 5 | 306 |
| Purpureocillium lilacinum Lipase | 307 |

### VI. Polyurethanases

The present disclosure also provides polyurethanases polypeptides. Polyurethanases polypeptides are enzymes that degrade polyurethanes and may include urethan bond hydrolysis. In specific embodiments, a lipase may be further described as having polyurethane activity. Current research studies regarding polyurethanases are primarily focused on their use in bioremediation and recycling of polyurethanes to reduce environmental pollution caused by these materials. Polyurethanases exhibiting insecticidal, nematicidal, or pesticidal activity are not believed to be known in the art. The present disclosure provides for the first-time methods and compositions for protecting a plant from a pathogen or pest comprising a polyurethanase described herein. In certain embodiments, the polyurethanase may comprise a sequence having at least 85% sequence identity to a sequence identifier (SEQ ID NO) provided in the Table 6 below. In addition, the polyurethanase can comprise an amino acid sequence having at least 70%, 75%, 80%, 90%, 95%, 96%, 97% 98%, or 99% sequence identity to any one of SEQ ID NOs: 101-121, 308, and 309 and exhibiting insecticidal, nematicidal, or pesticidal activity.

TABLE 6
Exemplary Polypeptide Sequences for Polyurethanases

| Enzyme | SEQ ID NO: |
|---|---|
| Pseudomonas atagonensis Polyurethanase | 101 |
| Pseudomonas fluorescens Polyurethanase 1 | 102 |
| Pseudomonas chlororaphis Polyurethanase 1 | 103 |
| Serratia marcescens Polyurethanase 1 | 104 |
| Serratia liquefaciens Polyurethanase 1 | 105 |
| Serratia liquefaciens Polyurethanase 2 | 106 |
| Serratia marcescens Polyurethanase 2 | 107 |
| Serratia liuefaciens Polyurethanase | 108 |
| Pseudomonas chlororaphis Polyurethane 2 | 109 |
| Pseudomonas protegens Polyurethanase 1 | 110 |
| Pseudomonas fluorescens Polyurethanase 2 | 111 |
| Pseudomonas atagonenis Polyurethanase | 112 |
| Pseudomonas fluorescens Polyurethanase 3 | 113 |
| Pseudomonas fluorescens Polyurethanase 4 | 114 |
| Serratia marcescens Polyurethanase 3 | 115 |
| Serratia liquefaciens Polyurethanase 3 | 116 |
| Serratia liquefaciens Polyurethanase 4 | 117 |
| Serratia marcescens Polyurethanase 4 | 118 |
| Serratia marcescens Polyurethanase 5 | 119 |
| Pseudomonas protegens Polyurethanase 2 | 120 |
| Pseudomonas fluorescens Polyurethanase 5 | 121 |
| Pseudomonas chlororaphis Polyurethanase 3 | 308 |
| Serratia marcescens Polyurethanase 6 | 309 |

### VII. Recombinant Microorganisms

The present disclosure provides novel recombinant microorganisms, e.g. a recombinant microorganism expressing an enzyme, wherein the enzyme comprises a sequence having at least 85% sequence identity to a sequence selected from SEQ ID NOs: 1-208, 247-260, and 287-328 described herein. In still further embodiments, the enzyme is expressed during vegetative growth of the recombinant microorganism; further comprises a signal peptide that results in secretion of the enzyme; or is not otherwise bound to the recombinant microorganism or fragment thereof. In any of the recombinant microorganisms that express an enzyme described herein, the enzyme may be expressed under the control of a heterologous promoter. In some embodiments, the promoter can be a constitutive promoter or an inducible promoter. For any of the recombinant microorganisms described herein, the recombinant microorganism can comprise a bacterium of the genus Bacillus, a bacterium of the genus Paenibacillus, a bacterium of the genus Lysinibacillus, a fungus of the genus Penicillium, a bacterium of the genus Glomus, a bacterium of the genus Pseudomonas, a bacterium of the genus Arthrobacter, a bacterium of the genus Paracoccus, a bacterium of the genus Rhizobium, a bacterium of the genus Bradyrhizobium, a bacterium of the genus Azosprillium, a bacterium of the genus Enterobacter, a bacterium of the genus Escherichia, or a combination of any thereof. In some embodiments, the recombinant microorganism comprises a recombinant spore-forming microorganism, the recombinant spore-forming microorganism can comprise a bacterium of the genus Bacillus, a bacterium of the genus Paenibacillus, a bacterium of the genus Lysinibacillus, a fungus of the genus Penicillium, a fungus of the genus Glomus, or a combination of any thereof. In specific embodiments, the recombinant microorganism can comprise Bacillus mycoides, Bacillus pseudomycoides, Bacillus cereus, Bacillus thuringiensis, Bacillus megaterium, Bacillus subtilis, Bacillus aryabbattai, Bacillus amyloliquefaciens, Bacillus circulans, Bacillus flexus, Bacillus nealsonii, Bacillus pumulis, Lysinibacillus macroides, Lysinibacillus sphericus, Lysinibacillus fusiformis, or a combination of any thereof.

In some embodiments, the recombinant microorganism described herein, may further express a chaperone protein, e.g. a chaperon protein can comprise a sequence having at least 85% sequence identity to a sequence selected from SEQ ID NOs: 261-266 described herein. Protein chaperones (also known as molecular chaperone, modulator, activator, helper protein) may assist with proper protein folding in vivo. Chaperones function by preventing off-pathway reactions, such as aggregation, and aid newly synthesized proteins to fold correctly. In particular, the present disclosure provides polypeptides that function as lipase chaperone proteins that assist in folding the lipase into an active conformation as well as secreting the lipase. The present disclosure thus provides recombinant microorganisms expressing a lipase amino acid sequence disclosed herein; and expressing a chaperone protein. For example, a chaperon protein comprising a sequence having at least at least 85%, at least 90%, at least 95%, at least 98%, at least 99%, or 100% sequence identity to a sequence selected from SEQ ID NOs: 261-266. For ease of reference, descriptions of amino acid sequences for illustrative chaperone proteins that can be expressed to facilitate proper folding and secretion of the lipase enzyme from a recombinant microorganism in which the lipase is expressed are provided below in Table 7. Additionally, these sequences can also be useful for secretion of esterases, e.g. SEQ ID NOs: 83-100, 248, 252-255, and 287-289. In some embodiments, the present disclosure therefore provides a recombinant microorganism that expresses an enzyme and a chaperone protein, wherein expression of the enzyme and/or chaperone protein is increased as compared to the expression level of the enzyme and/or chaperone protein in a wild-type microorganism of the same kind under the same conditions.

TABLE 7
Exemplary Polypeptide Sequences for Chaperone Proteins

| Chaperone | SEQ ID NO: |
|---|---|
| Burkholderia sp. Lipase secretion chaperone | 261 |
| Burkholderia cepacian Lipase secretion chaperone | 262 |
| Burkholderia cepacian Lipase secretion chaperone | 263 |
| Burkholderia sp. Lipase secretion chaperone | 264 |
| Burkholderia ambifaria Lipase secretion chaperone | 265 |
| Burkholderia oklahomensis Lipase secretion chaperone | 266 |

In some embodiments, the recombinant microorganism may comprise a plant-growth promoting strain of bacteria, an endophytic strain of bacteria, or a strain of bacteria that is both plant-growth promoting and endophytic. An endophytic microorganism can be used for expression of the enzymes. While many microorganisms of the rhizosphere have a symbiotic relationship with the plant, only a small subset of these microorganisms are capable of being internalized into the plant and growing endophytically. Several Bacillus cereus family member strains and several non-Bacillus cereus family member bacterial strains have been isolated from corn seedlings and found to have the ability to grow endophytically in plants. Other endophytic microorganisms would also be useful including, but not limited to, bacterial endophytes from genera: Cellulomonas, Clavibacter, Curtobacterium, Pseudomonas, Paenibacilllus, Enterobacter, Bacillus, Klebsiella, Arthrobacter, Lysinibacillus, Pantoea, Actinomyces, Streptomyces, Alcaligenes, and Microbacterium. Fungal endophytes can also be used, including fungal endophytes from the genera: Neotyphodium, Gliocadium, Acremonium lolii, Clavicipitaceae, Ascomycetes, Idriella, Xylariaceous, Ascomycotina, Deuteromycotina, Aspergillus, Phomopsis, Wardomyces, Fusarium, Dreschrella, Pestalotia, Curvularia, Humicola, Nodulisporium, and Penicillium.

Many microorganisms can colonize, live next to, live on, or become endophytic to a plant. These microorganisms would provide a useful delivery mechanism of target enzymes to the plant, the seed, the vicinity of the plant, or the plant growth medium. Microorganisms selected that can colonize the roots or become endophytic can be screened, recombinantly modified to express or overexpress an enzyme, and produced commercially and applied on the seed, to the plant, or the vicinity around the plant in order to have the strain produce the target enzymes in situ (at or near the plant). These microorganisms can also be enhanced through point mutations or through genetic engineering to express higher or target enzymes to benefit the plants. Point mutations can be screened by mutating the host microorganism and selecting for mutants with higher enzyme expression levels through enzyme assays or using selective media that identifies high enzyme expressing strains. Common strains that are beneficial producers of enzymes as well as colonizers/endophytic species include: Bacillus argri, Bacillus aizawai, Bacillus albolactis, Bacillus amyloliquefaciens, Bacillus cereus, Bacillus coagulans, Bacillus endoparasiticus, Bacillus endorhythmos, Bacillus kurstaki, Bacillus lacticola, Bacillus lactimorbus, Bacillus firmus, Bacillus lactis, Bacillus laterosporus, Bacillus lentimorbus, Bacillus licheniformis, Bacillus megaterium, Bacillus medusa, Bacillus metiens, Bacillus natto, Bacillus nigrificans, Bacillus popillae, Bacillus pumilus, Bacillus siamensis, Bacillus sphearicus, Bacillus subtilis, Bacillus thuringiensis, Bacillus unifagellatu, other Bacillus species or a combination thereof plus those listed in the category of Bacillus Genus in Bergey's Manual of Systematic Bacteriology, First Ed. (1986), hereby incorporated in full by reference. Other potential strains could include, but are not limited to: Cellulomonas, Clavibacter, Curtobacterium, Pseudomonas, Paenibacilllus, Enterobacter, Bacillus, Klebsiella, Arthrobacter, Lysinibacillus, Pantoea, Actinomyces, Saccharomyces, Rhizobium, Bradyrhizobium, Candida, Streptomyces, Alcaligenes, Chromatiales, Rhizobium, Bradyrhizobium, Rhodospiralles, Rhizobiales, Rhizobacteracae, and Microbacterium.

In specific embodiments, the recombinant microorganisms described herein may be inactivated. Inactivation results in microorganisms that are unable to reproduce. Inactivation of microorganisms can be advantageous, for example because it allows for delivery of the microorganism to a plant or a plant growth medium while reducing or eliminating any detrimental effects that the live microorganism may have on a plant or on the environment. The recombinant microorganism can be inactivated by any physical or chemical means, e.g., by heat treatment, gamma irradiation, x-ray irradiation, UV-A irradiation, UV-B irradiation, or treatment with a solvent such as gluteraldehyde, formaldehyde, hydrogen peroxide, acetic acid, bleach, chloroform, or phenol, or combination of any thereof.

The recombinant microorganisms of the present disclosure, capable of expressing at least one polypeptide described herein, may be produced using standard molecular biology methods known in the art. For example, a gene encoding an enzyme can be amplified by polymerase chain reaction (PCR). Where a signal sequence is used, the gene coding for the enzyme can be ligated in an operable manner to DNA coding for the signal sequence. The gene can then be cloned into any suitable vector, for example a plasmid vector. The vector suitably comprises a multiple cloning site into which the DNA molecule encoding the polypeptide can be easily inserted. The vector also suitably contains a selectable marker, such as an antibiotic resistance gene, such that bacteria transformed, transfected, or mated with the vector can be readily identified and isolated. Where the vector is a plasmid, the plasmid suitably also comprises an origin of replication. Alternatively, DNA coding for the enzyme can be integrated into the chromosomal DNA of the microorganism host.

### VIII. Signal Peptides

Any signal peptide can be used to modify any of the polypeptides described herein such that the enzyme will be secreted from a host microorganism in which it is expressed. The type of signal peptide used will depend primarily on the identity of the host microorganism, since the secretion machinery of different microorganisms will vary in their ability to recognize specific signal peptides. Illustrative signal peptide sequences are provided below in Table 8, together with the bacterial species in which the signal peptides are found in nature. The signal peptides will result in secretion of a protein to which they are linked in the genus of bacteria in which they are found as well as closely related genera. For example, a signal sequence from Bacillus thuringiensis will cause secretion of a protein in bacteria of the genus Bacillus, as well as bacteria of the genera Paenibacillus and Lysinibacillus. For ease of reference, descriptions of amino acid sequences for illustrative signal peptides that can be added to any of the polypeptides described herein to facilitate secretion of the polypeptide from a microorganism in which it is expressed are provided below in Table 8. Any of the signal peptides listed in Table 8 below can be added or operably linked at the amino or carboxy terminus of any of the polypeptides described herein to cause secretion of the polypeptide. Thus, provided herein is a polypeptide sequence or recombinant polypeptide sequence comprising a signal peptide sequence (e.g. SEQ ID NO:209) operably linked to an esterase (e.g. SEQ ID NO:83), a chitinase (e.g. SEQ ID NO:179), a protease (e.g. SEQ ID NO: 122), a lipase (e.g. SEQ ID NO:82), a polyurethanase (e.g. SEQ ID NO:101), a collagenase sequence (e.g. SEQ ID NO:328), or a variant thereof.

TABLE 8
Exemplary sequences for signal peptides

| Source Species for Signal Peptide | SEQ ID NO: |
|---|---|
| Bacillus thuringiensis | 209 |
| Bacillus thuringiensis serovar israelensis 4Q7 | 210 |
| Bacillus cereus ATCC 10987 | 211 |
| Clostridium perfingens | 212 |
| Streptomyces chromofuscus | 213 |
| Bacillus subtilis subsp. subtilis str. 168 | 214 |
| Caldicellulosiruptor saccharolyticus | 215 |
| Bacillus subtilis subsp. subtilis str. 168 | 216 |
| Bacillus subtilis subsp. subtilis str. 168 | 217 |
| Geobacillus stearothermophilus (Bacillus | 218 |
| stearothermophilus) | |
| Bacillus subtilis subsp. subtilis str. 168 | 219 |
| Bacillus subtilis subsp. subtilis str. 168 | 220 |
| Bacillus subtilis subsp. subtilis str. 168 | 221 |
| Bacillus circulans | 222 |
| Bacillus circulans | 223 |
| Bacillus subtilis subsp. subtilis str. 168 | 224 |
| Bacillus subtilis subsp. subtilis str. 168 | 225 |
| Bacillus subtilis subsp. subtilis str. 168 | 226 |
| Bacillus subtilis subsp. subtilis str. 168 | 227 |
| Bacillus subtilis subsp. subtilis str. 168 | 228 |
| Bacillus thuringiensis | 229 |
| Bacillus thuringiensis | 230 |
| Bacillus thuringiensis | 231 |
| Bacillus pseudomycoides | 232 |
| Bacillus thuringiensis serovar israelensis 4Q7 | 233 |
| Bacillus cereus | 234 |
| Burkholderia cepacia | 235 |
| Pseudomonas fluorescens | 236 |
| Streptomyces species N174 | 237 |
| Paenibacillus species | 238 |
| Aspergillus saitoi | 239 |
| Bacillus sp. | 240 |
| Aspergillus japonicus | 241 |
| Triticum aestivum | 242 |
| Triticum aestivum | 243 |
| Triticum aestivum | 244 |
| Triticum aestivum | 245 |
| Bacillus amyloliquefaciens | 246 |
| Bacillus subtilis | 267 |
| Bacillus subtilis | 268 |
| Bacillus thuringiensis | 269 |
| Cellulosimicrobium cellulans | 270 |
| Bacillus subtilis | 271 |
| Klebsiella aerogenes | 272 |
| Bacillus subtilis | 273 |
| Bacillus thuringiensis | 274 |
| Bacillus subtilis | 275 |
| Bacillus subtilis | 276 |
| Trichoderma lixii | 277 |
| Streptomyces coelicolor | 278 |
| Bacillus subtilis | 279 |
| Bacillus subtilis | 280 |
| Bacillus subtilis | 281 |
| Bacillus licheniformis | 282 |
| Bacillus licheniformis | 283 |
| Bacillus subtilis | 284 |
| Bacillus subtilis | 285 |
| Bacillus subtilis | 286 |

The signal peptide may also comprise an amino acid sequence having at least 70%, at least 75%, at least 80%, at least 85%, at least 90%, at least 95%, at least 98%, at least 99%, or 100% sequence identity with any one of SEQ ID NOs: 209-246, and 267-286. Furthermore, the microorganism in which the enzyme is expressed can suitably comprises a bacterium of the genus Bacillus, a bacterium of the genus Paenibacillus, a bacterium of the genus Lysinibacillus, a bacterium of the genus Pseudomonas, or a combination of any thereof. For example, the microorganism can comprise Bacillus mycoides, Bacillus pseudomycoides, Bacillus cereus, Bacillus firmus, Bacillus thuringiensis, Bacillus megaterium, Bacillus subtilis, Bacillus aryabbattai, Bacillus amyloliquefaciens, Bacillus circulans, Bacillus flexus, Bacillus nealsonii, Bacillus pumulis, Bacillus licheniformis, Lysinibacillus macroides, Lysinibacillus sphericus, Lysinibacillus fusiformis, or a combination of any thereof. In certain embodiments, the microorganism preferably comprises Bacillus thuringiensis, Bacillus cereus, Bacillus pseudomycoides, Bacillus mycoides, Lysinibacillus macroides, Lysinibacillus fusiformis, Lysinibacillus sphericus, or a combination of any thereof.

Additionally, or alternatively, an enzyme provided herein having insecticidal, nematicidal, or pesticidal activity can comprise an amino acid sequence having at least one amino acid substitution or deletion relative to the sequence of a wild-type enzyme from the host species, wherein the amino acid substitution or deletion retains the catalytic residues of the wild-type enzyme and results in the same or increased enzymatic activity as compared to the activity of the wild-type enzyme under the same conditions.

Enzyme variants of the present disclosure may comprise one or more conservative mutations compared with a base sequence from which they are derived. For example, amino acids may be classified according to the structure, size, electric charge, and influence on the solubility of amino acids in water into five groups: nonpolar aliphatic (glycine, alanine, valine, leucine, isoleucine, and proline), aromatic (phenylalanine, tyrosine, tryptophan), polar uncharged (serine, threonine, cysteine, methionine, asparagine, glutamine), negatively charged (aspartate, glutamate), and positively charged (lysine, arginine, histidine). Enzyme variants of the present invention may therefore comprise conservative mutations compared with any of SEQ ID NOs: 1-208, 247-260, and 287-328 in which a nonpolar aliphatic residue is replaced with a different nonpolar aliphatic residue, an aromatic residue is replaced with a different aromatic residue, a polar uncharged residue is replaced with a different polar uncharged residue, a negatively charged residue is replaced with a different negatively charged residue, or a positively charged residue is replaced with a different positively charged residue. Enzyme variants of the present invention may also comprise conservative mutations compared with any of SEQ ID NOs: 1-208, 247-260, and 287-328 in which an amino acid residue is replaced with a different amino acid residue having a similar R-group, for example serine/threonine, aspartate/glutamate, asparagine/glutamine, or leucine/isoleucine. Enzyme variants comprising conservative mutations may exhibit the same, greater, or less enzymatic activity than the base sequence from which they are derived.

Enzymes provided herein may be further defined by one or more conserved residues, regions, or domains associated with lipase, esterase, polyurenthanase, protease, chitinase, or collegenase activity, or a conservative substitution thereof. For example, esterases may comprise a serine residue at the position corresponding to residue 114 of SEQ ID NO: 248. Esterase sequences and esterase variants may comprise an aspartic acid or glutamic acid residue at the position corresponding to residue 168 of SEQ ID NO: 248. In addition, esterase sequences and esterase variants may comprise a histidine residue at the position corresponding to residue 199 of SEQ ID NO: 248. Ser114, Asp168, and His199 are involved in the catalytic esterase activity provided by SEQ ID NO: 248. Similarly, an esterase may comprise a serine residue at the position corresponding to residue 106 of SEQ ID NO:88. Esterase sequences and esterase variants may comprise an aspartic acid or glutamic acid residue at the position corresponding to residue 162 of SEQ ID NO:88. In addition, esterase sequences and esterase variants may comprise a histidine residue at the position corresponding to residue 185 of SEQ ID NO:88. Ser106, Asp162, and His185 are involved in the catalytic esterase activity provided by SEQ ID NO:88. Furthermore, an esterase may comprise a serine residue at the position corresponding to residue 78 of SEQ ID NO: 89. Esterase sequences and esterase variants may comprise an aspartic acid or glutamic acid residue at the position corresponding to residue 134 of SEQ ID NO:89. In addition, esterase variants may comprise a histidine residue at the position corresponding to residue 157 of SEQ ID NO: 89. Ser78, Asp134, and His157 are involved in the catalytic esterase activity provided by SEQ ID NO: 89.

As another example, lipases may comprise a serine residue at the position corresponding to residue 131 of SEQ ID NO:32. Lipase sequences and lipase variants may comprise an aspartic acid or glutamic acid residue at the position corresponding to residue 308 of SEQ ID NO:32. In addition, lipase sequences and lipase variants may comprise a histidine residue at the position corresponding to residue 330 of SEQ ID NO:32. Ser131, Asp308, and His330 are involved in the catalytic lipase activity provided by SEQ ID NO:32.

Additionally, polyurethanases may comprise a serine residue at the position corresponding to residue 207 of SEQ ID NO: 105. Polyurethanases sequences and polyurethanases variants may comprise an aspartic acid or glutamic acid residue at the position corresponding to residue 256 of SEQ ID NO:105. In addition, polyurethanases sequences and polyurethanases variants may comprise a histidine residue at the position corresponding to residue 314 of SEQ ID NO: 105. Ser207, Asp256, and His314 are involved in the catalytic polyurethanases activity provided by SEQ ID NO:105.

As a further example, a protease may comprise a histidine residue at the position corresponding to residue 337 of SEQ ID NO: 127. Protease sequences and protease variants may comprise a glutamic acid residue at the position corresponding to residue 338 of SEQ ID NO:127. In addition, protease sequences and protease variants may comprise a histidine residue at the position corresponding to residue 422 of SEQ ID NO:127. His337, Glu338, and His422 are involved in the catalytic protease activity provided by SEQ ID NO:127. As a further example, serine proteases may comprise an aspartic or histidine acid residue at the position corresponding to residue 142 of SEQ ID NO:158. Serine protease sequences and serine protease variants may comprise a histidine or aspartic acid residue at the position corresponding to residue 172 of SEQ ID NO: 158. In addition, serine protease sequences and serine protease variants may comprise a serine residue at the position corresponding to residue 326 of SEQ ID NO:158. Asp142, His172, and Ser326 are involved in the catalytic protease activity provided by SEQ ID NO: 158. As a further example, metalloproteases may comprise a histidine acid residue at the position corresponding to residue 364 of SEQ ID NO:130. Metalloproteases sequences and metalloprotease variants may comprise a histidine acid residue at the position corresponding to residue 368 of SEQ ID NO: 130. In addition, metalloprotease sequences and metalloprotease variants may comprise a glutamic residue at the position corresponding to residue 388 of SEQ ID NO:130. His364, His368, and Glu388 are involved in the catalytic protease activity provided by SEQ ID NO:130. Similarly, other enzyme classes disclosed herein comprise conserved catalytic residues, conserved motifs, domain structures, or a combination thereof. For ease of reference, descriptions of the exemplary catalytic motifs are provided below in Table 9.

TABLE 9
Exemplary Catalytic Residues by Enzyme Classes

| Class | Subclass | Conserved Catalytic Residues |
|---|---|---|
| Esterases | | S [ . . . ] D/E [ . . . ] H |
| Lipases | | |
| Polyurethanases | | |
| Chitinase | GH18 | DxDxE [ . . . ] D |
| Chitinase | GH19 | E [ . . . ] E [ . . . ] S |
| Protease | Serine Proteases | H [ . . . ] D [ . . . ] S |
| | | or |
| | | D [ . . . ] H [ . . . ] S |
| Protease | Metalloproteases | HxxxH [ . . . ] E |
| | | or |
| | | HE [ . . . ] H |

Enzyme variants may be synthetically produced or manipulated polypeptides or may be produced through the fusion of two or more heterologous polypeptides. Methods of producing modified enzyme variants or DNA sequences encoding enzyme variants are well known in the art. Because of the degeneracy of the genetic code, a variety of different polynucleotide sequences can encode the polypeptides disclosed herein. All possible triplet codons (and where U also replaces T) and the amino acid encoded by each codon is well-known in the art. In addition, it is well within the capability of one of skill in the art to create alternative polynucleotide sequences encoding the same, or essentially the same, mutant polypeptides of the subject disclosure. Allelic variants of the nucleotide sequences encoding a wild-type or mutant polypeptide of the present disclosure are also encompassed within the scope of the disclosure. As such, the invention further provides recombinant DNA molecules encoding the enzymes disclosed herein or enzyme variants thereof. Said recombinant DNA molecules can be operably linked with a promoter or other regulatory element. In certain embodiments, the promoter may be heterologous with respect to the recombinant DNA molecule. As used herein, the term “heterologous” refers to the combination of two or more DNA molecules when such a combination is not normally found in nature. For example, the two DNA molecules may be derived from different species and/or the two DNA molecules may be derived from different genes, e.g., different genes from the same species or the same genes from different species. A regulatory element is thus heterologous with respect to an operably linked transcribable DNA molecule if such a combination is not normally found in nature, i.e., the recombinant DNA molecule does not naturally occur operably linked to the promoter.

As used herein, a “recombinant polypeptide” is a polypeptide comprising a combination of polypeptides that would not naturally occur together without human intervention. For instance, a recombinant polypeptide may be a polypeptide that is comprised of at least two polypeptides heterologous with respect to each other, a polypeptide that comprises a polypeptide sequence that deviates from polypeptide sequences that exist in nature, a polypeptide that comprises a synthetic polypeptide sequence or a polypeptide expressed by a recombinant DNA sequence that has been incorporated into a host cell's DNA by genetic transformation or gene editing.

As used herein, the term “operably linked” refers to a first molecule joined to a second molecule, wherein the molecules are so arranged that the first molecule affects the function of the second molecule. For example, a signal peptide (first molecule) can be joined to an enzyme (second molecule), wherein the signal peptide causes secretion of the enzyme when expressed in a recombinant microorganism. In some embodiments, a recombinant polypeptide may comprise a signal peptide sequence operably linked to an enzyme sequence disclosed herein. Furthermore, provided herein is a recombinant microorganism that expresses a signal peptide sequence operably linked to an enzyme sequence disclosed herein, wherein expression of the signal peptide operably linked to the enzyme results in secretion of the enzyme from the recombinant microorganism.

For enzymes described herein, “sequence identity” or “percent sequence identity” or “% sequence identity” is determined by aligning the entire length of the sequences in such a way as to obtain optimal matching so that the minimal number of edit operations (e.g., inserts, deletions and substitutions) are needed in order to transform the one sequence into an exact copy of the other sequence being aligned. The EMBOSS Needle Pairwise Sequence Alignment, which is an algorithm that is available through the European Bioinformatics Institute (EMBL-EBI) website, is one example of such analysis. Thus, one embodiment of the invention is a polypeptide sequence that when optimally aligned to a reference sequence, provided herein as SEQ ID NOs: 1-208, 247-260, and 287-328, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having insecticidal, nematicidal, and pesticidal activity. In still further embodiments, a polypeptide sequence that when optimally aligned to a reference sequence, provided herein as SEQ ID NOs: 1-208, 247-260, and 287-328, may comprise conserved catalytic residues and otherwise have at least about 80 percent identity, at least about 85 percent identity, at least about 90 percent identity, at least about 95 percent identity, at least about 98 percent identity, or at least about 99 percent identity to the reference sequence. For example, provided herein is an esterase comprising a serine residue at the position corresponding to residue 114 of SEQ ID NO: 248, an aspartic acid residue at the position corresponding to residue 168 of SEQ ID NO: 248, and a histidine residue at the position corresponding to residue 199 of SEQ ID NO: 248 and otherwise having at least about 8% sequence identity to SEQ ID NO:248.

### IX. Formulations and Compositions

The terms “formulation” and “composition” are used interchangeably herein to refer to a mixture of two or more chemical or biological substances (for example, e.g., a mixture of an enzyme and an agriculturally acceptable carrier or a mixture of a recombinant microorganism and an agriculturally acceptable carrier). Formulations and compositions are further provided comprising at least one polypeptide provided herein or a recombinant microorganism expressing said polypeptide. A formulation or composition provided herein may further comprise an agriculturally acceptable carrier, or an agrochemical. Enzymes of the present disclosure can be formulated in many ways. Common goals for formulation enzyme products include enhancing shelf life, preserving the product from microorganisms, and enhancing enzyme activity. Enzyme products can be lyophilized to extend the shelf life of most enzymes by freeze drying, spray drying, or otherwise removing the liquid aspect of the enzyme product. Liquid and lyophilized products are often bulked out with additives, such as buffers, stabilizers, antimicrobial agents, and volume additives. Enzymes can often be encapsulated or granulated to make the final product safer and easier to use. Granulated products can have enhanced shelf life and have little enzyme activity exposed to the outside surface of the granules. Enzymes may also be attached to organic or inorganic platforms, such as plastic beads, dolomite, clays, charcoals, biochar, nanoparticles, alginates, silica beads help bind them and keep them in an easy-to-use form. Often, enzymes are immobilized on matrices to allow for longer activity and shelf life of the enzyme products. Common matrices include carbon, nanocarbons, agarose, alginates, cellulose and cellulosic material, silica, plastic, stainless steel, glass, polystyrene, and ceramics.

Many formulations or compositions of the enzymes can be used to prolong enzymatic activity or shelf life of the products. These include but are not limited to preservatives, biocides, stabilizers, color enhancers, odor reduction, surfactants, detergents, buffers, cofactors, ions, and other modification to the formulation to enhance the performance of the enzymes.

In one embodiment, to reduce the likelihood of resistance development, a formulation or composition comprising a polypeptide provided herein can further comprise at least one additional polypeptide that exhibits insecticidal, nematicidal, or pesticidal activity against the same pest species, but which is different from the first polypeptide exhibiting exhibit insecticidal, nematicidal, or pesticidal activity.

Other embodiments such as topically applied pesticidal chemistries that are designed for controlling pests that are also controlled by the polypeptides disclosed herein to be used with polypeptides in seed treatments, spray on, drip on, or wipe on formulations can be applied directly to the soil (a soil drench), applied to growing plants expressing the proteins disclosed herein, or formulated to be applied to seed. Such formulations for use in seed treatments can be applied with various stickers and tackifiers known in the art. Such formulations can contain pesticides that are synergistic in mode of action with the polypeptides disclosed, so that the formulation pesticides act through a different mode of action to control the same or similar pests that can be controlled by the polypeptides disclosed, or that such pesticides act to control pests within a broader host range or plant pest species that are not effectively controlled by the polypeptides provided herein.

The aforementioned compositions/formulations can further comprise an agriculturally acceptable carrier, such as a bait, a powder, dust, pellet, granule, spray, emulsion, a colloidal suspension, an aqueous solution, a gel, an aerogel, a hydrogel, a Bacillus spore/crystal preparation, a seed treatment, or bacterium transformed to express one or more of the polypeptides. Depending on the level of pest inhibitory or pest inhibition inherent in the recombinant polypeptide and the level of formulation to be applied to a plant or diet assay, the compositions/formulations can include various by weight amounts of the recombinant polypeptide, e.g., from 0.0001% to 0.001% to 0.01% to 1% to 99% by weight of the recombinant polypeptide.

### X. Treated Seeds

Treated plant seeds are further provided. The plant seed may be treated with an isolated enzyme or a recombinant microorganism expressing any of the polypeptides provided herein, such as an enzyme exhibiting insecticidal, nematicidal, or pesticidal activity.

Treated plant seeds are further provided which may be treated with any of the compositions described herein. Plant seeds treated with any of the formulations described herein are further provided. In any of the treated plant seeds provided, the plant seed may be coated with an isolated enzyme or recombinant microorganism expressing an enzyme provided herein. The isolated enzymes, recombinant microorganisms, or formulations thereof may be used as seed treatments, e.g., seed coatings or dressings. Seed coating or dressing formulations may be in the form of a liquid carrier formulation, a slurry formulation, or a powder formulation. In some embodiments, coating the seed with the isolated enzyme recombinant microorganism, or formulation thereof can be expressed as a use rate or application rate. For example, a plant seed can be coated with the isolated enzyme or recombinant microorganism, or formulation thereof at a rate of at least about 0.01 fl. oz. per unit of seed, at least about 0.05 fl. oz. per unit of seed, at least about 0.1 fl. oz. per unit of seed, at least about 0.25 fl. oz. per unit of seed, at least about 0.5 fl. oz. per unit of seed, at least about 1 fl. oz. per unit of seed, at least about 1.25 fl. oz. per unit of seed, at least about 1.5 fl. oz. per unit of seed, at least about 1.75 fl. oz. per unit of seed, at least about 2 fl. oz. per unit of seed, at least about 2.5 fl. oz. per unit of seed, at least about 5 fl. oz. per unit of seed, at least about 10 fl. oz. per unit of seed, or at least about 20 fl. oz. per unit of seed. In other embodiments, use or application rates can be expressed in units, including but not limited to, mL/kg seed, fl. oz./lb., fl. oz/cwt, or fl. oz/kernel.

Seed coating or dressing formulations may be applied with conventional additives that are provided to make the seed treatment have sticky qualities to stick to and coat the seeds. Suitable additives comprise: tales, graphites, gums, stabilizing polymers, coating polymers, finishing polymers, slip agents for seed flow and planting ability, cosmetic agents, and cellulosic materials such as carboxymethyl cellulose and the like. In specific embodiments, the seed coating or dressing formulation may comprise polyvinyl alcohol (PVA) or polyvinylpyrrolidone (PVP). PVP/PVA may provide a favorable environment for the enzyme or recombinant microorganism because of its high water-binding capacity.

The seed treatment formulations may further comprise colorant agents, other additives, and/or a seed finisher. A “seed finisher” refers to a powder or dry seed coating applied to seed for one or more of the following purposes: absorbing excess liquid adhering to the seed surface after treatment, improving seed lubrication, improving seed flowability, or improving seed appearance.

The seed treatment formulations(s) may be applied to seeds in a suitable carrier such as water or a powder. The seeds can then be allowed to dry and planted in conventional fashion. The isolated enzyme, recombinant microorganism, or formulation thereof can be applied directly to the seed as a solution or in combination with other commercially available additives. For example, the isolated enzyme, recombinant microorganisms, or formulation thereof can be applied in combination with seedling-acceptable carrier(s) (e.g., a liquid carrier or a solid carrier). In some embodiments, applying the isolated enzyme, recombinant microorganisms, or formulation thereof to the plant seed comprises: (a) applying the isolated enzyme, recombinant microorganisms, or formulation thereof to the plant seed at the time of planting; or (b) coating the plant seed with the isolated enzyme, recombinant microorganisms, or formulation thereof. In certain embodiments, the seed treatment formulation comprises a polyvinylpyrrolidone (PVP) film or pulverized film (powder) allowing extended release of an enzyme from a seed coat. Thus, provided herein is a seed coating formulation allowing delayed or extended release of an enzyme from a seed coating, a treated seed, the soil, the surface of a plant, or within a solution.

Solutions containing the isolated enzyme, recombinant microorganism, or formulation thereof can be sprayed or otherwise applied to the seed (e.g., in a seed slurry or a seed soak). Solid or dry materials containing isolated enzymes, recombinant microorganisms, or formulations thereof are also useful to promote effective seedling germination, growth, and protection during early seedling establishment.

The isolated enzyme, recombinant microorganism, or formulation thereof can be used with a solubilizing carrier such as water, a buffer (e.g., citrate or phosphate buffer), other treating agents (e.g., alcohol or another solvent), and/or any soluble agent. In addition, small amounts of drying agent enhancers, such as lower alcohols, etc. can be used in seed coating formulations. Surfactants, emulsifiers, and preservatives can also be added at relatively low (e.g., about 0.5% w/v or less) levels to enhance the stability of the seed coating product.

Seeds can be treated using a variety of methods including, but not limited to, pouring, pumping, drizzling, or spraying an aqueous solution containing the isolated enzyme, recombinant microorganism, or formulation thereof on or over a seed; or spraying or applying the isolated enzyme, recombinant microorganism, or formulation thereof onto a layer of seeds either with or without the use of a conveyor system. Mixing devices useful for seed treatment include but are not limited to tumblers, mixing basins, mixing drums, and fluid application devices that include basins or drums used to contain the seed while coating.

After seed treatment, the seed may be air-dried, or a stream of dry air may be optionally used to aid in the drying of the seed coatings. Seed treatments containing the isolated enzyme, recombinant microorganism, or formulation thereof can be applied using any commercially available seed treatment machinery or can also be applied using any acceptable non-commercial method(s) such as the use of syringes or any other seed treatment device.

XI. Methods for Protecting a Plant from a Pathogen or Pest and Methods for Stimulating Plant Growth and/or Promoting Plant Health

Methods for protecting a plant from a pathogen or pest are provided. In some embodiments, the methods comprise applying at least one isolated enzyme to a plant growth medium, a plant, a plant seed, or an area surrounding a plant or a plant seed, wherein the enzyme is selected from an esterase, a chitinase, a protease, a lipase, and combinations of any thereof. In particular embodiments, the esterase comprises a sequence having at least 80% sequence identity to a sequence selected from SEQ ID NO:83-100, 248, 252-255, and 287-289; the chitinase comprises a sequence having at least 85% sequence identity to a sequence selected from SEQ ID NO: 179-205, and 249; the protease comprises a sequence having at least 85% sequence identity to a sequence selected from SEQ ID NO:122-178, 247, and 310-327; or the lipase comprises a sequence having at least 85% sequence identity to a sequence selected from SEQ ID NO: 1-82, 250, 251, 256-260, and 290-307, wherein the enzyme exhibits insecticidal, nematicidal, or pesticidal activity.

In other embodiments, the methods comprise applying a recombinant microorganism to a plant growth medium, a plant, a plant seed, or an area surrounding a plant or a plant seed. The recombinant microorganism expresses an enzyme disclosed herein, wherein expression of the enzyme is increased as compared to the expression level of the enzyme in a wild-type microorganism of the same kind under the same conditions. In particular embodiments, the enzyme is expressed during vegetative growth of the recombinant microorganism. The enzyme may further comprise a signal peptide that results in secretion of the enzyme from the recombinant microorganism.

Yet another method for protecting a plant from a pathogen or pest is provided. The method comprises applying a formulation provided herein to a plant growth medium, a plant, a plant seed, or an area surrounding a plant or a plant seed. The formulation can comprise any of the formulations described herein.

In some embodiments, the enzyme or recombinant microorganism can be applied in-furrow or can be included in a soil amendment. Alternatively, or in addition, the enzyme or recombinant microorganism can be impregnated onto a dry particle, a vermiculite or other matrix, a plastic polymer, a peat moss or potting mix, prior to application to the plant growth medium. The enzyme or recombinant microorganism can also be applied to the plant growth medium via a water source, a drip irrigation line, a broadcast liquid application to the soil, or a broadcast dry application to the soil.

In any of the methods described herein, the method can comprise applying the enzyme or the recombinant microorganism to the plant, the roots of the plant, the plant seed, the soil surrounding the plant or plant seed, or a foliar application.

In any of the methods described herein, the method can further comprise inactivating the recombinant microorganism prior to applying the recombinant microorganism to the plant growth medium, the plant, the plant seed, or the area surrounding the plant or the plant seed.

In any of the methods described herein, the method can comprise applying the isolated enzyme or recombinant microorganism, or formulation thereof to the plant growth medium. In any of the methods described herein involving the use of a plant growth medium, the plant growth medium can comprise soil, water, an aqueous solution, sand, gravel, a polysaccharide, mulch, compost, peat moss, straw, logs, clay, soybean meal, yeast extract, or a combination thereof.

In any of the methods described herein, the method can comprise applying the isolated enzyme or recombinant microorganism, or formulation thereof to the plant. For example, the method can comprise applying the isolated enzyme, recombinant microorganism, or formulation thereof to roots of the plant or the soil surrounding the plant. Alternatively, or in addition, the method can comprise applying the isolated enzyme, recombinant microorganism, or formulation thereof as a foliar application. In some embodiments, applying the isolated enzyme or recombinant microorganism, or formulation thereof can be expressed as a use rate or application rate. For example, the isolated enzyme, recombinant microorganism, or formulation thereof can be applied at a rate of at least about 0.05 fl oz/A, at least about 0.1 fl oz/A, at least about 0.2 fl oz/A, at least about 0.5 fl oz/A, at least about 0.75 fl oz/A, at least about 1 fl oz/A, at least about 1 fl oz/A, at least about 2.5 fl oz/A, at least about 5 fl oz/A, at least about 7.5 fl oz/A, at least about 10 fl oz/A, at least about 12 fl oz/A, at least about 15 fl oz/A, at least about 20 fl oz/A, at least about 25 fl oz/A, or at least about 30 fl oz/A. In other embodiments, the isolated enzyme, recombinant microorganism, or formulation thereof can be applied at a rate of at least about 0.01 mL/plant, at least about 0.05 mL/plant, at least about 0.1 mL/plant, at least about 0.25 mL/plant, at least about 0.5 mL/plant, at least about 1 mL/plant, at least about 2.5 mL/plant, at least about 5 mL/plant, or at least about 10 mL/plant. In still other embodiments, use or application rates can be expressed in units, including but not limited to, mL/kg seed, fl. oz./lb, fl. oz/cwt, or fl. oz/A furrow.

In any of the methods described herein, the method can comprise applying the isolated enzyme, recombinant microorganism, or formulation thereof to the plant seed. Where the method comprises applying the isolated enzyme, recombinant microorganism, or formulation thereof to the plant seed, applying the isolated enzyme, recombinant microorganism, or formulation thereof to the plant seed can comprise: (a) applying isolated enzyme, recombinant microorganism, or formulations thereof to the plant seed at the time of planting; or (b) coating the plant seed with the isolated enzyme, recombinant microorganism, or formulations thereof.

In any of the methods described herein, the plant pests may include phytoparasitic pests from the phylum Nematoda, for example, Achylsiella spp., Aglenchus spp., Anguina spp., Aphelenchoides spp., Belonolaimus spp., Bursaphelenchus spp., Cacopaurus spp., Criconemella spp., Criconemoides spp., Ditylenchus spp., Dolichodorus spp., Globodera spp., Helicotylenchus spp., Hemicriconemoides spp., Hemicycliophora spp., Heterodera spp., Hoplolaimus spp., Longidorus spp., Lygus spp., Meloidogyne spp., Meloinema spp., Mesocriconema spp., Merlinius spp Nacobbus spp., Neotylenchus spp., Paralongidorus spp., Paraphelenchus spp., Paratrichodorus spp., Paratylenchus spp., P Pratylenchus spp., Pseudohalenchus spp., Psilenchus spp., Punctodera spp., Quinisulcius spp., Radopholus spp., Rotylenchulus spp., Rotylenchus spp., Scutellonema spp., Subanguina spp., Trichodorus spp., Tylenchulus spp., Tylenchorhynchus spp., Xiphinema spp.

In any of the methods described herein, plants grown in the presence of the isolated enzyme, microorganism, or formulation thereof can exhibit decreased susceptibility to a pest, such as nematodes, as compared to plants grown in the absence of the enzyme or the microorganism, under the same conditions. In any of the methods described herein, plants grown in the presence of the isolated enzyme, microorganism, or formulation thereof can exhibit decreased nematode damage, including reduced galling, swelling, lesions or stunting, reduced cysts, and/or reduced nematodes, as compared to plants grown in the absence of the enzyme or the microorganism, under the same conditions. For example, reduced nematodes may be measured by nematodes per weight of root tissue, nematodes per plant, nematodes per volume of soil, or nematodes per weight of foliar leaf tissue. In any of the methods described herein, plants or the locus in which the plant is grown, such as soil, to which the isolated enzyme, microorganism, or formulation thereof has been applied can exhibit reduced nematode eggs and/or reduced nematodes per volume of soil, as compared to plants grown in the absence of the enzyme or the microorganism, under the same conditions. In any of the methods described herein, an isolated enzyme, a recombinant microorganism, or a formulation applied to nematodes, nematode eggs, or cysts can result in direct killing, degradation, behavioral modification, or prevention of egg hatch.

In one embodiment, the isolated enzyme, recombinant microorganism, composition, or formulation of the present invention decreases damage caused by a plant pathogen or pest by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12%, or by at least about 20%, or by at least about 25%, or by at least about 30%, or by at least about 40%, or by at least about 50% when compared to plants produced under the same conditions but without treatment.

In any of the methods described herein, plants grown in the presence of the isolated enzyme, recombinant microorganism, or formulation thereof can exhibit decreased susceptibility to a pathogen or pest as compared to plants grown in the absence of the enzyme or the microorganism, under the same conditions.

Methods for stimulating plant growth and/or promoting plant health are also provided. The methods comprise applying an isolated esterase or recombinant microorganism expressing an esterase provided herein to a plant growth medium, a plant, a plant seed, or an area surrounding a plant or a plant seed.

Yet another method for stimulating plant growth and/or promoting plant health is provided. The method comprises applying a formulation comprising an isolated esterase or recombinant microorganism expressing esterase to a plant growth medium, a plant, a plant seed, or an area surrounding a plant or a plant seed.

In any of the methods for stimulating plant growth and/or promoting plant health described herein, the method can comprise applying an isolated esterase or recombinant microorganism expressing an esterase provided herein to the plant growth medium. In certain embodiments, the plant growth medium can comprise soil, water, an aqueous solution, sand, gravel, a polysaccharide, mulch, compost, peat moss, straw, logs, clay, soybean meal, yeast extract, or a combination thereof.

The plant growth medium can comprise a fertilizer.

In any of the methods described herein for stimulating plant growth and/or promoting plant health, the method can comprise applying an isolated esterase or recombinant microorganism expressing an esterase, or formulation thereof to the plant. For example, the method can comprise applying the isolated esterase or recombinant microorganism expressing esterase, or formulation thereof to roots of the plant. Alternatively, or in addition, the method can comprise applying the isolated esterase or recombinant microorganism expressing an esterase, or formulation as a foliar application. In still further embodiments, the method can comprise applying an isolated esterase or recombinant microorganism expressing esterase, or formulation thereof to the plant seed or the soil surrounding a plant or plant seed.

In any of the methods described herein, plants grown in the presence of the isolated enzyme, recombinant microorganism, or formulation thereof can exhibit increased growth as compared to plants grown in the absence of the enzyme or the microorganism under the same conditions.

In any of the methods described herein, seeds to which the isolated enzyme, recombinant microorganism, or formulation thereof have been applied can exhibit increased germination rates as compared to seeds to which the enzyme or microorganism has not been applied, under the same conditions.

In any of the methods described herein, plants grown in the presence of the isolated enzyme, recombinant microorganism, or formulation thereof can exhibit increased nutrient uptake as compared to plants grown in the absence of the enzyme or the microorganism, under the same conditions.

In any of the methods described herein, plants grown in the presence of the isolated enzyme, recombinant microorganism, or formulation thereof can exhibit decreased susceptibility to an environmental stress (e.g., drought, flood, heat, freezing, salt, heavy metals, low pH, high pH, or a combination of any thereof) as compared to plants grown in the absence of the enzyme or the microorganism, under the same conditions.

In any of the methods described herein, plants grown in the presence of the isolated enzyme, recombinant microorganism, or formulation thereof can exhibit increased root nodulation as compared to plants grown in the absence of the enzyme or the microorganism, under the same conditions.

In any of the methods described herein, plants grown in the presence of the isolated enzyme, recombinant microorganism, or formulation thereof can exhibit greater crop yield as compared to plants grown in the absence of the enzyme, or the microorganism, under the same conditions. In one embodiment, the isolated enzyme, recombinant microorganism, or formulation of the present invention increases yield or total plant weight by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12%, or by at least about 20%, or by at least about 25%, or by at least about 30%, or by at least about 40%, or by at least about 50% when compared to plants produced under the same conditions but without treatment. In another embodiment, the isolated enzyme, recombinant microorganism, or formulation of the present invention improves some aspect of plant vigor, such as germination, by at least about 0.5%, or by at least about 1%, or by at least about 2%, or by at least about 3%, or by at least about 5%, or by at least about 6%, or by at least about 7%, or by at least about 8%, or by at least about 9%, or by at least about 10%, or by at least about 11%, or by at least about 12%, or by at least about 20%, or by at least about 25%, or by at least about 30%, or by at least about 40%, or by at least about 50% when compared to plants produced under the same conditions but without treatment.

In any of the methods described herein, plants grown in the presence of the isolated enzyme, recombinant microorganism, or formulation thereof can exhibit altered leaf senescence as compared to plants grown in the absence of the enzyme or the microorganism, under the same conditions.

### XII. Carriers and Agrochemicals

As described above, the formulations described herein comprise an agriculturally acceptable carrier.

The agriculturally acceptable carrier can comprise a dispersant, a surfactant (e.g., a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcohol, an alkyl phosphate, or a combination of any thereof), an additive (e.g., an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutane dioate, a benzene acetonitrile derivative, a proteinaceous material, or a combination of any thereof), water, a thickener (a long chain alkylsulfonate of polyethylene glycol, a polyoxyethylene oleate, or a combination of any thereof), an anti-caking agent (e.g., sodium salt, a calcium carbonate, silica, silicate, diatomaceous earth, or a combination of any thereof), a residue breakdown product, a composting formulation, a granular application, diatomaceous earth, an oil, a coloring agent, a stabilizer, a preservative, a polymer, a coating, or a combination of any thereof.

Where the agriculturally acceptable carrier comprises a surfactant, the surfactant can comprise a non-ionic surfactant.

Where the agriculturally acceptable carrier comprises an additive and the additive comprises a proteinaceous material, the proteinaceous material can comprise a milk product, wheat flour, soybean meal, soybean powder, soybean flour, blood, albumin, gelatin, alfalfa meal, yeast extract, or a combination of any thereof.

Where the agriculturally acceptable carrier comprises an anti-caking agent and the anti-caking agent comprises a sodium salt, the sodium salt can comprise a sodium salt of monomethyl naphthalene sulfonate, a sodium salt of dimethyl naphthalene sulfonate, a sodium sulfite, a sodium sulfate, or a combination of any thereof.

The agriculturally acceptable carrier can comprise vermiculite, charcoal, sugar factory carbonation press mud, rice husk, carboxymethyl cellulose, peat, perlite, fine sand, calcium carbonate, flour, alum, a starch, talc, graphite, wax esters, polyvinyl pyrrolidone, polyvinyl alcohol, or a combination of any thereof.

Any of the formulations described herein can comprise a seed coating formulation (e.g., an aqueous or oil-based solution for application to seeds or a powder or granular formulation for application to seeds), a liquid formulation for application to plants or to a plant growth medium (e.g., a concentrated formulation or a ready-to-use formulation), or a solid formulation for application to plants or to a plant growth medium (e.g., a granular formulation or a powder agent).

The agriculturally acceptable carrier may comprise a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising isolated enzymes or recombinant microorganisms to improve efficacy, stability, and physical properties, usability and/or to facilitate processing, packaging and end-use application. Such formulation ingredients may include inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments,” Annu. Rev. Phytopathol., 28:321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids, and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or fragments thereof may be used directly with or without water as the diluent without any other formulation preparation. In a particular embodiment, a wetting agent, or a dispersant, is added to a dried concentrate of the whole broth resulting from the fermentation, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, NJ); siloxanes such as BREAK-THRU® (Evonik, Germany); nonionic compounds, such as ATLOX™ 4894 (Croda Inc., Edison, NJ); alkyl polyglucosides, such as TERWET® 3001 (Huntsman International LLC, The Woodlands, Texas); C12-C14 alcohol ethoxylate, such as TERGITOL® 15-S-15 (The Dow Chemical Company, Midland, Michigan); phosphate esters, such as RHODAFAC® BG-510 (Rhodia, Inc.); and alkyl ether carboxylates, such as EMULSOGEN™ LS (Clariant Corporation, North Carolina).

As described above, any of the formulations described herein can comprise an agrochemical. The agrochemical can comprise a fertilizer, a micronutrient fertilizer material, a nitrogen stabilizer, an insecticide, a nematicide, an herbicide, a plant growth amendment, a fungicide, an insecticide, a molluscicide, an algicide, a bacterial inoculant, a fungal inoculant, a plant hormone, or a combination of any thereof. In some embodiments, the agrochemical comprises an insecticide, the insecticide comprising an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination of any thereof. In other embodiments, the agrochemical may comprise a fungicide. The fungicide may comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metalaxyl, thiamimefon, triforine, cyproconazole, picoxystrobin, pyraclostrobin, prothioconazole, fluopyram, metalaxyl, mefenoxam, thiamethoxam, azoxystrobin, tritconazole, thiram, trifloxystrobin, fludioxinil, tebuconazole, flupyram, pydiflumetofen, Burkholderia spp., thiabendazole, flutriafol, fluxapyroxad, ethaboxam, penflufen, sedaxane, ipconazole, thiophanate-methyl, oxathiapiprolin, inpyrfluxam, benzovindiflupir, carboxin, picarbutrazox, Bacillus spp., mefentrifluconazole, metalaxyl, myclobutanil, captan, pentachloronitrobenzene, acibenzolar-S-methyl, fluxofenim, carbendazim, or a combination of any thereof. In other embodiments, the agrochemical may comprise an insecticide, the insecticide may include without limitation, comprising a clothianidin, imidacloprid, cyantraniliprole, spinetoram, chlorantraniliprole, malathion, dimethoate, sevin, baythroid, danitol, lannate, admire, assail, pencycuron, ctacyflutrhin, fipronil, fludioxinil, thiamethoxam, carbofuran, carbosulfan, an organophosphate, a carbamate, a pyrethroid, an neonicotinoid or a combination of any thereof. In still further embodiments, the agrochemical may comprise a nematicide, which include without limitation fluopyram, pydiflumetofen, aldicarb, acephate, aldoxycarb, acibenzolar-S-methyl, azadirachtin, carbosulfan, carbofuran, chlorfenapyr, oxamyl, benfuracarb, thiodicarb, fanamiphos, fenamiphos, fensulfothion, thoprofos, cadusafos, terbufos, fosthiazate, phorate, imicyafos, abamectin, cyclobutrifluram, spirotetramat, fufural, fluensulfone, fluazaindolizine, iprodione, carbon disulfide, dimethyl disulfide, sodium tetrathiocarbonate, methyl bromide, methyl iodide, 1,2-dibromo-3-chloropropane, ethylene dibromine, ethylene dibromide, 1,3-dichloropropene, chloropicrin, dazometallyl isothiocyanate, allyl isothiocyanate, ivermectin, 1,2-dibromo-3-chloropropane; ethoprop, thiodicarb, metam potassium, metam sodium, tioxazafen, chitin, chitosan, curcumin, harpin protein, cis-jasmone, Quillaja extract, sesame oil, mustard seed meal, 1,4-naphthoquinone, juglone, a biologically-based nematicide, Bacillus-based nematicides, Purpureocillium-based nematicides, Paccilomyces-based nematicides, Pasteuria-based nematicides, Pochonia-based nematicides, Burkholderia-based nematicides, Streptomyces-based nematicides, Trichoderma-based nematicides, Myrothecium-based nematicides, a bacterial extract, a fungal extract, a botanical extract, or a combination of any thereof. In other embodiments, the agrochemical may comprise a nitrogen stabilizer, the nitrogen stabilizer comprising a nitrification inhibitor, a urease inhibitor, or a nitrogen leaching preventative agent. The nitrogen stabilizer can further comprise N (n-butyl) thiophosphoric acid triamide (NBPT), N (n-propyl) thiophosphoric acid triamide (NPPT), nitropyrin, dicyandiamide (DCD), ammonium thiosulfate (ATS), calcium heteropolysaccharide, or poly coated ureas.

### XIII. Plants

In any of the methods described herein relating to plants, the plant can be a dicotyledon, a monocotyledon, a gymnosperm, or an angiosperm. Likewise, for any of the seeds described herein the seed can be a seed of a dicotyledon, a monocotyledon, a gymnosperm, or an angiosperm.

For example, where the plant is a dicotyledon or the seed is a seed of a dicotyledon, the dicotyledon can be selected from the group consisting of bean, pea, tomato, pepper, squash, alfalfa, almond, aniseseed, apple, apricot, arracha, artichoke, avocado, bambara groundnut, bect, bergamot, black pepper, black wattle, blackberry, blueberry, bitter orange, bok-choi, Brazil nut, breadfruit, broccoli, broad bean, Brussels sprouts, buckwheat, cabbage, camelina, Chinese cabbage, cacao, cantaloupe, caraway seeds, cardoon, carob, carrot, cashew nuts, cassava, castor bean, cauliflower, celeriac, celery, cherry, chestnut, chickpea, chicory, chili pepper, chrysanthemum, cinnamon, citron, citrus, clementine, clove, clover, coffee, cola nut, colza, corn, cotton, cottonseed, cowpea, crambe, cranberry, cress, cucumber, currant, custard apple, drumstick tree, earth pea, cchium, eggplant, endive, fennel, fenugreek, fig, filbert, flax, geranium, gooseberry, gourd, grape, grapefruit, guava, hemp, hempseed, henna, hop, horse bean, horseradish, indigo, jasmine, Jerusalem artichoke, jute, kale, kapok, kenaf, kiwi, kohlrabi, kumquat, lavender, lemon, lentil, lespedeza, lettuce, lime, liquorice, litchi, loquat, lupine, macadamia nut, mace, mandarin, mangel, mango, medlar, melon, mint, mulberry, mustard, nectarine, niger seed, nutmeg, okra, olive, opium, orange, papaya, parsnip, pea, peach, peanut, pear, pecan nut, persimmon, pigeon pea, pistachio nut, plantain, plum, pomegranate, pomelo, poppy seed, potato, sweet potato, prune, pumpkin, quebracho, quince, trees of the genus Cinchona, quinoa, radish, ramie, rapeseed, raspberry, rhea, rhubarb, rose, rubber, rutabaga, safflower, sainfoin, salsify, sapodilla, Satsuma, scorzonera, sesame, shea tree, soybean, spinach, squash, strawberry, sugar beet, sugarcane, sunflower, swede, sweet pepper, tangerine, tea, teff, tobacco, tomato, trefoil, tung trec, turnip, urena, vetch, walnut, watermelon, yerba mate, wintercress, shepherd's purse, garden cress, peppercress, watercress, pennycress, star anise, laurel, bay laurel, cassia, jamun, dill, tamarind, peppermint, oregano, rosemary, sage, soursop, pennywort, calophyllum, balsam pear, kukui nut, Tahitian chestnut, basil, huckleberry, hibiscus, passionfruit, star apple, sassafras, cactus, St. John's wort, loosestrife, hawthorn, cilantro, curry plant, kiwi, thyme, zucchini, ulluco, jicama, waterleaf, spiny monkey orange, yellow mombin, starfruit, amaranth, wasabi, Japanese pepper, yellow plum, mashua, Chinese toon, New Zealand spinach, bower spinach, ugu, tansy, chickweed, jocote, Malay apple, paracress, sowthistle, Chinese potato, horse parsley, hedge mustard, campion, agate, cassod tree, thistle, burnet, star gooseberry, saltwort, glasswort, sorrel, silver lace fern, collard greens, primrose, cowslip, purslane, knotgrass, terebinth, tree lettuce, wild betel, West African pepper, yerba santa, tarragon, parsley, chervil, land cress, burnet saxifrage, honeyherb, butterbur, shiso, water pepper, perilla, bitter bean, oca, kampong, Chinese celery, lemon basil, Thai basil, water mimosa, cicely, cabbage-tree, moringa, mauka, ostrich fern, rice paddy herb, yellow sawah lettuce, lovage, pepper grass, maca, bottle gourd, hyacinth bean, water spinach, catscar, fishwort, Okinawan spinach, lotus sweetjuice, gallant soldier, culantro, arugula, cardoon, caigua, mitsuba, chipilin, samphire, mampat, ebolo, ivy gourd, cabbage thistle, sea kale, chaya, huauzontle, Ethiopian mustard, magenta spreen, good king henry, epazole, lamb's quarters, centella plumed cockscomb, caper, rapini, napa cabbage, mizuna, Chinese savoy, kai-lan, mustard greens, Malabar spinach, chard, marshmallow, climbing wattle, China jute, paprika, annatto seed, spearmint, savory, marjoram, cumin, chamomile, lemon balm, allspice, bilberry, cherimoya, cloudberry, damson, pitaya, durian, elderberry, feijoa, jackfruit, jambul, jujube, physalis, purple mangosteen, rambutan, redcurrant, blackcurrant, salal berry, satsuma, ugli fruit, azuki bean, black bean, black-eyed pea, borlotti bean, common bean, green bean, kidney bean, lima bean, mung bean, navy bean, pinto bean, runner bean, mangetout, snap pea, sweet pea, broccoflower, calabrese, nettle, bell pepper, raddichio, daikon, white radish, skirret, tat soi, broccolini, black radish, burdock root, fava bean, broccoli raab, lablab, lupin, sterculia, velvet beans, winged beans, yam beans, mulga, ironweed, umbrella bush, tjuntjula, wakalpulka, witchetty bush, wiry wattle, chia, beech nut, candlenut, colocynth, mamoncillo, Maya nut, mongongo, ogbono nut, paradise nut, and cempedak.

Where the plant is a monocotyledon or the seed is a seed of a monocotyledon, the monocotyledon can be selected from the group consisting of corn, wheat, oat, rice, barley, millet, banana, onion, garlic, asparagus, ryegrass, millet, fonio, raishan, nipa grass, turmeric, saffron, galangal, chive, cardamom, date palm, pineapple, shallot, leek, scallion, water chestnut, ramp, Job's tears, bamboo, ragi, spotless watermeal, arrowleaf elephant car, Tahitian spinach, abaca, areca, bajra, betel nut, broom millet, broom sorghum, citronella, coconut, cocoyam, maize, dasheen, durra, durum wheat, edo, fique, formio, ginger, orchard grass, esparto grass, Sudan grass, guinea corn, Manila hemp, henequen, hybrid maize, jowar, lemon grass, maguey, bulrush millet, finger millet, foxtail millet, Japanese millet, proso millet, New Zealand flax, oats, oil palm, palm palmyra, sago palm, redtop, sisal, sorghum, spelt wheat, sweet corn, sweet sorghum, sugarcane, taro, teff, timothy grass, triticale, vanilla, wheat, and yam.

Where the plant is a gymnosperm or the seed is a seed of a gymnosperm, the gymnosperm can be from a family selected from the group consisting of Araucariaceae, Boweniaceae, Brassicaceae, Cephalotaxaceae, Cupressaceae, Cycadaceae, Ephedraceae, Ginkgoaceae, Gnetaceae, Pinaceae, Podocarpaceae, Taxaceae, Taxodiaceae, Welwitschiaceae, and Zamiaceae.

The plants and plant seeds described herein may include transgenic plants or plant seeds, such as transgenic cereals (wheat, rice), maize, soybean, potato, cotton, tobacco, oilseed rape and fruit plants (fruit of apples, pears, citrus fruits and grapes, including wine grapes). Preferred transgenic plants include corn, soybeans, potatoes, cotton, tobacco, sugar beet, sugarcane, and oilseed rape.

Plant seeds as described herein can be genetically modified (e.g., any seed that results in a genetically modified plant or plant part that expresses herbicide tolerance, tolerance to environmental factors such as water stress, drought, viruses, and nitrogen production, or resistance to bacterial, fungi or insect toxins). Suitable genetically modified seeds include those of core crops, vegetables, fruits, trees, fiber crops, oil crops, tuber crops, coffee, flowers, legume, cereals, as well as other plants of the monocotyledonous and dicotyledonous species. Preferably, the genetically modified seeds include peanut, tobacco, grasses, wheat, barley, rye, sorghum, rice, rapeseed, sugarbeet, sunflower, tomato, pepper, bean, lettuce, potato, and carrot. Most preferably, the genetically modified seeds include cotton, soybean, and corn (sweet, field, seed, or popcorn).

Particularly useful transgenic plants which may be treated according to the invention are plants containing transformation events, or a combination of transformation events, which are listed for example in the databases from various national or regional regulatory agencies (see for example www.gmoinfo.jrc.it/gmp_browse.aspx and www.agbios.com/dbase.php).

Having described the invention in detail, it will be apparent that modifications and variations are possible without departing from the scope of the invention defined in the appended claims.

### EXAMPLES

### Example 1

Seed treatment of soybean with a protease reduces presence of multiple independent genera of plant parasitic nematodes in the surrounding soil

The nematicidal enzyme neutral protease (SEQ ID NO: 127) was produced by fermentation in Bacillus subtilis. The enzyme was cloned into a Bacillus expression plasmid p1797 with isopropyl β-d-1-thiogalactopyranoside (IPTG)-inducible expression. The plasmid was transformed into B. subtilis, and the gene sequence was confirmed. The strain was grown in rich media until an optical density (OD) of 0.2-0.5 was reached. Enzyme expression was then induced by addition of 1 mM IPTG. Following the culture harvest, cells were removed by filtration through a 0.22 μM filter. The culture filtrate containing the secreted enzyme was then collected and used for further applications.

The catalytic domain of the nematicidal enzyme serine protease 2 (SEQ ID NO: 247) was produced by fermentation in Bacillus thuringiensis. The enzyme was cloned into plasmid p1864 which was introduced into Bacillus thuringiensis by electroporation. Single transformed colonies were isolated by plating on nutrient broth plates containing tetracycline (10 μg/mL). Individual positive colonies were used to inoculate brain heart infusion broth containing tetracycline (10 μg/mL) and incubated overnight at 30° C., 300 rpm. Verified colonies were grown overnight in brain heart infusion broth with 10 μg/mL tetracycline and induced to sporulate through incubation in a spore-induction medium at 30° C. for 48 hours. Following the culture harvest, cells were removed by filtration through a 0.22 μM filter. The culture filtrate containing the free enzyme was collected and used for further applications.

Seeds of commercial varieties 3555XF and 3230E3 were treated with a base slurry consisting of the agrochemicals Maxim® 4FS (40.3% fludioxinil), Proline 480SC (41.0% Prothioconazole), Gaucho® (48.7% Imidacloprid) & Sensient Red colorant or with base plus one of the two test treatments including filtrate of the nematicidal neutral protease (SEQ ID NO: 127) expressed in Bacillus subtilis or free enzyme from the nematicidal serine protease 2 catalytic domain (SEQ ID NO: 247) expressed in Bacillus thuringiensis. Seed treatment was performed in a Wintersteiger Hege 11 liquid seed treater by applying 3800 seeds per batch. Nematicidal enzymes (Serine protease 2 catalytic domain SEQ ID NO: 247; Neutral protease SEQ ID NO: 127) were applied at a final use rate of 2 fl. oz. per unit of seed. Seeds were planted in native soil at three independent trial locations in Missouri, Illinois, and Indiana between 31 May 2022 and 1 Jun. 2022. For each trial location, four replicates per treatment were arranged in a randomized complete block design. Seeds were planted in two 5.3 m rows at 76 cm apart, at a depth of 2.6 cm.

To track accretion of plant parasitic nematodes over time in soil surrounding nematicidal seed treatments, soil samples were collected at the following time points: A) 27 to 36 days post-planting and B) 34 to 41 days post planting. Soil sampling was conducted by collecting core samples from the top 8 inches of soil between rows. For each plot, 6 subsamples were collected following a zig-zag pattern between the plant rows. These subsamples were combined into one composite sample per plot to determine the number of nematodes per 100 cc of soil. To quantify nematodes per 100 cc of soil, samples were mixed thoroughly then nematodes were extracted by semi-automatic elutriation and centrifugal flotation. Nematode identification to the genus level was performed through examination at 40× magnification under a dissecting microscope. A multi-site area under the progress curve (AUPC) analysis was conducted. AUPC provided a quantitative summary of nematode presence intensity over time. AUPC was calculated using the following formula, where t is sampling time points in days, y is nematode count, and Aₖ is the total area under the curve at t=tₖ.

$${A}_{k} = {\sum }_{i = 1}^{{N}_{\text{?} + 1}} \frac{( {y}_{i} + {y}_{i + 1} )}{2} ( {t}_{\text{?} + 1} - {t}_{\text{?}} )$$

$$\text{?} \text{indicates text missing or illegible when filed}$$

TABLE 10
Soybean Seed Treatment with Proteases Reduced Soil
Presence of Plant Parasitic Nematode Species

| | | | RKN | |
|---|---|---|---|---|
| | Cyst | | (Root knot | RKN |
| | (Heterodera | Cyst | nematode, | (Root knot |
| | spp.) | (Heterodera | Meloidogyne | nematode, |
| | AUPC for | spp.) | spp.) | Meloidogyne |
| | Nematodes | % | AUPC for | spp.) |
| | per 100 cc | Reduction | Nematodes per | % Reduction |
| Treatment | of soil | in AUPC | 100 cc of soil | in AUPC |
| Base Seed Treatment | 1988 | 100% | 139 | 100% |
| Control | | | | |
| Seed Treatment | 1658 | 83% (−17%) | 106 | 76% (−24%) |
| Composition 1 | | | | |
| Serine protease 2 | | | | |
| catalytic domain | | | | |
| (SEQ ID NO: 247) | | | | |
| [2 fl oz/unit] + | | | | |
| base seed treatment | | | | |
| Seed Treatment | 1413 | 71% (−29%) | 93 | 67% (−33%) |
| Composition 2 | | | | |
| Neutral protease | | | | |
| (SEQ ID NO: 127) | | | | |
| [2 fl oz/unit] + | | | | |
| base seed treatment | | | | |

As shown in Table 10, nematicidal enzyme seed treatment compositions 1 & 2 reduced nematode presence in the soil surrounding treated plants as compared to the base seed treatment control. Nematicidal enzyme compositions 1 & 2 reduced accretion of nematodes from the genera Heterodera and Meloidogyne. Furthermore, these results demonstrate that nematicidal enzymes can reduce soil presence of nematodes in combination with agrochemicals present in the base seed treatment.

### Example 2

### Reduction of Soybean Cyst Nematode Reproduction in the Presence of Nematicidal Enzymes

Enzymes were produced by fermentation in Bacillus subtilis. Each enzyme was cloned into Bacillus expression plasmid p1797 with isopropyl β-d-1-thiogalactopyranoside (IPTG)-inducible expression. The plasmids were transformed into B. subtilis, and the strains were grown in rich media until an optical density (OD) of 0.2-0.5 was reached. Then, enzyme expression was induced by addition of 1 mM IPTG. Following the culture harvest, for treatments in which cell-free filtrates were used, the cells were removed by filtration through a 0.22 μM filter. Culture whole cell broth or filtrate containing secreted enzymes was then used for further applications.

To investigate effects of enzymes on soybean cyst nematode (SCN, Heterodera glycines), treatments were applied to soybean seed (variety P18A98X) with PI88788 resistance to SCN. Enzyme treatments were added at a rate of 2 fl. Oz. per unit of seed, mixed in a slurry with a colorant and Peridiam® 1006 and applied to seeds while vortexing in a tube. For each experiment, a base seed treatment control was included that contained only colorant and Peridiam® 1006. Additionally, enzyme treatments were compared to seed treated with the commercialized nematicidal product IIEVO® (49.02% fluopyram) applied at a rate of 0.15 mg active ingredient per seed. Seeds were then planted in soil in cone-tainers.

At the same time, eggs were obtained from SCN HG type 1.2.5.7, race 2. This population can reproduce on soybean plants with PI88788 resistance. Eggs were sterilized with 0.4% bleach and rinsed 3 times with sterile water. Eggs were divided evenly into the wells of a 6-well plate and resuspended into 5 mL of 3 mM ZnSO₄. The plate was sealed with parafilm, covered with aluminum foil, and placed in a plant growth chamber at 25° C. and 80-90% relative humidity.

At 6-7 days later, an estimate of hatched SCN juveniles was obtained from each well and averaged to determine the overall number of juveniles per μL. Around 500-1000 juveniles were applied to the base of 20-30 plants per seed treatment. This inoculation solution also contained unhatched eggs. Inoculated plants were placed in the growth chamber at 25° C. and 80-90% relative humidity with a 14 hour: 10 hour day: night cycle.

Three weeks after inoculation, cysts on each plant were counted. Plants and soil were removed from the containers. Soil was washed away from each root system and rinsed through nested sieves (#20 on top, #100 on the bottom) to collect particles between 150 μM and 850 μM along with cysts. The washed root systems were collected and dried in a 40° C. incubator for 5-6 days until tissue was dry. The soil particles containing cysts were mixed with an equal volume of a sucrose solution (454 g/L). Cysts float in this solution, so after soil particles settle to the bottom of the container, the liquid was poured off into a beaker. Particles were allowed to settle to the bottom a second time. Cysts floating to the top of the liquid were counted and recorded. Once a dry root mass was obtained for each plant, a final value of cysts per gram of root tissue was determined. To avoid the opportunity for any bias toward specific treatments during cyst counting, seed coating was performed by independent researchers and the treatment identities were blinded to the researchers who quantified the cysts.

To determine whether there were numerical or statistically significant differences in cysts per gram of root tissue between treatments, all experiments were analyzed in R (v4.1.1). For datasets in which variances were unequal, the data were transformed before analysis to meet assumptions of an analysis of variance (ANOVA). A one-way ANOVA was performed to detect differences among treatments for parameters that differed significantly (P<0.05). If the ANOVA indicated a significant difference existed among treatments, Tukey's HSD post hoc test was used to perform a pairwise contrast between each set of treatments. Different letters denote statistically significant differences at P<0.05.

Data for average cysts per gram of root tissue and comparison to base control can be found in Tables 11-13. Trypsin-like protease (SEQ ID NO: 126), neutral protease (SEQ ID NO: 127), esterase B (SEQ ID NO: 89), chitinase 19F (SEQ ID NO: 182), a Trichoderma viride chitinase, and Clostridium histolyticum collagenase A each showed a reduction of average cysts per gram of root tissue relative to the control treatment. Statistically significant differences were observed between control and trypsin-like protease, neutral protease, esterase B, and collagenase A.

TABLE 11
Average Cysts Per Gram of Root Tissue Compared to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 71.4 | 100% | 85.0 | n/a | a |
| Trypsin-like protease | 18.6 | 26% | 16.0 | <.0001 | b |
| (SEQ ID NO: 126) | | | | | |
| Neutral protease | 20.1 | 28% | 12.9 | .0018 | bc |
| (SEQ ID NO: 127) | | | | | |
| Esterase B | 19.6 | 27% | 12.4 | .0001 | b |
| (SEQ ID NO: 89) | | | | | |
| Chitinase 19F | 48.8 | 68% | 28.3 | .9708 | ac |
| (SEQ ID NO: 182) | | | | | |
| ILEVO ® | 26.6 | 37% | 26.3 | .0008 | b |

TABLE 12
Average Cysts Per Gram of Root Tissue Compared to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 376.9 | 100% | 254.9 | n/a | a |
| Chitinase 19F | 296.9 | 79% | 108.8 | .9997 | a |
| (SEQ ID NO: 182) | | | | | |
| Trichoderma viride | 325.5 | 86% | 175.4 | .9996 | a |
| chitinase | | | | | |
| ILEVO ® | 147.5 | 39% | 79.8 | .0002 | b |

TABLE 13
Average Cysts Per Gram of Root Tissue Compared to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 400.2 | 100% | 157.2 | n/a | a |
| Clostridium histolyticum | 105.6 | 26% | 25.7 | <.0001 | b |
| collagenase A | | | | | |

### Example 3

### Reduction of Soybean Cyst Nematode Reproduction by Recombinant Strain Expressing a Protease

A similar experimental protocol as described in Example 2 was performed with the exception that soybean seed (variety P18A98X) was treated with whole cell broth of enzyme produced via fermentation in Bacillus subtilis as described. Data for average cysts per gram of root tissue and comparison to control can be found in Table 14. Whole cell broth comprising neutral protease (SEQ ID NO: 127) showed a statistically significant reduction of average cysts per gram of root tissue relative to the base control treatment. Whole cell broth comprising esterase B (SEQ ID NO: 89) was similarly tested, and a reduction of average cysts per gram of root tissue was observed relative to control treatment (Table 15).

TABLE 14
Average Cysts Per Gram of Root Tissue Compared to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 286.6 | 100% | 134.1 | n/a | a |
| Neutral protease | 40.3 | 14% | 25.7 | .0001 | b |
| (SEQ ID NO: 127) | | | | | |

TABLE 15
Average Cysts Per Gram of Root Tissue Compared to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 59.8 | 100% | 35.8 | n/a | a |
| Esterase B | 56.3 | 94% | 32.6 | .9996 | a |
| (SEQ ID NO: 89) | | | | | |

### Example 4

Esterase Enzymes with Efficacy Against Soybean Cyst Nematode

To demonstrate efficacy of an additional esterase enzyme against nematodes, a controlled environment trial was performed with 7-day old soybean plants grown in pots. A Pseudomonas sp. esterase free enzyme (SEQ ID NO: 248) was produced and treated on seeds as described in Example 2. A concentrated version of the free enzyme was made by centrifuging fermentation broth in an Amicon Ultra 4K filter unit, resulting in a 4× volumetric concentration of the solution. Plant inoculation and quantification of cysts per gram of root tissue was conducted as described in Example 2, except that no bleach sterilization was performed during hatchery setup and soybean variety 2830E3 was used. The results were analyzed as in in Example 2 and can be found in Table 16. The Pseudomonas sp. esterase free enzyme (SEQ ID NO: 248) and concentrated free enzyme both reduced SCN reproduction on host plants. These results demonstrate that multiple esterase enzymes exhibit efficacy against plant nematode infection.

TABLE 16
Free Esterase Enzymes Reduce Average Cysts Per
Gram of Root Tissue Relative to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 244.3 | 100% | 142.5 | n/a | a |
| Pseudomonas sp. esterase | 220.2 | 90% (−10%) | 142.0 | .9997 | a |
| free enzyme | | | | | |
| (SEQ ID NO: 248) | | | | | |
| Pseudomonas sp. esterase | 182.1 | 75% (−25%) | 140.1 | .9295 | a |
| free enzyme concentrate | | | | | |
| (SEQ ID NO: 248) | | | | | |

### Example 5

### In-Vitro Reduction of Soybean Cyst Nematode Viability by an Additional Esterase Free Enzyme

To identify an additional esterase enzyme with efficacy in reducing SCN viability, we performed an in-vitro plate assay with a Pseudomonas sp. esterase (SEQ ID NO: 248) free enzyme direct treatment. The Pseudomonas sp. esterase (SEQ ID NO: 248) was produced by fermentation in Bacillus subtilis. The enzyme was cloned into a Bacillus expression plasmid p1797 with isopropyl β-d-1-thiogalactopyranoside (IPTG) inducible expression. The plasmid was transformed into B. subtilis, and the gene sequence was confirmed. The strain was grown in rich media until an optical density (OD) 0.2-0.5 was reached. Enzyme expression was then induced by addition of 1 M IPTG. Following the culture harvest, cells were removed by filtration through a 0.22 μM filter. The culture filtrate containing the secreted enzyme was then collected and used for further applications.

SCN eggs were obtained from SCN HG type 1.2.5.7, race 2. Eggs were rinsed 4 times with sterile water. Eggs were transferred to a 6-well plate and resuspended in 5 mL of 3 mM ZnSO₄. The plate was sealed with parafilm, covered with aluminum foil to prevent light from entering the plate, and placed in a plate growth chamber at 25° C. and 80% relative humidity. After 6-7 days of incubation, an estimate of hatched SCN juveniles was obtained from the well to determine the overall number of juveniles per 10 μL. The concentration of the hatchery solution was adjusted until it consisted of 20-30 juveniles per 10 μL.

Potato dextrose agar media containing chloramphenicol and chlortetracycline was prepared and added to sterile twenty-four-well cell culture plates (1 mL per well). The plates were divided according to the pre-determined N-value for each treatment (N=12-24). Each treatment being tested was added to the agar surface within each well at a rate of 40 μL per well. Without allowing the treatments to dry, 10 μL of the prepared nematode mixture was added to the agar surface within each well.

To quantify the treatments' effects on nematode viability, the nematodes were examined under a dissecting microscope at 20× magnification. Initial (T0) values for the number of live juveniles, number of dead juveniles and total number of juveniles were recorded immediately after preparation of each well. The plates were then wrapped in parafilm around the open edge, encased in aluminum foil, and placed in secondary containment. The secondary containment was placed in a plant growth chamber at 25° C. and 80% relative humidity. At approximately 24 hours post addition of juveniles, the plates were evaluated, and the number of live juveniles, dead juveniles and total number of juveniles were recorded (T24).

The difference in percent death at T0 vs T24 was calculated by subtracting the percent dead at TO [(#dead juveniles/total #juveniles)×100] from the percent dead at T24 [(#dead juveniles/#total juveniles originally present)×100]. To determine whether there were numerical or statistically significant differences, experiments were analyzed in R (v4.3.1). A one-way ANOVA was performed to detect differences among treatments for parameters that differed significantly (P<0.05). If the ANOVA indicated a significant difference existed among treatments, Tukey's HSD post hoc test was used to perform a pairwise contrast between each set of treatments. Different letters denote statistically significant results at P<0.05.

As seen in Table 17, the Pseudomonas sp. esterase (SEQ ID NO: 248) significantly reduced the viability of SCN juveniles after a 24-hour period as compared to the control treatment. Together, these and other results presented herein demonstrate that multiple esterase enzymes exhibit efficacy in reducing nematode viability.

TABLE 17
An Esterase Enzyme Increases Difference in Percent Death
of Nematodes for 0 to 24 Hours Compared to Water Control

| | Difference in | | |
|---|---|---|---|
| | Percent Death of | | |
| | Nematodes for 0 to | p-Value Relative | Pairwise |
| Treatment | 24 Hours | to Water Control | Contrast |
| Control (water) | 4% | n/a | a |
| Pseudomonas sp. | 22% | <0.0001 | b |
| esterase | | | |
| (SEQ ID NO: 248) | | | |

### Example 6

Free Chitinase Enzymes with Efficacy Against Soybean Cyst Nematode

To identify an additional chitinase enzyme with efficacy against SCN, we performed a controlled environment trial with chitinase C (SEQ ID NO: 249) free enzyme as a seed treatment. The chitinase enzyme was produced as in Example 2. Plant inoculation and quantification of cysts per gram of root tissue was conducted as described in Example 2. Results were analyzed as in Example 2 and can be found in Table 18. The chitinase C (SEQ ID NO: 249) seed treatment led to a reduction of SCN reproduction on host plants. These results indicate an additional chitinase enzyme has efficacy against SCN, in addition to the chitinases identified previously.

TABLE 18
Free Chitinase Reduces Average Cysts Per Gram
of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Base Control | 58.3 | 100% | 44.8 | n/a | a |
| Chitinase C | 45.7 | 78% (−22%) | 47.1 | .2296 | a |
| (SEQ ID NO: 249) | | | | | |

### Example 7

Identification of Lipase Enzymes with Efficacy Against Soybean Cyst Nematode

To identify lipase enzymes that-exhibit efficacy against pathogenic nematodes, we tested Bacillus thuringiensis lipase 1 (SEQ ID NO: 250) and Bacillus thuringiensis lipase 2 (SEQ ID NO: 251). The free enzyme was produced as in Example 2. Seed treatment and trial setup was performed as in Example 2, with the exception that soybean variety 2830E3 was used and no bleach sterilization was performed for the SCN hatchery. Cysts per gram of root tissue was determined as in Example 2. The results were analyzed as in Example 2 and can be found in Tables 19 and 20. Both lipase enzymes (SEQ ID NO: 250) and (SEQ ID NO: 251) reduced SCN reproduction on host plants. Thus, multiple lipase enzymes exhibit efficacy against a pathogenic nematode.

TABLE 19
Free Lipase Reduces Average Cysts Per Gram of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Base Control | 442.5 | 100% | 264.5 | n/a | a |
| Bacillus thuringiensis | 332.0 | 75% (−25%) | 290.8 | .7797 | a |
| Lipase 1 | | | | | |
| (SEQ ID NO: 250) | | | | | |

TABLE 20
Free Lipase Enzyme Reduces Average Cysts Per
Gram of Root Tissue Relative to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 459.7 | 100% | 308.8 | n/a | a |
| Bacillus thuringiensis | 264.3 | 57% (−43%) | 274.8 | .0512 | a |
| Lipase 2 | | | | | |
| (SEQ ID NO: 251) | | | | | |

### Example 8

### In-Vitro Reduction of Soybean Cyst Nematode Viability by a Collagenase Free Enzyme

The Bacillus thuringiensis collagenase A (SEQ ID NO: 122) was produced by fermentation in Bacillus subtilis. The enzyme was cloned into a Bacillus expression plasmid p1797 with isopropyl β-d-1-thiogalactopyranoside (IPTG) inducible expression. The plasmid was transformed into B. subtilis, and the gene sequence was confirmed. The strain was grown in rich media until an optical density (OD) 0.2-0.5 was reached. Enzyme expression was then induced by addition of 1 M IPTG. Following the culture harvest, cells were removed by filtration through a 0.22 μM filter. The culture filtrate containing the secreted enzyme was then collected and used for further applications.

SCN eggs were obtained from HG type 1.2.5.7, race 2. Eggs were rinsed four times with sterile water. Eggs were transferred to a 6-well plate and resuspended in 5 mL of 3 mM ZnSO₄. The plate was sealed with parafilm, covered with aluminum foil to prevent light from entering the plate, and placed in a plate growth chamber at 25° C. and 80-90% relative humidity. After 6-7 days of incubation, an estimate of hatched SCN juveniles was obtained from each well to determine the overall number of juveniles per 10 μL. The concentration of the hatchery solution was adjusted until it consisted of 20-30 juveniles per 10 μL.

Potato dextrose agar media containing chloramphenicol and chlortetracycline was prepared and added to sterile twenty-four-well cell culture plates (1 mL per well). The plates were divided according to the pre-determined N-value for each treatment (N=24). Each treatment being tested was added to the agar surface within each well at a rate of 40 μL per well. Without allowing the treatments to dry, 10 μL of the prepared nematode mixture was added to the agar surface within each well.

To quantify the treatments' effects on nematode viability, the nematodes were examined under a dissecting microscope at 20× magnification. Initial (T0) values for the number of live juveniles, number of dead juveniles and total number of juveniles were recorded. The plates were then wrapped in parafilm around the open edge, encased in aluminum foil, and placed in secondary containment. The secondary containment was placed in a plant growth chamber at 25° C. and 80-90% relative humidity. Approximately 24 hours after the addition of juveniles, the plates were evaluated again and the number of live juveniles, dead juveniles, and total number of juveniles were recorded (T24).

The difference in percent death at T0 vs T24 was calculated by subtracting the percent dead at TO [(#dead juveniles/total #juveniles)×100] from the percent dead at T24 [(#dead juveniles/#total juveniles originally present)×100]. To determine whether there were numerical or statistically significant differences, experiments were analyzed in R (v4.3.1). A one-way ANOVA was performed to detect differences among treatments for parameters that differed significantly (P<0.05). If the ANOVA indicated a significant difference existed among treatments, Tukey's HSD post hoc test was used to perform a pairwise contrast between each set of treatments. Different letters denote statistically significant results at P<0.05.

As seen in Table 21, the Bacillus thuringiensis Collagenase A (SEQ ID NO: 122) numerically reduced the viability of SCN juveniles after a 24-hour period as compared to the water control. Together, these and other results presented here demonstrate that multiple collagenase enzymes exhibit efficacy in reducing nematode viability.

TABLE 21
A Collagenase Enzyme Increases Difference in Percent Death
of Nematodes for 0 to 22 Hours Compared to Water Control

| | Difference in Percent | p-Value | |
|---|---|---|---|
| | Death of Nematodes | Relative to | Pairwise |
| Treatment | for 0 to 22 Hours | Water Control | Contrast |
| Control (Water) | 3% | N/A | a |
| Bacillus | 9% | 0.2228 | a |
| thuringiensis | | | |
| Collagenase A | | | |
| (SEQ ID NO: 122) | | | |
| ILEVO ® | 67% | <0.0001 | b |

### Example 9

Identification of an Additional Collagenase Enzyme with Efficacy Against Soybean Cyst Nematode

To demonstrate that an additional collagenase has efficacy against pathogenic nematodes, we examined collagenase B (SEQ ID NO: 208) in a controlled environment seed treatment trial using SCN. The free enzyme was produced as in Example 2. Seed treatment and trial setup was performed as in Example 2, with the exceptions that soybean variety 2830E3 was used and no bleach sterilization was used for the SCN hatchery. Cysts per gram of root tissue was determined as in Example 2. The results were analyzed as in Example 2 and can be found in Table 22. The collagenase B free enzyme (SEQ ID NO: 208) reduced SCN reproduction on host plants relative to the base control. These results indicated multiple collagenases exhibit efficacy against pathogenic nematodes.

TABLE 22
An Additional Collagenase Enzyme Reduce Average Cysts
Per Gram of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Base Control | 470.7 | 100% | 309.6 | n/a | a |
| Collagenase B | 70.9 | 15% (−85%) | 45.5 | <.0001 | b |
| (SEQ ID NO: 208) | | | | | |

### Example 10

Steinernema Feltiae Nematode Death Following Direct Treatment with Enzymes

To determine the effects of enzymes on an additional soil-dwelling nematode species (Steinernema feltiae), a NemAttack™ package containing approximately 5 million S. feltiae juveniles was purchased from ARBICO Organics. The nematodes arrived already hatched and dispersed within a powder. To prepare the nematodes for use, an aliquot of the dry nematode mixture was rehydrated in water. The liquid nematode mixture was adjusted to a concentration of 20-30 nematode juveniles per 10 μL. The free enzymes Esterase B (SEQ ID NO: 89) and Chitinase 19F (SEQ ID NO: 182) were produced as described in Example 2.

Potato dextrose agar media containing chloramphenicol and chlortetracycline was prepared and added to sterile twenty-four-well cell culture plates (1 mL per well). The plates were divided according to the pre-determined N-value for each treatment (N=12-24). Each treatment being tested was added to the agar surface within each well at a rate of 40 μL per well except where 20 μL was added per well for ILEVO® (48.4% fluopyram), a commercial nematocidal, for use as a positive control. Without allowing the treatments to dry, 10 μL of the prepared nematode mixture was added to the agar surface within each well.

To quantify the treatments' effects on nematode viability, the nematodes were examined under a dissecting microscope at 20× magnification. Initial (T0) values for the number of live juveniles, number of dead juveniles and total number of juveniles were recorded immediately after preparation of each well. The plates were then wrapped in parafilm around the open edge, encased in aluminum foil to prevent light from entering the plate and placed in secondary containment. The secondary containment was placed in a plant growth chamber at 25° C. and 80-90% relative humidity. Approximately 20-22 hours post addition of juveniles, the plates were evaluated, and the number of live juveniles, dead juveniles and total number of juveniles were recorded.

The difference in percent dead nematodes between the start and end of the experiment was calculated by subtracting the percent dead at the start [(#dead juveniles/total #juveniles)×100] from the percent dead at the end [(#dead juveniles/#total juveniles originally present)×100]. To determine whether there were numerical or statistically significant differences, experiments were analyzed in R (v4.3.1). A one-way ANOVA was performed to detect differences among treatments for parameters that differed significantly (P<0.05). If the ANOVA indicated a significant difference existed among treatments, Tukey's HSD post hoc test was used to perform a pairwise contrast between each set of treatments. Different letters denote statistically significant results at P<0.05. The results can be found in Tables 23 and 24. The enzymes Esterase B (SEQ ID NO: 89) and Chitinase 19F (SEQ ID NO: 182) both significantly increased the difference in percentage of S. feltiae juveniles that died during the experiment. Thus, esterase and chitinase enzymes can decrease viability of an additional nematode species, apart from SCN as shown in previous examples.

TABLE 23
Esterase Treatment Increased Difference in Percent
Death of Nematodes Relative to Control

| | Difference in Percent | p-value | |
|---|---|---|---|
| | Death of Nematodes | relative to | Pairwise |
| Treatment | for 0 to 20 Hours | water only | contrast |
| Control (Water) | 3% | n/a | a |
| Esterase B(SEQ | 33% | <.0001 | b |
| ID NO: 89) | | | |
| ILEVO ® | 31% | <.0001 | b |

TABLE 24
Chitinase Treatment Increased the Difference in
Percent Death of Nematodes Relative to Control

| | Difference in Percent | | |
|---|---|---|---|
| | Death of Nematodes | p-value relative | Pairwise |
| Treatment | for 0 to 22 Hours | to water only | contrast |
| Control (Water) | 7% | n/a | a |
| Chitinase 19F | 17% | .0304 | b |
| (SEQ ID NO: 182) | | | |
| ILEVO | 29% | <.0001 | c |

### Example 10

### Reduction of Soybean Cyst Nematode Reproduction by a Dried Esterase

A dried formulation of esterase B (SEQ ID NO: 89) was created by combining the enzyme produced in Bacillus subtilis fermentation media as in Example 2 with 5.66% v/v polyvinylpyrrolidone. The mixture was air-dried at room temperature, and the film produced was ground into small particles with a mortar and pestle. These particles were mixed directly into a soybean seed treatment slurry containing colorant and Peridiam® 1006 (saxagliptin hydrochloride/dapagliflozin propanediol). The dried formulation was tested at 0.04 units of enzyme activity per seed, while the liquid fermentation media with enzyme was added in a separate treatment at 2 fl oz per unit of seed. The effects of seed treatments against SCN infection were investigated as described in Example 2, except that soybean variety 3230E3 was used and no bleach sterilization was performed for the SCN hatchery. The results for average cysts per gram of root tissue were analyzed in Example 2 and can be found in Table 25. Both the liquid and dry formulations of esterase B (SEQ ID NO: 89) decreased cysts relative to the base control. Thus, a dry formulation of an esterase enzyme can provide efficacy in controlling a pathogenic nematode.

TABLE 25
Average Cysts Per Gram of Root Tissue Relative to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 289.9 | 100% | 262.5 | n/a | a |
| Esterase B dried | 121.4 | 42% | 66.8 | .1450 | a |
| formulation (SEQ ID NO: | | | | | |
| 89) | | | | | |
| Esterase B liquid | 233.2 | 80% | 186.6 | .9977 | a |
| formulation (SEQ ID NO: | | | | | |
| 89) | | | | | |

### Example 11

Protease Seed Treatment Reduces the Root Gall Index of Soybeans Inoculated with Meloidogyne incognita Plant Parasitic Nematodes

Nematicidal neutral protease (SEQ ID NO: 127) was produced by fermentation in Bacillus subtilis. The enzyme was cloned into Bacillus expression plasmid p1797 with isopropyl β-d-1-thiogalactopyranoside (IPTG)-inducible expression. The plasmid was transformed into B. subtilis, and this strain was grown in rich media until an optical density (OD) of 0.2-0.5 was reached. Then, enzyme expression was induced by addition of 1 mM IPTG. Following the culture harvest, cells were removed by filtration through a 0.22 μM filter. Culture filtrate containing secreted protease was then used for seed treatment. Bacillus subtilis fermentation filtrate expressing neutral protease was mixed directly into a seed treatment slurry containing colorant and Peridiam® 1006 (saxagliptin hydrochloride/dapagliflozin propanediol) at a rate of 2 fl oz per unit of seed and coated onto Bossier variety soybean seed.

To investigate the effects of neutral protease on galling due to root knot nematode infestation, treated seeds were planted in soil in cone-tainers in a greenhouse. Seven days after planting, each cone-tainer was inoculated with 1250 eggs from the Dick Meloidogyne incognita population. Eight weeks post-inoculation, roots were excavated and washed. Root galling was visually rated on a gall index (GI) scale of 1-5 (1=<10% GI; 2=11-20%, 3=21-30%, 4=31-40%, 5=>40% GI). Fifteen plants were evaluated per treatment and their average root gall ratings were recorded. To determine whether there were numerical or statistically significant differences in average root gall ratings between treatments, pairwise Student's t-tests were performed. Different letters denote statistically significant differences at P<0.05.

Data for average root gall rating of neutral protease liquid seed treatment and comparison to base control can be found in Table 26. Neutral protease (SEQ ID NO: 127) displayed a reduction of average visual gall rating relative to the control treatment at eight weeks after inoculation with the plant parasitic nematode Meloidogyne incognita. These results demonstrate that a nematicidal protease provides efficacy against a second pathogenic nematode species, apart from SCN as shown in previous examples.

TABLE 26
Protease seed treatment reduces the root gall index of soybeans
inoculated with Meloidogyne incognita plant parasitic nematodes

| | | % | p-value | Pairwise t- |
|---|---|---|---|---|
| | Average root | Comparison | relative to | test |
| Treatment | gall rating | to Control | control | comparison |
| Base seed treatment control | 4.7 | 100% | n/a | a |
| Neutral protease seed | 3.5 | 74% (−26%) | <0.05 | b |
| treatment | | | | |
| (SEQ ID NO: 127) | | | | |

### Example 12

Reduction of Soybean Cyst Nematode Reproduction by Chitinase Free Enzyme in Combination with an Agrochemical

To determine whether a chitinase free enzyme exhibits efficacy against SCN in combination with an agrochemical, a potted-plant greenhouse trial was conducted using soybean variety P31T64E. Seed treatment was conducted as in Example 2 with the exception that Maxim® 4FS fungicide (fludioxonil 40.3%), was added to the base seed treatment. The chitinase 19F (SEQ ID NO: 182) free enzyme was added in combination with Maxim® 4FS in a separate treatment. For each treatment, approximately seven days after planting each soybean plant was inoculated with 1000 nematode juveniles from a SCN HG type 1.2.5.7, race 2 population. Thirty days post-experimental setup, the aboveground soybean was removed at the soil line, and soybean roots were washed clean to remove the soil. SCN cysts were dislodged from roots using a high-pressure spray nozzle and collected on a 250-um sieve under an 850-um sieve. Cysts per experimental unit were counted using a dissecting microscope. Aboveground and root system systems were weighed fresh and dried (24 hours at 120° F.). Twelve plants were evaluated per treatment and their average cysts per gram of root tissue was recorded. The results were analyzed as in Example 2 and can be found in Table 27. The chitinase free enzyme in combination with Maxim® 4FS reduced SCN reproduction on host plants. These results indicated a nematicidal enzyme can exhibit efficacy against a pathogenic nematode in combination with an agrochemical.

TABLE 27
Chitinase Free Enzyme in Combination with an Agrochemical Reduces
Average Cysts Per Gram of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Maxim ® 4FS Control | 290.8 | 100% | 185.2 | n/a | a |
| [0.004 mg AI/seed] | | | | | |
| Maxim ® 4FS | 266.6 | 92% (−8%) | 128.7 | 1.000 | a |
| [0.004 mg AI/seed] + | | | | | |
| Chitinase 19F | | | | | |
| (SEQ ID NO: 182) | | | | | |
| [2.0 fl oz/unit] | | | | | |

### Example 13

In-Vitro Reduction of Soybean Cyst Nematode Viability by an Esterase Free Enzyme in Combination with Fertilizer

The Esterase B (SEQ ID NO: 89) was produced by fermentation in Bacillus subtilis. The enzyme was cloned into a Bacillus expression plasmid p1797 with isopropyl B-d-1-thiogalactopyranoside (IPTG) inducible expression. The plasmid was transformed into B. subtilis, and the gene sequence was confirmed. The strain was grown in rich media until an optical density (OD) 0.5-1.0 was reached. Enzyme expression was then induced by addition Of 1 M IPTG. Following the culture harvest, cells were removed by filtration through a 0.22 μM filter. The culture filtrate containing the secreted enzyme was then collected and used for further applications.

To determine whether an esterase enzyme in combination with an agrochemical can reduce viability of SCN, a fertilizer was obtained to apply to plates with the free enzyme esterase filtrate. An ammonium polyphosphate liquid fertilizer (Plant Food Co, 10-34-0; 10% Nitrogen, 34% Phosphate and 0% Potassium) was ordered from Reinders in a 2.5-gallon jug quantity. This 10-34-0 fertilizer was diluted in milli-Q water to achieve an application rate of 3% v/v fertilizer to represent a field application rate.

SCN eggs were obtained from SCN HG type 1.2.5.7, race 2. Eggs were rinsed 4 times with sterile water. Eggs were transferred to a 6-well plate and resuspended in 5 mL of 3 mM ZnSO₄. The plate was sealed with parafilm, covered with aluminum foil to prevent light from entering the plate, and placed in a plate growth chamber at 25° C. and 80-90% relative humidity. After 6-7 days of incubation, an estimate of hatched SCN juveniles was obtained from the well to determine the overall number of juveniles per 10 μL. The concentration of the hatchery solution was adjusted until it consisted of 20-30 juveniles per 10 μL.

Potato dextrose agar media containing chloramphenicol and chlortetracycline was prepared and added to sterile twenty-four-well cell culture plates (1 mL per well). The plates were divided according to the pre-determined N-value for each treatment (N=12-24). Each treatment being tested was added to the agar surface within each well at a rate of 40 μL per well, for a total of 80 μL of combined treatment volume added. Without allowing the treatments to dry, 10 μL of the prepared nematode mixture was added to the agar surface within each well.

To quantify the treatments' effects on nematode viability, the nematodes were examined under a dissecting microscope at 20× magnification. Initial (T0) values for the number of live juveniles, number of dead juveniles and total number of juveniles were recorded immediately after preparation of each well. The plates were then wrapped in parafilm around the open edge, encased in aluminum foil, and placed in secondary containment. The secondary containment was placed in a plant growth chamber at 25° C. and 80-90% relative humidity. Approximately 24 hours post addition of juveniles, the plates were evaluated, and the number of live juveniles, dead juveniles and total number of juveniles were recorded (T24).

The difference in percent death at T0 vs T24 was calculated by subtracting the percent dead at TO [(#dead juveniles/total #juveniles)×100] from the percent dead at T24 [(#dead juveniles/#total juveniles originally present)×100]. To determine whether there were numerical or statistically significant differences, experiments were analyzed in R (v4.3.1). A one-way ANOVA was performed to detect differences among treatments for parameters that differed significantly (P<0.05). If the ANOVA indicated a significant difference existed among treatments, Tukey's HSD post hoc test was used to perform a pairwise contrast between each set of treatments. Different letters denote statistically significant results at P<0.05.

As shown in Table 28, treatment with Esterase B (SEQ ID NO: 89) applied in combination with the 10-34-0 ammonium polyphosphate liquid fertilizer provided a statistically significant reduction in viability of SCN juveniles, when observed over a twenty-four-hour timeframe, compared to either treatment applied individually. The observed difference in percent death over twenty-four hours for the combination treatment exceeded the difference in percent death for either treatment, a −7% and −6% reduction for Esterase B alone and 10-34-0 fertilizer alone, respectively, demonstrating increased benefit in reducing nematode viability for the combination of the esterase B enzyme with the agrochemical 10-34-0 fertilizer. In summary, these results indicate that a nematicidal enzyme can exhibit efficacy in reducing nematode viability when combined with an agrochemical.

TABLE 28
Combination of an Esterase with an Agrochemical (Fertilizer)
Increases Difference in Percent Death of Nematodes
for 0 to 24 Hours Compared to Water Control

| | Difference in Percent Death of | |
|---|---|---|
| Treatment | Nematodes for 0 to 24 Hours | Pairwise Contrast |
| Esterase B | 3% | a |
| (SEQ ID NO: 89) | | |
| Fertilizer 10-34-0 | 4% | a |
| Esterase B | 10% | b |
| (SEQ ID NO: 89) + | | |
| Fertilizer 10-34-0 | | |

### Example 14

Reduction of Soybean Cyst Nematode Reproduction by Recombinant Strain Expressing an Enzyme in Combination with an Agrochemical

To determine whether an esterase-expressing recombinant strain exhibits efficacy against SCN in combination with an agrochemical, we conducted a controlled environment seed treatment trial as described in Example 2, with the exception that Maxim® 4FS fungicide (fludioxonil 40.3%), was added to the base seed treatment. The esterase B recombinant strain (SEQ ID NO: 89) was produced as in Example 2 and was added to a seed treatment in combination with Maxim® 4FS. Plant inoculation and quantification of cysts per gram of root tissue was conducted as described in Example 2, except that no bleach sterilization was performed for the SCN hatchery. Cysts per gram of root tissue for each treatment was determined as in Example 2. The results were analyzed as in Example 2 and can be found in Table 29. The esterase-expressing recombinant strain (SEQ ID NO: 89) seed treatment led to a decrease in SCN reproduction on host plants. These results indicate that a recombinant strain expressing a nematicidal enzyme can exhibit efficacy against a pathogenic nematode when combined with an agrochemical.

TABLE 29
Combination of an Agrochemical with a Recombinant Strain Expressing an Enzyme
Reduces Average Cysts Per Gram of Root Tissue Relative to Base Control

| | Average cysts | % | | p-value | |
|---|---|---|---|---|---|
| | per g of root | Comparison | Standard | relative to | Pairwise |
| Treatment | tissue | to Control | deviation | Control | contrast |
| Maxim ® 4FS Control | 219.2 | 100% | 103.41 | n/a | a |
| Maxim ® 4FS + | 205.6 | 94% (−6%) | 121.93 | .9999 | a |
| Esterase B | | | | | |
| recombinant strain | | | | | |
| (SEQ ID NO: 89) | | | | | |

### Example 15

Combination of a Nematicidal Enzyme with a Commercial Chemical Nematicide (Agrochemical) Provides Benefit of Increased Efficacy

Nematicidal neutral protease was produced by fermentation in Bacillus subtilis. The enzyme was cloned into Bacillus expression plasmid p1797 with isopropyl β-d-1-thiogalactopyranoside (IPTG)-inducible expression. The plasmid was transformed into B. subtilis, and this strain was grown in rich media until an optical density (OD) of 0.2-0.5 was reached. Then, enzyme expression was induced by addition of 1 mM IPTG. Following the culture harvest, cells were removed by filtration through a 0.22 μM filter. A dried formulation of neutral protease H184 (SEQ ID NO: 127) was created by combining the above filtrate with 5.66% v/v polyvinylpyrrolidone (PVP). This mixture was poured into a thin film, air-dried at room temperature, then the film was broken into small particles with a mortar and pestle. The resulting dried particles comprised of fermented protease filtrate+PVP were mixed directly into a soybean seed treatment slurry containing colorant and Peridiam® 1006 (saxagliptin hydrochloride/dapagliflozin propanediol) and coated onto soybean seed of the P31T64E variety.

To test for additive and/or synergistic effects between combinations of protease and a commercial nematicidal agrochemical seed treatment (Saltro®; 41.7% pydiflumetofen) in providing protection against soybean cyst nematode infestation, a greenhouse trial was conducted in potted plants. For each treatment, approximately seven days after planting each soybean plant was inoculated with 1000 nematode juveniles from a SCN HG type 1.2.5.7, race 2 population. Thirty days post-experimental setup, the aboveground soybean was removed at the soil line, and soybean roots were washed clean to remove the soil. SCN females were dislodged from roots using a high-pressure spray nozzle and collected on a 250-um sieve under an 850-um sieve. SCN females per experimental unit were counted using a dissecting microscope. Aboveground and root system systems were weighed fresh and dried (24 hours at 120° F.). Twelve plants were evaluated per treatment and their average females/cysts per gram of root tissue was recorded.

Seed treatment of a nematicidal enzyme (dried neutral protease) in combination with a commercial nematicidal agrochemical seed treatment (Saltro®; 41.7% pydiflumetofen) provided increased plant protection against SCN infection over the base seed treatment; females per gram of root tissue were reduced by 18%. The observed 18% reduction in females per gram of root tissue for the combination exceeded the reductions for each composition alone, −5% and −7% for dried protease formulation and Saltro® (41.7% pydiflumetofen), respectively, demonstrating synergistic benefit for combination of the nematicidal enzyme (dried neutral protease) with a nematicidal agrochemical seed treatment as shown in Table 30.

TABLE 30
Increased Efficacy Effect Observed for a Dried Nematicidal Enzyme in Combination
with a Commercial Chemical Nematicide, Saltro ® (41.7% pydiflumetofen)

| | Average females | % | | p-value | |
|---|---|---|---|---|---|
| | (cysts) per g of | Comparison | Standard | relative to | Pairwise |
| Treatment | root tissue | to Control | deviation | Control | contrast |
| Base Control | 371.7 | 100% | 197.9 | n/a | a |
| Neutral protease dried | 345.2 | 93% (−7%) | 134.8 | 1.000 | a |
| formulation | | | | | |
| (SEQ ID NO: 127) | | | | | |
| [0.04 U/seed] | | | | | |
| Saltro ® [0.075 mg | 353.1 | 95% (−5%) | 107.1 | 1.000 | a |
| AI/seed] | | | | | |
| Neutral protease dried | 303.3 | 82% (−18%) | 108.7 | .9942 | a |
| formulation | | | | | |
| (SEQ ID NO: 127) | | | | | |
| [0.04 U/seed] + | | | | | |
| Saltro ® [0.075 mg | | | | | |
| AI/seed] | | | | | |

### Example 16

Combination of a Nematicidal Enzyme with a Different Commercial Chemical Nematicide (Agrochemical) Provides Benefit of Increased Efficacy

Nematicidal neutral protease (SEQ ID NO: 127) was produced by fermentation in Bacillus subtilis. The enzyme was cloned into Bacillus expression plasmid p1797 with isopropyl β-d-1-thiogalactopyranoside (IPTG)-inducible expression. The plasmid was transformed into B. subtilis, and this strain was grown in rich media until an optical density (OD) of 0.2-0.5 was reached. Then, enzyme expression was induced by addition of 1 mM IPTG. Following the culture harvest, cells were removed by filtration through a 0.22 μM filter. A dried formulation of neutral protease (SEQ ID NO: 127) was created by combining the above filtrate with 5.66% v/v polyvinylpyrrolidone (PVP). This mixture was poured into a thin film, air-dried at room temperature, then the film was broken into small particles with a mortar and pestle. The resulting dried particles comprised of fermented protease filtrate+PVP were mixed directly into a soybean seed treatment slurry containing colorant and Peridiam® 1006 (saxagliptin hydrochloride/dapagliflozin propanediol) and coated onto soybean seed of the Becks variety 2830E3. To test for additive and/or synergistic effects between combinations of protease and a commercial nematicidal agrochemical seed treatment (ILEVO®; 48.4% fluopyram) in providing protection against SCN infestation, a grow chamber experiment was conducted in cone-tainers containing a loamy sand soil mixture (Hummert International: 80% sand, 15% silt, 5% clay). SCN inoculation, determination of cysts per gram of root tissue, and data analysis were performed as in Example 2

Seed treatment of a nematicidal enzyme (dried neutral protease) in combination with a commercial nematicidal agrochemical seed treatment (ILEVO®; 48.4% fluopyram) provided increased plant protection against SCN infection over the base seed treatment; cysts per gram of root tissue were reduced by 85%. The observed 85% reduction in cysts per gram of root tissue for the combination exceeded the reductions for each composition alone, −10% and −80% for dried protease formulation and ILEVO® (48.4% fluopyram), respectively, demonstrating increased benefit for combination of the nematicidal enzyme (dried neutral protease) with a nematicidal agrochemical seed treatment as shown in Table 31.

TABLE 31
Increased Efficacy Observed for a Dried Nematicidal Enzyme in Combination with
a Commercial Chemical Nematicide, ILEVO ® (48.4% fluopyram)

| | | % | | p-value | |
|---|---|---|---|---|---|
| | Average cysts per | Comparison | Standard | relative to | Pairwise |
| Treatment | g of root tissue | to Control | deviation | Control | contrast |
| Base Control | 425.7 | 100% | 342.3 | n/a | a |
| Neutral protease dried | 384.2 | 90% (−10%) | 198.5 | 1.000 | a |
| formulation | | | | | |
| (SEQ ID NO: 127) | | | | | |
| [0.04 U/seed] | | | | | |
| ILEVO ® [0.025 mg | 84.8 | 20% (−80%) | 79.3 | <.0001 | b |
| AI/seed] | | | | | |
| Neutral protease dried | 65.9 | 15% (−85%) | 41.0 | <.0001 | b |
| formulation | | | | | |
| (SEQ ID NO: 127) | | | | | |
| [0.04 U/seed] + | | | | | |
| ILEVO ® [0.025 mg | | | | | |
| AI/seed] | | | | | |

### Example 17

In-Vitro Reduction of Soybean Cyst Nematode Viability by an Esterase Free Enzyme in Combination with a Chitinase Free Enzyme

To determine whether combination of an esterase with a chitinase provides enhanced efficacy in reducing SCN viability, we performed an in-vitro plate assay with direct treatment. The Pseudomonas sp. esterase (SEQ ID NO: 248) and chitinase 19F (SEQ ID NO: 182) were produced by fermentation in Bacillus subtilis. The enzymes were each separately cloned into a Bacillus expression plasmid p1797 with isopropyl β-d-1-thiogalactopyranoside (IPTG) inducible expression. The plasmids were transformed into B. subtilis, and the gene sequences were confirmed. The strains were grown in rich media until an optical density (OD) 0.2-0.5 was reached. Enzyme expression was then induced by addition 0 of 1 mM IPTG. Following the culture harvest, cells were removed by filtration through a 0.22 μM filter. Each of the culture filtrates containing the secreted enzymes were then collected and used for further applications.

SCN eggs were obtained from SCN HG type 1.2.5.7, race 2. Eggs were rinsed 4 times with sterile water. Eggs were transferred to a 6-well plate and resuspended in 5 mL of 3 mM ZnSO₄. The plate was sealed with parafilm, covered with aluminum foil to prevent light from entering the plate, and placed in a plate growth chamber at 25° C. and 80-90% relative humidity. At 6-7 days later, an estimate of hatched SCN juveniles was obtained from the well to determine the overall number of juveniles per 10 μL. The concentration of the hatchery solution was adjusted until it consisted of 20-30 juveniles per 10 μL.

Potato dextrose agar media containing chloramphenicol and chlortetracycline was prepared and added to sterile twenty-four-well cell culture plates (1 mL per well). The plates were divided according to the pre-determined N-value for each treatment (N=12-24). Each treatment being tested was added to the agar surface within each well at a typical rate of 40 μL per well (40 μL for each treatment of the combination for a total volume of 80 μL). Without allowing the treatments to dry, 10 μL of the prepared nematode mixture was added to the agar surface within each well.

To quantify the treatments' effects on nematode viability, the nematodes were examined under a dissecting microscope at 20× magnification. Initial (T0) values for the number of live juveniles, number of dead juveniles and total number of juveniles were recorded immediately after preparation of each well. The plates were then wrapped in parafilm around the open edge, encased in aluminum foil, and placed in secondary containment. The secondary containment was placed in a plant growth chamber at 25° C. and 80-90% relative humidity. Approximately 24 hours post addition of juveniles, the plates were evaluated again, and the number of live juveniles, dead juveniles and total number of juveniles were recorded (T24).

The difference in percent death at T0 vs T24 was calculated by subtracting the percent dead at TO [(#dead juveniles/total #juveniles)×100] from the percent dead at T24 [(#dead juveniles/#total juveniles originally present)×100]. To determine whether there were numerical or statistically significant differences, experiments were analyzed in R (v4.3.1). A one-way ANOVA was performed to detect differences among treatments for parameters that differed significantly (P<0.05). If the ANOVA indicated a significant difference existed among treatments, Tukey's HSD post hoc test was used to perform a pairwise contrast between each set of treatments. Different letters denote statistically significant results at P<0.05.

As seen in Table 32, treatment consisting of a combination of an esterase with a chitinase effectively reduced nematode viability compared to the water control. The observed 25% difference in percent death during 24 hours for the combination, exceeded the percent death for either composition alone (24% and 22% for chitinase 19F (SEQ ID NO: 182) and the Pseudomonas sp. esterase (SEQ ID NO: 248), respectively), demonstrating increased benefit for combination of an esterase and a chitinase. Furthermore, these results indicate that a combination of two nematicidal enzymes can provide increased nematicidal efficacy.

TABLE 32
Treatment with a Combination of an Esterase and a Chitinase
Increases Difference in Percent Death of Nematodes
for 0 to 24 Hours Compared to Water Control

| | Difference in Percent | p-Value | |
|---|---|---|---|
| | Death of Nematodes | Relative to | Pairwise |
| Treatment | for 0 to 24 Hours | Water Only | Contrast |
| Water only (Control) | 4% | n/a | a |
| Chitinase 19F (SEQ | 24% | <.0001 | b |
| ID NO: 182) | | | |
| Pseudomonas sp. | 22% | <.0001 | b |
| esterase free enzyme | | | |
| (SEQ ID NO: 248) | | | |
| Pseudomonas sp. | 25% | <.0001 | b |
| esterase free enzyme | | | |
| (SEQ ID NO: 248) + | | | |
| Chitinase 19F | | | |
| (SEQ ID NO: 182) | | | |

### Example 18

### Reduction of Soybean Cyst Nematode Reproduction by Enzymes Applied to Soil

To determine the efficacy of nematicidal enzymes applied to soil, a controlled environment trial was performed with 7-day old soybean plants (variety 2830E3) grown in pots. Enzymes were produced as described in Example 2. Prior to inoculation, 0.5 mL of neutral protease (SEQ ID NO: 127), esterase B (SEQ ID NO: 89), or chitinase 19F (SEQ ID NO: 182) free enzyme solution was applied to the soil around the base of each plant, or 0.5 mL of water was applied for the control (30 plants per treatment). Then, hatched SCN juveniles were applied to the soil around each plant as described in Example 2, and three weeks later, cysts per gram of root tissue was determined as described in Example 2. The data was analyzed as in Example 2 and appears in Tables 33 and 34. The results indicate that treatment of neutral protease (SEQ ID NO: 127), esterase B (SEQ ID NO: 89), and chitinase 19F (SEQ ID NO: 182) free enzymes on soil surrounding a plant leads to reduced SCN reproduction.

TABLE 33
Soil Application of Protease Reduces Average Cysts
Per Gram of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Control (Water) | 52.4 | 100% | 50.2 | n/a | a |
| Neutral protease free | 35.7 | 68% (−32%) | 27.3 | .9964 | a |
| enzyme [0.5 mL/plant] | | | | | |
| (SEQ ID NO: 127) | | | | | |

TABLE 34
Soil Application of Esterase and Chitinase Reduces Average
Cysts Per Gram of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Control (Water) | 139.8 | 100% | 88.9 | n/a | a |
| Esterase B free enzyme | 117.3 | 84% (−16%) | 69.3 | .9945 | a |
| [0.5 mL/plant] | | | | | |
| (SEQ ID NO: 89) | | | | | |
| Chitinase 19F free enzyme | 95.6 | 68% (−32%) | 57.4 | .8120 | a |
| [0.5 mL/plant] | | | | | |
| (SEQ ID NO: 182) | | | | | |

### Example 19

### Reduction of Soybean Cyst Nematode Reproduction by a Recombinant Strain Applied to Soil

To determine the efficacy of recombinant strains applied to soil, a controlled environment trial was performed as described in Example 18 above (Reduction of soybean cyst nematode reproduction by enzymes applied to soil), with the exception that a recombinant strain expressing neutral protease (SEQ ID NO: 127) was applied instead of a free enzyme solution. The recombinant strain was produced as in Example 3, and the treatment applied to soil as a whole cell broth. The results were analyzed as in Example 2 and can be found in Table 35. The application of a recombinant strain expressing neutral protease to soil surrounding plants leads to reduction of SCN reproduction.

TABLE 35
Soil Application of a Recombinant Strain Expressing an Enzyme Reduces
Average Cysts Per Gram of Root Tissue Relative to Base Control

| | | % | | p-value | |
|---|---|---|---|---|---|
| | Average cysts per | Comparison | Standard | relative to | Pairwise |
| Treatment | g of root tissue | to Control | deviation | Control | contrast |
| Water only control | 139.8 | 100% | 88.9 | n/a | a |
| Neutral protease | 55.7 | 40% (−60%) | 36.9 | .0211 | b |
| recombinant strain | | | | | |
| [0.5 mL/plant] | | | | | |
| (SEQ ID NO: 127) | | | | | |

### Example 20

### Reduction of Soybean Cyst Nematode Reproduction by a Recombinant Strain Expressing a Protease

Nematicidal enzymes were produced by fermentation in Bacillus subtilis. Each enzyme was cloned into Bacillus expression plasmid p1797 with isopropyl β-d-1-thiogalactopyranoside (IPTG)-inducible expression. The plasmids were transformed into B. subtilis, and the strains were grown in rich media until an optical density (OD) of 0.2-0.5 was reached. Then, enzyme expression was induced by addition of 1 mM IPTG. Following the culture harvest, culture whole cell broth containing secreted enzymes was then used for further applications.

To investigate effects of enzymes on SCN, treatments were applied to soybean seed with PI88788 resistance to SCN. Enzyme treatments were added at a rate of 2 fl. oz. per unit of seed, mixed in a slurry with a colorant and Peridiam® 1006 (saxagliptin hydrochloride/dapagliflozin propanediol) and applied to seeds while vortexing in a tube. For each experiment, a base seed treatment control was included that contained only colorant and Peridiam® 1006 (saxagliptin hydrochloride/dapagliflozin propanediol). These seeds were then planted in soil in cone-tainers containing a loamy sand soil mixture (Hummert International: 80% sand, 15% silt, 5% clay). Plant inoculation, determination of cysts per gram of root tissue, and data analysis were performed as in Example 2

Data for average cysts per gram of root tissue for seed treatment with a recombinant strain expressing neutral protease and comparison to base control can be found in Table 36. Neutral protease recombinant strain (SEQ ID NO: 127) displayed an 86% reduction of average cysts per gram of root tissue relative to the control treatment at three weeks after inoculation with the plant parasitic nematode SCN, indicating that protease recombinant strain seed treatment reduces SCN reproduction on treated plants.

TABLE 36
Reduction of Soybean Cyst Nematode Reproduction
by a Recombinant Strain Expressing a Protease

| | | % | | p-value | |
|---|---|---|---|---|---|
| | Average cysts per | Comparison | Standard | relative to | Pairwise |
| Treatment | g of root tissue | to Control | deviation | Control | contrast |
| Base Control | 286.6 | 100% | 134.1 | n/a | a |
| Neutral protease | 40.4 | 14% (−86%) | 25.7 | .0008 | b |
| recombinant strain | | | | | |
| (SEQ ID NO: 127) | | | | | |

### Example 21

### Reduction of Soybean Cyst Nematode Reproduction by a Recombinant Strain Expressing an Esterase

Nematicidal enzymes were produced by fermentation in Bacillus subtilis. Each enzyme was cloned into Bacillus expression plasmid p1797 with isopropyl β-d-1-thiogalactopyranoside (IPTG)-inducible expression. The plasmids were transformed into B. subtilis, and the strains were grown in rich media until an optical density (OD) of 0.2-0.5 was reached. Then, enzyme expression was induced by addition of 1 mM IPTG. Following the culture harvest, culture whole cell broth containing secreted enzymes was then used for further applications.

To investigate effects of an esterase enzyme on SCN, treatments were applied to soybean seed with PI88788 resistance to SCN. Esterase enzyme treatment was added at a rate of 2 fl. oz. per unit of seed, mixed in a slurry with a colorant and Peridiam® 1006 (saxagliptin hydrochloride/dapagliflozin propanediol) and applied to seeds while vortexing in a tube. For each experiment, a base seed treatment control was included that contained only colorant and Peridiam® 1006 (saxagliptin hydrochloride/dapagliflozin propanediol). These seeds were then planted in soil in cone-tainers containing a loamy sand soil mixture (Hummert International: 80% sand, 15% silt, 5% clay). Plant inoculation, determination of cysts per gram of root tissue, and data analysis were performed as in Example 2

Data for average cysts per gram of root tissue for seed treatment with a recombinant strain expressing Esterase B and comparison to base control can be found in Table 37. Esterase B recombinant strain (SEQ ID NO: 89) displayed a 55% reduction of average cysts per gram of root tissue relative to the control treatment at three weeks after inoculation with the plant parasitic nematode SCN indicating that esterase recombinant strain seed treatment reduces SCN reproduction on treated plants.

TABLE 37
Reduction of Soybean Cyst Nematode Reproduction
by a Recombinant Strain Expressing an Esterase

| | | % | | p-value | |
|---|---|---|---|---|---|
| | Average cysts per | Comparison | Standard | relative to | Pairwise |
| Treatment | g of root tissue | to Control | deviation | Control | contrast |
| Base Control | 111.0 | 100% | 95.8 | n/a | a |
| Esterase B recombinant | 49.8 | 45% (−55%) | 26.5 | <.0001 | b |
| strain | | | | | |
| (SEQ ID NO: 89) | | | | | |

### Example 22

### Reduction of Soybean Cyst Nematode Reproduction by an Enzyme in a Dried Form

A dried formulation of neutral protease (SEQ ID NO: 127) was created by combining the enzyme produced in Bacillus subtilis fermentation media (see Example 2) with 5.66% v/v polyvinylpyrrolidone. The mixture was air-dried at room temperature, and the film produced was broken into small particles with a mortar and pestle. These particles were mixed directly into a soybean seed treatment slurry containing colorant and Peridiam® 1006 (saxagliptin hydrochloride/dapagliflozin propanediol). This formulation was tested at 2 rates (0.32 mg/seed and 0.64 mg/seed), and the liquid formulation consisting of the enzyme in fermentation media was also used for a seed treatment at 2 fl oz. per unit of seed. ILEVO® (48.4% fluopyram) was included as a commercial nematicide positive control. The effects of the seed treatments against SCN infection were investigated as described in Example 2, except that soybean variety 2830E3 was used and no bleach sterilization was used for the SCN hatchery. Data for the average cysts per gram of root tissue was analyzed as in Example 2 and can be found in Table 38. The liquid formulation shows a statistically significant reduction relative to the control, and both rates of the dried formulation show a further significant reduction below that of the liquid formulation. The dried formulation at both rates performed in a manner that was statistically similar to ILEVO®(48.4% fluopyram). These data indicate that a dried enzyme can reduce SCN reproduction on host plants.

TABLE 38
Application of Dried Enzyme Reduces Average Cysts
Per Gram of Root Tissue Relative to Base Control

| | | % | | p-value | |
|---|---|---|---|---|---|
| | Average cysts per g | Comparison | Standard | relative to | Pairwise |
| Treatment | of root tissue | to Control | deviation | Control | contrast |
| Base Control | 358.2 | 100% | 188.1 | n/a | a |
| Neutral protease liquid | 208.8 | 58% (−42%) | 218.8 | .0014 | b |
| formulation | | | | | |
| [2 fl oz/unit] | | | | | |
| (SEQ ID NO: 127) | | | | | |
| Neutral protease dried | 70.3 | 20% (−80%) | 63.1 | <.0001 | c |
| formulation | | | | | |
| ([0.32 mg/seed] | | | | | |
| (SEQ ID NO: 127) | | | | | |
| Neutral protease dried | 62.2 | 17% (−83%) | 40.4 | <.0001 | c |
| formulation | | | | | |
| [0.64 mg/seed] | | | | | |
| (SEQ ID NO: 127) | | | | | |
| ILEVO | 68.8 | 19% (−81%) | 60.7 | <.0001 | c |

### Example 23

An Esterase-Expressing Recombinant Strain Exhibits Efficacy in Reducing Soybean Cyst Nematode Reproduction in Combination with a Fertilizer

To determine whether an esterase-expressing recombinant strain can reduce SCN reproduction in combination with a fertilizer, we conducted a controlled environment seed treatment trial with esterase B recombinant strain (SEQ ID NO: 89) and liquid 10-34-0 fertilizer. The recombinant strain was produced as in Example 3, and the unfiltered whole cell broth was applied to seeds as in Example 2. Fertilizer was applied to seeds at 3% of slurry volume in the seed treatment. A combination treatment was performed in which both fertilizer and the recombinant strain were applied to the same seeds. The plants were inoculated as in Example 2, except that soybean variety 3230E3 was used and no bleach sterilization was used for the SCN hatchery. Because fertilizer treatments promote plant root growth, this trial was evaluated on a cysts per plant basis rather than cysts per gram of root tissue to enable a fair comparison between treatments. The results were analyzed as in Example 2 and can be found in Table 39. The esterase-expressing recombinant strain (SEQ ID NO: 89) reduced SCN reproduction both alone and in combination with the fertilizer. Thus, an esterase recombinant strain can be combined with fertilizer and exhibit efficacy against pathogenic nematodes.

TABLE 39
Application of a Fertilizer in Combination with a Recombinant Strain Expressing
an Esterase Reduces Average Cysts Per Plant Relative to Base Control

| | | | | p-value | |
|---|---|---|---|---|---|
| | | % | | relative | |
| | Average cysts | Comparison | Standard | to | Pairwise |
| Treatment | per plant | to Control | deviation | Control | contrast |
| Base Control | 9.9 | 100% | 7.2 | n/a | a |
| Fertilizer 10-34-0 | 8.1 | 82% (−18%) | 5.1 | .4522 | a |
| Esterase B recombinant | 5.6 | 57% (−43%) | 5.1 | .0218 | b |
| strain (SEQ ID NO: 89) | | | | | |
| Esterase B recombinant | 8.8 | 89% (−11%) | 7.1 | .9967 | a |
| strain (SEQ ID NO: 89) + | | | | | |
| Fertilizer 10-34-0 | | | | | |

### Example 24

### Increase of Nematode Death Following Foliar Application on Leaves

To determine whether a nematicidal enzyme can cause nematode death when sprayed as a foliar treatment, a trial was performed using chitinase 19F (SEQ ID NO: 182). The free enzyme was produced as described in Example 2. A SCN hatchery was set up as described in Example 2, except that no bleach sterilization step was performed. Six days later, the concentration of hatched nematodes was determined. Leaves from Quinault strawberry plants (Fragaria× ananassa) were cut into square sections and placed in the wells of a 24-well plate with the abaxial side up. Twenty-four wells were set up for each treatment. Spray solutions were created with water alone and water plus fermentation broth with chitinase 19F (SEQ ID NO: 182). For the base strain control and chitinase solutions, 0.094 mL of fermentation broth was added to 15 mL of water (0.6% solution) to model a field spray application of 8 fl oz. per acre. Treatments were added to a small spray bottle and each leaf segment was sprayed approximately 3 times. After the leaves had dried, approximately 100 nematodes were added to each leaf segment. To assess the number of nematodes that were alive at the start of the experiment hours, 500 μL of sterile water was added to 4 wells of each treatment, and the leaf segment was washed and removed. The number of alive and dead nematodes were counted for each well, and the average percentage of dead nematodes were calculated for each treatment. The same procedure was performed 24 hours after the start of the experiment for the remaining 20 wells for each treatment. The results are below in Table 40. A low background rate of nematode death is observed at the start of the experiment for each treatment. At 24 hours, the average percentage of dead nematodes in the chitinase 19F (SEQ ID NO: 182) treated leaves increased significantly above the control treatments. The statistical analysis was performed as in Example 2. These results indicate that spray application of a nematicidal enzyme applied to leaf surfaces can lead to increased rates of death for a pathogenic nematode.

TABLE 40
Foliar Application of an Enzyme Reduces Average Nematode
Death Percentage Relative to Water Only (Control)

| | | | p-value relative | Pairwise |
|---|---|---|---|---|
| | Average percentage | Average percentage | to water only | contrast |
| | of dead nematodes | of dead nematodes | (24 hour | (24 hour |
| Treatment | at 0 hours | at 24 hours | averages) | averages) |
| Water only (Control) | 4% | 5% | n/a | a |
| Chitinase 19F | 3% | 12% | .0001 | b |
| recombinant strain | | | | |
| (SEQ ID NO: 182) | | | | |

### Example 25

### Methods for Producing and Preparing Isolated Enzymes

Production of recombinant enzymes of the present invention was carried out. In brief, coding sequences were cloned into expression vectors suitable for inducible protein production in Escherichia coli or Bacillus subtilis. Transformed bacteria were cultured in rich media to an optical density of 0.5-0.8 before induction. Protein expression was induced with 0.1 mM or 1 mM isopropyl β-d-1-thiogalactopyranoside (IPTG) in E. coli or in B. subtilis, respectively. Enzymes produced in E. coli were expressed with a six-histidine tag and they were extracted, and affinity purified using standard procedure. Enzymes produced in B. subtilis were secreted and cell-free cultures were obtained by pelleting cells by centrifugation and filtered sterilizing supernatants using a 0.2-micron filter.

The free enzymes as described below were diluted or resuspended in water at the indicated volume or mass per mL water, respectively. To eliminate solid in suspension, suspensions were centrifuged and supernatant free of solids was collected. Finally, supernatants were filter sterilized using a 0.2-micron filter.

### Example 26

### Evaluation of Insecticidal Activity of Enzymes Using Diet-Surface Overlay (DSO) Bioassay

Diet-surface overlay bioassays are conducted with artificial diet mixes without antibiotics in 1.5 cm² cell bioassay trays(s). Artificial diets are commercially available from Southland Products Inc. Diets were prepared by dispersing 0.5 mL of molten diet to each 1.5 cm² cell. After solidification, 0.1 mL enzyme treatments were dispersed on top of diet in each cell and the tray was gently rocked in an orbital shaker to spread the liquid uniformly over the diet surface. Trays were placed under clean airflow until the surface of each cell was uniformly dried. One or two 16-cell sections were used per treatment (the number in larvae treated was 16 or 32).

One healthy first instar larva was placed into each treatment cell, each 16-cell section was covered with a re-sealable lid and incubated at 27° C. and light-dark period of 16:8 hrs. After 7 days, and according to the species, the number of individuals in the following developmental categories was recorded.

Diamondback moth (Plutella xylostella): Dead Larva (lack of movement), Larva Stage I (≤50% relative size), Larva Stage II (100% relative size, full size), Early Pupa (light green color), Late Pupa (light-dark brown color), and Adult.

Fall armyworm (Spodoptera frugiperda): Dead Larva (lack of movement), Larva Stage I (≤25% relative size), Larva Stage II (50% relative size), Larva Stage III (75% relative size), and Larva Stage IV (100% relative size, full size).

Negative/untreated controls were treated with filtered water and positive controls were treated with lethal doses of Bacillus thuringiensis subsp. Kurstaki (Btk) spores at 1000 ppm.

Proteases on Diamondback Moth (Plutella xylostella)

Acid Protease, alkaline Protease, aminopeptidase, neutral protease, and keratinase, were resuspended in water at 0.25 g/mL. Recombinant protease neutral protease from Bacillus sp. (SEQ ID NO: 127) were produced as indicated in Example 9. Culture of D505 (untransformed strain use for production of recombinant proteases) was included as negative control. The U of enzyme activity was determined as the amount of enzyme required to breakdown 1 μmol of substrate per minute. Treatments and scoring followed the DSO bioassay as described above. Results are shown below in Table 41. The present results demonstrate that the proteases tested delay larva-pupa development and result in a low number of individuals reaching adulthood, if any.

TABLE 41
Effect of proteases on survival and development of Diamondback moth (n = 16)

| | | | Larva | Larva | | | | |
|---|---|---|---|---|---|---|---|---|
| | U/mL | Dead | Stage | Stage | Early | Late | | p- |
| Treatment | diet | Larva | I | II | Pupa | Pupa | Adult | Value* |
| Water | 0 | 0 | 0 | 0 | 3 | 3 | 10 | |
| D505 (Negative | 0 | 0 | 0 | 0 | 3 | 2 | 11 | 1.00 |
| Control) | | | | | | | | |
| Neutral Protease | 0.206 | 2 | 0 | 0 | 8 | 6 | 0 | <0.005 |
| (SEQ ID NO: 127) | | | | | | | | |
| Acid Protease | 2.5 | 0 | 0 | 0 | 6 | 4 | 6 | 0.43 |
| Alkaline Protease | 190 | 1 | 0 | 0 | 2 | 13 | 0 | <0.005 |
| Aminopeptidase | nd** | 0 | 0 | 0 | 4 | 11 | 1 | <0.005 |
| Neutral Protease | 20 | 1 | 0 | 0 | 6 | 9 | 0 | <0.005 |
| Keratinase | 26 | 1 | 0 | 0 | 11 | 4 | 0 | <0.005 |
| Sporulated | 0 | 16 | 0 | 0 | 0 | 0 | 0 | <0.005 |
| B. thuringiensis | | | | | | | | |
| Kurstaki | | | | | | | | |

*One-sided Fisher's exact test for frequency distribution on different stages in water control and corresponding treatment. A p < 0.05 value is considered statistically significant.
**Value not accurately quantified with Azocasein assay

To further demonstrate these effects, three proteases were selected, and the effect of 10× higher protease activity rate was tested on a Btk resistant strain. Results are shown below in Table 42. The keratinase showed more than 50% mortality and strong stunting of survival larvae. In contrast, Both neutral proteases showed a small number of dead larvae. However, both neutral proteases reduced the number of individuals reaching adult stage.

TABLE 42
Effect of proteases on survival and development
of Btk resistant Diamondback moth (n = 32)

| | | | Larva | Larva | | | | |
|---|---|---|---|---|---|---|---|---|
| | U/mL | Dead | Stage | Stage | Early | Late | | p- |
| Treatment | Diet | Larva | I | II | Pupa | Pupa | Adult | Value* |
| Water | 0 | 1 | 0 | 0 | 1 | 22 | 8 | |
| Neutral | 2 | 4 | 0 | 0 | 6 | 22 | 0 | <0.005 |
| Protease | | | | | | | | |
| (SEQ ID | | | | | | | | |
| NO: 127) | | | | | | | | |
| Neutral | 200 | 3 | 1 | 1 | 3 | 24 | 0 | <0.005 |
| Protease | | | | | | | | |
| Keratinase | 260 | 19 | 11 | 2 | 0 | 0 | 0 | <0.005 |
| Sporulated | 0 | 32 | 0 | 0 | 0 | 0 | 0 | <0.005 |
| B. | | | | | | | | |
| thuringiensis | | | | | | | | |
| Kurstaki | | | | | | | | |

*One-sided Fisher's exact test for frequency distribution on different stages in water control and corresponding treatment. A p < 0.05 value is considered statistically significant.

Proteases on Fall Armyworm (Spodoptera frugiperda)

The effect of Keratinase was also tested on Fall armyworm. Treatments and scoring followed the DSO bioassay as described above. Results are shown below in Table 43. As can be seen, Keratinase was lethal to Fall armyworm larva.

TABLE 43
Effect of proteases on survival and
development of Fall armyworm (n = 32)

| | | | Larva | Larva | Larva | Larva | |
|---|---|---|---|---|---|---|---|
| | U/mL | Dead | Stage | Stage | Stage | Stage | p- |
| Treatment | Diet | Larva | I | II | III | IV | Value* |
| Water | 0 | 2 | 0 | 0 | 0 | 30 | |
| Keratinase | 260 | 32 | 0 | 0 | 0 | 0 | <0.005 |
| Sporulated | 0 | 32 | 0 | 0 | 0 | 0 | <0.005 |
| B. thuringiensis | | | | | | | |
| Kurstaki | | | | | | | |

*One-sided Fisher's exact test for frequency distribution on different stages in water control and corresponding treatment. A p < 0.05 value is considered statistically significant.

Lipases on Diamondback Moth (Plutella xylostella)

Recombinant lipase from Bacillus thuringiensis (SEQ ID NO: 76) was produced as indicated in Example 9. The U of enzyme activity was determined as the amount of enzyme required to breakdown 1 μmol of substrate per minute. Treatments and scoring followed the DSO bioassay as described above. As can be seen below in Table 44, lipase delayed larva-pupa development as most individuals were at late pupa stage and no individuals reached adulthood.

TABLE 44
Effect of Lipases on survival and development
of Diamondback moth (n = 16)

| | | | Larva | Larva | | | | |
|---|---|---|---|---|---|---|---|---|
| | U/mL | Dead | Stage | Stage | Early | Late | | p- |
| Treatment | Diet | Larva | I | II | Pupa | Pupa | Adult | Value * |
| Water | 0 | 0 | 0 | 0 | 3 | 3 | 10 | |
| Lipase | 0.14 | 0 | 0 | 0 | 3 | 13 | 0 | <0.005 |
| (SEQ ID | | | | | | | | |
| NO: 76) | | | | | | | | |
| Sporulated | 0 | 16 | 0 | 0 | 0 | 0 | 0 | <0.005 |
| B. | | | | | | | | |
| thuringiensis | | | | | | | | |
| Kurstaki | | | | | | | | |

* One-sided Fisher's exact test for frequency distribution on different stages in water control and corresponding treatment. A p < 0.05 value is considered statistically significant.

The effect of lipase (10× enzyme rate) and free lipases were tested on a Diamondback Moth Btk resistant strain. Free Lipases from Burkholderia cepacia (Bc) and Pseudomonas fluorescens (Pf) were tested along with Lipase (SEQ ID NO: 76) and the results are shown below in Table 45. The results demonstrate a clear delay of larva-pupa development by lipase, with Bc and Pf Lipases showing a lethal effect as most or all larvae were dead.

TABLE 45
Effect of Lipases on survival and development
of Diamondback moth (n = 32)

| | | | Larva | Larva | | | | |
|---|---|---|---|---|---|---|---|---|
| | U/mL | Dead | Stage | Stage | Early | Late | | p- |
| Treatment | Diet | Larva | I | II | Pupa | Pupa | Adult | Value* |
| Water | 0 | 3 | 0 | 0 | 7 | 22 | 0 | |
| Lipase | 1.4 | 1 | 9 | 12 | 10 | 0 | 0 | <0.005 |
| (SEQ ID | | | | | | | | |
| NO: 76) | | | | | | | | |
| Bc Lipase | 3.8 | 27 | 2 | 3 | 0 | 0 | 0 | <0.005 |
| Pf Lipase | 5.2 | 32 | 0 | 0 | 0 | 0 | 0 | <0.005 |
| Sporulated | 0 | 32 | 0 | 0 | 0 | 0 | 0 | <0.005 |
| B. | | | | | | | | |
| thuringiensis | | | | | | | | |
| Kurstaki | | | | | | | | |

*One-sided Fisher's exact test for frequency distribution on different stages in water control and corresponding treatment. A p < 0.05 value is considered statistically significant.

Lipases on Fall Armyworm (Spodoptera frugiperda)

The effect of free lipases were tested on Fall armyworm. Lipases from Burkholderia cepacia (Bc), Pseudomonas fluorescens (Pf), porcine pancreas Type II (PpII), Rhizomucor miehei (Rm), and lipase were tested. Results are shown below in Table 46. Pf lipase showed a lethal effect, and all other tested lipases strongly delayed larva development as most larvae were stunted and dead.

TABLE 46
Effect of Lipases on survival and development
of Fall armyworm (n = 32)

| | | | Larva | Larva | Larva | Larva | |
|---|---|---|---|---|---|---|---|
| | U/mL | Dead | Stage | Stage | Stage | Stage | p- |
| Treatment | Diet | Larva | I | II | III | IV | Value* |
| Water | 0 | 2 | 0 | 0 | 0 | 30 | |
| Bc Lipase | 3.8 | 6 | 5 | 12 | 7 | 2 | <0.005 |
| Pf Lipase | 5.2 | 32 | 0 | 0 | 0 | 0 | <0.005 |
| PpII Lipase | 0.4 | 0 | 2 | 5 | 15 | 10 | <0.005 |
| Rm Lipase | 2.8 | 8 | 23 | 1 | 0 | 0 | <0.005 |
| Lipase | nd** | 5 | 2 | 10 | 15 | 0 | <0.005 |
| Sporulated | 0 | 32 | 0 | 0 | 0 | 0 | <0.005 |
| B. thuringiensis | | | | | | | |
| Kurstaki | | | | | | | |

*One-sided Fisher's exact test for frequency distribution on different stages in water control and corresponding treatment. A p < 0.05 value is considered statistically significant.
**Value not accurately quantified, large variability in multiple assays

Chitinases on Diamondback Moth (Plutella xylostella)

Recombinant chitinases from Streptomyces coelicolor (SEQ ID NO: 182), and from Akanthomyces lecanii (SEQ ID NO: 185) were produced as indicated in Example 9. The U of enzyme activity was determined as the amount of enzyme required to breakdown 1 μmol of substrate per minute. Treatments and scoring followed the DSO bioassay as described above and the results are shown below in Table 47. In particular, treatment with chitinases resulted in a reduction in larva-pupa development with fewer individuals reaching adulthood.

TABLE 47
Effect of Chitinases on survival and development
of Btk resistant Diamondback moth (n = 32)

| | | | Larva | Larva | | | | |
|---|---|---|---|---|---|---|---|---|
| | mU/mL | Dead | Stage | Stage | Early | Late | | p- |
| Treatment | Diet | Larva | I | II | Pupa | Pupa | Adult | Value* |
| Water | 0 | 1 | 0 | 0 | 0 | 24 | 7 | |
| Chitinase | 3.7 | 2 | 2 | 1 | 4 | 22 | 1 | 0.01 |
| (SEQ ID NO: 182) | | | | | | | | |
| Chitinase | 0.6 | 2 | 2 | 2 | 8 | 17 | 1 | <0.005 |
| (SEQ ID NO: 185) | | | | | | | | |
| Sporulated | 0 | 32 | 0 | 0 | 0 | 0 | 0 | <0.005 |
| B. thuringiensis | | | | | | | | |
| Kurstaki | | | | | | | | |

*One-sided Fisher's exact test for frequency distribution on different stages in water control and corresponding treatment. A p < 0.05 value is considered statistically significant.

Chitinases on Fall Armyworm (Spodoptera frugiperda)

The effect of recombinant chitinases from Streptomyces coelicolor (SEQ ID NO: 182), from Akanthomyces lecanii (SEQ ID NO: 185), and free chitinases from Streptomyces griseus (Sg) and Trichoderma viride (Tv) was tested. Chitinases were produced or prepared as indicated in Example 9. The U of enzyme activity was determined as the amount of enzyme required to breakdown 1 μmol of substrate per minute. Treatments and scoring followed the DSO bioassay as described above and the results are shown below in Table 48. All tested chitinases showed delay of larva development. A strong reduction of individuals reaching Larva Stage IV by some chitinases was observed. Tv Chitinase showed noticeable stunting effect.

TABLE 48
Effect of Chitinases on survival and
development of Fall armyworm (n = 32)

| | | | Larva | Larva | Larva | Larva | |
|---|---|---|---|---|---|---|---|
| | mU/mL | Dead | Stage | Stage | Stage | Stage | p- |
| Treatment | Diet | Larva | I | II | III | IV | Value* |
| Water | 0 | 1 | 0 | 0 | 0 | 31 | |
| Chitinase | 3.7 | 3 | 4 | 14 | 5 | 6 | <0.005 |
| (SEQ ID | | | | | | | |
| NO: 182) | | | | | | | |
| Chitinase | 0.6 | 7 | 4 | 5 | 8 | 8 | <0.005 |
| (SEQ ID | | | | | | | |
| NO: 185) | | | | | | | |
| Sg Chitinase | 1.9 | 0 | 0 | 9 | 15 | 8 | <0.005 |
| Tv Chitinase | 5.4 | 1 | 0 | 0 | 10 | 21 | <0.005 |
| Sporulated | 0 | 32 | 0 | 0 | 0 | 0 | <0.005 |
| B. thuringiensis | | | | | | | |
| Kurstaki | | | | | | | |

*One-sided Fisher's exact test for frequency distribution on different stages in water control and corresponding treatment. A p < 0.05 value is considered statistically significant.

Esterases on Diamondback Moth (Plutella xylostella)

Recombinant esterases estB from Bacillus subtilis (SEQ ID NO: 89), and Free esterases were from Pseudomonas fluorescens (Pf), and Rhizopus oryzae (Ro) were produced or prepared as indicated in Example 9. The U of enzyme activity was determined as the amount of enzyme required to breakdown 1 μmol of substrate per minute. Treatments and scoring followed the DSO bioassay as described above. As can be seen below in Table 49, estB delayed larva-pupa development of 47% of individuals, 34% of which were at larva stage. For other esterases there was numerical increases in dead larva and early pupa delayed onsets with Pf and Ro esterases.

TABLE 49
Effect of Esterases on survival and development
of Diamondback moth (n = 32)

| | | | Larva | Larva | | | | |
|---|---|---|---|---|---|---|---|---|
| | U/mL | Dead | Stage | Stage | Early | Late | | p- |
| Treatment | Diet | Larva | I | II | Pupa | Pupa | Adult | Value* |
| Water | 0 | 0 | 0 | 0 | 1 | 31 | 0 | |
| estB | 0.5 | 0 | 3 | 8 | 4 | 17 | 0 | <0.005 |
| (SEQ ID | | | | | | | | |
| NO: 89) | | | | | | | | |
| Pf Esterase | 7.1 | 1 | 0 | 0 | 4 | 27 | 0 | 0.20 |
| Ro Esterase | 0.01 | 1 | 0 | 0 | 5 | 26 | 0 | 0.10 |
| Sporulated | 0 | 32 | 0 | 0 | 0 | 0 | 0 | <0.005 |
| B. | | | | | | | | |
| thuringiensis | | | | | | | | |
| Kurstaki | | | | | | | | |

*One-sided Fisher's exact test for frequency distribution on different stages in water control and corresponding treatment. A p < 0.05 value is considered statistically significant.

Esterases on Fall Armyworm (Spodoptera frugiperda)

The effect of recombinant and free esterases was also tested on Fall armyworm. Recombinant esterases estB from Bacillus subtilis (SEQ ID NO: 89), H6-pnbA from Bacillus subtilis (SEQ ID NO: 94), and H6-pytH from Sphingobium wenxiniae (SEQ ID NO: 95), and esterases were from Pseudomonas fluorescens (Pf), and Rhizopus oryzae (Ro) were produced or prepared as indicated in Example 9. The U of enzyme activity was determined as the amount of enzyme required to breakdown 1 μmol of substrate per minute. Treatments and scoring followed the DSO bioassay as described above. Results are shown below in Table 50. For H6-tagged proteins PBS was checked and showed no effect. Similar to the results observed in the case of Diamondback Moth, estB (SEQ ID NO: 89) delayed larva-pupa development of 67% of individuals. An effect was also observed using Pf Esterase, with delay of development of 40% individuals and a 25% of larva mortality. An additional effect was observed for Ro Esterase.

TABLE 50
Effect of Esterases on survival and
development of Fall armyworm (n=32)

| | | | Larva | Larva | Larva | Larva | |
|---|---|---|---|---|---|---|---|
| | U/mL | Dead | Stage | Stage | Stage | Stage | p- |
| Treatment | Diet | Larva | I | II | III | IV | Value* |
| Water | 0 | 0 | 2 | 1 | 8 | 21 | |
| PBS | 0 | 1 | 0 | 0 | 9 | 22 | 0.62 |
| H6-pnbA | 19.5 | 0 | 0 | 0 | 15 | 17 | 0.09 |
| (SEQ ID NO: 94) | | | | | | | |
| H6-pytH | 2.3 | 0 | 0 | 0 | 13 | 19 | 0.22 |
| (SEQ ID NO: 95) | | | | | | | |
| estB | 0.5 | 1 | 3 | 4 | 14 | 10 | 0.05 |
| (SEQ ID NO: 89) | | | | | | | |
| Pf Esterase | 7.1 | 8 | 4 | 1 | 8 | 11 | 0.007 |
| Ro Esterase | 0.01 | 0 | 0 | 0 | 16 | 16 | 0.06 |
| Sporulated | 0 | 32 | 0 | 0 | 0 | 0 | <0.005 |
| B. thuringiensis | | | | | | | |
| Kurstaki | | | | | | | |

*One-sided Fisher's exact test for frequency distribution on different stages in water control and corresponding treatment. A p < 0.05 value is considered statistically significant.

### Example 27

### Free Esterase on MAP on Wheat

Esterase A (EstA) from Bacillus subtilis (SEQ ID NO:254) was produced via fermentation of a recombinantly produced enzyme in Bacillus subtilis. 1 fl oz of fermentation product was applied onto 15 lb P/A (phosphate equivalent per acre) monoammonium phosphate (MAP) fertilizer in a concrete mixer and allowed to mix for 1 minute. A MAP control treatment without fermentation media was also created. MAP, alone or with fermentation media was distributed across 4 small replicated plots prior to planting of winter wheat with 5 replicates of 300 ft² each at each site. Upon harvesting the four sites, the data was compiled and averaged and the results are shown in Table 51 below.

TABLE 51
Yield and Vigor effects of Esterase
on winter wheat on MAP fertilizer

| | | | Vigor Score |
|---|---|---|---|
| | Yield | Yield | (1 to 5 scale, |
| Fertilizer Treatment | (Bu/A) | (Kg/ha) | 5 is highest) |
| MAP (15 lb/A Phosphate) | 61.33 | 4124.5 | 4.0 |
| (Base Control) | | | |
| MAP Base Control + 1 fl oz/A | 62.82 | 4224.7 | 4.13 |
| EstA fermentation | | | |

The addition of the EstA fermentation increased the yield in the trial above the MAP control alone. Seedling Vigor Rating describes the health of the seedling. The addition of the EstA fermentation also increased the vigor of the young wheat seedlings in the trial.

### Example 28

### Free Esterase on Corn on MAP and DAP

Esterase A (EstA) from Bacillus subtilis (SEQ ID NO:254) was produced via fermentation of a recombinantly produced enzyme in Bacillus subtilis. 2 fl oz of fermentation product was applied onto either 72 lb P/A (phosphate equivalent per acre) monoammonium phosphate (MAP) fertilizer or diammonium phosphate (DAP) fertilizer in a concrete mixer, and allowed to mix for 1 minute. A MAP/DAP control treatment without fermentation media was also created. MAP or DAP, alone or with fermentation media was distributed across 4 small replicated plots each prior to planting of corn with 5 replicates of 400 ft2 each at each site (4 site with MAP, 4 sites with DAP, combined in data in Table 52 below). Upon harvesting the eight sites, the data was compiled and averaged and the results are shown in Table 52 below.

TABLE 52
Yield effects of Esterase on corn on MAP/DAP fertilizer

| | Yield | Yield |
|---|---|---|
| Fertilizer Treatment | (Bu/A) | (Kg/ha) |
| MAP/DAP (72 lb/A Phosphate) (Base Control) | 202.3 | 12798 |
| MAP/DAP Base Control + 2 fl oz/A | 206.6 | 13077 |
| EstA fermentation | | |

The addition of the EstA fermentation increased the yield in the trial above the MAP/DAP control alone.

### Example 29

### Free Esterase on Urea on Wheat

Esterase A (EstA) from Bacillus subtilis (SEQ ID NO:254) was produced via fermentation of a recombinantly produced enzyme in Bacillus subtilis. 1 fl oz or 2 fl oz of fermentation product was applied onto 15 lb N/A (nitrogen equivalent per acre) urea fertilizer in a concrete mixer and allowed to mix for 1 minute. A urea control treatment without fermentation media was also created. urea, alone or with fermentation media was distributed across 4 small replicated plots prior to planting of winter wheat with 5 replicates of 300 ft² each at each site. Upon harvesting the four sites, the data was compiled and averaged and the results are shown in Table 53 below.

TABLE 53
Yield effects of Esterase on winter wheat on urea fertilizer

| | Yield | Yield |
|---|---|---|
| Fertilizer Treatment | (Bu/A) | (Kg/ha) |
| Urea (15 lb/A Nitrogen) (Base Control) | 76.1 | 5117.8 |
| Urea Base Control + 1 fl oz/A EstA fermentation | 79.6 | 5353.2 |
| Urea Base Control + 2 fl oz/A EstA fermentation | 79.9 | 5375.4 |

The addition of the EstA fermentation increased the yield in the trial above the urea control alone.

### Example 30

### Free Esterase on Corn on DAP

Esterase A (EstA) from Bacillus subtilis (SEQ ID NO:254) was produced via fermentation of a recombinantly produced enzyme in Bacillus subtilis. 1, 2, or 3 fl oz of fermentation product was applied onto 67.5 lb P/A (phosphate equivalent per acre) diammonium phosphate (DAP) fertilizer in a concrete mixer, and allowed to mix for 1 minute. A DAP control treatment without fermentation media was also created. DAP, alone or with fermentation media was distributed across 4 small replicated plots each prior to planting of corn with 4 replicates of 400 ft² each at each site. Upon harvesting the four sites, the data was compiled and averaged and the results are shown in Table 54 below.

TABLE 54
Yield effects of Esterase on corn on DAP fertilizer

| | Yield | Yield |
|---|---|---|
| Fertilizer Treatment | (Bu/A) | (Kg/ha) |
| DAP (67.5 lb/A Phosphate) (Base Control) | 196.0 | 12319.5 |
| DAP Base Control + 1 fl oz/A EstA fermentation | 201.8 | 12684.4 |
| DAP Base Control + 2 fl oz/A EstA fermentation | 204.2 | 12834.7 |
| DAP Base Control + 3 fl oz/A EstA fermentation | 202.6 | 12734.7 |

The addition of the EstA fermentation increased the yield in the trial above the DAP control alone.

### Example 31

### Free Esterase on Corn on Urea and on Nitrogen Stabilizers

Esterase A (EstA) from Bacillus subtilis (SEQ ID NO:254) was produced via fermentation of a recombinantly produced enzyme in Bacillus subtilis. 1, 2, or 3 fl oz of fermentation product was applied onto 135 lb N/A (nitrogen equivalent per acre) urea fertilizer in a concrete mixer, and allowed to mix for 1 minute. A urea control treatment without fermentation media was also created. An addition treatment that contained 64 fl oz/ton N-Butyl-thiophosphoric triamide (NBPT) nitrogen stabilizer or 64 fl oz/ton NBPT nitrogen stabilizer with 3 fl oz/A EstA fermentation product was also included in the trial. Urea, alone or with fermentation media was distributed across 4 small replicated plots each prior to planting of corn with 4 replicates of 400 ft2 each at each site. Upon harvesting the four sites, the data was compiled and averaged and the results are shown in Table 55 below.

TABLE 55
Yield effects of Esterase on corn on
urea with or without NBPT fertilizer

| | Yield | Yield |
|---|---|---|
| Fertilizer Treatment | (Bu/A) | (Kg/ha) |
| Urea (135 lb/A Nitrogen) (Base Control) | 188.2 | 11830.4 |
| Urea Base Control + 1 fl oz/A EstA fermentation | 205.0 | 12887.5 |
| Urea Base Control + 2 fl oz/A EstA fermentation | 195.8 | 12309.9 |
| Urea Base Control + 3 fl oz/A EstA fermentation | 195.6 | 1229.8 |
| Urea Base Control + NBPT (64 fl oz/ton) | 191.2 | 12022.3 |
| Urea Base Control + NBPT (64 fl oz/ton) + 3 fl oz/A | 194.6 | 12232.0 |
| EstA fermentation | | |

The addition of the EstA fermentation increased the yield in the trial above the urea control and NBPT containing treatments alone.

### Example 32

### Free Esterase on Seed Treatment on Soy

1 fl oz/unit of Feruloyl Esterase (SEQ ID NO:91) was coated onto soybean seeds treated with Fludioxinil fungicide, metalaxyl fungicide, thiamethoxam fungicide, Seedwork 1037L polymer, and Sensient Red (colorant). A control treatment without esterase enzyme was also created. The seed treated with esterase and control seed was planted in replicated trials distributed across 5 replicated plots with 4 replicates of 30 ft² each at each site. Upon harvesting the 5 sites, the data was compiled and averaged and the results are shown in Table 56 below.

TABLE 56
Yield effects of Esterase on soybean seed treatment

| Treatment | Yield (Bu/A) | Yield (Kg/ha) |
|---|---|---|
| Control Base Package | 63.99 | 4303.4 |
| Control Base Package with | 64.86 | 4361.9 |
| Feruloyl Esterase 1.0 fl oz/unit | | |

The addition of the Feruloyl esterase enzyme increased the yield in the trial above the base seed treatment package alone.

### Example 33

### Free Esterase on Corn Seed Treatment

0.5 fl oz/unit of PythH Esterase from recombinant Bacillus subtilis fermentation (SEQ ID NO: 255) was coated onto Hybrid corn seeds treated with Fludioxinil fungicide, ipconazole fungicide, peridium 1006 polymer, and Sensient Red (colorant). A control treatment without esterase enzyme was also created. The seed treated with esterase and control seed was planted in replicated trials distributed across 10 replicated plots with 4 replicates of 100 ft2 each at each site. Upon harvesting the 10 sites, the data was compiled and averaged and the results are shown in Table 57 below.

TABLE 57
Yield effects of Esterase on corn seed treatment

| Treatment | Yield (Bu/A) | Yield (Kg/ha) |
|---|---|---|
| Control Base Package | 205.5 | 13820.1 |
| Control Base Package with | 208.8 | 14042.0 |
| PytH Esterase 0.5 fl oz/unit | | |

The addition of the PytH esterase enzyme increased the yield in the trial above the base seed treatment package alone.

### Example 34

### Free Esterase on Soybean Seed Treatment

0.5 fl oz/unit of Esterase (carboxylesterase) from Geobacillus/Bacillus stearothermophilus (SEQ ID NO:252) was coated onto soybean seeds treated with Evergol Energy (prothioconazole, metalaxyl, and penflufen fungicides) at 1 fl oz/cwt, Alias 4F (imidacloprid, insecticide) at 1.25 fl oz/unit, Sensient Red (colorant) at 0.5 fl oz/cwt, and Seedwork 4037L polymer at 1.5 fl oz/cwt. A control treatment without esterase enzyme was also created. The seed treated with esterase and control seed was planted in replicated trials distributed across 6 replicated plots with 4 replicates of 60 ft2 each at each site. Upon harvesting the 6 sites, the data was compiled and averaged and the results are shown in Table 58 below.

TABLE 58
Yield effects of Esterase on soybean seed treatment

| Treatment | Yield (Bu/A) | Yield (Kg/ha) |
|---|---|---|
| Control Base Package | 70.90 | 4768.1 |
| Control Base Package with | 72.36 | 4866.3 |
| Carboxyesterase 0.5 fl oz/unit | | |

The addition of the Geobacillus sterothermophilus esterase enzyme increased the yield in the trial above the base seed treatment package alone.

### Example 35

### Free Esterase on Corn Seed Treatment

2.5 fl oz/unit of EstA Esterase from recombinant Bacillus subtilis fermentation (SEQ ID NO: 254) was coated onto hybrid corn seeds treated with Fludioxinil, Azoxystrobin, mefonoxam, and thiabendaz fungicides, cyantraniliproleinsecticide, peridium 1006 polymer, and (colorant). A control treatment without esterase enzyme was also created. The seed treated with esterase and control seed was planted in replicated trials distributed across 16 replicated plots with 4 replicates of 80 ft2 each at each site. Upon harvesting the 16 sites, the data was compiled and averaged and the results are shown in Table 59 below.

TABLE 59
Yield effects of Esterase on corn seed treatment

| Treatment | Yield (Bu/A) | Yield (Kg/ha) |
|---|---|---|
| Control Base Package | 229.63 | 15442.9 |
| Control Base Package with | 230.87 | 15526.3 |
| Esterase 2.5 fl oz/unit | | |

The addition of the EstA esterase enzyme increased the yield in the trial above the base seed treatment package alone

### Example 36

### Free Esterase on Corn Seed Treatment

Rhizopus oryzae Esterase (carboxylesterase, SEQ ID NO:253) was coated onto 6049 V2 hybrid corn seeds. A water only control treatment without esterase enzyme was also created. The seed treated with esterase and control seed was planted in replicated greenhouse trials on promix soil with a fluctuating day/night temperature of 78° F./75° F., and 13 hours of light daily. 3 replicates of each experiment were performed and the averages shown in the Table 60 below. Plant heights were measured on day 10 for each plant emerged from the soil.

TABLE 60
Plant growth effects of Esterase on corn as a seed treatment

| Treatment | Height as % of Control |
|---|---|
| Water Control | 100% |
| Esterase from Rhizopus oryzae, 0.5 fl oz/unit | 103.7% |

The addition of the Rhizopus oryzae Esterase increase the growth rate of corn in this assay.

### Example 37

### Evaluation of Antifeedant and Molluscicide Activity of Enzymes

### Garden Snail Care and Preparation

Two-week old garden snails (Cornu aspersum) and full-size black gloss snails (Zonitoides nitidus) were maintained in a semi-darkened tank at 68-70° F. at high humidity, provided with a wet sponge in a petri dish. Snails were transferred to fresh tank every 2-3 days, and they were feed ad libitum with cucumber slices supplemented with calcium carbonate tablets. Individuals were reared in the laboratory for at least a week to fully acclimate to laboratory conditions prior to testing. The individuals ranged between 3 weeks to 2 months old at the time of bioassay.

### Artificial Diet Preparation

Filter-sterilized recombinant enzymes including proteases and lipases were prepared as indicated in Example 25. Corresponding controls (e.g., buffer, fermentation of non-recombinant strain, water) were be prepared accordingly. Enzymes were dried by mixing them with Polyvinylpyrrolidone K30 (Thermo Sci Chem, Cat #227545000) at a ratio of 60 mg PVP per mL of enzymes, dispersed on a petri dish and allowed to dry in a bio-safety hood for 24-48 hours.

The treatments were prepared by mixing 0.4 g of dry enzyme and 1 gram of artificial diet mix. The artificial diet mix consisted of flour supplemented with 30 mg sucrose per gram of flour. Control diet contained 0.4 g of PVP and 1 g of artificial diet mix.

### Bioassay

12 snails were randomly assigned to each treatment, labeled with permanent marker on the top of the shell for identification, and their weight (mg) was recorded before treatment. Then snails were transferred to dry plastic containers containing two petri dishes. Humidity was maintained with a folded and soaked light duty tissue paper in one petri dish. 1 g of treatment was provided as a thin layer on the other petri dish and was replaced every other day. At the time of treatment replacement, treatment consumption, weight, and mortality were recorded.

Proteases on Garden Snails (Cornu aspersum)

Acid protease, alkaline protease, aminopeptidase, neutral protease, and keratinase, were prepared as indicated above and their antifeedant and molluscicidal effects were tested. Untreated diet was included as negative control. The units (U) of enzyme activity was determined as the amount of enzyme required to breakdown 1 μM of substrate per minute. Treatments and scoring followed the bioassay as described above. Results are shown in Table 61. Acid protease, alkaline protease, and aminopeptidase treatment resulted in lower diet consumption. Alkaline protease treatment resulted in 58% snail mortality and decrease in diet consumption. Acid protease treatment led to a reduction in diet consumption, and the aminopeptidase and neutral protease also reduced consumption in the snails.

TABLE 61
Effect of proteases on diet consumption, survival,
and weight gain of garden snail (n = 12, 14 days)

| | | Diet | |
|---|---|---|---|
| | | consumption | Dead |
| Treatment | U/g diet | (g) | Snails |
| Control Diet | 0 | 1.19 | 0 |
| Acid Protease | 160 | 0.73 | 1 |
| Alkaline Protease | 15200 | 0.45 | 7 |
| Aminopeptidase | nd*** | 0.63 | 1 |
| Neutral Protease | 4400 | 1.00 | 0 |

***Value not accurately quantified with Azocasein assay.

Lipases on Garden Snails (Cornu aspersum)

Lipases from Burkholderia cepacia, Pseudomonas fluorescens, porcine pancreas, type II (PPII) were prepared as described above and their antifeedant and molluscicidal effects were tested. Untreated diet was included as negative control. The units (U) of enzyme activity was determined as the amount of enzyme required to breakdown 1 μM of substrate per minute. Treatments and scoring followed the bioassay as described above. Results are shown in Table 62. Pf Lipase, Bc Lipase, and PPII lipaseall showed consistent reduction in food consumption.

TABLE 62
Effect of lipases on diet consumption, survival,
and weight gain of garden snail (n = 12, 12 days)

| | | Diet | |
|---|---|---|---|
| | | consumption | Dead |
| Treatment | U/gr diet | (gr) | Snails |
| Control Diet | 0 | 1.16 | 0 |
| Bc Lipase | 16 | 0.61 | 0 |
| Pf Lipase | 62 | 0.24 | 0 |
| PPII Lipase | 63 | 0.82 | 0 |

Alkaline Protease and Pf Lipases on Black Gloss Snails (Zonitoides nitidus)

Alkaline Protease and Keratinase and Lipases from Pseudomonas fluorescens (Pf,), were prepared as described above and their antifeedant and molluscicidal effects were tested. Untreated diet was included as negative control. The units (U) of enzyme activity was determined as the amount of enzyme required to breakdown 1 μM of substrate per minute. Treatments and scoring followed the bioassay as described above. Results are shown in Table 63. The three enzymes tested showed reduction in diet consumption.

TABLE 63
Effect of lipases on diet consumption, survival,
and weight gain of garden snail (n = 12, 7 days)

| Treatment | U/g diet | Diet consumption (g) |
|---|---|---|
| Control Diet | 0 | 2.50 |
| Alkaline Protease | 7600 | 0.72 |
| Keratinase | 520 | 0.70 |
| Pf Lipase | 36 | 1.51 |

1. A composition for protecting a plant from a pest or pathogen comprising at least one isolated enzyme selected from an esterase, a chitinase, a protease, a lipase, and combinations of any thereof; wherein:
a) the chitinase comprises a sequence having at least 85% sequence identity to a sequence selected from SEQ ID NOs: 179-205, and 249;
b) the protease comprises a sequence having at least 85% sequence identity to a sequence selected from SEQ ID NOs: 122-178, 247, and 310-327; or
c) the lipase comprises a sequence having at least 85% sequence identity to a sequence selected from SEQ ID NOs: 1-82, 250, 251, 256-260, and 290-307; and
wherein the composition exhibits insecticidal, nematicidal, or pesticidal activity.

2. The composition of claim 1, wherein:
the esterase comprises a sequence having at least 85% sequence identity to a sequence selected from SEQ ID NOs: 83-100, 248,252, 253, 255, and 287-289;
the pest or pathogen is defined as an insect, a mollusk, an arachnid, or a nematode; or
the composition further comprises at least one:
agriculturally acceptable carrier; or
agrochemical.

3. (canceled)

4. A plant seed coated with the composition of claim 1.

5. (canceled)

6. The composition of claim 2, wherein:
the agrochemical is an insecticide, or the agriculturally acceptable carrier is a surfactant; or
the agrochemical is a nematicide.

7. (canceled)

8. The composition of claim 5, wherein the nematicide is fluopyram or pydiflumetofen.

9. A method for protecting a plant from a pest or pathogen comprising applying at least one isolated enzyme to a plant growth medium, a plant, a plant seed, or an area surrounding a plant, or a plant seed, wherein the enzyme is selected from an esterase, a chitinase, a protease, a lipase, and combinations of any thereof; and wherein:
a) the chitinase comprises a sequence having at least 85% sequence identity to a sequence selected from SEQ ID NOs: 179-205, and 249;
b) the protease comprises a sequence having at least 85% sequence identity to a sequence selected from SEQ ID NOs: 122-178, 247, and 310-327; or
c) the lipase comprises a sequence having at least 85% sequence identity to a sequence selected from SEQ ID NOs: 1-82, 250, 251, 256-260, and 290-307.

10. The method of claim 9, wherein:
the esterase comprises a sequence having at least 85% sequence identity to a sequence selected from SEQ ID NOs: 83-100, 248,252, 253, 255, and 287-289;
the method further comprises applying at least one agrochemical or agriculturally acceptable carrier;
the method comprises:
applying the enzyme to the area surrounding the plant or plant seed; or
a foliar application to the plant; or
the method comprises (a) applying the enzyme to the plant or plant area; (b) applying the enzyme to the plant seed at the time of planting; or (c) coating the plant seed with the enzyme.

11. (canceled)

12. The method of claim 10, wherein:
the agriculturally acceptable carrier comprises a surfactant or a preservative; or
applying the enzyme to the area surrounding the plant or plant seed comprises applying the enzyme to soil.

13-15. (canceled)

16. A recombinant microorganism that expresses an enzyme, wherein expression of the enzyme is increased as compared to the expression level of the enzyme in a wild-type microorganism of the same kind under the same conditions; and
the enzyme is selected from an esterase, a chitinase, a protease, a lipase, and combinations of any thereof; and wherein:
a) the chitinase comprises a sequence having at least 85% sequence identity to a sequence selected from SEQ ID NOs: 179-205, and 249;
b) the protease comprises a sequence having at least 85% sequence identity to a sequence selected from SEQ ID NOs: 122-178, 247, and 310-327; or
c) the lipase comprises a sequence having at least 85% sequence identity to a sequence selected from SEQ ID NOs: 1-82, 250, 251, 256-260, and 290-307; and
wherein the recombinant microorganism exhibits insecticidal, nematicidal, or pesticidal activity.

17. A composition for protecting a plant from a pest or pathogen, comprising the recombinant microorganism of claim 16.

18. The recombinant microorganism of claim 16, wherein the esterase comprises a sequence having at least 80% sequence identity to a sequence selected from SEQ ID NOs: 83-100, 248,252, 253, 255, and 287-289.

19. A plant seed coated with the composition of claim 17.

20. The composition of claim 17, wherein the composition further comprises at least one:
agriculturally acceptable carrier; or
agrochemical.

21. The composition of claim 20, wherein the agriculturally acceptable carrier comprises a surfactant or a preservative.

22. A fermentation product of the recombinant microorganism of claim 16.

23. A formulation comprising the fermentation product of claim 22 and at least one agriculturally acceptable carrier.

24. A formulation for promoting plant growth or plant nutrient uptake comprising an esterase.

25. A plant seed coated with the formulation of claim 24.

26. A formulation for protecting a plant from a pest or pathogen comprising a fertilizer and an esterase.

27. The formulation of claim 24, wherein;
the formulation comprises an agrochemical; or
the formulation comprises a fertilizer or a nitrogen stabilizer.

28-29. (canceled)

30. The formulation of claim 27, wherein:
the nitrogen stabilizer comprises N-(n-butyl) thiophosphoric triamide (NBPT); or
the fertilizer comprises monoammonium phosphate, di-ammonium phosphate, urea, or a combination of any thereof.

31. A method for promoting plant growth or plant nutrient uptake comprising applying at least one isolated esterase to a plant growth medium, a plant, a plant seed, or an area surrounding a plant or a plant seed.

32. A method for promoting plant growth or plant nutrient uptake comprising treating a fertilizer with at least one isolated esterase, and applying the treated fertilizer to a plant, a plant seed, or an area surrounding a plant or a plant seed.

33. The method of claim 32, wherein:
the fertilizer comprises monoammonium phosphate, di-ammonium phosphate, urea, or a combination of any thereof;
the fertilizer is further treated with a nitrogen stabilizer; or
the esterase comprises a sequence having at least 80% sequence identity to SEQ ID NO: 254.

34-35. (canceled)

36. The method of claim 33, and the nitrogen stabilizer comprises N-(n-butyl) thiophosphoric triamide.

37. A formulation for protecting a plant from a pest or pathogen comprising a fertilizer and a recombinant microorganism that expresses an esterase, wherein expression of the esterase is increased as compared to the expression level of the enzyme in a wild-type microorganism of the same kind under the same conditions.

38. A formulation for promoting plant growth or plant nutrient uptake comprising a fertilizer and a recombinant microorganism that expresses an esterase, wherein expression of the esterase is increased as compared to the expression level of the enzyme in a wild-type microorganism of the same kind under the same conditions.

39. A plant seed coated with the formulation of claim 38.

40. A method for protecting a plant from a pest or pathogen comprising applying at least one recombinant microorganism that expresses an enzyme to a plant growth medium, a plant, a plant seed, or an area surrounding a plant or a plant seed, wherein expression of the enzyme is increased as compared to the expression level of the enzyme in a wild-type microorganism of the same kind under the same conditions; wherein the enzyme is selected from an esterase, a chitinase, a protease, a lipase, and combinations of any thereof; and wherein:
a) the chitinase comprises a sequence having at least 85% sequence identity to a sequence selected from SEQ ID NOs: 179-205, and 249;
b) the protease comprises a sequence having at least 85% sequence identity to a sequence selected from SEQ ID NOs: 122-178, 247, and 310-327; or
c) the lipase comprises a sequence having at least 85% sequence identity to a sequence selected from SEQ ID NOs: 1-82, 250, 251, 256-260, and 290-307.

41. The method of claim 40, wherein:
the esterase comprises a sequence having at least 80% sequence identity to a sequence selected from SEQ ID NOs: 83-100, 248,252, 253, 255, and 287-289;
the method comprises:
applying the recombinant microorganism to the area surrounding the plant or plant seed; or
a foliar application to the plant; or
the method comprises (a) applying the recombinant organism to the plant or plant area; (b) applying the recombinant organism to the plant seed at the time of planting; or (c) coating the plant seed with the recombinant organism.

42. (canceled)

43. The method of claim 41, wherein applying the recombinant microorganism to the area surrounding the plant or plant seed comprises applying the recombinant microorganism to soil.

44. (canceled)

45. The method of claim 31, wherein the esterase comprises a sequence having at least 80% sequence identity to a sequence selected from SEQ ID NOs: 83-100, 248,252, 253, 255, and 287-289.

46. The composition of claim 1, wherein the composition or formulation further comprises a glucanase.

47. The composition of claim 1, wherein the composition or formulation comprises at least two isolated enzymes.

48. The composition of claim 47, wherein the at least two enzymes are present in synergistically effective amounts.

49. A formulation for promoting plant growth or plant nutrient uptake comprising a recombinant microorganism that expresses an esterase to a plant growth medium, a plant, a plant seed, or an area surrounding a plant or a plant seed, wherein expression of the esterase is increased as compared to the expression level of the enzyme in a wild-type microorganism of the same kind under the same conditions.

50. The formulation of claim 49, wherein:
the formulation comprises an agrochemical; or
the formulation comprises a fertilizer or a nitrogen stabilizer.

51. (canceled)

52. The formulation of claim 50, wherein:
the fertilizer comprises monoammonium phosphate, di-ammonium phosphate, urea, or a combination of any thereof; or
the nitrogen stabilizer comprises N-(n-butyl) thiophosphoric triamide (NBPT).

53. (canceled)

54. A method for promoting plant growth or plant nutrient uptake comprising applying at least one recombinant microorganism that expresses an esterase to a plant growth medium, a plant, a plant seed, or an area surrounding a plant or a plant seed, wherein expression of the esterase is increased as compared to the expression level of the enzyme in a wild-type microorganism of the same kind under the same conditions.

55. A method for promoting plant growth or plant nutrient uptake comprising treating a fertilizer with at least one recombinant microorganism that expresses an esterase, wherein expression of the esterase is increased as compared to the expression level of the enzyme in a wild-type microorganism of the same kind under the same conditions, and applying the treated fertilizer to a plant, a plant seed, or an area surrounding a plant or a plant seed.

56. The method of claim 55, wherein:
the fertilizer comprises monoammonium phosphate, di-ammonium phosphate, urea, or a combination of any thereof; or
the fertilizer is further treated with a nitrogen stabilizer.

57. (canceled)

58. The method of claim 56, wherein the esterase comprises a sequence having at least 80% sequence identity to SEQ ID NO:254 and the nitrogen stabilizer comprises N-(n-butyl) thiophosphoric triamide.

59. A method of producing a formulation for protecting a plant from a pest or pathogen, comprising mixing the recombinant microorganism of claim 16 with at least one agrochemical or agriculturally acceptable carrier.

60. The method of claim 59, wherein the method comprises mixing the recombinant microorganism with an agrochemical and an agriculturally acceptable carrier.

61. A method of producing a composition for protecting a plant from a pest or pathogen, comprising:
a) obtaining a recombinant microorganism that expresses an enzyme; wherein the enzyme is selected from an esterase, a chitinase, a protease, a lipase, and combinations of any thereof; and wherein:
i) the esterase comprises a sequence having at least 80% sequence identity to a sequence selected from SEQ ID NOs: 83-100, 248,252, 253, 255, and 287-289;
ii) the chitinase comprises a sequence having at least 85% sequence identity to a sequence selected from SEQ ID NO: 179-205, and 249;
iii) the protease comprises a sequence having at least 85% sequence identity to a sequence selected from SEQ ID NOs: 122-178, 247, and 310-327; or
iv) the lipase comprises a sequence having at least 85% sequence identity to a sequence selected from SEQ ID NOs: 1-82, 250, 251, 256-260, and 290-307;
b) purifying the enzyme from the recombinant microorganism; and
c) combining the purified enzyme with an agrochemical or agriculturally acceptable carrier.

62. The method of claim 61, wherein:
purifying the enzyme comprises:
i) lyophilizing;
ii) spray drying; or
iii) freeze drying;
the enzyme; or
the purified enzyme is combined with a liquid agrochemical.

63. (canceled)

64. A method of producing a composition for protecting a plant from a pest or pathogen, comprising:
a) obtaining a recombinant microorganism that expresses an enzyme, wherein expression of the enzyme is increased as compared to the expression level of the enzyme in a wild-type microorganism of the same kind under the same conditions; and the enzyme is selected from an esterase, a chitinase, a protease, a lipase, and combinations of any thereof; and wherein:
i) the esterase comprises a sequence having at least 80% sequence identity to a sequence selected from SEQ ID NOs: 83-100, 248,252, 253, 255, and 287-289;
ii) the chitinase comprises a sequence having at least 85% sequence identity to a sequence selected from SEQ ID NOs: 179-205, and 249;
iii) the protease comprises a sequence having at least 85% sequence identity to a sequence selected from SEQ ID NOs: 122-178, 247, and 310-327; or
iv) the lipase comprises a sequence having at least 85% sequence identity to a sequence selected from SEQ ID NOs: 1-82, 250, 251, 256-260, and 290-307;
wherein the recombinant microorganism exhibits insecticidal, nematicidal, or pesticidal activity;
b) purifying the recombinant microorganism; and
c) combining the purified recombinant microorganism with an agrochemical or agriculturally acceptable carrier.

65. The method of claim 64, wherein:
purifying the recombinant microorganism comprises:
i) freeze drying; or
ii) spray drying;
the recombinant microorganism; or
the purified recombinant microorganism is combined with a liquid agrochemical.

66. (canceled)

67. A method for controlling a plant pest or plant pest infestation, said method comprising contacting the pest with an effective amount of an isolated enzyme selected from an esterase, a chitinase, a protease, a lipase, and combinations of any thereof, wherein:
i) the chitinase comprises a sequence having at least 85% sequence identity to a sequence selected from SEQ ID NOs: 179-205, and 249;
ii) the protease comprises a sequence having at least 85% sequence identity to a sequence selected from SEQ ID NOs: 122-178, 247, and 310-327; or
iii) the lipase comprises a sequence having at least 85% sequence identity to a sequence selected from SEQ ID NO:1-82, 250, 251, 256-260, and 290-307; and
wherein the enzyme exhibits insecticidal, nematicidal, or pesticidal activity.

68. The method of claim 67, wherein:
the esterase comprises a sequence having at least 80% sequence identity to a sequence selected from SEQ ID NOs: 83-100, 248,252, 253, 255, and 287-289;
the plant pest comprises Black armyworm (Spodoptera cosmioides), Black cutworm (Agrotis ipsilon), Corn earworm (Helicoverpa zea), Cotton leaf worm (Alabama argillacea), Diamondback moth (Plutella xylostella), European corn borer (Ostrinia nubilalis), Fall armyworm (Spodoptera frugiperda), Cry1Fa1 resistant Fall armyworm (Spodoptera frugiperda), Old World bollworm (OWB, Helicoverpa armigera), Southern armyworm (Spodoptera eridania), Soybean looper (Chrysodeixis includens), Spotted bollworm (Earias vittella), Southwestern corn borer (Diatraea grandiosella), Sugareane borer (Diatraea saccharalis), Sunflower looper (Rachiplusia nu), Tobacco budworm (Heliothis virescens), Tobacco cutworm (Spodoptera litura, also known as cluster caterpillar), Western bean cutworm (Striacosta albicosta), and Velvet bean caterpillar (Anticarsia gemmatalis) Garden snails (Cornu aspersum) or slugs (Deroceras reticulatum); or
the plant pest comprises a nematode species from the genera Heterodera and Meloidogynes.

69-70. (canceled)

71. The method of claim 68, wherein the plant pest comprises a nematode species selected from the group consisting of: Aglenchus spp., Anguina spp., Aphelenchoides spp., Belonolaimus spp., Bursaphelenchus spp., Cacopaurus spp., Criconemella spp., Criconemoides spp., Ditylenchus spp., Dolichodorus spp., Globodera spp., Helicotylenchus spp., Hemicriconemoides spp., Hemicycliophora spp., Heterodera spp., Hoplolaimus spp., Longidorus spp., Lygus spp., Meloidogyne spp., Meloinema spp., Nacobbus spp., Neotylenchus spp., Paralongidorus spp., Paraphelenchus spp., Paratrichodorus spp., Pratylenchus spp., Pseudohalenchus spp., Psilenchus spp., Punctodera spp., Quinisulcius spp., Radopholus spp., Rotylenchulus spp., Rotylenchus spp., Scutellonema spp., Subanguina spp., Trichodorus spp., Tylenchulus spp., Tylenchorhynchus spp., and Xiphinema spp.


SEQUENCE LISTING (328 sequences)

SEQ ID NO: 1 - protein, 413 aa, Bacillus cereus
  source 1..413: note = Lipase
MGKLFLKVCFFALVTVCSFAAKTTYAEERQQNNYPIILVNGFAGWGREEMLGVKYWGGVHDIQEDLKRNGYTVHTAAVGPVSSNWDRACELYAQINGGIVDYGAAHAEKHGHNRFGRTYSGFAPNWSETNKVHLVGHSMGGQTTRTLVQLLKEGSYEEKDYVKNHPNTKISPLFEGGKSYVHSVTTLATPHNGTTLADGSLLLPFVKDLLITAASLGGNNNLSLYDFKLDQWGIKKNAGESFFQYTDRILNSSIWENTKDISQWDLSTDGAKELNNWVKTQSDVYYLSYSGHASQAAPITGLHLPHITMNKVLMGNAFFLGSYARYEENRPLIDTSWWQNDGVVNTNSMIAPASNTFVNNNESLQIGKWNHIETKANWDHLDMVGLSVSDTLGFSNIQEFYRTIVGKLSRLPK

SEQ ID NO: 2 - protein, 240 aa, Bacillus sp.
  source 1..240: note = Lipase
MRTPLSFDKDTAILLASCCELTYEQYKQNGIFEIPDGFQYVQGFQGKAIQKTEWFGFILESEDTIIVAFRGTQTDTDWIIDSLVNQRPYPYALNSGNVHNGFLSIYESCRDSIMDMLVSLPAHKKLLATGHSLGGALATLHILDARINTAFAQYGLYTFASPKVGDIAFRNYYKLQVASSFRFVNLFDVVPLLPPRNVHFNEQDWEYAHVHHNMTFTKNTKSITNNHAMTTYKTCLTSHF

SEQ ID NO: 3 - protein, 293 aa, Bacillus sp.
  source 1..293: note = SGNH/GDSL Hydrolase/Lipase
MKIAFRKFVWYNYGQSIGAKRGFDMRSKLVKVILLITIASFCLFAYGFVSGVNDVLNPKASKLITKTDVLEKEKKKTGTLQVVSLGDSLTRGVGDKEGIGYIGRMKENLQKDYKQKVALSNLAVSGAKMPDLLKQIESSGAQYSIKQADVIVLTIGGNDLFPGWESLGKIDLETYRPDTETFQNEAKKIIEEIRKLNTDSPIFWLGLYNPFEDVEDLRGSSNIVVDWNASLEKLALNDKNVYITPTFDLFQNRGKDLLYSDHFHPNEVGYTYMAERLVQNVVSKLKLEQGGIK

SEQ ID NO: 4 - protein, 315 aa, Bacillus sp.
  source 1..315: note = Alpha/Beta Hydrolase/Lipase
MLKKVQLEKAAQEFAEATANPPYLFDLGPEKGREAVDEVQSDPIDKLPVDIEDVMISGGPSGQVSVRIMRPQNAPAHLPVIVYIHGAGWVFGNEHTHDRLIRELAVGAQAAIVFPNYTLSPEAKYPTAIEEIYAVVKWASENGRENGMIPESLTIAGDSVGGNMAAAVTLMAKARGDLNIRQQLLFYPVTDASFDTESYHQFAAGYFLRKDAMMWFWDQYTTNSNERAEITASPLRATAEQLQGLPSALIITAEADVLRDEGEAYANKLREAGVPVTAVRFQGIIHDFVMLNALAKTEAARGAIDLAATWLRKGF

SEQ ID NO: 5 - protein, 214 aa, Bacillus sp.
  source 1..214: note = Triacylglycerol Lipase
MKHIKSKILVILTVCMLSVISVFAFQPTVSKASSGHNPVVMVHGIGGASFNFAGIKTYLASQGWSRKEMYAIDFLDKTGNNRHNAPRLSNYVKKVLSETGAKKVDIVAHSMGGANTLYYIKNLDGGDKIANVVTLGGANGLVTNRALPGTDPNQKILYTSIYSSADLIVLNPLSRLIGGKNVQIHGVGHIGLLMNSQVNGLIKEGLNGGGQNTN

SEQ ID NO: 6 - protein, 250 aa, Priestia megaterium
  source 1..250: note = Lipase
MMRIEPLFYRPLALDLLYACVLAYKQYAQGGTFDVPEGYRLIKSFKASAVGVQEWFGFILESDDSIVIAFRGTQSEADWIADARIRQRPYPYNQQAGLVHEGFLAVYESCRDEIFETYQSLTPKPLYITGHSLGGALAALHALDVATNASFPEVTMYNYGAPRVGDPQFVQTYTNLVSNSRCFVNTTDTVPKIPPKRLYSPSTKQTIYYDQDPLQLSFTIQTGSTVGNHNPQTYSVGIWMMSDYPILVQK

SEQ ID NO: 7 - protein, 210 aa, Priestia megaterium
  source 1..210: note = Triacylglycerol Lipase
MKKTFVALILALSLVVSALGIQPSNAKAESNHNPVVLVHGISGASYNFIAIKNYLIAQGWDRNDLYAIDFVDKTGNNLNNGPQLARYIDQVLKETGAKKVDIVAHSMGGANTLYYIKYLGGGNKIENVVTLGGANGLVSSTALPGTDLNQKILYTSIYSINDQIVANSLSRLQGARNIQLYGIGHIGLLSNSQVNAYIKEGLNGGGQNTN

SEQ ID NO: 8 - protein, 281 aa, Cytobacillus firmus
  source 1..281: note = Lipase 1
MNNSVFKEKFSTEEAILLAGMIHQAYQLFETGTLSLPRGFRLRHIIRALAGVEEPEEEVFGFIAESAEDIVVTFRGTRTFKDNESDQDLYQVPYPYVRDAGFTHRGFTCIYSSTRNELIRELNRLSTGKRVLIAGHSLGGSLAVLAALDIEVNTRFKDPIIYTYGSPRTGDPLFASFFSQTVQNSIRIVNIHDIIPTLPARVYPPPFTKDGLYYRHVNRKYPVSFQLNSLPVRNHEIVCYFHNLSMQNPYYTRTLCAGSQGFCPDTVMCVPFIGGCSGEIP

SEQ ID NO: 9 - protein, 271 aa, Cytobacillus firmus
  source 1..271: note = Lipase 2
MKKIINSEAAILLAAFSYQTYPLYLNGSLTLPKGYELRYIIRAFADVENPAENIYGFLAESKDKIIIAFRGYAAYPSDLLASYDIFQIPYPYVPDGGKTSRGFTCLYQSARNNLMEQLNKLSSSKKLFVTGHNYGGALSVLAALDMAVNSKFKKPFVYTYGSPRIGDPEFVMKFDQKVKNSIRVVNIHDSFPTFPEVRYPPPFTEEGINYQHVKTEYRLSFQLNNTPRNDSINCYFKSLRSLNPNFADILCSENPGFCPDTEVCVPFQRTC

SEQ ID NO: 10 - protein, 213 aa, Bacillus subtilis
  source 1..213: note = Lipase (LipC)
MVLRYTALGDSLTTGRGSGLFSPGFVQRFGDMMEADLKTRTAINIFARSGLNTEEILGLLSYPYVQKCIRDADMITITGCGNDLIDSVLAYQTSKDETIFSRVSAHCHENFEKMIAKVAEIKGENPSPYAIRVFNLYNPFPEIDIAGKWITSFNSHLETLASAPHVKIADAYSIFKGKEQEYLSFDGVHPNSKGYQAMAEAVHKLGYKELSVS

SEQ ID NO: 11 - protein, 533 aa, Bacillus cereus
  source 1..533: note = Lipase (Lip 1)
MRLMRRCVALLIVFFIMAPMISTNVRAGVVKELKTGFPDQEVFTPGEWFLGQKPANYDENKPPILFVQGRNGNADSWYGKTVYHDINDMYDYALKAGYQTVFIQLYDAAGKGSASQWDNGKLLAQKLEEIYNHFGKKVNIVAHSKGGIDTQAALVGYGANRFVGNVITLATPHHGSNLADLSYSWWAGWLASILGQKDDGTYSLQMGEMAKFRSTIDNNPAAKLNRYYTVTGTSWGPVFSALSMGGLYLSSYGSNDGLVNECSAKLPYGTHLFTDSRFDHDNIRKGSAVFARIEPYLRTANVPVPALVASSTSSNENIEQLNTTSNQSILGGELPQNQWIEQTVAVDKKAEGIVSILTASSDVEVQMISPKGKVYTNKDSVITTGEGESFFGGATIRTFKFDKMDVGEWKVKMMTKQSKDAYLVVSDYKTGAPFVLQMPTKVKANKSEYKLKKSPVAPEMKGDLSITVRVVNKEGKLVSEFNELQNVNNNTFTGALKNIKQPGVYNVTMDIKGMNKEGKSYSRTIVKSVYVEK

SEQ ID NO: 12 - protein, 487 aa, Bacillus licheniformis
  source 1..487: note = Lipase
MKKLVLLMLVLLLVYPHVSKAGGFKGGGGNPGYWFAGDPVEHPDPAKPPIVFVHGLNGSSSAWFDENDMAEQAWKNGYDAAFIDLHPDKDMQDNGAMLAAKLREIYQYFGRKVILVSYSKGGIDSQSALIHHNAYHYVERVITLGTPHHGSQLADLAYSNWAGWLADILGQKNDAVYSLQTGFMKSFRDQTDNHPNRLKTKYFTLAGNKIGGFGSALFFGGVYLNMFGENDGAVTEKNARLPYATNLDTGKWDHFSIIKGNLTFPVFMPLLTIQANANETAATKENLSYPFIRGGENHGLREEEFAVEKGVKEITVHWLSNHSSGNIKLTDPRGKPFKDFSIAKTADVFEGGFVHSAAIKNPAAGTWKIASSVKQKEAFLFIVTFDSPLNQQIKNAVTRESSNLANVKASVRSIRYENGKQAEKKSLKPASINALQNSLSFKKAGMYSVTIDLSGKTADNSPFNRTIIRSIYVNDKGEKFENSPLSD

SEQ ID NO: 13 - protein, 546 aa, Beauveria bassiana
  source 1..546: note = Triacylglycerol Lipase
MMLLMQLMCVAGALGAAVALSADIDGAPLIHLDYATYRGSRVAGAGVDQFLGMRFAKPPLGDLRFRAPEEPFHEDQVQDATKLGALCVGTVDGSGVSPNDVFGEDCLFANLFKPANATTASKLPVWVFIQGGGYAHNSNGNYNGTQIVQQSGGNVVFVSFNYRVGVLGFLASEEVRKDGALNAGLLDQRLFLKWVQKYIGQFGGDPNHVVIHGDSAGGGSVSHHLTAYGGNDKENLFVGGIPESPFWPTQRKVDQMEFQYTRLLRDTKCDSLDCLRKLDVGALGNASQPSPFPEAHTEDPAPLWYWLPVIDGDLVQDNMYKQFLTGRFKRVPLLVGADTDEGTLFAVNASTETEVSSFLRANYPRLQEWQLDIVSTIYPKMKPLPKHAAYFPSAAAVYGDSTFVCAGNTMSDAISAFVGPDKLWNYRYNVADPEQVAEGFGVPHVSELGAIFGPDNIRGHEAKSLRTTNAPIVPVTMAYFTSFVRFLDPNTSRQPGSPLWQNWGAFVGEQGQRMRLETNNTAMETVPFDLVTKCALWKLLAESMDV

SEQ ID NO: 14 - protein, 558 aa, Beauveria bassiana
  source 1..558: note = Lipase 1
MHTLTQTHARIMMLLMQLMCVAGALGAAVALSADIDGAPLIHLDYATYRGSRVAGAGVDQFLGMRFAKPPLGDLRFRAPEEPFHEDQVQDATKLGALCVGTVDGSGVSPNDVFGEDCLFANLFKPANATTASKLPVWVFIQGGGYAHNSNGNYNGTQIVQQSGGNVVFVSFNYRVGVLGFLASEEVRKDGALNAGLLDQRLFLKWVQKYIGQFGGDPNHVVIHGDSAGGGSVSHHLTAYGGNDKENLFVGGIPESPFWPTQRKVDQMEFQYTRLLRDTKCNSLDCLRKLDVGALGNASQPSPFPEAHTEDPVPLWYWLPVIDGDLVQDNMYKQFLTGRFKRVPLLVGADTDEGTLFAVNASTETEVSSFLRANYPRLQKWQLDIVSTIYPKMKPLPKHAAYFPSAAAVYGDSTFVCAGNTMSDAISAFVGPDKLWNYRYNVADPEQVAEGFGVPHVSELGAIFGPDNIRGHEAKSLRTTNAPIVPVTMAYFTSFVRFLDPNTSRQPGSPLWQNWGAFVGEQGQRRMRLETNNTAMETVPFDLVTKCALWKLLAESMDV

SEQ ID NO: 15 - protein, 326 aa, Beauveria bassiana
  source 1..326: note = Lipase 2
MTAWTRERLLALGTPSPQLTRNQPKRFDLDVLAQLRANLAGSRLPLDSAILETDLVCPVRDGTTIKLRWYRPAMPPTPGLPLVVHYHGGGYILGDLNTSSPFCQALVRDHGAVVIDVDYRLAPEHPFPTGKNDAWDAMVWVSKNYAVLGADPSLGFLVAGLSAGGNIAASMALVARDEKLEPALTGTYLCVPELVSSHQKLPDKYQSLWLSRVQNKAAPILDQPFLDFIKGKQCYKPKLESHYHAPAHHPQGHAGLPKTYIDVCGLDPFRDDGIIYEQLLQDAEVLTRMDIYPGMPHGWWVVLPELEGTEEYHRKRREGLKWLLAK

SEQ ID NO: 16 - protein, 440 aa, Beauveria bassiana
  source 1..440: note = Lipase 3
MASILSTLGLILSKIPLLLKTIVFYLASLSPTARKWDLGTEVAVTMLRDFLGNAPPSTVSAQQALFMRRDTGARGRIWDVSGEWVGVRHGVSRWAAEPARVSERDKFARLTAEAQTDVTVLYIHGGANYLMDPVSARPVALAYATHGKCRVFSVRYRLAPQGPFPSALLDVFVAYLSLLYPPPGAWHSPVSASSLIVTGDSAGGNLSTSLLQLLLQLHRAAGAGAALPPTVRFHGRDVPVPLPAGVGLNSPSLDLTRSMQWSESFLQYDYLPPPHLSPVTSPPCAVWPADPPRVHLYCEGGALAHPLVSPMAAASWEGAPPVFFVCGEELLADEAKTVACRMARQGVPVVWEQWEAMPHCFSMLLTWTEASRVSYRGWADFVRDAVRGEVQTKGCYVEVKTLKRREVDVKTLTTISDEDVLEGMAAGRKRIEDKFAYLLK

SEQ ID NO: 17 - protein, 618 aa, Beauveria bassiana
  source 1..618: note = Lipase 4
MAVPFIGRLHPMEYIALVGSFFLVFLEAIIRICTLALPSFLVAYFYRASRRLFLRFSSPEDKRVNRKTKCISDSVRDAADFVELCRFWGYEAEEHIVQTKDGYLLGLHRLQWKRGEEGQRVNNGPKSIRKRVIYMHHGLLMNSEVWVCQTDEKRCLAFELVEQGFDVWFGNNRGNKYSKKSIHSSPNSTRFWDFSIDEFAFHDIPDSIEYILESTRQQSLSYIGFSQGTAQAFATLAIHPKLNHQVNVFIALAPAMAPAGLSNGIVDSLMKASPSALYLMFGRRSILSSATTWESIMYPPLFARSIDMGLSFLFNWKTLNISYSQKLAAYPHLYSFTSTKSVVHWFQIIRNRSFQLYDDDVHQPISVTTSRKSSKVAKYPTRNIRTPVVLVYGGSDSLVDINSMLRELPPQTVATEIPHYEHLDFLWARDVDVQVFQHVFDALDSFTDAEHTVEEYDRYHIVRSESLMGSGLLVNANSTTDSDASVLGSKSHGHLPVPQPHVAREPAVRSRAATVGSSPSTSFKGVKFEPLPTKKPQLDSSVPGSNSDKLRGVGVRREPEQLTGLNVSVLRDGRGISPGQANAVGGVVADTSGVERADVISSSGEEKRAEKKTLKKRR

SEQ ID NO: 18 - protein, 374 aa, Beauveria bassiana
  source 1..374: note = Lipase-like 1
MQPTAFFLLTGATSALASPAVSVRQATPDKASPAEFELFSQYAQAAYCTGLYDTTINTAVCADNRGSICDGFKGTVTVKEFTNLEFGTIAGYVATNPSKKQIVVAFKGTNPLSYVDVTSDLIKNLVAANNLFPKCGGCSIHNGFMRAFSSVRAELEQTLKAELAKPGQESFRVVITGHSLGGAVATVAAPYLRTQGIACDLYTYGSPRVGNQEFANLITNDSNFSARITNGNDFVASVPFGSLFQLGFYAHTYPEYWYKDGLLGTAKGYETTATKCTSRKQCAGPTCGRARNVLEFPVARFTCSIGDHMNYVDPALLLCKDGRKGKSSEPSITAEEFQQFLEAQEKEEEEKKEGEEEKKEGEEEKKEGEEKQEQ

SEQ ID NO: 19 - protein, 377 aa, Beauveria bassiana
  source 1..377: note = Lipase-like 2
MWLLLFACLLHVVVAQQAPGVSVSMFASLERLARLVDITYCIGNTGIGKPFSCLSRCSDFPNVTLSRTWSTGVFLTDSCGYVAVDETPASSLAVDSHGAIIVAFRGTYSIANTVIDLSTVPQEYVPYPSQGDAPAHKCTNCTVHMGFFQTWQSARESVIPELVQLRKTYPSKPIHLVGHSLGGAVACLAALELKTSLGLDNVVVTTFGEPRVGNDGLVDFIDRVFNLNDKGDLEKRSYRRVTHANDPVPLLPPGEWGYKSHAGEIHITKPDLSPAEEDLQLCIGDQDNSCSAGQDSTSQGLARLTQFYQGVKGSQLQKRGISTRLNLWQLFFAHRDYFWRLGLCFPGGDPFDWGRRKLIEKLPEAEGLEMEAEYQEL

SEQ ID NO: 20 - protein, 338 aa, Beauveria bassiana
  source 1..338: note = Lipase-like 3
MRVALFSLVAAAAASPIVNNQTLLESRSVDNNFMNAFKFYAQHAAAAYCNAEGRSPGNAISCAGGECNDVMRNGATIINTFQGANTGIAGYVSVDRTRQEIVFAARGSNNLRNFITNLIFTQRDCDFASGCKVHDGFAASWDEISVAATAAIRSGLQANPGYRLVITGHSLGGAIGTLAGVYLRRAGYQAAIYTFGAPRIGNEVFANFASRQRGGLYRMTHIDDPVPRLPPMIFGYRHGGTEYWLSNGQAEQINYQGNDVKVCPGIDSVGCNAGTIGFDLPAHLHYLTNTAGCAPPATKWKRDPEPSDEELEERFTNWSKQDQKLVNSCYYCYTVEVV

SEQ ID NO: 21 - protein, 472 aa, Beauveria bassiana
  source 1..472: note = Lipase 5
MQITIWSVARTILLLTASLNSAYGRTINNARDLIPNPSQDPFYVPPSGFESASPGAVLRSRKVASSFFSIIPNPIETYQILYRTTAVDGSAIATVTTVFKPVLGKRDRFVTWSSAYDSAATQCNPSYSYRLGSSIVPSILTTNAVEQLIIEIYLLKGYIVSSPDYEGPDAAFAAGRLSGMGVLDSMRAVSNFGRTLGLTTDKPSIVGAGYSGGGLATAWAAALQPSYAPELPVKGWTAGGIPANLTYIFEFIDGSLLSGVEIVALAGLMKPSAYGATLKPFIDGIGTSALQKVLQIANTQCFIPNLLVFPFQSLLDTKYQSLGKDVLTNATMLSALQDNTLGVDKAQKPTAPFLMFHAKPDELVPYAPAAKVHDTWCDNGATVKFVEYANGGHVTTAVLGLVDAFKFADDAFNNQVASGCTSKTVLDDTLNPLAFGVALEPLTVGLINYLVALGRKDASWLAGIKKGEPIKL

SEQ ID NO: 22 - protein, 292 aa, Beauveria bassiana
  source 1..292: note = Lipase class 2a
MIFNVAAVVGGLIAVVSASPAASVPALIRNRVAKHNDFSCQPSAEHPNPVILLHGLGATWYEDIYLLELWLQTKNFCTFSLTYGAYDGFPFVGGLRPIDESSREIAALISEVHAKTNASKVDLVGHSEGGFQALYVPKFREGIAALVENIVAIAPPTHGTTFAHLWTLVQLFGKNARKDVGTVLEKLGCAACNEISVDGPSIAKLNDGEPIVQAGNKVTVITSEFDELVTPTDTAFVREPGVKNIYVQDYCPFDPVGHIGEAYDQNVWNLVLNSLEKQVGRRFFCLVGAPGK

SEQ ID NO: 23 - protein, 505 aa, Beauveria bassiana
  source 1..505: note = Lipase 6
MRRLTRCLAALVCLVPLATLAEQVANFDVSQATAESHDCGSACRARLALTIPADRDAVGRDFDVNFYATAANFSRASSQPGDVLKLEPVDPKTLTLDGGATTYRFQYVSVDADGSVVPVTGFIAFPFTPHIPADGGDGRAPLKYRLAAFAHGTVGIFRGCAPSNGAALYDYSSWQPALRRGYAVVATDYAGLGNNYTDHKYLFLPAHASDVHYAVVAARRLFGARLTDEWVSFGHSQGGGAAWKLAESVYVRNDTGYLGTVAIAPATYAADQIYAAAVDGLSGGGGGNSSSSNSSRKGGAGVGFLPLIPTAVQRVIPSYNESILSPTLRSRVAMAEEAQLCLEGVLGLTLDLGLADVVSVAGALRDRPTLRRWQDMAAPAQGDVSPAPILVVQGQNDTAVRWQTTEEAWARACACAAGNEVHLRLFPTQGHRPSLAAGAPEWLAWMDGLFDEQRMKAAGGGGGACRVREKKKCTRYVREPFDGEYVRAPTDVDLKPYLGPLNQTM

SEQ ID NO: 24 - protein, 338 aa, Beauveria bassiana
  source 1..338: note = Triacylglycerol Lipase
MSRCAIHQRPLPLPLLLRRRRIHGFRTFCTSRRLGKDPRIPGAGREIIDDFAQLREHYATPKYPIVLAHGLFGFSELRLVPRLPAVQYWHGIQDALRANGATVFATSVPPSSSIAVRAATLADGIVAAQEAQEKACASPPPPVNIIAHSMGGLDARYMISRLLPRPDVQVASLTTIATPHRGSPVADYLVETDSRSPIHLPQLYALLARAGLGTEAFEQLTTRYMTGTFNPETPDVDGVRYFSYGADMKEVPSRLSVFRPPWQVMQDAGEGPNDGLVSVASSRWGEYRGTLVGVGHLDLINWSNRMRWALREMMGMKRTFNAVAFYLDIADMLAREGL

SEQ ID NO: 25 - protein, 305 aa, Beauveria bassiana
  source 1..305: note = Lipase class 2b
MHVTIASTAALAGLILATTFADGQALQTTSIGKHNDFSCQPSANHPRPVVLLHGLGATYYDDLHVLEAFLQIRGFCTFSLTYGAVKGSPFLGGFAPIDESSRQIADFIYEVHAKTVGYSDGGGKIDIVGHSEGGFQALYVPKFRDGVAALVDHIVAIAPPTHGTPIANLLSLSPPLDHDAGLLEVIGCAACDQLSIGGASVVKLNDGKAIVQPGNKVTVIISNLDAIVIPPKSAFIYEDGVDNISVQDYCPLDIVGHISEPYDVNIWNLVLNSLENQVGRKFPCLVGVHIYKYIFTNVELRSAST

SEQ ID NO: 26 - protein, 443 aa, Beauveria bassiana
  source 1..443: note = Lipase 7
MRLAFGLRLLAAAAVVSAWRRMFPVPPSSDSFYHVPRLISNYAPGSIINYRLVPNDVGAFGVKTNLAAAYQILYRTNDGNDQATATVLTVLIPPNANHSAVLTFVMAEDAVSIDCAPSYGILLESELLRRTNSATTQLQLLLIEAAMAQGWVVIIPDFEGPQAAFVDSKLGGQATLDGVRAALASGWLTNIQRDATLTLWGYSAGASVALQAAGMRPTYAGELNIAGAALGGMASPSLSLSIFTDINKGPHAGLIPVSLIALGRADATFQASLDKHLRASARNKFYLPTKQCLDANLATFNNADIFAMFECWDCVVADLVTAIGTHHEPTPVPRSGQIFVYHCVHDELIPIDGIDKQVQDFCRDSAIVHYQRDNGPNLNHRNYGVIGAPHAIEWLRGIYNGTTRQTTCSQETVTSVDLPDWFLDTYPNRVRDPLVKLVAGATK

SEQ ID NO: 27 - protein, 496 aa, Beauveria bassiana
  source 1..496: note = Lipase 8
MQFVRALTPLLALLLCPFVLADADCDDAQPPSQDPWYTLPKGVDCSGAPGKVLRLRRAPKTARESLRGIQDAWNILFCSTDSAGEPTFSVTTVLVPHDADSKALVTYPPAYDSASVDSSPSYALSNLVGSSVEQAIFTPLLVSKRWVVNMPDYEGPRAAYLAGNNSGHGLLDSIRAVRSLHDLFGLCPDFQHAVYGYSGGAFASQWAAQLQPSYAPELAIGVMAYGGLAVNATSVIPAINNGPAAGLIVLALLGTTAQSQEARMILAKNLRKTGLYNSTTFFSALKMDSQTATALFQNQDISKYFVEGFSFLNEPAFVRAFYDDTLAGRGDTPTIPMYVFHGVKDLISPIKDMDELVKKYCAAGTSIEYRRNVVGDHGGTGIAEGIRALNWLGGFFAGAHQPEKRCHVNDVTSALGSDSVALAFIVFSKQGYRIAQFLKIHALPGVPSIPPNATNAEVFGALRLTTRASLAKIGLNDQALAQFHVSEQDLAELGIH

SEQ ID NO: 28 - protein, 454 aa, Beauveria bassiana
  source 1..454: note = Lipase 9
MLAQGLLSAAIVVAHALVASAAPPMPHDPRTAPILPSQDPFYAVPDSISAAAPGAILRHRKPPSSIAVFGFDSVNLAETVQIQYRTADNLGNATAAVLTVLVPHHADMSKVLSYQVAEDAASADCAPSYTLQRESKSGPHLGTLFEQAEILLIEAVLEQGWVVVVPDYEGPNALYLAWRLAGQATLDGIRAALASEEITGIKADATVAMWGYSAGSMATAWAAEIQPQYAPELRIAGAAFGGTTPNITNVVGATNKGRFAGLLPTGIMGLSKQYPAIEAMVDRELKPEFREVVEQTKHQCLVADLAVFSDRDVTAMFTDPRFMYNDPDAVKVLAEIDLGQAVPEIPIYWYKSVHDEVSPVADSDAVYAKYCAGGATVEYLRDEASEHASAMATGAPRAVAWLKNIMNGGKPKPGCSKETTLTSLWDLSTIEVLPGFILDALLDLAGKPVGPWLA

SEQ ID NO: 29 - protein, 568 aa, Metarhizium anisopliae
  source 1..568: note = Lipase 1
MQPPFTTTAVLLMASNALAGVVQPISVPAVHIRNGTVVGKYIESHNQDAFLGIPFAQPPIGDLRFNAPQSISEGWKSPLNATAYGAHCINYLLGLPLDPADLATRYPQSEDCLTINVVRPSGTKPNARLPVLTYIYGGGFQEGGSADARYNTTALVDKSVQIGQPSIVVTMNYRLQGWGFLAGDEARKQGLLNLGIQDQRLALRWIQENIEAFGGDHRRVTIQGESAGALSVGFHLLANGGRDDGLFNAAICQSGGPYNALSFPSDAQSQKTYESVLKAINCTDASDTLKCLRAAPFDTLNTAFASLSFLPVIDGTLVPEYASTALASGRFVKVPLLIGANTDEGKVFAGMGVNTTEEFAGFIEKYPYVHTTTNATIRDLLEAYPEPGTNSTHGQSDDTLPVSAPYGAQFLRAARYTGDVMFIAGRRYTCETWAHYGVPCYSYRFNTIPGATDPLYLGATHFEEVAFVFDNVLGLGMPSNAFDVEPAERKQSYKQLGDTMSRMWMSFSATHSPNNHRVKSMRTLWPAYNLKNPQNMVFDGNITSFVEKDDWRIDALKLIIERSSDFSR

SEQ ID NO: 30 - protein, 366 aa, Metarhizium anisopliae
  source 1..366: note = Lipase 2
MLLKTVLGFVATAAAAQLTAVDHIKALVTREHLQAGTERISNRDFSAIRFYSQHAAAAYCNFNAAPGTRITCQNNACPLVMRNQPVVVASAIGDALGVGAYVAVDYVRREIVLSFRGSNNIRNFIADLAFAWSDCNLTQGCKLHTGFAQAWYDISDAITKAVRSARSSNPNFRVVATGHSLGAAVATLSAAYLRRDGLAVDLYTYGSPRVGNKNFATWFLTQRGVQWRVTNGDDPIPRLPPLVFGYNHISPELWRPGGDVQTVWQPSTTAICKGVDNTDCNGSGLSPDPWAHRNYFGSVDACAGSSLTLRDARGLPEDLINRLTDWSRQDRQLSGSLGHHGDVEFLLKPLDESPASAPGKHEYTIV

SEQ ID NO: 31 - protein, 343 aa, Metarhizium anisopliae
  source 1..343: note = Lipase 3
MLFKTALGFAAIAAASAPIAADDSAKALETRWGARISNNDFANIRFYAQHSAAAYCNIGVPAGQQIACGNNACPLVARNRVKVVASVTGDLTGTGAYVAIDTFRREIVVSIRGSNNIRNYITNLIFSWTDCNFTKQCQVHAGFAQAWDEIKVAVNRAITNARRRYPQYAIVFTGHSLGGAVATIGAANLRRSGLWVNLYTYGSPRVGNDWFASWFSNVQGGQWRVTHEDDPVPRLPPIFSGYRHITPEYWLSGGNGGNTYKTDYTIANIKVCEGIASTQCNAGRDVTDINAHLYYFGAIASCAPSSLQLRDADAQDDPLPKDLSERLTDWSRKDQEFVKSENV

SEQ ID NO: 32 - protein, 364 aa, Burkholderia cepacia
  source 1..364: note = Triacylglycerol Lipase 1
MAKSMRSRVMAGAVACAMSVAPFAGTTALTTLATTHAAMAATAPADDYATTRYPIILVHGLTGTDKYAGVLDYWYGIQEDLQQHGATVYVANLSGFQSDDGPNGRGEQLLAYVKTVLAATGATKVNLVGHSQGGLTSRYVAAVAPDLVASVTTIGTPHRGSEFADFVQDVLAYDPTGLSSTVIAAFVNVFGILTSSSHNTNQDALASLKTLTTAQAATYNQNYPSAGLGAPGSCQTGAPTETVGGNTHLLYSWAGTAIQPTLSLFGVTGATDTSTVPVVDPANVLDPSTLALFGTGTVMINRGSGENDGLVSKCSALYGKVLSTRYKWNHIDEINQLLGVRGAYAEDPVAVIRTHANRLKLAGV

SEQ ID NO: 33 - protein, 364 aa, Pseudomonas sp.
  source 1..364: note = Lipase 1
MARTMRSRVVAGAVACAMSIAPFAGTTAVMTLATTHAAMAATAPADGYAATRYPIILVHGLSGTDKYAGVVEYWYGIQEDLQQNGATVYVANLSGFQSDDGANGRGEQLLAYVKTVLAATGATKVNLVGHSQGGLTSRYVAAVAPDLVASVTTIGTPHRGSEFADFVQNVLAYDPTGLSSSVIAAFVNVFGILTSSSHNTNQDALAALQTLTTARAATYNQNYPSAGLGAPGSCQTGAPTETVGGNTHLLYSWAGTAIQPTLSVFGITGATDTSTVPLVDLANVLDPSTLALFGTGTVMINRGSGQNDGLVSKCSALYGKVLSTSYKWNHLDEINQLLGVRGAYAEDPVAVIRTHANRLKLAGV

SEQ ID NO: 34 - protein, 218 aa, Pseudomonas sp.
  source 1..218: note = Lipase 2
MTEPLILQPAKPADACVIWLHGLGADRYDFMPVAEALQESLLTTRFVLPQAPTRPVTINGGYEMPSWYDIKAMSPARSISLEELEVSANMVTDLIEAQKRTGIDASRIFLAGFSQGGAVVFHTAFINWQGPLGGVIALSTYAPTFGDELELSASQQRIPALCLHGQYDDVVQNAMGRSAFEHLKSRGVTVTWQEYPMGHEVLPQEIHDIGAWLAARLG

SEQ ID NO: 35 - protein, 296 aa, Pseudomonas fluorescens
  source 1..296: note = Lipase 1
MSQDSATRYPLVLVPGMLGFIRLVLYPYWYGIIKALRRGGATVIAVQVSPLNSTEVRGEQLLTRIDEILRETGAAKVNLFGHSQGSLTARYAAAKRPDLVASVTSVAGPNHGSELADYLAKYYPADSAKGRILEALLRSVGWLMALLETGYHGPKLPVDIHASHHSLTTEGVALFNQLYPQGLPQTWGGHGPEEVNGVRYYSWSGTLQPGKTDRGGNLFDGTNRSCRLFAKTFVREPGQCDGMVGRYSSHLGTVIGDDYPMDHFDIVNQSLGLVGKGADPVRLFVEHAARLKAAGV

SEQ ID NO: 36 - protein, 296 aa, Pseudomonas fluorescens
  source 1..296: note = Lipase 2
MSQSTATRYPLVLVPGMLGFIRLLLYPYWYGIIKALRRGGATVIAVQVSPLNSTEVRGEQLLARIDEILRETGAARVNLFGHSQGSLTARYAAAKRPDLVASVTSVAGPNHGSELADYLQQHYPANTAKGRLLEALLRLIGWLMARLETGYHGPKLPVDIHASHQSLTREGVALFNQRYPQGLPETWGGQGPEVVNGVRYYSWSGTLQPGKTDRGGNLFDGTNRSCRLFAKTFVREPGQCDGMVGRYSSHLGTVIGDDYPLDHFDIVNQSLGLVGKGADPVRLFVEHAARLKAAGL

SEQ ID NO: 37 - protein, 617 aa, Pseudomonas fluorescens
  source 1..617: note = Lipase 3
MGVYDYKNFGTADSKALFSDAMAITLYSYHNLDNGFAAGYQHNGFGLGLPATLVTALLGGTDSQGVIPGIPWNPDSEKLALDAVKKAGWTPITASQLGYDGKTDARGTFFGEKAGYTTAQVEILGKYDAQGHLTEIGIAFRGTSGPRENLILDSIGDVINDLLAAFGPQDYAKNYVGEAFGNLLNDVVAFAKANGLSGKDVLVSGHSLGGLAVNSMADLSGGKWGGFFADSNYIAYASPTQSTTDKVLNVGYETTRCSAPLTARPSPAPRSGVHDAPKESATDNIVSFNDHYASTAWNLLPFSILNIPTWISHLPTAYGDGMNRIIESKFYDLTSKDSTIIVANLSDPARANTWVQDLNRNAETHKGSTFIIGSDSNDLIQGGSGNDYLEGRAGNDTFRDAGGYNVILGGAGNNTLELQKSVNTFDFANDGAGNLYIRDANGGISITRDIGSIVTKEPGFLWGLFKDDVTHSVTASGLKVGSNVTQYESSVTGTSGADTLKAHAGGDWLFGLDGNDHLIGGAGNDVFVGGAGNDLMESGGGADTFLFNGAFGQDRVLGFTANDKLVFLGVQGVLPNDDFRAHASTVGQDTVLKFGGDSVTLVGVALNSLSADGIVIA

SEQ ID NO: 38 - protein, 718 aa, Pseudomonas fluorescens
  source 1..718: note = Lipase 4
MRQEAWKQAFFSSKMPVCALKGHWVSLCLVDEAGSGAAYGGLAYTIRDSAGHQYKGRLTGEGYAKLQGFYCGPVVLILEGLYSGEEQPYLELSTRRAYRLPITELQIRAEKTRFSAADGQRIEGNPAQRQADRFYQVEVRDLVRHVAHLPPLAPRTYQPQRHALKMMDELGFGPPQPTLSGVVLFPNLHTVLEVRPLRALRPILSIDDRFCALNLYQLALMSTLSYCGFGQQPPKHTVDQVSFPLDPSVGNVFAEKLSGFQQAWKVDRDQVQRFYPIYEDVPYSHRFEIVPFDPQLYLRNRQELGEAQEHPARVHFFDDEAYGTDTQAFISHHSDVILIAVRGTASGADALRDANAHQVPFSEGVGKAHEGFYQAYRAMRDFVLHYLDLFHSGQRIVICGHSLGGAIALLLAEGLRRVPGSQYNILLYTYGAPRAADSEFADGASSLIHHRIVNHNDPVPSVPATWMNTTAKLWVPGAVTLFRAPAAGGLLFAAGLVRVGGNAYQHHGEQQHFMPLKLPDGTRSSVLWKPGCESILEAGCNRALQLQGDMPGRDNLLKQLFQANQHFMTASYIPAAWATLRRWQQTLESQGPLVTPREFELLDLALETMTRQLRDKNRELERHRPANQRGYGHEHNQALNAEIDRLHTSRRRLESLRWRRLDARDVYGSQADSLQLQPSLKRWFSHRENRELAQVASIPAPIHGGPLNPQPLDIDSIV

SEQ ID NO: 39 - protein, 296 aa, Pseudomonas sp.
  source 1..296: note = Triacylglycerol lipase 1
MPESLATRYPLVLVPGMLGFVRLGLFPYWYGIVPALQAGGAQVFPVQVAPLDSSEVRGEQLLLQIERIRRDTGAEKVNLIGHSQGSLTARYAAAKRPEWVASVTSVAGPNQGSELADYFHSHYPPASWKGRLVSALFHLAAWVMGVLETGYRGPRFKADLQASHQSLTCEGVALFNRQFPQGLPQTWGGQGAEVVNGVRYYSWSGTLQPGKTDRGRNLLDGTNRSCRLFARSFVREKGQCDGMVGRYSSHLGVVIGDDYALDHFDIVNQSLGLVGKGAEPIRLFVEHAQRLKAAGV

SEQ ID NO: 40 - protein, 476 aa, Pseudomonas fluorescens
  source 1..476: note = Lipase 5
MGIFDYKNLGTEGSKALFADAMAITLYSYHNLDNGFAVGYQHNGLGLGLPATLVGALLGSTNSQGVIPGIPWNPDSEKAALEAVQNAGWTPISASTLGYGGKVDARGTYFGEKAGYTTAQVEVLGKYDDAGKLLEIGIGFRGTSGPRETLISDSIGDLVSDLLAALGPKDYAKNYAGEAFGGLLKNVADYAAAHGLTGKDVVVSGHSLGGLAVNSMADLSTNKWSGFYTDANYVAYASPTQSAGDKVLNIGYENDPVFRALDGSSFNLSSLGVHDKPHESTTDNIVSFNDHYASTLWNVLPFSIVNLPTWVSHLPTAYGDGMTRILDSGFYDQMTRDSTVIVANLSDPARATTWVQDLNRNAEPHKGNTFIIGSDGNDLIQGGKGADFIEGGKGNDTIRDNSGHNTFLFSGQFGNDRVIGYQTTDKLVFQDVQGSTDLRDHAKVVGADTVLTFGADSVTLVGVGHGGLWADGVSIG

SEQ ID NO: 41 - protein, 296 aa, Pseudomonas fluorescens; identical to SEQ ID NO: 39
  source 1..296: note = Triacylglycerol Lipase 1

SEQ ID NO: 42 - protein, 718 aa, Pseudomonas fluorescens; identical to SEQ ID NO: 38
  source 1..718: note = Lipase class 3

SEQ ID NO: 43 - protein, 296 aa, Pseudomonas fluorescens; identical to SEQ ID NO: 39
  source 1..296: note = Lipase 6

SEQ ID NO: 44 - protein, 288 aa, Pseudomonas fluorescens
  source 1..288: note = Monoacylglycerol Lipase
MSHYEIDLGEGDAGFVLGEGPVGILLIHGLTGTPTELRQVAKGLAKAGNCTVYVPTLAGHCGDNSDLQATGWLDWYEGVRKTFAQVKQRHAQVFVGGLSMGAVMSMYVAAEHPGQVAGLLMYSTTLKYDGWSINKLAFLTPLLMKIPFGVHICRFEEKPPYGIKNERLRAIVERQMKEGESSEAGLLTMEGITVRELHRMNAVVKKRMPEVKVPALVLHSIEDDITSRWNADYVERHLGGPVTKILLDNCYHMITVDLQYRRVIELSAEFVEQHAATAQVSEDYRQRA

SEQ ID NO: 45 - protein, 718 aa, Pseudomonas fluorescens; identical to SEQ ID NO: 38
  source 1..718: note = Lipase 7

SEQ ID NO: 46 - protein, 333 aa, Pseudomonas fluorescens
  source 1..333: note = Lipase 8
MGIFDYKNLGTEGSKALFADAMAITLYSYHNLDNGFAVGYQHNGLGLGLPATLVGALLGSTNSQGVIPGIPWNPDSEKAALEAVQNAGWTPISASTLGYGGKVDARGTYFGEKAGYTTAQVEVLGKYDDAGKLLEIGIGFRGTSGPRETLISDSIGDLVSDLLAPWAPRITQRTTPAKRSAGCSKTSPITPLPMAFTGKDVVVSGHSLGGLAVNSMADLSTNKWSGFYTDANYVAYASPTQSAGDKVLNIGYENDPVFRALDGSSFNLSSLGVHDKPHESTTDNIVSFNDHYASTLWNVLPFSIVNLPTWVSHLPTAYGDGMTRILDSGSMTR

SEQ ID NO: 47 - protein, 295 aa, Pseudomonas fluorescens
  source 1..295: note = Lipase 9
MSAFIDKTQSFTSPDDSLVGSRQAYSQMCRAFTPPCPPTLAIEDFELAGVAVRAYYPQRAAPLAGWPCVLYLHGGGWVVGDLDSHDFICMELAAELGAWVVAVDYRLAPEHPFPAGFEDCLAVWRHLADAPFAIDPRRRLVVGDSAGGNLAAALCLALRDAGQPLPRAQVLIYPALGGPADLPSRHECFDAPLLSSRDLERYEALYLGGARPSPYAMPLLADNLDGLPPALVAVAQWDPLRDDGVLYCERLNAAGGDAVLYVGNGLVHGCLRGRGQVPEVDQMYRTLLAYLADML

SEQ ID NO: 48 - protein, 725 aa, Pseudomonas fluorescens
  source 1..725: note = Lipase 10
MTSFQKELESRLSSRNLTCPVGGRWTSFQLVDEWGSGEPYAGLAYVTIDSEGQRYSGNLDTRGIGKVNNHYAGPIALTFEQPYQGEEKLYIELQRRAHYPLKITELQVRAEQTRYLNPNATRTRENPAQACADDFFQVEVRHLVEHVAHLPPEVYRHYPHPAGPARLMGEHGKLGVAVMPQKHTVLEIRPLRALRPMLSTASAFCALNLYQLALMSTLTYCAFGQKPDKAPITTAVSFTRQPSVGNWFADALAKSEELGECYPGQRKPYYPLYEDMPYSRRLEIVPFDPALYAVNDPELGIHQEHPANVHFLDDTGEEDSTDTQAYITHNDELILIAVRGTQQLADFLRDADARQVPFEEGVGQVHRGFYGAAKKTAAFVTSYLDRFYAGQKLLICGHSLGGAVALLLAEMLRRRPEGYTLQLYTYGAPRAADATFTKNAEPLVHYRMVNHNDPIPSVPGSWMNTKGNVYGTGLALAFANVPVGLSVFVAGINRWTGEPYEHHGILRHFLPVEFDRKERSSILWMPRCDTITQHAACEVALEHKHGLPDRPALLAQLIQTGNHFMLPSYIPACWATLRRWQEAHITGSPLATKREYEWIDNALARITQQLRDEERSLARRDAPNGRSESDINIALIREVDKLHSTRARLATLRTASVSEKDVYGSLSEHPEQLAQDLARWQAHPENREAEQLAMAPTRDDDSELIRGAHGYAIGEPYTLDLDSIV

SEQ ID NO: 49 - protein, 296 aa, Pseudomonas fluorescens
  source 1..296: note = Triacylglycerol Lipase 2
MPQSLATRYPLVLVPGMLGFVRLGWFPYWYGIVPALRAGGAQVFAVQVAPLDSSEVRGEQLLAHIERIRRETGADKVNLIGHSQGALTARYAAARVPEWVASVTSVAGPNHGSELADHIHTHYPAESVRGRIMSALFHWVAWVMGVLETGYRGPRFKADLHASHHSLTSVGVAAFNRAYPQGLPATWGGQGAAEVNGVRYYSWSGTLQPGKTDRGRNLLDGTNRSCRLFARSFVREKGQCDGMVGRYSSHLGVVIGDDYALDHFDIVNQSLGLVGKGAEPIRLFVEHAQRLKAAGV

SEQ ID NO: 50 - protein, 718 aa, Pseudomonas fluorescens
  source 1..718: note = Lipase 11
MKLDAWKQPFFNDKMLACSMRGHWVSFCLVDEAGSGKAYGGLSFTAYDSTGCQYEGRLDGEGFAKLQDFYCGPVILLLDERYSGTEEPYARLSKRLTYKLPITELQTRAEQSHFSNADGRRIDGNPARQQADRFYQVEVSDLVRHIAHLPAVVPTACRPQPHALKMMADLGFGPPQSTLSGIVLFPNQHSVLEVRPLRALRPLLSIDDRFCALNLYQLALMSALSYCDFGQHPAERPVDQVKFPFDPSVGNLFADKLSGYREAWRIDPEQAQRFYPVYEDVPYSKRFEILPFDPELYPQNRPELKDDQEHPARMHFFDDEAFGTDTQAFITHHDEVILIAVRGTYSHTDVLRDANAHQVVFADGVGKVHEGFYLAYRAMRDFVLRYLDQFYEGQRIVICGHSLGGAIALLLSEGLRRVPEVNYDVLLYTYGAPRAADSKFTDGASALVHHRIVNHNDPVPSVPAPWMNTTAKLWIPGAVTLFSAPTPGGLLFAAGLVRVGGNPYQHHGEQQHFMPITLEDGTQASVLWKPGCESIQEAACNRALQLHGDMPDRDNLLKQLFQADQHFMTASYIPAAWATLRRWQHTLESQGPLVTPREYALLDHALETMRQQLRDKNRELEQRRPPNQRGQAHEHNQALNAEIDRLNTSRERLHSLRWRRLEVRDVYGSHSHSASLQPSLKRWFSHRENRERSPVASVPSPVHGDRGRVHSLDIDSIV

SEQ ID NO: 51 - protein, 474 aa, Pseudomonas fluorescens
  source 1..474: note = Lipase 12
MGVFDYKNLGAEGSKALFADAMAITLYTYHNLDNGFAVGYQHNGLGLGLPATLVGALLGSTDSQGVIPGIPWNPDAEKAALDAVQQAGWTPISASALGYTGKVDARGTFFGEKAGYTTAQVEVLGKYDDAGKLLEIGIGFRGTSGPRETLISDSIGDLVSDLLAALGPKDYAKNYAGEAFGGLLKNVADYASAHGLSGHDVVVSGHSLGGLAVNSMADLSSGKWAGFYQDANYVAYASPTQSAGDKVLNIGYENDPVFRALDGSSFNWSSLGVHDQPHESTTDNIVSFNDHYASTLWNVLPFSITNLPTWISHLPTGYGDGMTRIVESGFYAQMSRDSTIIVANLSDPARANTWVQDLNRNAEAHKGNTFIIGSDGDDLIQGGKGADFIEGGKGNDTIRDSSGHNTFLFNGHFGQDRIIGYQATDKLVFTDVQGSADYRDHAKVVGADTVISFGADSVTLVGVVGLSGEGMVIA

SEQ ID NO: 52 - protein, 364 aa, Burkholderia cepacia
  source 1..364: note = Triacylglycerol Lipase 2
MARSMRSRVVAGAVACAMSVAPFAGTTALMTLATTHAAMAATAPADNYAATRYPIILVHGLTGTDKYAGVLEYWYGIQEDLQQHGATVYVANLSGFQSDDGPNGRGEQLLAYVKTVLAATGAAKVNLVGHSQGGLTSRYVAAVAPDLVASVTTIGTPHRGSEFADFVQSVLAYDPTGLSSTVIAAFVNVFGILTSSSHNTNQDALASLKTLTTSQAATYNQNYPSAGLGAPGSCQTGAPTETVGGNTHLLYSWAGTAIQPTLSLFGVTGAQDTSTIPLVDPANVLDPSTLALFGTGTVMINRGSGQNDGLVSKCSALYGKVLSTSYKWNHIDEINQLLGVRGAYAEDPVAVIRTHANRLQLAGV

SEQ ID NO: 53 - protein, 370 aa, Burkholderia cepacia
  source 1..370: note = Lipase 1
MHDIDIDIARRAAPVRRLWRACARLLAGAALLACVGLAAPAAGAQTAAGAALDDYAATRYPIILVHGLTGTDDYFGVVPYWYGMQADLQRHGATVYVADLSGFQSDLGPNGRGEQLLAYVKQVLAVTGAAKVNLIGHSQGGLTSRYVASVAPALVASVTTIATPHRGSQFADFVLAALKLDPTGISTPVFGALLNLFGVLTSDTHNTNQDAIAALDALGTARTTRFNQLFPSPGLGAGACRTGAPAETIGGNVHLLYSWSGAAYQPVSLLGIVTGAADTSVIPVIDPANLLDPSTLVFLTAGNVMALASAGPNDGFTSTCSSMFGTVISTRYHWNHFDAINQLLGIRGAYAEDPVAALRVHANRLKLQGV

SEQ ID NO: 54 - protein, 364 aa, Burkholderia cepacia
  source 1..364: note = Lipase 2
MARTMRSRVVAGAVACAMSIAPFAGTTAVMTLATTHAAMAATAPAAGYAATRYPIILVHGLSGTDKYAGVLEYWYGIQEDLQQNGATVYVANLSGFQSDDGPNGRGEQLLAYVKTVLVATGATKVNLVGHSQGGLSSRYVAAVAPDLVASVTTIGTPHRGSEFADFVQNVLAYDPTGLSSSVIAAFVNVFGILTSSSHNTNQDALAALQTLTTARAATYNQNYPSAGLGAPGSCQTGAPTETVGGNTHLLYSWAGTAIQPTLSVFGVTGATDTSTLPLVDPANVLDLSTLALFGTGTVMINRGSGQNDGLVSKCSALYGKVLSTNYKWNHLDEINQLLGVRGAYAEDPVAVIRTHANRLKLAGV

SEQ ID NO: 55 - protein, 363 aa, Burkholderia cepacia
  source 1..363: note = Lipase 3
MSRSIRAKAVATVVALAMNAAPAASVGTVLSLAGTQAASAATTAVDDYAATRYPIILVHGLTGTDKYGGVVEYWYRIPEDLRAHGAAVYVANLSGFQSDDGPNGRGEQLLAFVKQVLAATGAQKVNLIGHSQGGLTSRYVASVAPELVASVTTISTPHWGSQFADFVQQLLQTDPTGLSSAVLGAFANALGTLTSSNFNTNQNAIQALSVLTTAKAAAYNQKFPSAGLGAPGSCQTGAPTETVGGNTHLLYSWGGTAIQPTATVAGVTGAVDTSVSGVTDPANALDPSTLALLGSGTVMINRSAGPNDGVVSQCSARFGQVLGTYHWNHTDAINQILGVLGANVEDPVAVIRTHANRLKNQGV

SEQ ID NO: 56 - protein, 195 aa, Burkholderia cepacia
  source 1..195: note = Lipase 4
MNPRDYALLAQEAYSAAPDIGKADSASRAIVRHTDAGLVIAFRGSDNADSWEADFDIETIDVPGAGKFHAGFWSAWKAIATDVLAAINGQPVTLVGHSLGSALAIAAALDMTLSGNVPAAVYCFEPPRTSPDLSVRTALAHVQLHSYVNGLDIVPCLPPDWHHAGLLTHIGKPALPILNTLDHAIGRVIEALTVA

SEQ ID NO: 57 - protein, 315 aa, Burkholderia cepacia
  source 1..315: note = Lipase 5
MTQPSPRDLIHPSLRAFADAAAARPSLAQMSADDARAGIAARTATRAPGPRVDKVFDLAIPGPDGGIPVRIYHPDGAVGVTMAFHGGGWRMGNLDSFDAVCRHLANDSGLAVISVDYRLAPEHPFPAALEDVWAATCWVTEHGHEHGLSTGRLTVFGESAGGNLAAVVCLLAREQTHPRILAQALVYPATDATQQHASRSVYAEGFVQRTADVAHAFREYALDHGVSPSVWTLSPFHAASHADLPPALVITAECDVLCDDGEAYARKLAEAGVATTCVRYAGMLHTFYGMRGEVGAAALAQRQVADMLRAAVHAG

SEQ ID NO: 58 - protein, 410 aa, Burkholderia cepacia
  source 1..410: note = Lipase 6
MLGTPIRSFVMLALGAAAGTWALAAEPLPVGRDAGVPAFYAWTDTVPATPGQLLRSEPLTPEQSLAEAGKNIRILYTSTDGVDGRTPIVVSGALFLPKGTPPQGGWPMMAWAHGTVGSAGVCAPSLARRSERDTRYLNDWLAHGYAIVATDYQGLGAPGVHPYGLTRPLAYGVLDSIRAVQHGDFDLTSRAVVFGQSQGARAAFATAVFAKSYAPELAIAGVVATGTPYAAVHIHAADAERAHAHQSVVPTFAYDMLRISTAALLDPAFVPADYLNDKAMPAFELSRHACLHALEDRIVADGLTYDSSFKRSPKPAFEAIQREAAYPALTSNIPIFIGTGGKDRDVFVPDQIALATDACRAGDRIEWHFYPALDHSGTVNGSLPDSTRFVARAFAGERLDGNCASLPSKP

SEQ ID NO: 59 - protein, 438 aa, Burkholderia cepacia
  source 1..438: note = Triacylglycerol Lipase 3
MSSRRFMLAAAAVSAALAVAASPASAGAPAVSDPFYTYTGTTPLASIPPGTVLKTRNVTYHVAGIPTALTAQQLLYRTNNALNQPVVNVTSVIRSQVSNGRAISYQSAYDSLNPYDEPSQVIAGDRDVTKIINVGTLLYSAESIPLSTLLLLGYNVIVPDTEGQTADFAAGPEYGMTTLDSIRAALNTPSTGLSPSSKVAMIGYSGGAIATNWAAQLAPSYAPEINRQLVGAAEGGVLVDPAHNLRYVDGSIVWGGVAAAALAGLSRGYGFDLTPYLSDTGVAVFNDIQSQSLAYILPKYTGLHWGTLFKPQYANDINSIPAYVTYANKVNAGLAASPTIPMFIGQGTAGALDGTFSSQVGDGVMLAYDVRALAQKFCASGTPVIYNEYPLEHAGAIVPWVAGMLPWLYDRFNGKAAPSNCWLTSLLPSNSLAPETLH

SEQ ID NO: 60 - protein, 364 aa, Burkholderia cepacia
  source 1..364: note = Triacylglycerol Lipase 4
MARTMRSRVVAGAVACAMSIAPFAGTTAVMTLATTHAAMAATAPAAGYAATRYPIILVHGLSGTDKYAGVLEYWYGIQEDLQQNGATVYVANLSGFQSDDGPNGRGEQLLAYVKTVLAATGATKVNLVGHSQGGLSSRYVAAVAPDLVASVTTIGTPHRGSEFADFVQDVLAYDPTGLSSSVIAAFVNVFGILTSSSHNTNQDALAALQTLTTARAATYNQNYPSAGLGAPGSCQTGAPTETVGGNTHLLYSWAGTAIQPTLSVFGVTGATDTSTLPLVDPANVLDLSTLALFGTGTVMINRGSGQNDGLVSKCSALFGKVLSTNYKWNHLDEINQLLGVRGAYAEDPVAVIRTHANRLKLAGV

SEQ ID NO: 61 - protein, 344 aa, Burkholderia cepacia
  source 1..344: note = Lipase 7
MTARGGRAPLARRAVVYGAVGLAAVAGVAMWSGAGRHGGTGASGEPPDASAARGPAAAPPQAAVPASTSLPPSLAGSSAPRLPLDAGGHLAKSRAVRDFFDYCLTAQSDLSAAGLDAFVVREIAAQLDGTVAQAEALDVWHRYRAYLDALAKLRDAGAVDKSDLGALQLALDQRASIAYRTLGDWSQPFFGAEQWRQRYDLARLKIAQDPALTDAQKAERLAALEQQMPADERAAQQRVDRQRAAIDQIAQLQKSGATPDAMRAQLTQTLGPEAAVRVAQMQQDDASWQSRYADYAAQRAQIESAGLSPQDRDAQIAALRQRMFTKPGEAVRAASLDRGAGSAR

SEQ ID NO: 62 - protein, 189 aa, Burkholderia cepacia
  source 1..189: note = Lipase 8
MTPRDYVLLAQEAYSAKPDIGNADSASRAIVRQTAGGLIVAFPGTDNLDCVVADLDAHPIDVIVIGQVRHGFWKAWGAITVDVLAAIDGRPVTLVGHSLDAAIAIMAAAAIVIGGNPPSAVYGFEPSRLSTNGSVGIVLAKVPLNLYKNASDVVPELPLNWRHAGAIQQIGRPMFPFPNVTDHAIAALS

SEQ ID NO: 63 - protein, 351 aa, Burkholderia cepacia
  source 1..351: note = Lipase 9
MACAMSIAPFAGTTAVMTLATTHAAMAATTPADGYAATRYPIILVHGLSGTDKYAGVLEYWYGIQEDLQQNGATVYVANLSGFQSDDGPNGRGEQLLAYVKTVLAATGATKVNLVGHSQGGLSSRYVAAVAPDLVASVTTIGTPHRGSEFADFVQNVLAYDPTGLSSSVIAAFVNVFGILTSSSHNTNQDALAALQTLTTARAATYNQNYPSAGLGASGSCQTGAPTETVGGNTHLLYSWAGTAIQPTLSVFGVTGATDTSTLPLVDPANVLDLSTLALFGTGTVMINRGSGQNDGLVSKCSALYGKVLSTSYKWNHLDEINQLLGVRGAYAEDPVAVIRTHANRLKLAGV

SEQ ID NO: 64 - protein, 363 aa, Burkholderia cepacia
  source 1..363: note = Triacylglycerol Lipase 5
MSRSIRAKVVATVVALAMNAAPAASVGTVLSVAGAQAASAATTAVDDYAATRYPIILVHGLTGTDKYGGVVEYWYRIPEDLRAHGAAVYVANLSGFQSDDGPNGRGEQLLAFVKQVLAATGAQKVNLIGHSQGGLTSRYVASVAPELVASVTTISTPHWGSQFADFVQQLLQTDPTGLSSTVLGAFANALGTLTSSNFNTNQNAIQALSVLTTAKAAAYNQKFPSAGLGAPGSCQTGAPTETVGGNTHLLYSWGGTAIQPTATVAGVTGAVDTSVSGVTDPANALDPSTLALLGSGTVMINRSAGPNDGVVSQCSARFGQVLGTYHWNHTDAINQILGVLGANVEDPVAVIRTHANRLKNQGV

SEQ ID NO: 65 - protein, 344 aa, Burkholderia territorii
  source 1..344: note = Lipase
MTSSKLGQMQVNLTLAYVLNNAATDKFGHGIQHPTAPQVKKIIVDRLNGSRMTRNDQFAIVWGPAIVSDRNGYAANITAVLQSTATGEYSVVTSGTNFESPLDLMGDFSFGTLEPFSAYVRDCPSDARISAGTNGALFYVLDTPDSSRVKLADFLRTVQPSSVVNVIGHSLGGALASAIVLYLKNQNGLQAHTYNCQTFAGPTAGNAAFADYFNQQMRDRAVRVFNSRDIVPMAWNADTILQVKDVYSDAGLDTPLDTKIAVDAVSAATRPLDYAQWGTADLSASVTTPMQYRLDGTVNGHIRDFHTQVGYQHIYEYMTCLDMALSDIDIPGPGPGQVASPIVR

SEQ ID NO: 66 - protein, 284 aa, Paraburkholderia acidiphila
  source 1..284: note = Lipase
MPTIPYDPSLDALDSPGHRPTVLTAEGNYEIDGLCAEFARLAYIAYEEGDAERQQLDRALALVGFQPAVAFNFADDDTHGFGAMRPSDGLAVLVFRGTDPQSLSDLADDASFNLTPWTETAGQVHAGFAGAARGLRGAVETWLKATASDRTQLILTGHSLGAAIATLFASLCTPDALVTIGSPRVGDATFVATLKASRITRYVDCCDLITDLPPPFSGYVHAGAANYITRLGALAPHADDAFVQADRSAAREDYLVNYAWRIGDVMVRDLADHAPINYVRSLVP

SEQ ID NO: 67 - protein, 340 aa, Paraburkholderia atlantica
  source 1..340: note = Lipase 1
MTLGDTIKGMVGMDPTGHADPDMKAVLDALKSLNPKPIEDCTVAEARQQPTPADAVKKLMSDPAFSARPALELEAVRTTDLMIPGAAGSNPARVYTPQGEGPFPVILYFHGGGWVLADLDTYDATPRSMAAQSRAIVVSAHYRQAPEHRLPAAHEDAFAAWRWLVGHAASLGGDAGKIAIMGESAGANLALNVAIHARDTGMRMPMHQALIYPVASNNIVSISYEENRNARPLNKPMMLWFLHNVINSESDLTSELIDVVSADLSRLPPAVVVTAGIDPLRSDGEKLAQKLHGAGVPVEHRNYRGATHEFFGMAAVVQAARDAQAFVSLAQRQAFGGAPL

SEQ ID NO: 68 - protein, 330 aa, Paraburkholderia atlantica
  source 1..330: note = Lipase 2
MLEPEIAAFIERASALYPSHHAAMTPREQRDLYDRYAATLTPPLPSELTTQDANFRTDAGHAIPLRLYRHRAKASEHGTIRGTVLYFHGGGFVLGSLNSHQMVTARLAADTGLDVIAVDYRLAPEHRAPAAHDDCLAITLAALNNRLPFDLPDHAGLQLAGDSAGGTLAASVALRLRDDGVSGVRGIALVYPMLGTEPQLPARDTEANAPMLTLADVHKFRNAYWSTVGGNSGERDEEYGDPTPGPAWTIPLAATRFDGLPPTLAIGAEHDPLRDDARVFVERIREAGGDARLLIGTGLVHGCWRALETSPGVQAMHRAVCDFLGEQARD

SEQ ID NO: 69 - protein, 342 aa, Paraburkholderia atlantica
  source 1..342: note = Lipase 3
MKIHKPLLIAAAIVASTTSFAVGPTTSATPDAATTHFLDALNSQKGPGLETLTPEKARQVLTDAQNSVRVDLSGIDVSERTIEQDGMGVPVTIVRPQGAIGALPVFMFFHGGGWILGDFPTHERLVRDLVVQSGAVAVFVNYTPSPEAHYPVAINQAYAATKWVAAHGTEIGVDGSRLAVVGNSVGGNMAAVVSLMAKNAGAPAIRFQGLLWPVTNHGFSDGSYDAYQQGYFLTRPMMKWFWDAYTKDQAQRSEIYASPLRATIDQLKGLPPAMIEVAEFDVLRDEGEAYGRKLDAAGVEAATIRYDGTIHDFGLLNALANDAATKAAIKAMAGELRSRLAQ

SEQ ID NO: 70 - protein, 358 aa, Burkholderia plantarii
  source 1..358: note = Triacylglycerol lipase
MVRSMRSRVAARAVAWALAVMPLAGAAGLTMAASPAAVAADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV

SEQ ID NO: 71 - protein, 311 aa, Pseudomonas sp.
  source 1..311: note = Triacylglycerol Lipase 2
MKKKSLLPLGLAIGLASLAASPLIQASTYTQTKYPIVLAHGMLGFDNILGVDYWFGIPSALRRDGAQVYVTEVSQLDTSEVRGEQLLQQVEEIVALSGQPKVNLIGHSHGGPTIRYVAAVRPDLIASATSVGAPHKGSDTADFLRQIPPGSAGEAVLSGLVNSLGALISFLSSGSTGTQNSLGSLESLNSEGAARFNAKYPQGIPTSACGEGAYKVNGVSYYSWSGSSPLTNFLDPSDAFLGASSLTFKNGTANDGLVGTCSSHLGMVIRDNYRMNHLDEVNQVFGLTSLFETSPVSVYRQHANRLKNASL

SEQ ID NO: 72 - protein, 451 aa, Beauveria bassiana
  source 1..451: note = Lipase 10
MRISAAFLAAALAGTAHAIPASALLHRARPTLPSKDSFYFPTGDLSNHQPGAILQQRPPPAAVAAFGLVAAKLNRTQQLLYRTSDAHGRPIATVATILIPHNADLGKLLSYQIAVDAASIDCAPSYALQQGSERGPLAGTLLSQAEFLLIEAALEQGWAVVVPDFQGPQSSYLANTLAGYATLDGIRAALASTATTGIRSDARVAMWGYSGGSLASQWAAELQPSYAPELHVAGAAVGGTVPDVASVIKAINGQPLAGFIPTGVLGLVSQYPEAAPLVDDALRPEAKQLFYSPLKQCLAANAAGFLYKDVLGMFKDRDFLLKNEVLVRISQENNLGKTTPKIPLFWYKSTMDEVSPVGDSDRLVDKYCDEGADVEYVRDVLSNHGSYAVIGAPLAFSWLKKVMNGKDGVPRKGCSKKTQLSGVLDRTQFEVLPKLIADALLDLLGKDVGPI

SEQ ID NO: 73 - protein, 309 aa, Pseudomonas sp.
  source 1..309: note = Lipase LipC
MNKNKTFLAAALVALAASLPVHAATDYTRTRYPIVLSHGLFGFKSVGPVDYWHAIVPALEKDGAKVFATSQSPVNSNEVRGEQLLAQVEEVLALTGAEKVNLIGHSQGGMTVRYVAGVAPQLVASVTTMGTPHKGTPVADAVTGFSEFLGPIGTEVIASAVEALFSVVDIVDGGEWVKGDALAALNSLNTPGTARFNQRFPQAIPASACGQGAETVAGVRYYSMSGTGSLTNALDPSSAGLAVTGLLFGEANDGLVGQCSSHLGSVVKDNYRMDHLDEVNQLLGLVSLFESDPTQVYRQHANRLRNVGL

SEQ ID NO: 74 - protein, 277 aa, Pseudomonas fragi
  source 1..277: note = Lipase-like
MDDSVNTRYPILLVHGLFGFDRIGSHHYFHGIKQALNECGASVFVPIISAANDNEARGDQLLKQIHNLRRQVGAQRVNLIGHSQGALTARYVAAIAPELIASVTSVSGPNHGSELADRLRLAFVPGRLGETVAAALTTSFSAFLSALSGHPRLPQNALNALNALTTDGVAAFNRQYPQGLPDRWGGMGPAQVNAVHYYSWSGIIKGSRLAESLNLLDPLHNALRVFDSFFTRETRENDGMVGRFSSHLGQVIRSDYPLDHLDTINHMARGSAGASTR

SEQ ID NO: 75 - protein, 613 aa, Serratia marcescens
  source 1..613: note = Lipase
MGIFSYKDLDENASKALFSDALAISTYAYHNIDNGFDEGYHQTGFGLGLPLTLITALIGSTQSQGGLPGLPWNPDSEQAAQDAVNNAGWSVIDAAQLGYAGKTDARGTYYGETAGYTTAQAEVLGKYDSEGNLTAIGISFRGTSGPRESLIGDTIGDVINDLLAGFGPKAMRRYTLKAFGNLLGDVAKFAQAHGLSGEDVVISGHSLGGLAVNSMAAQSDATWGGFYAQSNYVAFASPTQYEAGGKVINIGYENDPVFRALDGTSLTLPSLGVHDAPHTSATNNIVNFNDHYASDAWNLLPFSILNIPTWLSHLPFFYQDGLMRVLNSEFYSLTDKDSTIIVSNLSNVTRGSTWVEDLNRNAETHSGPTFIIGSDGNDLIKGGKGNDYLEGRDGDDIFRDAGGYNLIAGGKGHNIFDTQQALKNTEVAYDGNTLYLRDAKGGITLADDISTLRSKETSWLIFSKEVDHQVTAAGLKSDSGLKAYAAATTGGDGDDVLQARSHDAWLFGNAGNDTLIGHAGGNLTFVGGSGDDILKGVGNGNTFLFSGDFGRDQLYGFNATDKLVFIGTEGASGNIRDYATQQNDDLVLAFGHSQVTLIGVSLDHFNPDQVVLA

SEQ ID NO: 76 - protein, 533 aa, Bacillus thuringiensis; identical to SEQ ID NO: 11
  source 1..533: note = Lipase (Lip1 H192)

SEQ ID NO: 77 - protein, 413 aa, Bacillus tropicus
  source 1..413: note = Lipase (Lip2 H193)
MGKLFLKICFFALVAVCSLVAKITYAEESQQNNYPIILVNGFAGWGREEMLGVKYWGGVHDIQEDLKRNGYTVHTAAVGPVSSNWDRACELYAQINGGTVDYGAAHAEKHGHNRFGRTYSGFAPNWSETNKVHLVGHSMGGQTIRTLVQLLKEGSFEEKNYVKNHPNTKISPLFEGEKSYVHSVTTLATPHNGTTLADGSLLLPFVKDLLITAASFGGNDNLSLYDFKLDQWDIKKNVGESFFQYSNRILNSSLWKNTKDISQWDLSTDGAKELNNWIKTQPNVYYLSYSGHASQAAPITGLHLPHITMNKVLMGNAFFLGSYARYEENRPLIDTTWWQNDGVVNTSSMIAPSSNATVNNNESLQIGKWNHIETKANWDHLDMVGLSISDTLGFSNIQEFYRTIAEKLSRLPK

SEQ ID NO: 78 - protein, 340 aa, Rhizomucor miehei
  source 1..340: note = Lipase 1
MVPIKRQSNSTVDSLPPLIPSRTSAPSSSPSTTDPEAPAMSRNGPLPSDVETKYGMALNATSYPDSVVQAMSINGGIRAATSQEINELKYYTILSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATVLLCALDLYQREEGLSSSNLFLYTQGQPRVGDPAFANYVVSTGIPYRRTVNERDIVPHLPPAAFGFLHAGEEYWITDNSPITVQVCTSDLETSDCSNSIVPFTSVLDHLSYFGINTGLCT

SEQ ID NO: 79 - protein, 197 aa, Rhizomucor miehei
  source 1..197: note = Triacylglycerol Lipase 2
MVLKQRANYLGFLIVFFTAFLVEAVPIKRQSNSTVDSLPPLIPSRTSAPSSSPSTTDPEAPAMSRNGPLPSDVETKYGMALNATSYPDSVVQAMSIDGGIRAATSQEINELTYYTTLSANSYCRTVIPGATWDCIHCDATEDLKIIKTWSTLIYDTNAMVARGDSEKTIYIVFRGSSSIRNWIAVSIHTERSFTLCM

SEQ ID NO: 80 - protein, 363 aa, Rhizomucor miehei
  source 1..363: note = Triacylglycerol Lipase 1
MVLKQRANYLGFLIVFFTAFLVEAVPIKRQSNSTVDSLPPLIPSRTSAPSSSPSTTDPEAPAMSRNGPLPSDVETKYGMALNATSYPDSVVQAMSIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALGLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 81 - protein, 363 aa, Rhizomucor miehei; identical to SEQ ID NO: 80
  source 1..363: note = Lipase 2

SEQ ID NO: 82 - protein, 363 aa, Rhizomucor miehei
  source 1..363: note = Triacylglycerol Lipase 3
MVLKQRANYLGFLIVFFTAFLVEAVPIKRQSNSTVDSLPPLIPSRTSAPSSSPSTTDPEAPAMSRNGPLPSDVETKYGMALNATSYPDSVVQAMSIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 83 - protein, 259 aa, Bacillus cereus
  source 1..259: note = SGNH/GDSL Esterase
MKKVILTIVCLLLLIISCSNFEKNDETKQKETEGKAEKTSAPSWIYKQTNDSFYHLVLGDSLAKGYGSTQGGFAELASRQIETQIHKPITVENLGINGLTTDRLAKKVQSEDVQQKIREANIITISIGGNNLFRLNRDVGVIDGIKMLNKEKARFETDVKNIVKTVRDQNPNALLILSELYNPLQLEDSIASYADMFLDGWNDSIYSISKAHQPSIVLPIRKLISNDKKELLFDQVHPNDNGYAIIANTFTKQVLSYKY

SEQ ID NO: 84 - protein, 246 aa, Bacillus sp.
  source 1..246: note = Carboxylesterase
MKIVTPKPFTFKGGDKAVLLLHGFTGNTADVRMLGRYLNERGYTCHAPQYKGHGVPPEELVHTGPEDWWKDVMDGYEYLRSEGYENIAACGLSLGGVFSLKLGYTVPIKGIVPMCAPMHIKSEEVMYEGVLSYARNYKKFEGKQPEQIEKEMKEFEKTPMNTLKSLQELIADVRKNVDMIYSPTFVVQARHDHMINTESANIIFNEVETDDKQLKWYEESGHVITLDKERDLVHQDVYEFLEKLDW

SEQ ID NO: 85 - protein, 264 aa, Priestia megaterium
  source 1..264: note = GDSL-type Esterase
MKAKGKWVWIVSFISIISCLLFAAGLGMSIYDYVSASKATVKEMPKPKQETSSSNKSTKNYTIVALGDSLTRGTGDAVGKGYVGYLKDNLEEKTKKKILLSNFGIKGQTSSQLASQVKQQQIQRQIKSADTVLITIGGNDLFQGGQTLQNLNKNNIQKLENDYLKNVDSILKSIRSANKDTTIFLIGLYNPFSNLQDAKTTTAIVREWNYKSSELSANYKQTVFVPTFDLFQLKVDDYLYTDKFHPNAKGYKLIAERVASLITW

SEQ ID NO: 86 - protein, 240 aa, Priestia megaterium
  source 1..240: note = GDSL-type Esterase
MSSTKKVVFISSIILNVLFLGVFAFYATSSSAMKNPLSSKDSSKQSYRDNPYYIERTTLFKQLSIPKESIVFLGDSITQRSEWSELFHNKQIVNRGVADDTTEGVLHRLKSITDAKPKKIFLMIGINDLNEQKSVKKTKANYTEILERIHKESPNTKVYVQSVLPVNNKKCGDAVDNKDVIALNNYLERLAKKHNDSYIDLYSKVSTHNQLKSNFTVDGLHLKGQGYAIWKQEVQAYVNQ

SEQ ID NO: 87 - protein, 237 aa, Cytobacillus firmus
  source 1..237: note = GDSL-type Esterase
MKNKIYLLAGIILLGIGIYLSITFLGDEKKAESSQMIVAFGDSLTYGYGDETEEGYIGRLQSKLDKEYQDKNYCITNHGVYGYKSSDVLQQMLKPKVADDIKNADMFIVYIGTNDLLKSNGGDLYPLHHENLVEAKDIYEDKLNGILETLETANSDAPVILIGLYNPYPDGDRIEKYIDHWNKSIRKKAAEDKHITYISTNELFKGKNKKQYFADSLHPNGKGYELIAEKIIDNYSF

SEQ ID NO: 88 - protein, 210 aa, Bacillus subtilis
  source 1..210: note = Esterase (EstB H191)
MKKVLMAFIICLSLILSVLAAPPSGAKAESVHNPVVLVHGISGASYNFFAIKNYLISQGWQSNKLYAIDFYDKTGNNLNNGPQLASYVDRVLKETGAKKVDIVAHSMGGANTLYYIKYLGGGNKIQNVVTLGGANGLVSSTALPGTDPNQKILYTSIYSLNDQIVINSLSRLQGARNIQLYGIGHIGLLSNSQVNGYIKEGLNGGGLNTN

SEQ ID NO: 89 - protein, 182 aa, Bacillus subtilis
  source 1..182: note = Esterase (EstB H191 without signal sequence)
ESVHNPVVLVHGISGASYNFFAIKNYLISQGWQSNKLYAIDFYDKTGNNLNNGPQLASYVDRVLKETGAKKVDIVAHSMGGANTLYYIKYLGGGNKIQNVVTLGGANGLVSSTALPGTDPNQKILYTSIYSLNDQIVINSLSRLQGARNIQLYGIGHIGLLSNSQVNGYIKEGLNGGGLNTN

SEQ ID NO: 90 - protein, 320 aa, Beauveria bassiana
  source 1..320: note = Feruloyl Esterase A
MGPRMLLFGLVAALAAATPLKRQERTTVDPAVYKKLVDYAAFPTMSATDPGSAALDANCSVTLVRYINQQSTDTQAAVWLRKSTREIIIGIPGTNGLRDALTDIEVLPIPYISPSIRCPGTCLVHAGFLLAWNSIEGQVIDAVSLALEQNPGFSVVISGHSLGGAIANLAFARLKNGPFNTTGAYTYGQPRVGNREFADYIDSLSKASDSEAGSYNRVTHAEDGVPHALPMFLGYQHPRGEYWESAHEPQSDTTYRCLERESSKCSRSTTTFLELAIKAIFETLGKPLLGFPFLPTIIGVFLNSDHGTYAGLTPLCKVAQ

SEQ ID NO: 91 - protein, 289 aa, Beauveria bassiana
  source 1..289: note = Feruloyl Esterase A2
MLSLLGFLGFAALAAAAPVRDQAFIPRTLVNRSIYDKLALYAGYTQLSNTGICARPSVPGVLYQFVESKDTDTQVSIWKVDAYKEFVIAIPGTSSARDNETNFDFALVPYQADNVRCPSCRVHKGYQAAWRSVMKQVQGNLTNLLGIHPDYTVTLTGHSLGGGLVSIAFPTLRNGPYNVTQAYTYGQPRAGNGAFANYVDGISGASDKEAGIFYRVTHANDLIPKLPPGIFGYKHSRTEYWESKPSMDNAASTYRCYGQEPADCNRNATGIDSKVHVTYAGMNVTCNKS

SEQ ID NO: 92 - protein, 319 aa, Rhizomucor miehei
  source 1..319: note = Esterase
MAPTVKLKPYCQNIADAATIDSTQYPPEVVRKAEAASIIDDPKALEGLPDVYLEEKTINRKNGSKIELTITRPLDTENQVLPPIVFFHGGGWVVGSKLTHRRTVYELTVRARAAVIFVNYSLSPEVRFPTALEECLDAVVWVAKEENAKSINVDPTKLVVAGDSAGGNLSAVVCIRAKQLGLNIIKGQVLIYPVTDDNFETDSYKQFAENYYLTRKLMVWFFDHYIPDKKDRQSIFACPLKASIDDLRVLPRALVITAEADVLREEGEAYARKLIEAGNDVTAVRYLGIIHGIFNLATLSPTGSEILDHIVAWLQKTWK

SEQ ID NO: 93 - protein, 324 aa, Rhizomucor miehei
  source 1..324: note = Esterase
MTVGNPPIHPVYAAAFAAMKERPPIHTLDLKVVRESSEARQLAANIKLPEVIEEDKVVESDGKTLKLTIVRPPGTEDQILPVLIFLHGGGFVFGSKYTHIKPVRDLTVKANVVTVFVDYSLSPEAKFPTAIEEIYAAILWVRENASSLNINAEALAVAGDSAGATLSAAVSIYAKEKGLSAAIKTQVLIYPATAVSHAKYESYKLFGNGDYILSAEDLKFFSNAYLPAPASELNDKLATLELATKADLEGLPPALLFTAESDVLRDEGEKYAQQLAEAGVDVAAVRVLGAVHGFITVPVETPQYRFTINTIVAHLRDIYAKYNA

SEQ ID NO: 94 - protein, 489 aa, Bacillus subtilis [sequence omitted]

SEQ ID NO: 95 - protein, 280 aa, Sphingobium wenxiniae
  source 1..280: note = Esterase (pytH)
MTVTDIILIHGALNRGACYDAVVPLLEARGYRVHAPDLTGHTPGDGGHLSVVDMEHYTRPVADILARAEGQSILLGHSLGGASISWLAQHHPDKVAGLIYLTAVLTAPGITPETFVLPGEPNRGTPHALDLIQPVDEGRGLQADFSRLERLREVFMGDYPGEGMPPAEQFIQTQSTVPFGTPNPMEGRALEIPRLYIEALDDVVIPIAVQRQMQKEFPGPVAVVSLPASHAPYYSMPERLAEAIADFADAPAEYRQTATKAGPDRPAGADGGRADRADLP

SEQ ID NO: 96 - protein, 210 aa, Methylobacterium sp.
  source 1..210: note = Esterase (msE1)
MTQDTTGFIHRFEPGPDTTRPLMLLHGTGGDENDLLPLGRMVAPEAALLAPRGGVSENGMPRFFRRLAEGVFDEADLRRRTGDLAAFVAASRARYGLGAPLALGFSNGANIAASLLMLRPETLTGAVLIRPMVPFAEPPAADLAGRPVLILSGAMDPIVPVENARRLAQQLSASGARVEHRILPAGHGLSQADVSQALAWLRSLPGPEAA

SEQ ID NO: 97 - protein, 181 aa, Bacillus pumilus
  source 1..181: note = Esterase_2_aa
AEHNPVVMVHGMGGASYNFAAIKRYLVSQGWDRNQLYAIDFIDKTGNNLNNGPRLSRYVKDVLAKTGAKKVDIVAHSMGGANTLYYIKNLDGGDKIENVVTLGGANGLVSLRALPGTDPNQKILYTSVYSSADLIVVNSLSRLIGARNVLIHGVGHIGLLASSQVNGYIKEGLNGGGQNTN

SEQ ID NO: 98 - protein, 182 aa, Bacillus pumilus
  source 1..182: note = Esterase_3_aa
ESNHNPVVLVHGISGASYNFIATKNYLIAQGWDRNDLYAIDFVDKTGNNLNNGPQLARYIDQVLKETGAKKVDIVAHSMGGANTLYYIKYLGGGNKIENVVTLGGANGLVSSTALPGTDLNQKILYTSIYSINDQIVANSLSRLQGARNIQLYGIGHIGLLSNSQVNADIKEGLNGGGQNTN

SEQ ID NO: 99 - protein, 277 aa, Pseudomonas fluorescens
  source 1..277: note = Esterase
MGALTWIRGFNGTVGRLAPRTVASKLRRTFMTPRNLPPRDWELPLLAQSERITLRFGLSALRWGQGPAVLLMHGWEGRPTQFASLITALVDNGYSVIALDGPAHGRSPGREAHVLLFARAMLEAAAELPPLQAVVGHSMGGASALLAVQLGLRTEALVSIAAPSRFLDVLRGFAGMVGLPARARAAFIREVELTFGMPLKHLDVAHYQMNLPGLIVHAEDDTFVPVKASQAIHDAWFDSRLLRLEQGGHQKVLADPRVIDGVLTLLAGCRLQARQTA

SEQ ID NO: 100 - protein, 329 aa, Pseudomonas fluorescens
  source 1..329: note = Esterase A
MQSSSNLFPVALISAERRGDLSEDVYRLKPGNSPDGTVELAVTRLGLADVPENRGTPVILVHGSFSNRRFWYSPKGIGLGAYLARQGFDVWIPEMRGHGLSKRNQHYASNRVADYARYDLPAIGAFVREQSAQIPHWIGHSLGGTALAAALGGQHLGAPAVASVALFGCQVSRTHWPLKIPPLEWTGRLLLKRFGQVSGSRLKRGPEDEPAGVMIEAMRWNGLFGRFGEGKQDWWKGLADVDVPLLAVSAAGDRQDPDWACRKLFEQVGSEHRQYLCLGRGQGFSGDFGHVEMLVSKAAQAEVWPLVAQWLKDPLTPLFGAQAQVAVTV

SEQ ID NO: 101 - protein, 617 aa, Pseudomonas atagonensis
  source 1..617: note = Polyurethanase
MGVYDYKNFGTADSKALFSDAMAITLYSYHNLDNGFAAGYQHNGFGLGLPATLVTALLGGTDSQGVIPGIPWNPDSEKLALEAVKKAGWTPITASQLGYDGKTDARGTFFGEKAGYSTAQVEILGKYDAQGHLTEIGIAFRGTSGPRENLILDSIGDVINDLLAAFGPKDYAKNYVGEAFGNLLNDVVAFAKANGLSGKDVLVSGHSLGGLAVNSMADLSGGKWGGFFADSNYIAYASPTQSSTDKVLNVGYENDPVFRALDGSNFTGASIGVHDAPKESATDNIVSFNDHYASTAWNLLPFSILNIPTWISHLPTAYGDGMNRVIESKFYDLTSKDSTIIVANLSDPARANTWVQDLNRNAETHKGSTFIIGSDASDLIQGGSGNDYLEGRAGNDTFRDSGGYNIILGGQGSNTLDLQKSVNTFDFANDGAGNLYIRDANGGISITRDIGAIVTKEPGFLWGLFKDDVTHSVTASGLKVGNNLTAYESSVKGSNGADTLKAHAGGDWLFGLDGNDHLIGGAGNDVFVGGAGNDLMESGGGADTFLFNGAFGQDRVVGFTSNDKLVFLGVQGVLPGDDFRAHASAAGQDTVLKIGGDSVTLVGVSLGSLSGDGIVIA

SEQ ID NO: 102 - protein, 617 aa, Pseudomonas fluorescens
  source 1..617: note = Polyurethanase 1
MGVYDYKNFGTAESKALFSDAMAITLYSYHNLDNGFAVGYQHNGFGLGLPATLVTALIGGTDSQGVIPGIPWNPDSEKAALDAVNKAGWTPITASQLGYEGKTDARGTFFGEKAGYTSAQVEILGKYDAQGHLTEIGIAFRGTSGPREILIGDSIGDVINDLLAAFGPKDYARNYVGEAFGNLMNAVVAFAKANGLSGKDVLVSGHSLGGLAVNSMADLSTSKWGGFFQDSNYIAYASPTQSSTDKVLNVGYENDPVFRALDGSTFTGASLGVHDAPKESATDNIVSFNDHYASAAWNVLPFSIVNIPTWISHLPTAYGDGMNRVIESKFYVLTSKDSTIIVANLSDPARANTWVQDLNRNAETHKGSTFIIGSDGNDLIQGGSGNDYLEGRSGNDTFRDGGGYNVILGGAGNNTLDLQQSVNKFDFANDGAGNLYICDANGGISITRDIGSIVTKEPGFLWGLFKDDVTHSVTATGLKVGNNVTQYESSVKGTSGADTLKAKASGDWLFGLDGNDHLIGGAGNDVFVGGAGNDLMESGGGADTFLFNGAFGQDRVVGYTANDKLVFLGVQGVLPGDDFRAHATAVGQDTVLKFGADSVTLVGVALNSLSADGIVIA

SEQ ID NO: 103 - protein, 617 aa, Pseudomonas chlororaphis
  source 1..617: note = Polyurethanase 1
MGVFDYKNFTASDSKALFSDALAITLYSYHNIDNGFAEGYQHNGFGLGLPATLVTALIGSGNSQGVIPGIPWNPDSEKAALDALHQAGWSTISAQQLGYDGKVDGRGTFFGEKAGYGTAQVEILGKYDAQGHLESIGIAFRGTSGPRESVISDTIGDVINDLLAALGPKDYAKNYAGEAFGKLLGDVAAFAQANGLSGKDVLVSGHSLGGLGVNSLADLSSERWSGFYKDSNYIAYASPTQSASDKVLNIGYENDPVFRALDGSTFNLSSLGVHDAHQDSATNNIVNFNDHYASTLWNVLPSSILNIPTWLSHLPTGYGDGLSRVLESKFYDFTSKDSTIVVANLSDPARASTWVQDLNRNAETHKGSTFIIGSDGNDLIQGGSGNDYLEGRAGNDTFRDSGGYNIILGGQGSNTLDLQQSVKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSGNDTVLTFGQDSVTLVGVSLEHLNGSGIVLA

SEQ ID NO: 104 - protein, 614 aa, Serratia marcescens
  source 1..614: note = Polyurethanase 1
MGIFSYKDLDENASKALFSDALAISTYAYHNIDDGFDEGYHQSGFGLGLPLTLITALIGSTQSQGGLPGIPWNPDSEQAAQEAVNNAGWSVISAAQLGYAGKTDARGTYYGEAAGYTTAQAEVLGKYDSEGNLTAIGISFRGTSGPRESLIGDTIGDAINDLLAGIGPKGYADGYTLNAFGNLLGDVAKFAQAHGLSGEDVVVSGHSLGGLAVNSMAAQSDANWGGFYAQSNYVAFASPTQYETGDKVINIGYENDPVFRVLDGTTLTGASLGVHDAPHTSATNNIVNFNDHYASDAWNLLPFSILNVPTWLSHLPFFYQDGLMRVLNSEFYSLTDKDSTIIVSNLSNVTRGNTWVEDLNRNAETHSGPTFIIGSDGNDLIKGGKGNDYLEGRDGDDIFRDAGGYNLIAGGKGHNIFDTQQALKNTEVAYDGNTLYLRDAKGGITLADDISTLRSKETSWLIFNKEVDHQVTAAGLKSDSGLKAYAAATTGGDGDDDLQARSHDAWLFGNAGNDTLIGHAGGNLTFVGGSGDDILKGVGNGNTFLFSGDFGRDQLYGFNATDKLVFIGTEGASGNIRDYATQQNDDLVLAFGHSQVTLIGVSLDHFNTDQVVLA

SEQ ID NO: 105 - protein, 615 aa, Serratia liquefaciens
  source 1..615: note = Polyurethanase 1
MGIFNYQGLDEAKSKTLFTDAMAISTYAYHNIDNGFDEGYHSSGFGLGLPFTLVTALIGSTQSQGGLPGIPWNPDSEQAALAAVNNAGWSLISADQLGYQGKTDARGTYYGETFGYTTAQAEVLGKYDSEGNLTGIGIAFRGTSGPRESLITDTIGDLVNDLLAGFGPNGYADNYSLKAFGTLLGDVAKFAQAHGLSGDDITISGHSLGGLAVNSMAALSDGNWGGFYAQSNYVAFASPTQYETGGKVINVGYENDPVFRALDGTTRTSATLGVHDAPQDSATNNIVNFNDHYASAAWNILPFSILNIPTWLSHLPFFYQDGLMRVLNSEFYSLTSKDSTVIVSNLSDVTRGNTWVEDLNRNAEQHSGPTFIVGSDGNDLIKGGTGNDYLEGRAGNDTFRDDGGFNIISGGEGHNTLDLQHALKNTEVAYDGNTLYLRDADGGITLANSIGTLKSKESSLLIFTKEVDHQVTDNGLLSTKGLTAYASSANGTAADDVLTAKESGSWLFGLEGNDQLFGGKGNDVFVGGAGNDVMHSQGGSNTFLFSGDFGQDLIYGYQAQDKLVFIGTEGSSSGGNCRDFVSEVNDNLVFNFGGNTVTLVGIGFDSLSDGQVVLA

SEQ ID NO: 106 - protein, 615 aa, Serratia liquefaciens
  source 1..615: note = Polyurethanase 2
MGIFNYQGLDEAKSKALFTDAMAISTYAYHNIDNGFDEGYHNTGFGLGLPFTLVTALIGSSQSQGGLPGIPWNPDSEKAALAAVNNAGWSLISADQLGYQGKTDARGTYYGETLGYTTAQAEVLGKYDSEGNLTGIGIAFRGTSGPRESLITDTIGDLVNDLLAGFGPSGYADNYSLKAFGTLLGDVAKFAQSHGLSGDDITISGHSLGGLAVNSMAALSDGNWGGFYAQSNYVAFASPTQYETGDKVINIGYENDPVFRALDGTTRTSATLGVHDAPQESATNNIVNFNDHYASTAWNILPFSILNVPTWLSHLPFFYQDGLMRVLNSEFYSLTSKDSTVIVSNLSDVTRGNTWVEDLNRNAEQHSGPTFIVGSDGNDLIKGGAGNDYLEGRAGNDTFRDDGGFNIISGGEGHNTLDLQHALKNTEVAYDGNTLYLRDADGGITLANSIGTLKSKESSLLIFTKEVDHQVTDNGLLSTKGLTAYASSANGTATDDVLTAKDSGSWLFGLEGNDQLFGGKGNDVFVGGAGNDVMHSQGGNNTFLFSGDFGQDLIYGYQARDKLVFIGTDGSSTGGNFRDFASEVNDNLVFNFGGNTVTLVGVGFDSLSDGQVVLA

SEQ ID NO: 107 - protein, 614 aa, Serratia marcescens
  source 1..614: note = Polyurethanase 2
MGIFSYKDLDENASKVLFSDALAISTYAYHNIDNGFDEGYHQTGFGLGLPLTLITALIGSTQSQGGLPGLPWNPDSEQAAQDAVNNAGWSVISAAQLGYAGKTDARGTYYGETAGYTTAQAEVLGKYDSEGNITAIGISFRGTSGPRESLIGDTIGDVINDLLAGFGPKGYADGYTLKAFGNLLGDVAKFAQAHGLSGEDVVVSGHSLGGLAVNSMAAQSDANWGGFYAQSNYVAFASPTQYENGGKVINIGYENDPVFRALDGTSLTLPSLGVHDAPHTSATNNIVNFNDHYASDAWNLLPFSILNIPTWLSHLPFFYQDGLMRVLNSEFYSLTDKDSTIIVSNLSNVTRGNTWVEDLNRNAETHSGPTFIIGSDGNDLIKGGKGNDYLEGRDGDDIFRDAGGYNLIAGGKGHNIFDTQQALKNTEVAYDGNTLYLRDAKGGITLADDINTLRSKETSWLIFNKEVDHQVTAAGLKSDSGLKAYATAATGGDGDDVLQARSHDAWLFGNAGNDMLIGHAGGNLTFVGGSGDDILKGVGNGNTFLFSGDFGRDQLYGFNASDKLVFIGTEGASGNIRDYATQQNDDLVLAFGHSQVTLIGVSLDHISTDQVVLA

SEQ ID NO: 108 - protein, 615 aa, Serratia liquefaciens
  source 1..615: note = Polyurethanase; note = Serratia liuefaciens
MGIFNYQGLDEAKSKALFTDAMAISTYAYHNIDNGFDEGYHNTGFGLGLPFTLVTALIGSSQSQGGLPGIPWNPDSEKAALAAVNNAGWSLISADQLGYQGKTDARGTYYGETLGYTTAQAEVLGKYDSEGNLTGIGIAFRGTSGPRESLITDTIGDLVNDLLAGFGPSGYADNYSLKAFGTLLGDVAKFAQSHGLSGDDITISGHSLGGLAVNSMAALSDGNWGGFYAQSNYVAFASPTQYETGDKVINIGYENDPVFRALDGTTRTSATLGVHDAPQESATNNIVNFNDHYASTAWNILPFSILNVPTWLSHLPFFYQDGLMRVLNSEFYSLTSKDSTVIVSNLSDVTRGNTWVEDLNRNAEQHSGPTFIVGSDGNDLIKGGAGNDYLEGRAGNDTFRDDGGFNIISGGEGHNTLDLQHALKNTEVAYDGNTLYLRDADGGITLANSIGTLKSKESSLLIFTKEVDHQVTDNGLLSTKGLTAYASSANGTATDDVLTAKDSGSWLFGLEGNDQLFGGKGNDVFVGGAGNDVMHSQGGSNTFLFSGDFGQDLIYGYQARDKLVFIGTDGSSTGGNFRDFASEVNDNLVFNFGGNTVTLVGVGFDSLSDGQVVLA

SEQ ID NO: 109 - protein, 617 aa, Pseudomonas chlororaphis; identical to SEQ ID NO: 103
  source 1..617: note = Polyurethane 2

SEQ ID NO: 110 - protein, 617 aa, Pseudomonas protegens
  source 1..617: note = Polyurethanase 1
MGVFDYKNFTASDSKALFSDALAITLYSYHNIDNGFAEGYQHNGFGLGLPATLVTALIGSGNSQGVIPGIPWNPDSEKAALDALHQAGWSTISAQQLGYDGKVDGRGTFFGEKAGYGTAQVEILGKYDAQGHLESIGIAFRGTSGPRESVISDTIGDVINDLLAALGPKDYAKNYAGEAFGKLLGDVAAFAQANGLSGKDVLVSGHSLGGLGVNSLADLSSERWSGFYKDSNYIAYASPTQSASDKVLNIGYENDPVFRALDGSTFNLSSLGVHDAHQDSATNNIVNFNDHYASTLWNVLPFSILNIPTWLSHLPTGYGDGLSRVLESKFYDFTSKDSTIVVANLSDPARASTWVQDLNRNAETHKGSTFIIGSDGNDLIQGGSGNDYLEGRAGNDTFRDSGGYNIILGGQGSNTLDLQQSVKNYSFASDGAGTLYLRDANGGISMTRDIGAIQSKEPGFLWGLFKDDVIHQVTDQGLKAGGQLTQYASSVRGDAGDNVLKAHAGGDWLFGLDGNDHLIGGQGNDVFVGGAGNDLMEAGGGNNTFLFTGHFGQDRILGYQGGDKLVFMGVSGVLPDQDYRAHASSSGNDTVLTFGQDSVTLVGVSLEHLNGSGIVLA

SEQ ID NO: 111 - protein, 617 aa, Pseudomonas fluorescens
  source 1..617: note = Polyurethanase 2
MGVYDYKNFGTADSKALFTDAMAITLYSYHNLDNGFATGYQHNGFGLGLPATLVTALIGGTDSQGVIPGIPWNPDSEKAALDAVQKAGWTPITATQLGYDGKVDARGTFFGEKAGYTSAQVEILGKYDAQGHLSEIGIAFRGTSGPRESQISDSIGDVINDLLAALGPKDYAKNYVGEAFGDLLGDVAAFARANGLSGKDVLVSGHSLGGLAVNSLADLSNDKWAGFYQDSNYIAYASPTQSSTDKVLNVGYENDPVYRALDGSSFNLSSVGVHDAAKVSATDNIVSFNDHYASTAWNVLPFSILNIPTWISHLPTAYGDGMNRVIESKFYDLTSKDSTIIVANLSDPARANTWVQDLNRNAETHKGSTFIIGSDGDDLIQGGKGNDYLEGRDGNDTFRDGGGYNILLGGKGNNVLDVQQSVKNFDFANDGAGNLYIRDANGGISMTRDIGSIVTKEPGFLWGLFKDDVIHSVTAEGLKAGSNLTQYASTVKGGAGVDTLTAHAGGDWLLGLDGNDHLLGGKGNDVFVGGAGNDVLESGGGIDTFLFSGAFGQDRVVGYQASDKLVFLGVQGVMPNDDYRAHATAVGHDTVLTFGGDSVTLVGVGLDSLSSSGIVIA

SEQ ID NO: 112 - protein, 617 aa, Pseudomonas atagonensis; identical to SEQ ID NO: 101
  source 1..617: note = Polyurethanase; note = Pseudomonas atagonenis

SEQ ID NO: 113 - protein, 617 aa, Pseudomonas fluorescens; identical to SEQ ID NO: 102
  source 1..617: note = Polyurethanase 3

SEQ ID NO: 114 - protein, 474 aa, Pseudomonas fluorescens; identical to SEQ ID NO: 51
  source 1..474: note = Polyurethanase 4

SEQ ID NO: 115 - protein, 614 aa, Serratia marcescens; identical to SEQ ID NO: 104
  source 1..614: note = Polyurethanase 3

SEQ ID NO: 116 - protein, 615 aa, Serratia liquefaciens; identical to SEQ ID NO: 105
  source 1..615: note = Polyurethanase 3

SEQ ID NO: 117 - protein, 615 aa, Serratia liquefaciens; identical to SEQ ID NO: 106
  source 1..615: note = Polyurethanase 4

SEQ ID NO: 118 - protein, 614 aa, Serratia marcescens; identical to SEQ ID NO: 107
  source 1..614: note = Polyurethanase 4

SEQ ID NO: 119 - protein, 615 aa, Serratia marcescens; identical to SEQ ID NO: 108
  source 1..615: note = Polyurethanase 5

SEQ ID NO: 120 - protein, 617 aa, Pseudomonas protegens; identical to SEQ ID NO: 110
  source 1..617: note = Polyurethanase 2

SEQ ID NO: 121 - protein, 617 aa, Pseudomonas fluorescens; identical to SEQ ID NO: 111
  source 1..617: note = Polyurethanase 5

SEQ ID NO: 122 - protein, 935 aa, Bacillus thuringiensis
  source 1..935: note = e0Collag-F1756
EEKVPYNVLKTKPVGIEKPVDEVGHVSKVDETLSFQERLKVGDFSQRPASITKKTAVKQVKESYSMADLNKMNNQELVETLGSIKWHQITDLFQFNEDTKAFYKDKGKMQVIIDELAHRGSTFTKDDSKGIQTFTEVLRSAFYLAFYNNELSELNERSFQDKCLPALKAIAKNPNFKLGTDEQDTVVSAYGKLISNASSDVETVQYASNILKQYNDNFTTYVNDRMKGQAIYDIMQGIDYDIQSYLVEARKEANETMWYGKVDGFINEINRIALLNEVTSENKWLVNNGIYFASRLGKFHSNPNKGLEVVTQAMHMYPHLSEPYFVAIEQITTNYNGKDYSGNTVDLEKIRKEGKEQYLPKTYTFDDGSIVFKTGDKVSEEKIKRLYWAAKEVKAQYHRVIGNDKALEPGNADDVLTIVIYNSPDEYQLNRQLYGYETNNGGIYIEETGTFFTYERTPEQSIYSLEELFRHEFTHYLQGRYEVPGLFGRGDMYQNERLTWFQEGNAEFFAGSTRTNNVVPRKSIISGLSSDPASRYTAERTLFAKYGSWDFYNYSFALQSYLYTHQFETFDKIQDLIRANDVKNYDAYRENLSKDPKLNKEYQEYMQQLINNQDTYTVPEVADDYLAEHATKSLTAVKKEISDTLPMKDTKMTKHNSQFFNTFTLEGTYTGSVTKGESEDWKAMSKRVNESLEQLAQKEWSGYKTVTAYFVNYRVNSSNEFEYDVVFHGIAKDDGENKAPTVNVNGPYNGVVKEGIQFKSDGSNDEDGKIVSYLWEFGDGSTSAEVNPVHVYEREGSYKVSLRVKDDKGKESRSETTVTIKDGSLTESEPNNRPEEANRIGLNSTIKGNLIGGDHTDVYTFNVASAKDIDISVLNEYGIGMTWVLHHESDMQNYAAYGQANGNHIEAKFNAKPGKYYLYVYKYDNGNGTYELSVK

SEQ ID NO: 123 - protein, 405 aa, Orbilia oligospora
  source 1..405: note = Aoz1-Cutinase-F1898; note = Orbilia oigospora
GPIRKVSNAGAAGAIADKYIVVLKKGLSDNAVSKHTNRISSFHSNVARDLTGARAHGVGRKFRFSSTGFNGYVGGFDKATLQEILNSPEVDYVEQDTVVTTYAEQTDSTWGLDRISHEDYSAPYTYEYDETAAGAGTTVYVIDTGIRISHDEFQTVNGSSRATWGFNSVDKTDSDGNGHGTHCAGTIAGKTYGVSKKAKVVAVKVLSAGGSGSTAGVVSGMNWVAENATPNFSVASMSLGGSKSAALNSAVDSIFNAGITIVVAAGNENQDAKNVSPASAPNAITVGAIDSSNKIASFSNWGTLIDVFAPGVGVLSSWATSDKETKTISGTSMACPHVAGLAAYYISASEGGADPAIITDKITSSRRQWSGHREHPWLSKQDRLQRICLNTHSPKTNHQVAIVAS

SEQ ID NO: 124 - protein, 389 aa, Trichoderma koningiopsis
  source 1..389: note = prb-Trich-F1902
APAALHKKPEAVPNKFIVTLKEGASIDTDSHLAWVNDIHTRSLTKRSTAGVEKTYNIHTWNAYAGEFDEETIEQIKSSPDVASVEPDYIMHLSDIVEDKRALTTQSGAPWGLGTVSHRTSGSTSYIYDSSAGAGTFAYVVDSGINTSHQQFGGRASLGYNAAGGQHVDTLGHGTHVSGTIAGSTYGVAKQASLISVKVFAGESASTSVILDGYNWAVNDIVSRNRASKSAINMSLGGPASSTWTTAINAAFNQGVLTIVAAGNGDSLGNPQPVSGTSPANVPNAITVAALDINWRTASFTNYGAGVDVFAPGVNILSSWIGSNTATNTISGTSMATPHVVGLALYLQSLEGLATPAAVTNRIKALATSGRVTGSLNGSPNVIIFNGNSS

SEQ ID NO: 125 - protein, 258 aa, Trichoderma lixii
  source 1..258: note = QYS92649-Trich-F1903
MAPVLAIASVLAALPALTMGAAITPRGSDIVGGTTAALGEFPYIVSLSTGGSHFCGGVLIDSRTVVTAGHCTIDQRASSVKVRAGTLTWASGGTQVGVSSLTLHPSYTVDSQGVPDNDVGVWHLATAIPTSSTIGYATLPASGSDPAAGTTLTVAGWGTTSENSNSLPSTLRKVSVPVVARATCDSDYDGEISNNMFCAAVAAGGKDSCSGDSGGPIIDPSGTLVGVVSWGQGCAERGFPGVYTRLGNYVSFINSNRG

SEQ ID NO: 126 - protein, 238 aa, Trichoderma lixii
  source 1..238: note = H182 trypsin-like protease
AAITPRGSDIVGGTTAALGEFPYIVSLSTGGSHFCGGVLIDSRTVVTAGHCTIDQRASSVKVRAGTLTWASGGTQVGVSSLTLHPSYTVDSQGVPDNDVGVWHLATAIPTSSTIGYATLPASGSDPAAGTTLTVAGWGTTSENSNSLPSTLRKVSVPVVARATCDSDYDGEISNNMFCAAVAAGGKDSCSGDSGGPIIDPSGTLVGVVSWGQGCAERGFPGVYTRLGNYVSFINSNRG

SEQ ID NO: 127 - protein, 494 aa, Bacillus sp.
  source 1..494: note = B16 neutral protease H184
AENPQLKENLTNFVPKHSLVQSELPSVSDKAIKQYLKQNGKVFKGNPSERLKLIDHTTDDLGYKHFRYVPVVNGVPVKDSQVIIHVDKSNNVYAINGELNNDASAKTANSKKLSANQALDHAFKAIGKSPEAVSNGNVANKNKAELKAAATKDGKYRLAYDVTIRYIEPEPANWEVTVDAETGKVLKKQNKVEHAAATGTGTTLKGKTVSLNISSESGKYVMRDLSKPTGTQIITYDLQNRQYNLPGTLVSSTTNQFTTSSQRAAVDAHYNLGKVYDYFYQTFKRNSYDNKGGKIVSSVHYGSKYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDHYKNYRNLPNTDAGDYGGVHTNSGIPNKAAYNTITKIGVKKAEQIYYHALTVYLTPSSSFKDAKAALIQSARDLYGSQDAASVEAAWNAVGL

SEQ ID NO: 128 - protein, 374 aa, Hirsutella rhossiliensis
  source 1..374: note = protease H181_ASP
APATKRDEPAPLLIPRGSKQLIADKYIVKFKDSMSIAAVDKTVKALSNKADHVYTNTFRGFAGQLSAKDVKALRERPDVEYVEQDSIVTINALKQQPGAPWGLGRISHREPGSTTYVYDSSAGQGTCAYVIDTGVEASHPEFEGRATMVRSFINGQATDGHGHGTHCAGTIGSKTYGVAKKTKIYGVKVLSNQGSGAVSVIIAGMDFAVQDSRKRSCPKGVVANMSLGGGFSAAINNAAASMIRSGVFLAVAAGNDNTDASGSSPASEPTVCTVGASTISDQRSSFSNYGSVVDIFAPGSDILSTWIGGRTNTISGTSMATPHIAGLGAYLAGLEGFSDPQALCSRIQELATSDVLTDIPDGTVNKLAFNGASQ

SEQ ID NO: 129 - protein, 352 aa, Bacillus sp.
  source 1..352: note = B16 protease H185 apr (Alkaline serine protease)
AGKSNGEKKYIVGFKQTMSTMSAAKKKDVISEKGGKVQKQFKYVDAASATLNEKAVKELKKDPSVAYVEEDHVAQAYAQSVPYGVSQIKAPALHSQGFTGSNVKVAVIDSGIDSSHPDLKVAGGASMVPSETNPFQDNNSHGTHVAGTVAALNNSVRVLGGAPSASLYAVKVLGADGSGQYSWIINGIEWAIANNMDVINMSLGGPSGSAALKAAVDKAVASGIVVVAAAGNEGTSGSSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 130 - protein, 521 aa, Bacillus subtilis
  source 1..521: note = protease NprE
MGLGKKLSVAVAASFMSLSISLPGVQAAEGHQLKENQTNFLSKNAIAQSELSAPNDKAVKQFLKKNSNIFKGDPSKRLKLVESTTDALGYKHFRYAPVVNGVPIKDSQVIVHVDKSDNVYAVNGELHNQSAAKTDNSQKVSSEKALALAFKAIGKSPDAVSNGAAKNSNKAELKAIETKDGSYRLAYDVTIRYVEPEPANWEVLVDAETGSILKQQNKVEHAAATGSGTTLKGATVPLNISYEGGKYVLRDLSKPTGTQIITYDLQNRQSRLPGTLVSSTTKTFTSSSQRAAVDAHYNLGKVYDYFYSNFKRNSYDNKGSKIVSSVHYGTQYNNAAWTGDQMIYGDGDGSFFSPLSGSLDVTAHEMTHGVTQETANLIYENQPGALNESFSDVFGYFNDTEDWDIGEDITVSQPALRSLSNPTKYNQPDNYANYRNLPNTDEGDYGGVHTNSGIPNKAAYNTITKLGVSKSQQIYYRALTTYLTPSSTFKDAKAALIQSARDLYGSTDAAKVEAAWNAVGL

SEQ ID NO: 131 - protein, 567 aa, Bacillus mycoides
  source 1..567: note = protease
MKNTKTLTKVALTTGLALTAVAPYGIGHAEETDQLQVQIQEDSFRSGELTQPSQKTPENVVKDALKEKAEQALSPNQVNGETGVDFKVLQKRGSYDGTTLVRMQQTYEGKEVYGHQLTAHVDNNGVIKSVSGDSAQNLKQEELKKPINLSKDEAKQYIYTKYGNDIKFISEPEVKEVIFVDENNGQASNAYQVTFEAATPNYVSGTYLVNAQNGDMLKNMVQESNLKASEKLVGALKESKKSSLTSLTGTGKDDLGISRSFGISKQSNGKYALADYTRGQGIETYDVNYRDITKEESYYPGTLATSTSTTFNDPKAVSAHYLATKVFDFYKDKYKRNSFDNKGQKVVSVVHAWDSEDTNDPKNWQNALSANNGSMLVYGDPIVKAYDVAGHEFTHAVTSSESNLEYYGESGAINEALSDIMGTSIEKYVNNGSFNWTMGEQTGSVFRDMENPASVPSSLGVPYPDDYSEFNDFNGWDQGGVHFNSSIINKVAYLIAKGGTHNGVTVKGIGEDKMFDIFYYANTDELNMTSNFKELKSACIRVATNKYGANTAEVQAVQKAFDAAKIK

SEQ ID NO: 132 - protein, 521 aa, Bacillus tequilensis
  source 1..521: note = protease NprE
MGLGKKLSVAVAASFMSLSISLPGVQAAESHQLKENQTHFLSKNAIAQSELPAPNDKAVKQFLKKNSGIFKGDPAKRLKLVESTTDALGYKHFRYAPVVNGVPVKDSQVIVHVDKSDNVYAINGELHNQSAAKTENSKKVSSEKALALAFKAIGKSPDAVSNGAAKNSNKAELKAVETKDGSYRLAYDVTIRYIEPEPANWEVLVDAETGSILKQQNKVEHAAATGSGTTLKGATVPLNISYESGKYVLRDLSKPTGTQIITYDLQNRKSSLPGTLVSSTTKTFTSSSQRAAVDAHYNLGKVYDYFYSNFKRNSYDNKGSKIVSSVHYGTKYNNAAWIGDQMIYGDGDGSFFSPLSGSLDVTAHEMTHGVTQETANLIYEKQPGALNESFSDVFGYFNDTEDWDIGEDITVSQPALRSLSNPTKYDQPDNYADYQNLPNTDEGDYGGVHTNSGIPNKAAYNTITKLGVSKSQQIYYRALTTYLTPSSTFKDAKAALIQSARDLYGSTDAAKVEAAWNAVGL

SEQ ID NO: 133 - protein, 538 aa, Bacillus subtilis
  source 1..538: note = protease NprB
MRNLTKTSLLLAGLCTAAQMVFVTHASAEESIEYDHTYQTPSYIIEKSPQKPVQNTTQKESLFSYLDKHQTQFKLKGNANSHFRVSKTIKDPKTKQTFFKLTEVYKGIPIYGFEQAVAMKENKQVKSFFGKVHPQIKDVSVTPSISEKKAIHTARRELEASIGKIEYLDGEPKGELYIYPHDGEYDLAYLVRLSTSEPEPGYWHYFIDAKNGKVIESFNAIHEAAGTGIGVSGDEKSFDVTEQNGRFYLADETRGKGINTFDAKNLNETLFTLLSQLIGYTGKEIVSGTSVFNEPAAVDAHANAQAVYDYYSKTFGRDSFDQNGARITSTVHVGKQWNNAAWNGVQMVYGDGDGSKFKPLSGSLDIVAHEITHAVTQYSAGLLYQGEPGALNESISDIMGAMADRDDWEIGEDVYTPGIAGDSLRSLEDPSKQGNPDHYSNRYTGTEDYGGVHINSSIHNKAAYLLAEGGVHHGVQVEGIGREASEQIYYRALTYYVTASTDFSMMKQAAIEAANDLYGEGSKQSASVEKAYEAVGIL

SEQ ID NO: 134 - protein, 566 aa, Bacillus thuringiensis
  source 1..566: note = protease
MKKKSLALVLATGMAVTTFGGTGSAFADSKNVLSTKKYNETVQSPEFVSGDLTEATGKKAETVVFDYLNAAKGDYKLGEKSAQDSFKVKQAKKDAVTDSTVLRLQQVYEGVPVWGSTQVAHVSKDGSLKVLSGTVAPDLDKKEKLKNKNKIEGAKAIEIAQKDLGVTPKYEVEPKADLYVYQNGEETTYAYVVNLNFLEPSPGNYYYFIEADSGKVLNKYNKLDHVANEDKSPVKQEAPKQEAKPAVKPVTGTNAVGTGKGVLGDTKSLNTTLSASSYYLQDNTRGATIFTYDAKNRTTLPGTLWVDADNVFNAAYDAAAVDAHYYAGKTYDYYKATFNRNSINDAGAPLKSTVHYGSKYNNAFWNGSQMVYGDGDGVTFTSLSGGIDVIGHELTHAVTEYSSDLIYQNESGALNEAISDVFGTLVEYYDNRNPDWEIGEDIYTPGKAGDALRSMSDPTKYGDPDHYSKRYTGTSDNGGVHTNSGIINKAAYLLANGGTHYGVTVNGIGKDKVGAIYYRANTQYFTQSTTFSQARAGLVQAAADLYGASSAEVAAVKQSYSAVGVN

SEQ ID NO: 135 - protein, 546 aa, Geobacillus sp.
  source 1..546: note = protease
MNKRAMLGAIGLAFGLMAWPFGASAKEKSMVWNEQWKTPSFVSGSLLKGEDAPEELVYRYLDQEKNTFQLGGQARERLSLIGKQTDELGHTVMRFEQRYRGIPVYGAVLVAHVNDGELSSLSGTLIPNLDKRTLKTEAAISIQQAEMIAKQDVADTVTKERPAAEEGKPTRLVIYPDGETPRLAYEVNVRFLTPVPGNWIYMIDAADGKVLNKWNQMDEAKPGGGQPVAGTSTVGVGRGVLGDQKYINTTYSSYYGYYYLQDNTRGSGIFTYDGRNRTVLPGSLWADVDNQFFASYDAAAVDAHYYAGVVYDYYKNVHGRLSYDGSNAAIRSTVHYGRGYNNAFWNGSQMVYGDGDGQTFLPFSGGIDVVGHELTHAVTDYTAGLVYQNESGAINEAMSDIFGTLVEFYANRNPDWEIGEDIYTPGIAGDALRSMSDPAKYGDPDHYSKRYTGTQDNGGVHTNSGIINKAAYLLSQGGVHYGVSVTGIGRDKMGKIFYRALVYYLTPTSNFSQLRAACVQAAADLYGSTSQEVNSVKQAFNAVGVY

SEQ ID NO: 136 - protein, 546 aa, Bacillus sp.
  source 1..546: note = Thermolysin
MDKRAMLGAIGLAFGLMAWPFGASAKEKSMVWNEQWKTPSFVSGSLLKGEDAPEELVYRYLDQEKNTFQLGGQARERLSLIGKQTDELGHTVMRFEQRYRGIPVYGAVLVAHVNDGELSSLSGTLIPNLDKRTLKTEAAISIQQAEMIAKQDVADAVTKERPAAEEGKPTRLVIYPDGETPRLAYEVNVRFLTPVPGNWIYMIDAADGKVLNKWNQMDEAKPGGGQPVAGTSTVGVGRGVLGDQKYINTTYSSYYGYYYLQDNTRGSGIFTYDGRNRTVLPGSLWADVDNQFFASYDAAAVDAHYYAGVVYDYYKNVHGRLSYDGSNAAIRSTVHYGRGYNNAFWNGSQMVYGDGDGQTFLPFSGGIDVVGHELTHAVTDYTAGLVYQNESGAINEAMSDIFGTLVEFYANRNPDWEIGEDIYTPGIAGDALRSMSDPAKYGDPDHYSKRYTGTQDNGGVHTNSGIINKAAYLLSQGGVHYGVSVTGIGRDKMGKIFYRALVYYLTPTSNFSQLRAACVQAAADLYGSTSQEVNSVKQAFNAVGVY

SEQ ID NO: 137 - protein, 546 aa, Geobacillus thermoleovorans
  source 1..546: note = protease
MNKRAMLGAIGLAFGLMAWPFGASAKEKSMVWNEQWKTPSFVSGSLLKGEDAPEELVYRYLDQEKNTFQLGGQARERLSLIGKQTDELGHTVMRFEQRYRGIPVYGAVLVAHVNDGELSSLSGTLIPNLDKRTLKTEAAISIQQAEMIAKQDVADAVTKERPAAEEGKPTRLVIYPDGETPRLAYEVNVRFLTPVPGNWIYMIDAADGKVLNKWNQMDEAKPGGGQPVAGTSTVGVGRGVLGDQKYINTTYSSYYGYYYLQDNTRGSGIFTYDGRNRTVLPGSLWADVDNQFFASYDAAAVDAHYYAGVVYDYYKNVHGRLSYDGSNAAIRSTVHYGRGYNNAFWNGSQMVYGDGDGQTFLPFSGGIDVVGHELTHAVTDYTAGLVYQNESGAINEAMSDIFGTLVEFYANRNPDWEIGEDIYTPGIAGDALRSMSDPAKYGDPDHYSKRYTGTQDNGGVHTNSGIINKAAYLLSQGGVHYGVSVTGIGRDKMGKIFYRALVYYLTPTSNFSQLRAACVQAAADLYGSTSQEVNSVKQAFNAVGVY

SEQ ID NO: 138 - protein, 316 aa, Bacillus paralicheniformis
  source 1..316: note = Protease _ Mpr
MVSKKSVKRGLITGLIGISIYSLGIHPAQAAPSPHSPVSSDPSYKAETSVTYDPNMKSDQYGSYSKAFTGTGKVNETKEKTAKESSAKAPYSIKSVIGSDDRTRVTNTSAYPYRAIVHISSSIGSCTGWMIGPKTVATAGHCIYDTSSGSFAGTATVSPGRNGTSYPYGSVKSTRYFIPSGWRSGNTNYDYGAIELSEPIGNTVGYFGYSYTTSSLVGTTVTISGYPGDKTTGTQWQHSGPIAISETYKLQYAMDTYGGQSGSPVFEQSSSRTNCSGPCSLAVHTNGVYGGSSYNRGTRITKEVFDNLSNWKNSAQ

SEQ ID NO: 139 - protein, 318 aa, Bacillus sonorensis
  source 1..318: note = Protease _ Mpr
MKKSVTRVLMAGLIGISIYSMGIDSAQAASSPHTPVSSDPSYKPDSSASYDPAIKTNKNGAYSKAFEGTGKLDAPLYQEKSKPTKKSPAGPRYSPKSVIGSDERTRVTNTTAYPYRAIVHISSSIGSCTGWLIGPKTVATAGHCIYDTASGSFAGTATVSPGRNGSTYPYGSVTSTRYFIPSGYRSGNSNYDYGAIELSQPIGNTVGYFGYSYTTSSLVGTSVTISGYPGDKTSGTQWQMSGNIAVSETYKLQYAIDTYGGQSGSPVYEASSSRTNCSGPCSLAVHTNGVYGGSSYNRGTRITKEVFDNLTNWKNSAQ

SEQ ID NO: 140 - protein, 303 aa, Bacillus amyloliquefaciens
  source 1..303: note = Protease 1
MKTVSKRLLSLTAFLLSVSLFAVSAEAAGSPQTSVSNSGEEFDAHQNQPVQTTQTSPAYEGTGLTKRQLAQNSKADIKSLKPSSIIGTDERIRISSTTSFPYRATAQLTVTYSGSTSTYGCTGFFVNADTIVTAGHCVYDQKNGWASKITAAPGRNGSSYPYGSYSGKTFYSVKGWTENGDTNYDYGAIKVNESPGNTVGWYGYRTTNSSSPAGLSSTVTGYPCDKVSGSMWSDSKPIRSAETYKLTYTTDTFGCQSGSPVYRNYSDTGQTAIAIHTNGGSSYNLGTRVTNSVFDNIQYWSNQ

SEQ ID NO: 141 - protein, 303 aa, Bacillus velezensis
  source 1..303: note = Protease 1
MKTVSKRLLSLTAFLLSVSLFAVSAEAEGSPQTSVSNSGEEFDAHLNQPVQTTQTSPAYEGTGLTKRQLAQNSKADIKSLKPSSIIGTDERIRISPTTSFPYRATAQLTVTYSGSTSTYGCTGFFVNADTIVTAGHCVYDQKNGWASKITAAPGRNGSSYPYGSYSGKTFYSVKGWTENGDTNYDYGAIKVNGSPGNTVGWYGYRTTNSSNPAGLSSTVTGYPCDKVSGSMWSDSKPIRSAETYKLTYTTDTFGCQSGSPVYRNYSDTGQTAIAIHTNGGSSYNLGTRVTNSVFDNIQYWSNQ

SEQ ID NO: 142 - protein, 312 aa, Bacillus mojavensis
  source 1..312: note = Protease
MRVVSRFRKKCIAGFTILCLGTAAAVSFGTPAKAAENPQTSVSNTGKEAEVSNNQTSKAELVSDPYEGTGKTSKTLQGLIESEKNIKSLKPSSVIGSDERVRISGTTSFPYRATVQLSIKYPNTSSTYGCTGFLVNPNTVVTAGHCVYSKDHGWASSITAAPGRNGSSYPYGTYSGTMFYSVKGWTESKDTNYDYGAIKLNGSPGNTVGWYGYRTTNSSSPVGLSTSVTGYPCDKTFGTMWSDTKPIRSAETYKLTYTTDTYGCQSGSPVYRNYSDTGQTAIAIHTNGGSSYNLGTRVTNDVFNNIQYWANQ

SEQ ID NO: 143 - protein, 312 aa, Bacillus halotolerans
  source 1..312: note = Protease
MRVVSRFRKKCFAGFMILCLATAAAVSFGTPAEAAENPQTSVSNTGKEAEVSNNQTSKAELVSDPYEGTGKTSKTLHELIESEKNIKSLKPSSVIGSDERVRISGTTSFPYRATVQLSVKYPNTSSTYGCTGFLVNPNTVVTAGHCVYSQDHGWASSITAAPGRNGSSYPYGTYSGTMFYSVKGWTESKDTNYDYGAIKLNGSPGNTVGWYGYRTTNSSSPVGLSTSVTGFPCDKTFGTMWSDTKPIRSAETYKLTYTTDTYGCQSGSPVYRNYSDTGQTAIAIHTNGGSSYNLGTRVTNDVFNNIQYWAKQ

SEQ ID NO: 144 - protein, 313 aa, Bacillus subtilis
  source 1..313: note = Protease _ Mpr
MKLVPRFRKQWFAYLTVLCLALAAAVSFGVPAKAAENPQTSVSNTGKEADATKNQTSKADQVSAPYEGTGKTSKSLYGGQTELEKNIQTLQPSSIIGTDERTRISSTTSFPYRATVQLSIKYPNTSSTYGCTGFLVNPNTVVTAGHCVYSQDHGWASTITAAPGRNGSSYPYGTYSGTMFYSVKGWTESKDTNYDYGAIKLNGSPGNTVGWYGYRTTNSSSPVGLSSSVTGFPCDKTFGTMWSDTKPIRSAETYKLTYTTDTYGCQSGSPVYRNYSDTGQTAIAIHTNGGSSYNLGTRVTNDVFNNIQYWANQ

SEQ ID NO: 145 - protein, 313 aa, Bacillus tequilensis
  source 1..313: note = Protease
MKLVLIFKKQWFVYLTVLCLALAAAVPFGARAQAAENPQTSVSNIGEEADASKNQTSKSEQVSAPYEGTGKTSKSLFGGQTELEKNIQTLQPSSIIGTDERTRISSTTSFPYRATVQLSIKYPNTSSTYGCTGFLVNPNTVVTAGHCVYSQDHGWASTITAAPGRNDSSYPYGTYAGTMFYSVKGWTENKNTNYDYGAIKLNGSPGNTVGWYGYRTTNSSSPAGLSSSVTGFPCDKTFGTMWSDTKPIRSAETYKLTYTTDTYGCQSGSPVYRNYSDTGQTAIAIHTNGGSSYNLGTRVTNDVFNNIKYWANQ

SEQ ID NO: 146 - protein, 915 aa, Bacillus mycoides
  source 1..915: note = Protease _ Vpr
MKKTTSTLLSMALVFSSFGALSAHAESLQKEKQFSPQLKANIEQWGENKIAQNVETKTSKEISVIVELQHAPLASQSNIQHAPDLQNSNVQSYHAQLKKAQEDTTKKIKEKAPKATIKETYSTLFSGFSISIPGDQIAALASLPEVKAIYPNLTYKLHETSKSLADQETPNVGGPTVGAPEAWNLKDPTGKSLDGKGMKVAIIDSGVDYTHPDLQANYIGGYDTVDEDNDPMDGNVHGTHVAGIIAGNGKIKGIAPNASILAYRVMNDGGTGTTEDIIQGIERAIQDGADVLNLSLGQDLNVPDQPVTMTLERAAKLGVTAVVSNGNDGPKPWSVDAPGNASSVISVGASTVSIPFPTFQVAGSNKSYQGLPLSKSDFQVGNDAQLIYVGYGNSSDYAKQDVKGKFALVLQGTSSTLVKAEQAKQAGALGVLLISNEKEINIMPEYFAREDVALPVMQLSNTNGEELKTLITKRKKNIKIGQPKQTELIGNFSSRGPSQGSWLIKPDIVAPGVQITSTVPRGGYESHNGTSMAAPQVAGAVALLRQMHPDWTTEQLKAALANTAKTLKDVNENTYPVMAQGSGLINIPKAAQTDALVKPNNVSFGLIKPNSGKVKLTQNITLQNLSNKKKNFSTRIELLDTKTKVQTSFSSSISVKPNSNIEKPFTITVDSSLPQGVYTGNLYVKEQGKTEEMRIPFTFSIDPKEYKRIDGLEIVNSTFSPNNDKILDDNLINYYLVTPVEDVTFHANLITKERVTYQGMIHQGKNETPGYKSFKWNGTKTDGSPLPDGLYQIEAVASNSGGETKQTGAVFVDRTAPKLSSEVDQENLIIRGKVDDILLDWMTESGWVAPGHPVTIQYEINGNGVWENAFLNHWEKNYEIYFDRNQLQQGKNTIHIVATDAAGNTSNLNVDLEVR

SEQ ID NO: 147 - protein, 734 aa, Gottfriedia luciferensis
  source 1..734: note = Protease
MKNYFIKIIICLVFLLIPFKGYLVNAHEKQKQVALISVKEGMITQALKNIGERYPNITVRQRYTETYYGFSVSGSLEELSQLASMPLVKDIHFVTTYRAHLEDSIPFIGADEAQLYKDQRGRKITGEGVKVGVIDTGVDYRHPDLKSNYGGGYDTIDLDKDPMETKLEEKSTFHGTHVAGVIAANGKRKGVAPKAKIFAYRALGPGGVGTSETVLAAIERAVKDKVDIINLSLGNDVNGPDWPTSIALDKAVEKGVLAITAAGNSGPGLWTVGSPATASKALTVGASYPPVLFPSIHVKNKETIITPMIGSVKWTKGTDGRFVFKGYGEKSDLKRVKNKIVLLERGKITFTDKVKNAQEAGAKAVIVFNNVNGRFVGQIKGKFSIPAATVSRKEGLWIKKVIEKRKTDAKTGHEKKVDVLAEFSSRGPVTGTWQMKPDLLAPGVQIKSTIPGGYLSLQGTSMASPHVAGAAALLKQLHPDWSTDDLKSVLVNTATPLYKNNKMMYRAYEQGAGRLDIEEALDTETTLYPTSLSFGVTRKVDQQVKKDFVVKVSNKSNKPKQVTAVNNQQTTGIQWVLPKPFSIPAHTSKNVKLSATVEAKDLKDGFYDGAITFSTDDKESLRMPYLLLQGEPNFPSVMGFHFGRVKGKNHYKYELYLPSKVEVYKIAVFESETFRLLGIIDEQKNSKRGIIKKELVLNDFPKNGIYKFVIFYKRKQYESATESYVNVAKVYVED

SEQ ID NO: 148 - protein, 803 aa, Bacillus amyloliquefaciens
  source 1..803: note = Protease Vpr-like
MKKGIIRYLLPAFVLSFTLSTSSQAAPATKPQTPDLEKAEVFGDIDMTTGKQTTVIVELKEKSLAEAKELGKAQTKSKLKSERSKVKKKALKTIKHGKINREYEQVFSGFSMKLPANEIPKLLSDQDVKAVYPNVTYHTDQLKDKDITLSKDAVSPQMDDSAPYIGANDAWKLGYTGKGVKVAIIDTGVEYKHPDLKKNFGQYKGYDFVDNDYDPEETPSGDPRGASTDHGTHVAGTVAANGTIKGVAPDATLLAYRVLGPGGSGTTENVIAGIERAVQDGADVMNLSLGNSVNNPDWATSTALDWAMSEGVTAVTSNGNSGPNNWTVGSPGTSREAISVGATQLPLNEYAVSFGSYSSAKVMGYNKEDDIKALNKKETELIEAGIGEQKDFEGKDLKGKVAVVKRGSIAFVDKADNAKKAGAIGMVVYNNAPGEIEANVPGMSVPTVKLSSEDGEKLVSQLKAGGTKATFHLSVAKSLTEQMADFSSRGPVMDTWMIKPDVSAPGVNIVSTIPTHDPADPYGYGSKQGTSMASPHVAGAAAVIKQAKPKWSPEQIKAALMNTAETLTDADGDVYPHNAQGAGSIRIMKAIKADSLVAPGSYSYGTFMKDKGNETKKETFTIENQSSIRKSYQLEYSFNGTGITVSGTDRVVIPAHQTGKVNAKVKVNAKKVKAGTYEGTVTVREGGKTVAKVPTLLIVKEPDYPRVTSIDVQDGTTQGTYQIETYLPAGAEELAFLVYDSNLDFVGQAGIYKKQDKGYQYFDWNGKVNGDTALPAGEYYMLAYAANKGKSSQVLTEKPFIIE

SEQ ID NO: 149 - protein, 803 aa, Bacillus velezensis
  source 1..803: note = Protease Vpr
MKKGIIRYLLPAFVLSFTLSTSSQAAPASKPQTPDLEKAEVFGDIDMTTGKQTTVIVELKEKSLAEAKELGKAQTKSKLKSERSKVKKKALKTIKHGKINREYEQVFSGFSMKLPANEIPKLLSDQDVKAVYPNVTYHTDQLKDKDITLSKDAVSPQMDDSAPYIGANDAWKLGYTGKGVKVAIIDTGVEYKHPDLKKNFGQYKGYDFVDNDYDPEETPSGDPRGASTDHGTHVAGTVAANGTIKGVAPDATLLAYRVLGPGGSGTTENVIAGIERAVQDGADVMNLSLGNSVNNPDWATSTALDWAMSEGVTAVTSNGNSGPNNWTVGSPGTSREAISVGATQLPLNEYAVSFGSYSSAKVMGYNKEDDIKALNKKETELIEAGIGEQKDFEGKDMKGKVAVVKRGSIAFVDKADNAKKAGAIGMVVYNNAPGEIEANVPGMSVPTIKLSSEDGEKLVSQLKAGGTKATFNLSVAKSLTEQMADFSSRGPVMDTWMIKPDVSAPGVNIVSTIPTHDPADPYGYGSKQGTSMASPHVAGAAAVIKQAKPKWSPEQIKAALMNTAETLTDADGDVYPHNAQGAGSIRIMKAIKADSLVAPGSYSYGTFMKDKGNETKKETFTIENQSSIRKSYQLEYSFNGTGITVSGTDRVVIPAHQTGKVNAKVKVNAKKVKAGTYEGTVTVREGGKTVAKVPTLLIVKEPDYPRVTSIDVQDGTMQGTYQIETYLPAGAEELAFLVYDSNLDFVGQAGIYKKQDKGYQYFDWNGKVNGDTALPAGEYYMLAYAANKGKSSQVLTEKPFIIE

SEQ ID NO: 150 - protein, 806 aa, Bacillus tequilensis
  source 1..806: note = Protease Vpr
MKHGIIRFLLLSFVLFFALSTGITGVQAAPVSSKSSAGLEKAEVFGDIDMTTSKKTTVIVELKEKSLAEAKEAGESQTKSKLKTARTKVKNKAVKAVKNGKVNREYEQVFSGFSMKLPANEIPKLLAVKDVKAVYPNVTYKTDNVKDKDVTISEDAVSPQMDDSAPYIGANDAWDLGYTGKGVKVAIIDTGVEYHHPDLKKNFGQYKGYDFVDNDYDPKETPTGDPRGAATDHGTHVAGTVAANGTIKGVAPDATLLAYRVLGPGGSGTTENVIAGVERAVQDGADVMNLSLGNSLNNPDWATSTALDWAMSEGVVAVTSNGNSGPNGWTVGSPGTSREAISVGATQLPLNEYAVTFGAYSSAKVMGYNKEDDVKALNNKNVELVEAGLGEAKDFEGKNLTGKVAVVKRGSIAFVDKAENAKKAGAIGMVVYNNLSGEIEANVPGMSVPTIKLSLEDGEKLISSIKAGETKTTFGLTVSKALGEQVADFSSRGPVMDTWMIKPDISAPGVNIVSTIPTHDPQNPYGYGSKQGTSMASPHIAGAVAVIKQAKPKWSVEQIKAAIMNTAVALKDGDGEVYPHNAQGAGSARIMNAIKADSLVSPGSYSYGTFLKENGNDTKKETFTIENQSSIRKSYTLEYTYNGSGISTSGTGRVVIPAHQTGKATAKVKVNTKKTKAGTYEGTVIVREGGKVVAKVPTLLIVKEPDYPRVTSLSVSEGSVQGTYQIETYLPAGAEELAFLIYDSNLDFAGQAGIYKNQDKGYRYFDWDGTINGGTKLQPGEYYLLAYAANKGKSSQVLTEEPFTVE

SEQ ID NO: 151 - protein, 1431 aa, Gottfriedia luciferensis
  source 1..1431: note = Protease _ Vpr-like
MKKKMKKNVTTLAISTGLVTSVFNPISITHNVHAQQVSKVEQVLSALTPAQREAIKQLKISDKEGLQVSPDLDLTSDAPTSVIVEFKDKPAQTAVMVAETQGTTVSEAEAQAKVDASHETFDKDLKGIFSKELKEKKNPYKIKRSYKKAFNGVSMTLPANKVKSLLKSNAVQAVWSDLQVQLDDPSTKVQDSGEQGPTHNMVTFPGIEKLHQEGYTGKGVKIGILDTGIDYNHPDLKGAFKGGYDFVDNDNDPMETTYADWKKSGMPETNGETYYTEHGTHVAGIIAGQGKNNADYAVTGVAPDADIYAYRVLGAYGSGSTSAILAGIDRAVTDGMDIISMSLGANYNDPLFPTAIAVNNAVLSGVTAVVAAGNSGNKMYSLGAPGNAPLAITVGASDTSATIPNYTATAKSTTGDLPANLKLAAQGLSDNIADFQGKTYQIVNVGTGAETAYYKRDANGNYTIPVDVTGKIVLAQRGTLGIEEIVKRAKSHGAKAVVLINTSGDTLQDVFLGNVYGYIPTFLINSAQGMNISIKIPLNEANKGKTLDFTFGDMSQTVTTGNKLADFSSRGPSRVLYDIKPEVTAPGVSVFSTVPSYMHKDDNGNPLPDYKYAYERLSGTSMATPNVSGVAALLKQAHPDMTPADIKASLMNSADPLNDKYSVYEVGAGLVNPYKAVHAQTEIQVKDQTKTLSSNAKNTDDSITLASKGIKTIKNITGALSFGEQAVEGTDFSDSRSMTIFNKSSEDKTFDVKVQFQTLDARFTNDSRADQDAAANGVTLDVKSSVKVKRYSSTNMDATINVPKTAKLGTYEGYVVYTNQTNPNETYKIPFAIHTVKEGIDYVTGNPAAFTLPYEAGSNATKWSVGVNFKLNSHMRTFDFFLVDPKTNEEIGYLGTADGMGADENTEYYFRGVLNNGQYSPLTGDPDNPVGFGFDEVTPGLWKIRMVGTNDNGETFTKDAPIFYGEKEPKVTMNYEAGQIVETANASDKTVTVSGNAFDKNIAEMQAAGITASQGENKMYYYNPNNSAEINIPVDENGNFNSTIPLATAGPNTLPITEYDFYDRDKSTVENYGSFKDVYFIKKGTAYTTAISDKQRINMGDSTTVTFSTKNIATPVKKAEYSFLYPAKFLDVTNVKPHGTYDGKLDVKYTNTTFNKTYNVMTITATATGDLATTGIAGDTSLVDLTFKAKDQYYKGAMQLEDPTGTSRFFKAVYTNVDNSTVTTQGIQPYFYVTPTYSLMRGAVQAEGLASGFDNLGNAIMNTKIDYNKAGSKISVKDSDGKEYGVSDPLGYSVTAPQGFTSRLPITDKPFTLSIDVPGHFTFKKDFTVGIKEDGKVTAGSKPVDYSYIPGGDVNKDNVIDVKDALYIQTYWGTNKRDADINYDGVVDEKDMQYVIDNYSMQNPWDDNSPKAVKKYKGKSLDDVLTDLGLE

SEQ ID NO: 152 - protein, 1384 aa, Gottfriedia acidiceleris
  source 1..1384: note = Protease _ Vpr-like
MNRLIKSTAILTLSTGVILSSFPVKHHTPLVKAAAPSSIEEVLSKLTPEQRQALNKLEATSQEGLQLPSSVDLESSKNLSVIVELKDKPAKVAVIEEAANGNSLSLSDAEQKADAAQETFKNDLQTIYKDDLKENKNVYKIKRTYTSSLNGVSMELPANKVKSLLKSKAVKAVWPNNVIQVEPPKATEGTQDTSKVATEVFPGINKLHDEGLTGKGIKVGVIDTGIDYNHPDLTDAYKGYRAQTGVDPKSINPNSVKGWDFVDNDADPMETTYYDWKKSGLAETSLSGATFYTEHGTHVSGIIAGRGKNNTDYAVTGVAPDADLYVYRVLGQYGSGTTANVLAGIDKSVADGMDVINLSLGSNSNDPLNIESLALNNAVISGVTAVVAAGNAGDRMYTLGAPGASALAITVGANDTKTEIATSKGTLHGTTDVATDLKLLGKGFGDNVDSLKGRNLQIVKVPNTGADADYKSIDVNGKIALVNRGTVSLVDKIKFAKNHGAVAVIITNNVANEGFIPTYLGASYGLVPTFTMSFEQANAITSKLGANPTFSFDEMGKITDAGSKIASFSSRGPARITYDIKPEVTAPGVAVFSTVPSYMHGADQNGNYQYAYDNLSGTSMATPNVAGVAALLLQANPNLTPAEIKETLMNTADPLNGDYSVFESGAGQVDPYEAIHSKTRIEVVDETKGISDKKGNLKTVNDLTGAVAFGVFTPAGQNISDQCSLNIYNNSNQAKTYDVTVTFQKDRRGSLDADANGVMIVTDKSIKVGANSKKKSTVAISVPKSAKLGIYEGFVTYTNHDNPEETFQVPFGIKTVEEGMGPVTMSPQAFTQVFDSAYSGQLLYTNANMSIKSHMRTLDVVLEDGKTGKDIGFIGTMDGLGMDENISYGISKIFNGYYYKFTGDEDQPIAYDTDLAKPGYYKLKIIGTNDAGKQFTSEGDFYFDNTAPKMTVNSPDVLEYKNGQSSVTITGSVYDKETEEMQKLGMNFNQGSTKLYYQDQLGGRAIAVPVNPDGTFTATIPTNPTRAMLLSFYAWDAASNKTFKEGREIFVVRDDVTYGYMQPEKRIAKTGETVHATLNLNKANDVKQAVYKFYHYSDVATMNINPTPNFTDKVNVKVEDKVWSGNLNEATVTITTKDGVAPLNGDLKLADIAFKMGTEYTPQPDIWSGYMSSVTVTDTNNITKAVSSGNPSIFFTEPTFSMFRGSNRAEAFLDPNGAATQTINYSKLGVSVKATNSNGTVYNGKIDASSVFQIQNLPLTDDQYELELDFPGHFTVHKDFTIGFHEDGKVVNQNLYYKLNPAIAGDVNKDDVIDVMDAIYIQTYWGTNKRAADINFDGTVDAKDMAFVEKNYLMQNPTIKYVPTPIKKYKSQTLETVKSALGIQ

SEQ ID NO: 153 - protein, 383 aa, Bacillus safensis
  source 1..383: note = Protease _ AprE
MCVKKKNVMTSVLLGVPLLFSAGFGGSMANAETVSKTDSQKSYIVGFKASATTNSSKKQAVIQNAGKLEKQYRLLNAAQVKMSEQAAKKLEHDPSIAYVQEDHKAEAYAQTVPYGIPQIKAPAIHAEGYKGANVKVAVLDTGIHAAHPDLNVAGGASFVPSEPNATQDFQSHGTHWAGTIAALDNTIGVLGVAPSASLYAVKVLDRNGDGQYSWIISGIEWAVANNMDVINMSLGGPNGSTALKNAVETANNRGVVVVAAAGNSGSFGSTSTVRYPAKKDSTIAVANVNGNNVRNSSSSAGPRLDVSAPGTSILSTVPSSGYTSRTATSMASPHVAGAAALILSKYPNLSTSQVRQRLENTATPLGDSFYYGKGLINVQAASN

SEQ ID NO: 154 - protein, 383 aa, Bacillus subtilis; identical to SEQ ID NO: 153
  source 1..383: note = Protease _ AprE

SEQ ID NO: 155 - protein, 381 aa, Bacillus subtilis
  source 1..381: note = Protease _ AprE 2
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 156 - protein, 379 aa, Bacillus licheniformis
  source 1..379: note = Protease _ keratinase
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMVSPHVAGAAALILSKHPNLSASQVRNRLSSTATYLGSSFYYGKGLINVEAAAQ

SEQ ID NO: 157 - protein, 359 aa, Bacillus glycinifermentans
  source 1..359: note = Protease
MAFSNPASAEQPAKDVEKDYIVGFKSSVKTAAVKKDVIKESGGKVDKQFKIINAAKATLDQDAVKELKNDPSVAYVEEDHVAHALAQTVPYGIPQIKADKVQAQGYKGANVKVGVIDTGIAASHSDLNVVGGASFVSGESYNTDGNGHGTHVAGTVAALDNSIGVLGVAPNVSLYAIKVLNSSGSGTYSAIVSGIEWATANNLDVINMSLGGTSGSTALKQAVDKAYASGVVVVAAAGNSGTSGSSSTIGYPAKYDSVIAVGAVNSSNQRASFSSVGPELDVVAPGVSIYSTYPSNTYATLNGTSMASPHVAGAAALILSKYPTLSASQVRDRLSSTATNLGDSFYYGKGLINVEAAAQ

SEQ ID NO: 158 - protein, 645 aa, Bacillus subtilis
  source 1..645: note = Protease Epr
MKNMSCKLVVSVTLFFSFLTIGPLAHAQNSSEKEVIVVYKNKAGKETILDSDADVEQQYKHLPAVAVTADQETVKELKQDPDILYVENNVSFTAADSTDFKVLSDGTDTSDNFEQWNLEPIQVKQAWKAGLTGKNIKIAVIDSGISPHDDLSIAGGYSAVSYTSSYKDDNGHGTHVAGIIGAKHNGYGIDGIAPEAQIYAVKALDQNGSGDLQSLLQGIDWSIANRMDIVNMSLGTTSDSKILHDAVNKAYEQGVLLVAASGNDGNGKPVNYPAAYSSVVAVSATNEKNQLASFSTTGDEVEFSAPGTNITSTYLNQYYATGSGTSQATPHAAAMFALLKQRDPAETNVQLREEMRKNIVDLGTAGRDQQFGYGLIQYKAQATDSAYAAAEQAVKKAEQTKAQIDINKARELISQLPNSDAKTALHKRLDKVQSYRNVKDAKDKVAKAEKYKTQQTVDTAQTAINKLPNGTDKKNLQKRLDQVKRYIASKQAKDKVAKAEKSKKKTDVDSAQSAIGKLPASSEKTSLQKRLNKVKSTNLKTAQQSVSAAEKKSTDANAAKAQSAVNQLQAGKDKTALQKRLDKVKKKVAAAEAKKVETAKAKVKKAEKDKTKKSKTSAQSAVNQLKASNEKTKLQKRLNAVKPKK

SEQ ID NO: 159 - protein, 466 aa, Bacillus siamensis
  source 1..466: note = Protease
MKQKMIAAIAAVSIMCGGAHTAGAAQPKSSEAERSQAMEKIEKAYELISNEYVENVDKEKLLEGAIQGMLSTLNDPYSVYMDKQTAKQFSDSLDSSFEGIGAEVGMEENKIIIVSPFKQSPAEKAGLKPNDEIISINGESMNGKDLNEAVLKIRGKKGSKVSIKIHRPGTEKQLSFRIKRAEIPLETVFASRKESGGHHVGYIAISTFSEHTAQDFAAALKKLEKQGIDGLVLDVRGNPGGYLQSVEQILKHFVTKDMPYIQIAERNGDKKQYFSTLKHKKPYPVNVITDKGSASASEILAGALKEAGHYDVVGDTSFGKGTVQQAVPMGDGSNIKLTLYKWLTPKGNWIHKKGITPTIAVSQPDYFAAGPLQLKQPLQPDMNSADVKHAQILLKGLGFDPGRADGYFSGTMKKAVMAFQDRYKLDKTAVIDKQTAEKMNQLMNEKKSDEKNDIQLQMALKSLFVK

SEQ ID NO: 160 - protein, 466 aa, Bacillus velezensis
  source 1..466: note = Protease 2
MKQKMIAVIAAVSIMCGGAHIAGAAQPNSSDAERSRAMEKIEKAYELISNEYVENVDKEKLLEGAIQGMLSTLNDPYSVYMDKQTAKQFSDSLDSSFEGIGAEVGMKDNKIIIVSPFKQSPAEKAGLKPNDEIISINGESMNGKGLNEAVLKIRGKKGSKVSIKIQRPGTDKQLSFRIKRAEIPLETVFASRKESGGHHVGYIGISTFSEHTAQDFAAALKKLEKQGIDGLVLDVRGNPGGYLQSVEQILKHFITKDMPYIQIAERNGDKKQYFSTLKHKKPYPVNVITDKGSASASEILAGALKEAGHYDVVGDTSFGKGTVQQAVPMGDGSNIKLTLYKWLTPKGNWIHKKGIAPTIAVSQPDYFAAGPLQLKQPLQPDMNSADVKHAQILLKGLGFDPGRADGYFSKTMKKAVLAFQDRYKLDKTAVIDKKTAEKMNQLMNEKKSDEKNDLQLQMALKSLFVK

SEQ ID NO: 161 - protein, 466 aa, Bacillus amyloliquefaciens
  source 1..466: note = Protease 2
MKQKMIAVIAAVSIMCGGAHIAGAAQPNSTDAERSRAMEKIEKAYELISNEYVENVDKEKLLEGAIQGMLSTLNDPYSVYMDKQTAKQFSDSLDSSFEGIGAEVGMKDNKIIIVSPFKQSPAEKAGLKPNDEIISINGESMNGKGLNEAVLKIRGKKGSKVSIKIQRPGTDKQLSFRIKRAEIPLETVFASRKESGGHHVGYIGISTFSEHTAQDFAAALKKLEKQGIDGLVLDVRGNPGGYLQSVEQILKHFITKDMPYIQIAERNGDKKQYFSTLKHKKPYPVNVITDKGSASASEILAGALKEAGHYDVVGDTSFGKGTVQQAVPMGDGSNIKLTLYKWLTPKGNWIHKKGIAPTIAVSQPDYFAAGPLQLKQPLQPDMNSADVKHAQILLKGLGFDPGRADGYFSKTMKKAVLAFQDRYKLDKTAVIDKKTAEKMNQLMNEQKSDEKNDLQLQMALKSLFVK

SEQ ID NO: 162 - protein, 466 aa, Bacillus velezensis
  source 1..466: note = Protease 3
MKQKMIAVIAAVSIMCGGAHIAGAAQPNSSDAERSRAMEKIEKAYELISNEYVENVDKEKLLEGAIQGMLSTLNDPYSVYMDKQTAKQFSDSLDSSFEGIGAEVGMKDNKIIIVSPFKQSPAEKAGLKPNDEIISINGESMNGKGLNEAVLKIRGKKGSKVSIKIQRPGTDKQLSFRIKRAEIPLETVFASRKESGGHHVGYIGISTFSEHTAQDFAAALKKLEKQGIDGLVLDVRGNPGGYLQSVEQILKHFITKDIPYIQIAERNGDKKQYFSTLKHKKPYPVNVITDKGSASASEILAGALKEAGHYDVVGDTSFGKGTVQQAVPMGDGSNIKLTLYKWLTPKGNWIHKKGIAPTIAVSQPDYFAAGPLQLKQPLQPDMNSADVKHAQILLKGLGFDPGRADGYFSKTMKKAVLAFQDRYKLDKTAVIDKKTAEKMNQLMNEQKSDEKNDLQLQMALKSLFVK

SEQ ID NO: 163 - protein, 266 aa, Bacillus paralicheniformis
  source 1..266: note = Protease 1
MKKRSLFFTLLLSASLIPGADGVAETGGHEHTHEAKGIYKEALPKPKEGYKDLTGKVHIEKTTKTKVLDEKGNETSTITFKKNVNNGKFSTQASTGKQKISVLAVADKQYRQKHKDWKTRIVNIVEEADEMFNRDHDIDFVVEAVAPWSSSGSNASDILDNLADKFEGRPYKFVAGFTANDNFEAGGIAFQYPVKPDGPAFSVTFDLGTENTAKAQRHEFSHNFGVEHDPQGSGIRCIMNYDYAYTVDVWDAEHNSLIEKNKVWYQ

SEQ ID NO: 164 - protein, 266 aa, Bacillus sonorensis
  source 1..266: note = Protease 1
MKKRSLFFTLLLSASLIPGAAGVAETGGHGHPHEAKGIHIEALPKPKDGFKDLAGKVHVEKTTKTKVLDEKGNTAGTMTFEKNVNNSKFSTQASTGKQKVSVLAVADKQFRTKYKDWKTRIVQIVEEADEMFNRDHDIDFVVEAVAPWNSSGKNASDILDNLAAKFEGQPYKFVAGFTANDNFEAGGIAFQYPVKPDGPAFSVTLDMGTENSAKAARHEFSHNFGVEHDPQGSGIRCIMNYDYAYTVDVWDEAHNELIEKNKVWYQ

SEQ ID NO: 165 - protein, 268 aa, Bacillus sonorensis
  source 1..268: note = Protease 2
MKKRSLFFSLLLSAGLLPGITAGAAKTSGHDHGHAHEAKGNYVESLPKANSFKDLNGKVQIERTTVTKILDEKGDVTGTKTFKKNTSGGEFSTQASTGTQKVSVLAVADAQYRAKYGDWQTRIVQIVEQADVMFNRDHSIDFVVEAVAPWTSIGGNSSQILSNLRSSFSGGKYKFVVGFTANSKFDAGGIAYVYSSKPSGSAYSVNLDQGTANTAKAATHEFSHNFGLNHDAQGSGIRCIMNYDYAYTADVWDSAHNSQISNNKAWYK

SEQ ID NO: 166 - protein, 268 aa, Bacillus licheniformis
  source 1..268: note = Protease
MKKRSLFLSLLLSVSLIPGVTAGAAKTGGHEHEHDHEARGTYIDSLPKAKDFKDLKGKVQIERTVNTKILDEKGNVTGTKTFKKNTGKGSFSTQAGTGSQKVSVLAVADAQYRAKYSDWQTRIVQIIEQADVMFNRDHDVDFVVEAVAPWTSSGSNSSQILSNLQSNFSGRSYKFVVGFTANSNFDAGGIAYVYNSKPNGSAFSVNLDQGTANTAKAATHEFSHNFGLNHDAQGSGIRCIMNYDYAYTVDVWDSAHNSQITKNKAWYK

SEQ ID NO: 167 - protein, 268 aa, Bacillus paralicheniformis
  source 1..268: note = Protease 2
MKKRSIFLSLLLSVSLIPGLTAGAAKVDGHDHEHGHEVKGTHIDSLPKAKDFKDLKGKVQIEKTVNTKILDEKGNVAGTKTFKKNTSKGTFSTQAGTGSQKVSVLAVADAQYRAKYSDWQTRIVQIIEQADVMFNRDHDIDFVVEAVAPWTSSGSNSSQILSNLQSNFSGRSYKFVVGFTANSNFDAGGIAYVYNSKPNGSAFSVNLDQGTANTAKAATHEFSHNFGLNHDAQGSGIRCIMNYDYAYTVDVWDSAHNSQITRNKAWYK

SEQ ID NO: 168 - protein, 269 aa, Bacillus atrophaeus
  source 1..269: note = Protease
MKMRSIAFSVLLTAGIIPGLTSAGNLEAGHVHHHHAEKAKTTYADSLPKAKDFKDYNGKVQIEQTVDTKILDENGKQVGKKRYKANVRENKASTKAGTGRQKVSVLAVADAEYRAKYSDWQTRIVQIIEQADVAFNRDHDIDFVVQAVQPWTSSGSNSSQILSNLQNSFRSQNYKFVAGFTAKSSFDAGGIAYVYNGKPSGPAFSVNLDQGTANTAKAAQHEFSHNFGLSHDAQGSGIRCIMNYDYSYTVDTWDSAHNRQITTNKAWYR

SEQ ID NO: 169 - protein, 266 aa, Bacillus amyloliquefaciens
  source 1..266: note = Protease 3
MKISSLVFPVLLAAGLIPGLPSAESHTHSHTHAEPAKTVYTDPLPKADHFKDLKGPVQIEQTVDTKILDENGKQVGTKRFNANVNQEKASTKASTGSQKVRVLAVADAEYRAKYSDWQTRIVQIVEQADVAFNRDHNIDFVVQAVQAWNSSGSNSSQILSNLQSSFSNQNYHFVVGFTAKSSFDAGGIAYVYNGKPSGPAFSVNLDQGTANTAKAAQHEFSHNFGLQHDAQGSGIRCVMNYDYAYSVDIWDSSHNRQIETNKAWYK

SEQ ID NO: 170 - protein, 266 aa, Bacillus velezensis
  source 1..266: note = Protease 4
MKISSLVFPVLLAAGLIPGLPSAESHTHSHTHAEPAKTVYTDPLPKADHFKDLKGPVQVEQTVDTKILDENGKQVGTKRFNANVSQDKASTKASTGSQKVRVLAVADAEYRAKYSDWQTRIVQIVEQADVAFNRDHNIDFVVQAVQAWNSSGSNSSQILSNLQGSFRNQNYHFVVGFTAKSSFDAGGIAYVYNGKPSGPAFSVNLDQGTANTAKAAQHEFSHNFGLQHDAQGSGIRCVMNYDYAYSVDIWDSSHNRQIATNKAWYK

SEQ ID NO: 171 - protein, 266 aa, Bacillus subtilis
  source 1..266: note = Protease
MKISSLVFPVLLAAGLIPGLPSAESHTHSHTHAEPAKTMYTDPLPKADHFKDLKGPVQVEQTVDTKILDENGKQAGTKRFNANVSQDKASTKASTGSQKVRVLAVADAEYRAKYSDWQTRIVQIVEQADVAFNRDHNIDFVVQAVQAWNSSGSNSSQILSNLQGSFRNQNYHFVVGFTAKSSFDAGGIAYVYNGKPSGPAFSVNLDQGTANTAKAAQHEFSHNFGLQHDAQGSGIRCVMNYDYAYSVDIWDSSHNRQIATNKAWYK

SEQ ID NO: 172 - protein, 792 aa, Gottfriedia luciferensis
  source 1..792: note = Protease _ inhA
MNKFVLSSFASVVVAGTLFSGIPLKQASSASQSKVHSELAKHSKSTLDLSLVNDEKLLAALVKRGVVSASASEAQKQAALQNYLEVKGQADLVTEKDPIANKVKKAESTKHSDFKKFNNGLLHGNGKKNGQLKGNPDPVQESASPGIVKKGKLLTLMVEYSDFPHNNIKKEETDNYYSDYTPQHYEDMIFGKDGVKGPNGETFLSQKQYYEQQSGGTYTVDGNAYGWLKVPGTAAFYGKDAKTGHDNVTPGGSKQLVIDAYAAAKAAGVPLQDYDLEDPHDLDGDGNFWEPDGLVDHLQIIHSGMGQEAGGGSLGDNAIWSHRSAKFVDPDGLGKGLPGFYDYTMMPEDGATGVFAHEYGHDLGLPDEYDTIYSGAGEAVEYWSIMSAGSWAGKIPGTEPTGFSPWAKSYFQSTLGGKWTNPTVVNWDDVSTKGTQFLLDQANSPNGQNNQAIQVNLPKKVVPINKPAAGTYEYWGGQADEMDNSMVTTVDLTGKSTATLTFDAWYDMEEQWDFAFVQVSTDNGATWSSLGNSNTRSDVVPEGYPTILNSMPGFTGNSNGWQAQSFDLSAYKGKKIQLRLRSATDWGSSFTGFFADNIKVVADGSTIVDDGAENATSPFTLQGFSKFDGNKYANHYYLLEWRNQKGVDEGLAHIKRGNSLLNYDGGLVVWYVDDSFTDNWTGVHPGDGFLGVVDAHDDTNLLWNTGAEASSRFHVADAAFGLNSTSGYNITYSNGQNLTSPSQESVSLFNDSKDYNNVFLPDAGRNIGHYGLKVRVNGQATDKSVGAIVVYK

SEQ ID NO: 173 - protein, 796 aa, Gottfriedia luciferensis
  source 1..796: note = Protease _ inhA 2
MAKPSYWKVLSSVSLVTALSMSILTSGITTKAAIPTESESKQLVESPSLDLNVVDETRLEKALTERGIIKSTDSPEEVKEKIREYITQKQGNKLNGSDVHNSEIAEKQRDTLEKRKEQLLKQLNKGHENYKKGKPNGVVKVDSAKQAKYDGTTREDNVLVILAEFADFKHNNVDKVDGYLYTSDFNKKHYEDMLFGKEDFTLEDGSKIKTFKQFYEEQSGGSYTVNGTVTNWVTVPGKAADYGDDNPAGGHDNKNPLGPRDFIKDAVKAAISSGVNLADFDKLDIYDIDGDGNTNEPDGLVDHLMVIHAGTGQEAGGGRLGDNAIWSHRWTLNGVTAFAGTQASVPYWSGGKMAVYDYTVEPEDGATGVFAHEFGHDLGLPDEYDTQYTGNGEPIASWSIMSGGSWNGKVAGTTPSSFSPQNKEFFQKTMGGNWANITEVDYKDIDEKGIATFIDQSVTKSENPGVVKVNLPKKEVKSKIQPQFGAKYYYSTKGDNLHTFMTSPVLDLTNATTANFAIKANYEVESNYDYVYVNVLDQNGTKLGTIDTIGDENTGSGFESSKGQWVDKSYDLSAYNGKKVKVQFEYVTDGGLAPEGFAFDEVKVTVNGSAILTDGAEGTPTFTLDKAVVSNGTDFKDHYYYLEWRNYAGADKSLEFARGVKYNTGLVVWYGDESFTDNWVGVHPGEGFLGVVDAHPAPMVGDLNGKPSVAQSTRYQVADAAFSYDKSAEFYINSPSRGIFDYRGLPGVTTFDDSQKYFDGSGLIPDAGRKVPNYGLKFQVIGEAKDNSAGAVWIHK

SEQ ID NO: 174 - protein, 795 aa, Bacillus mycoides
  source 1..795: note = Protease _ inhA
MKKKPFKVLSTIALTAVLGLSLGAGSQPTYAETPENKTATSPVDDHLIPEERLADALKKRGVIDSSASEKETKKAVEKYVENKKGENPGKEATNGDQLTKEASDFLKKVKDAKADTKEKLNQPATGTPAATGPVKGGLNGKVPTSSAKQKAYNGDVRKDKVLVLLVEYADFKHNNIDKEPGYMYSEDFNKEHYEKMLFGDEPFALEDGSKIETFKQYYEEQSGGSYTVDGTVTKWLTVPGKAADYGADAATGHDNKGPKGPRDLVKDALKAAVDSGLDLSQFDQFDQYDVNADGNQNQPDGLIDHLMIIHAGVGQEAGGGKLGDDAIWSHRWTVGPKPFAIEGTQAKVPYWGGKMAAFDYTIEPEDGAVGVFAHEYGHDLGLPDEYDTDYTGHGEPIQAWSVMSGGSWAGKIAGTTPTSFSPQNKEFFQKTIGGNWANIVEVDYEKLNKGIGLATYLDQSVTKSDRPGMIRVNLPDKDVKTIAPAFGKQYYYSTKGDNLHTKLETPLFDLTNATNAKFDFKSLYEIEAEYDFLEVHAVTEDGQQTLIERIGEKANSGNAATTNGKWIDKSYDLSQFKGKKVKLTFEYITDGGLALNGFLLDNASLTVDGKVAFSDDAEGTSQFKLDGFVVSSGTEKKKHNYYVEWRNHTGSDSALKFARGPEYNSGMVVWYADSAYSDNWVGLHPGHGFLGVVDSHPEAIVGTLNGKPTIDSSTRFQIADAAFSFDKTPAWKVVSPTRGTYTYNGLAGVPKFDDSKTYINQQIPDAGRILPKLGLKFEVVGQADDNSAGAVRLYR

SEQ ID NO: 175 - protein, 796 aa, Bacillus salacetis
  source 1..796: note = Protease _ inhA
MNKLFKTGLTTVVVAGTLFSGIPGISSVGAKELTEVTQKSKVSHSSHFYGDLDLALINDERLIASLIKQGKVSAKASQAKKQQALQDYIELKGKKTESDALGNDPIAPAAKAAESAKHENFKELTQNGKKGKLQGRTQHPKPVEESPSPGINKKGKLLTIMVEYSDFEHNSIQPHETDNYYEDYNTEHFEEMIFGDDGVTGPNGENFVSQKQFYEEQSGGTYTIEGQAYGWLKVPGTAKFYGADRASGGHDNVTPGGSKALVRDTYLAAIEAGIPLEDYDLEDPHDLDGDGNYWEPDGLVDHLQIIHSGMGQEAGGGSLMDDAIWSHRSASYVDMDALGDGTVGFYDYTMMPEDGATGVFAHEYGHDLGLPDEYDTQYTGTGEAVGYWSIMASGSWAGKIPGTEPTGFSPWAKAYLQSTLGGNWTVPTVVDFEDLNKKGASFLLDQANSTNGKNDQAIQVNLPQKKTLINNPKSGTYEYWGGQQDEMDTNMVTDVDLTGKSSATLTFDAWYDIEEQWDFGFVQVSTDGGSTWTSLGNENTRSDVVPEGYPTILNSMPGFTGNSNGWEAQTFDLSTYAGQEIKLRLRYATDWGSSFTGFFADNIKVVADGATIVEDGGESSESPFTLNGFTKSDGNKYVDHYYLLEWRNHEGVDEGLAHIARGNSLMSYDGGLVVWYVDDTYTDNWTGIHPGDGFLGVVDAHDDTNLVWSTGGQASTRYHIADAAFNFMPTSGLNLDYPGVQSLFLSPQRGIPLFNDNKDFNNIFMPDAGRNIENYGLKVLVNDQAKDKSVGSITIM

SEQ ID NO: 176 - protein, 793 aa, Bacillus haikouensis
  source 1..793: note = Protease _ inhA
MSKLLKTGLTSIVMAGTLFAGFPVSAAVQMESGVNVKEEKHASHFKGTLDLALINDERLMASLIKQGKISSKASEASKQKALDKYIELKGKKVEAASGKKDPIAGKVKAAESEKHAKFEKATKQGSKGKLQGSTKHPKPVEESPSPGVVKKGKLLTLMVEYSDFEHNNIQPNETDNYYSDYNVDHFEDMIFGDDGVEGPNGENFVSQKQFYEEQSGGTYTIEGNAYGWLKVPGTAKYYGADRADGGHDNVSPGGSKRLVADTYAAALAAGIPLEDYDLEDPHDLDVDGNYWEPDGLVDHLQIIHAGMGQEAGGGSLFDDAIWSHRSAAYVDADNLGEGKIGFYDYTMMPEDGATGVFAHEYGHDLGLPDEYDTIYSGAGDAVEYWSIMSAGSWAGKIPGTEPTGFSPWAKMYLQSTLGGNWTDPLVVDLEDLNKKGTNFLLDQANSPNGQNNQAIKVNLPKKKTPINTPASGSYEYWGGQADEIDHNMVMDLDLTGKSSAKLTFDAWYDIEEQWDFGFVQVSTDNGESWTSLGNDNTRSDAVPEAYPTILDSMPGFSGTSDGWEGQTFDLNEYTGQNIKLRLRYATDWGTSQTGFFADNIKVVADGATLVQDGAESESSSFALNGFERFDGNKLMDHYYLLEWRNHQGVDEGLAHIARGSSLMKYDGGLVVWYADDSFTENWTGLHPGDGFLGVVDAHDDTEMLWSDGAPASSRYHVADAAFNFMPTSGMNLVYPDMTLTLASQRGIPLFNDRKDYNNEFLPDAGRNLTKYGLKVLVTDQAKDKSVGSVKLSQ

SEQ ID NO: 177 - protein, 795 aa, Bacillus thuringiensis
  source 1..795: note = Protease _ inhA
MNKKPFKVLSSIALTAVLGLSFGAGGQSVYAETPVNKTATSPVDDHLIPEERLADALKKRGVIDSKASEKETKKAVEKYVENKKGENPGKEVTNGDPLTKEASDFLKKVKDAKADTKEKLDKPATGTPAATGPVRGGLNGKVPTSPAKQKAYNGDVRKDKVLVLLVEYADFKHNNIDKEPGYMYSEDFNKEHYEKMLFGDEPFTLDDGSKIETFKQYYEEQSGGSYTVDGTVTKWLTVPGKAADYGADAATGHDNKGPKGPRDLVKDALKAAVDSGLDLSEFDQFDQYDVNGDGNKNQPDGLIDHLMIIHAGVGQEAGGGKLGDDAIWSHRWTVGPKPFPIEGTQAKVPYWGGKMAAFDYTIEPEDGAVGVFAHEYGHDLGLPDEYDTQYSGHGEPVQAWSIMSGGSWAGKIAGTTPTSFSPQNKEFFQKTIGGNWANIVEVDYEKLNKGIGLATYLDQSVTKTNRPGMIRVNLPDKDIKTIDPAFGKQYYYSTKGDDLHTKLETPLFDLTNATTAKFDFKSLYEIEAEYDFLEVHAVTEDGQQTLIERLGEKANNGNADSTNGKWIDKSYDLSQFKGKKVKLTFDYITDGGLALNGFLLDNASLTVDGKVVFSDDAEGTPQFKLDGFAVSNGTEKKSHNYYVEWRNYAGSDNALKFARGPEYNTGMVVWYADSAYTDNWVGVHPGHGFLGVVDSHPEAIVGTLNGKPTVESSTRFQIADAAFSLDKTPAWKVVSPTRGTYTYNGLAGVPKFDDSKTYINQQIPDAGRILPNLGLKFEVVGQADDNSAGAVRLYR

SEQ ID NO: 178 - protein, 486 aa, Photorhabdus luminescens
  source 1..486: note = Protease _ Serralysin
MEKYMSLKKKISYSEDKDVSGSEKANELLKWLQAYIPGKDSNIVVEHEPSKDAAKELIRGDYRWGHQDDDKSKAFQLKYTFLESKPDDMPWHISEFSAFNEKQKAAAKLSIQSWADVANINFVETTDVKEANITFGFFDVSLTGSYAFAYLPRPEKTQLGTWYNAKSRTFSNNDIDVNGYGRQTFTHEIGHTLGLQHPADYNASDEVSPTYKNSATYFEDSRAYTVMSYFSEKNTGQDFKGIYSSAPLLNDISAIQAVYGANNTIRADDTVYGFNSNTDRDFYTAKDENSKLLFTAWDTGGNDTFDFSGFTQDQRINLNEASFSDVGGLKGNVSIARGVTIENAIGGSGNDVLIGNDAANTLKGGAGDDIIYGGLGADNLWGGEGKDTFVYLSAKESPPLERDWIHDFVSGEDKIDVSLFDLGEAGKGGVRFVREFTGEVGEAVLRYNTVDKVNDFAINLGGEFSYDDFWVKIVGEPILESDFILS

SEQ ID NO: 179 - protein, 563 aa, Serratia marcescens
  source 1..563: note = Chitinase (SmChiA)
MRKFNKPLLALLIGSTLCSAAQAAAPGKPTIAWGNTKFAIVEVDQAATAYNNLVKVKNAADVSVSWNLWNGDAGTTAKILLNGKEAWSGPSTGSSGTANFKVNKGGRYQMQVALCNADGCTASDATEIVVADTDGSHLAPLKEPLLEKNKPYKQNSGKVVGSYFVEWGVYGRNFTVDKIPAQNLTHLLYGFIPICGGNGINDSLKEIEGSFQALQRSCQGREDFKVSIHDPFAALQKAQKGVTAWDDPYKGNFGQLMALKQAHPDLKILPSIGGWTLSDPFFFMGDKVKRDRFVGSVKEFLQTWKFFDGVDIDWEFPGGKGANPNLGSPQDGETYVLLMKELRAMLDQLSAETGRKYELTSAISAGKDKIDKVAYNVAQNSMDQIFLMSYDFYGAFDLKNLGHQTALNAPAWKPDTAYTTVNGVNALLTQGVKPGKIVVGTAMYGRGWTGVNGYQNNIPFTGTATGPVKGTWENGIVDYRQIASQFMSGEWQYTYDATAEAPYVFKPSTGDLITFDDARSVQAKGKYVLDKQLGGLFSWEIDADNGDILNSMNASLGNSAGVQ

SEQ ID NO: 180 - protein, 486 aa, Photorhabdus luminescens
  source 1..486: note = Chitinase Q84F70-PhoLum-MetP+
MEKYMSLKKKISYSEDKDVSGSEKANELLKWLQAYIPGKDSNIVVEHEPSKDAAKELIRGDYRWGHQDDDKSKAFQLKYTFLESKPDDMPWHISEFSAFNEKQKAAAKLSIQSWADVANINFVETTDAKEANITFGFFDVSLTGSYAFAYLPRPEKTQLGTWYNAKSRTFSNNDIDVNGYGRQTFTHEIGHTLGLQHPADYNASDEVSPTYKNSATYFEDSRAYTVMSYFSEKNTGQDFKGIYSSAPLLNDISAIQAVYGANNTIRADDTVYGFNSNTDRDFYTAKDENSKLLFTAWDTGGNDTFDFSGFTQDQRINLNEASFSDVGGLKGNVSIARGVTIENAIGGSGNDVLIGNDAANTLKGGAGDDIIYGGLGADNLWGGEGKDTFVYLSAKESPPLERDWIHDFVSGEDKIDVSLFDLGEAGKGGVRFVREFTGEVGEAVLRYNTVDKVNDFAINLGGEFSYDDFWVKIVGEPILESDFILS

SEQ ID NO: 181 - protein, 285 aa, Aspergillus niger
  source 1..285: note = Chitinase-AspNig
MPDIRGATLTPSTSKADYLEALKEAVGPGGLRTLPPDSVDSPELQDFRRSLANSTATAPGRYTCSSEKPLPVTIRGINNRATLQGGFGGVIPRWKPDNPPQSINFAAFENGYPRPGLALLAANALRDAANDWNELDLGVKFQWVQDIEKASFVLSYAGNQGDVLAEAFFPNEDDLSYLNVYSSAFQPGTVQYLKNIFLHELGHVLGFRHEFAPELEDNKDDFTVQFGPRNPLSVMGYEFPPRLQTTDRDSAEAFYKFPGSHLGWKEARAPKEKRGMLRIKDCTAR

SEQ ID NO: 182 - protein, 266 aa, Streptomyces coelicolor
  source 1..266: note = Chitinase F (Chi19F)
ASCSSYHAWSADANYNTGDIVRYTDGKAYIAEHANPGYDPLISTWYWDPYACDGGSQTPVGNFVVSEAQFNQMFPGRNSFYTYSGLTAALSAYPGFANTGSDTTKKQEAAAFLANVSHETGGLVYIVEQNTANYPHYCDWNQPYGCPAGQAAYYGRGPIQLSWNFNYKAAGDALGIDLLGNPWLVQNDAAVAWKTGLWYWNTQSGPGTMTPHNAMVNGAGFGQTIRSINGSLECDGKNPAQVQSRVTKYQQFAQILGVSPGGNLYC

SEQ ID NO: 183 - protein, 401 aa, Lecanicillium psalliotae
  source 1..401: note = Chitinase 1 (Lpchi1)
TPIANDVAIEKRASGYANAVYFTNWGIYGRNFQPADLAASDITHILYSFMNLRADGTVFSGDTYADYEKHYPSDSWNDVGNNAYGCVKQLYLLKKQNRNMKVMLSIGGWTWSTNFPAAAASAATRKTFAQSAVGFLKDWGFDGIDIDWEYPADATQAQNMVLLLQAVRDELDSYAAQYAKGHHFLLSIAAPAGPDNYNKLKLADLGKVLDYVNLMAYDFAGSWSNYTGHDANLYPNAQNPNATPFNTDDAVKAYIAGGVPASKIVLGMPIYGRSFQQTDGIGKPYNGIGQGSWENGVWDYKALLKAGATVQCDDTAKGCYSYDTSTKELISFDTPAMISIKVSYLKGQGLGGSMFWEASADKKGSDSLIGTSRQGLGSLDSTQNYLDYPNSKYDNIKKGMN

SEQ ID NO: 184 - protein, 370 aa, Lecanicillium lecanii
  source 1..370: note = Acidic Chitinase 1 (Chit1)
MAKCTSPDRKCPAPLIDAPINSFMTDGWADTDIHWEGDSWNDSGTNIYGCLKQLNILKKHNRNFKVLLSIGGWTYSSNFPAPASTPEGRDTFAKSCVELVKNLGFDGIDIDWEYPKTAEEAGHFVELLAEVRKQLDEYSARAANNYHFELSVACPAGPDNFQKLNISGMDAYLDFWNLMAYDYAGSWDKNSGHQANLYPCKDAPGCTPFSTVAAVDYYTSNGVSPDKLVLGMPLYGRAFENCDGIGQPFQGVGQGTWEQGVYDYKKLPLEGAEEHKDDDSCATYCHNAESRTLVTYDTPELAKTKADFIKERGLGGGMWWESSADKAGDDSLIQVVVSELGGPEALKQQQNCISYPETKYDNLRDGLPDN

SEQ ID NO: 185 - protein, 370 aa, Akanthomyces lecanii; identical to SEQ ID NO: 184
  source 1..370: note = Acidic Chitinase 1 (Chit1-H195)

SEQ ID NO: 186 - protein, 2960 aa, Helicoverpa zea
  source 1..2960: note = Chitinase_ Chi10a
MGHNFTLWCALALALCAYAFASPRAGFTFARNAVETIPQHDPIAAPIRASVESIPLRSVRDEGDERLPLRDAVEKRPISLMEPGSSFKIVEDAEVLSYDDGLAGLKYYGAANHLNNSTRNDERLQLGNANLQAMQQTVAGPTARRLVVCYMESWAAYRAPPLAFTAGLVPNSCTHLHYAFATMHPHTYSVIPNNEDYDIVKGGYRIATGLKRRLQGLKVMISVGGEGTDRLFSEMVQEPVRRSMFIESAVNFMRDHDFDGLDLHWVYPGEDEKEKDLLTALLYELREKFSSYGFLLSTVLPPFRYQIEDGYDLSAVSGATDYTVLQAWDMTHGKREEAPSRALQHSALHRDPGAAARDQRYDNIEFMVKFILRNGMNAEKLVLGVPFFGRTYTLAPSTLPSPGAAVSGWGDEGAYTQTKGLLAFFEICMAEREGKGPTGTDEAGNTYAVFDNQWITYDNPTTVLEKMKFIVTTGLAGAAAWSIDMDDFRGLCGQPFPMLSAISTTLNGEKTTTDQSSLKIGSCESENAYLASDEDSCAHFHFCTGGISYRMSCEDERLYDPSTGFCGHQDVAKCMPGQSLRISVEDAARFLSQAYENDFEWNEPMKDMIASKDPERLIAEMDSKKSKQSDKLVVCYMTSWAFYRRGDGKFVPENVDTRLCTHVVYAYASLSPDELIAKEFDPWADVTNNLYERVTSLRDVKVILGLGGWTDSAGDKYSRLVSSEANRSKFIEKLIPFLRMHNFKGLHLDWNYPVCWQSNCKKGAVSDKANFAKFVQELSKALHGASMELGVAISGYKEVIESAYDLPVLSKAADFLSTMTYDYHGGWERSTGHHTPLVPSQRDALAYYSIEYAVKALISGGADPKKLLLGLSFYGQSYRLASMSGAKGPGSPADGPGEPGEFTKQPGMLAYYEICYRVKNLRWKTGRQENAGPYTYSDNQWVGYDDPKSITEKVEWALRQGMGGVTAWAIDLDDFSNRCCAEPSPLLRAAGRALGRSVPSPPTAACERPPEPVTPSPPTTTTVESDGSLGGGSHDHHHHEQTSTTWPGWNPSSTTPSTQQTWWPAATTPMTTTTSTTTTKRPTTTTKRPTTTKKPPADSGTLEGSSCNAGEYHAASGDCEGYLQCEGGAWRKHRCAPGLHWAAAVNRCDWPSFAKCTETASSAASTATTTLQPIASRPPPKPTTTTTTTTTTTTTKRPTMKPTTPAQSDSNSPSDGKPCVGEQYNPVPGDCNAYLHCDGSVWHMQHCAPGLHWSNSQKHCDWPKYAKCEVPKSTTAAPKPTRPSKPPTTTTTTTTTTTKAPASGSPTEDGACGGNDMHAPAQTCDSYLLCVGGRWRKQLCPPGLHWDRRTRRCDWVEFAMCEVGKPSKSPLPPTTTTTTTTTTTTTTTRRPITPRPTRPTTTKKPLADEAQVPGKGCTTGTYHAHPKCEKFYVCVNGVLIAQSCAPGLVWRPDRAQCDFPSASSCSDKRQSDSHDTDSSSQSTVMQQLNEAPPKYCESGAYAPLPSDCTRYLHCLFGTFQEFACSAGLHWNQEKQICDWPKNAKCKRKSSPPTTTTTTTTTSTTTPQPLDADDIAVVVPEHEHIHEHIHEHPQPTPVKPMALPSSGTQPEISSTPALLNSRYKLVCYYTNWSWYRPGIGKYSPEDIDPSLCTHIVYGFAVLSDDGLIMAHDSWSDYDNGFYEKVVAYKRYGIKVSLAIGGWNDSAGDKYSKLVNDPAARARFVTHVVQFLEKYGFDGLDLDWEYPKCWQVDCSKGPDSDKEGFADLVLELSAVFKPKGLLLSAAVSPNKKVIDAGYDVPVLARHLDWIAVMTYDYHGQWDKRTGHVAPLYYHPEDGTTYFNANYTIHYWMAKGAPANKLVMGMPMYGQTFTLGEGNDSPQYLSAYNEATGLNAPASAGGEAGEYTRAKGFLSYYEICDRIRYNGWKVVKDPQQRMGPYAYKGNQWVSFDDVEIIKKKVNFIKSLDLGGGMVWALDLDDFRNRCGQGKHPLVNAIKNGLLDPNVEEETILSDQIIEPPNEIDEGSDEDDIQVKPIAKPTQRPTQLTTQVQPVVTTTKKPVTTISTALEDRYKVVCYYTNWAWYRPGPGKFTPSDIDPSLCTHIVYAFAVLDTKRLVIKPHDIWLDVENKFYEKVVALKEKGVKVVLGLGGWDDSASDKYSRMVNSVAARRKFVIHAIDFIEQYEFDGLDLDWEYPKCWQVNCEKGPSSDKQGFASLVKELRAAFKPRGLSLSAAVSGSKRVIDAGYDVATLSKNLDWISLMSYDYHGQWDKKTGHVSPMYASDGDDDKTLNTNFTVHYWINKGASPEKLILGVPFYGQSFSLEENAGSGLGAPAYAGGEAGDETRARGFLSFYEICDRIRVNGWKVTRDPGGRMGPYASFEDQWVSFDDDFMVRHKAEYVRSMGLGGSMAWSLDLDDFSGRNCGCGKSPLLATINHVLRGLEAPPRCSLEELQPDNAEIDTDVSVVPIVTPESEIVEPDDIPAVSPVEEVDTDTNIDTDTDTDTDTDTDLDTENNGPSLAGTSCTGNVFKADENKCSKYYLCLNGQWMELECPSPLHWNKNHCDWPEKAHCKTKAALRLTGIEPEEIQDDKPIVGCYFTNWAFYRPGKGSFGPEQVDPTACTHIIYAWAYLDGNTFNLVPGNKELDLENDFYGKLTQLRQTGVKVIIGAGGLRDSEGRKWNKMVSSPQNRKTFVDSVLKFLQRWHFDGVQIAWQYPGCKQTPCTNATVDYKERDNFAKLLLELSRTIRPQGLELSAMVAASPEIAQSAYKLHILASELDWVSIAANDYYASSTGKTAHLVPLESSSTKGVSSMNSSLTYWTSVMPPRQVVLGLPAYARSYSLRSAASAALGAPVTGPGEPGPYTRVPGFLAYYEVADDLATHEWKESRTSEGTYSVHGKQWASYLSPLDVGRASAAARHAGARGAALWALDLDVRGPCALLGALRRALTEPDLPLDMCPAA

SEQ ID NO: 187 - protein, 2969 aa, Spodoptera frugiperda
  source 1..2969: note = Chitinase_ Chi10a
MGHNFTLWCALALALCAYVLASPRAGFAFTRSAVETVPQHDPIAAPIRASVESIPLRSVRDEGDERLPLRDAVEKRPISLIEPESAFKLIEDAEVISDEESVSGLKYYGAAGHLNVMTRNDERLQLGNANLQAMQQTVAGPTARRIVVCYMKSWAAYRAPPLAFTAALVPKSCTHLHYAFATMHPHTYSVIPNNEDYDIVKGGYRIATGLKKRLNGLKVMISVGGEGTDRLFSEMVQEPERRTMFIESAVNFMRDHDFDGLDLHWVYPGEDENERELLTALLYELREKFSSYGFLLSTVLPPFRYQIEDGYDLSAVSGATDYTVLQAWDMTHGKPDEPPSRAVQHSPLRRDPGATARDQRYDNIEFMVKFILRHGMNAEKLVLGIPFFGRTYSLASSARSVPGAAVTGWGEEGAYTQTKGLLSYFEICMAEREGKGSSNVDEAGNTYIVFDKEWVTYDTPVTVLEKMKFVVTTGLAGAAAWSIDMDDFRGLCGQPFPLLSAISTTLNGEAIQTDLSSLKIGSCESDNPYLASDVDSCAHFHFCTGGISYRMPCEDDRLYDPSTGFCGHQDVAKCMPGQSLRISVEDAARFLSQAYENDFEWNEPSKDMTANNDPERLIAVMDSKKSKQSDKLVVCYMTSWAFYRRGDGKFVPENIDTRLCTHVVYAYASLSPDELIAKEFDPWADVTNNLYERVTSLKDVKVLLGLGGWTDSAGDKYSRMVSSQASRSKFIEKLIPFLRMHNFKGLHLDWNYPVCWQSNCKKGAVSDKPNFAKFVQELSKALHGAGMELGVAISGYKEVIESAYDLPVLSKAADFLSTMTYDYHGGWEKATGHHTPLVPPSKEALAHYSVEYAVKALISGGADPKKLLLGLSFYGQSYRLNDMNGAKGPGSPASGPGEPGEFTKQPGMLAYYEICYRVKNLRWKTGRQDNAGPYTYSDNQWVGYDDPKSITEKVEWALRQGMGGVTAWAIDLDDFSNRCCAEPSPLLRAAGRALGRSVPSPPTTACERPPEPVTPAAPTTTTAEFDGSAGGSHDHHHHDQTSTTWPSWNPSTTTPSTAQTWWPAATTPMTTTSSTTTTKRPTTTKRPATTTKKPAADAGSLEGTPCNAGDYRAAPGDCEGYLQCEGGVWRKHRCAPGLHWAASVNRCDWPSFAKCTATASSEASTATSTLAPVASRPPPRPTTTTTRRPTTTTTTTRRPTTKPTTIGIQADEGPQEGDSCIGEQYSSVPGDCNAYLHCDGTSWHMQRCAPGLHWSNVQKHCDWPKYAKCEVPKSTTAAPKPTRPSRPTTTTSTTTTTTTRAPPSGSAFENAPCGGNDMHAPSTTCDTYLLCVGGRWRKQLCPPGLHWDKRTRRCDWVEFAMCEVGKPTKSTTSTTTTTTTTTTTTTARPTTTTRRPTTTTRRPTRPTTTKKPLADEAQVPGKGCNTGTYHAHPKCEKFYVCVNGMLIAQSCAPGLVWRPDRSQCDFPSSSSCTDKRQSDGPVSDSTNSQSTMMQQLNEEPPKYCESGAYAQLPSDCTRYLHCLFGKFEEFACSAGLHWNQEKQICDWPKNAKCKRKQGSSTTSTTTTTTSRPVQMDVIEPEHEHISEHGHDHSHHDHEHSHAKPTKPSASVSSIHTQPDISSKPALLNSRYKLVCYYTNWSWYRPGIGKFSPEDIDPTLCTHIVYGFAVLGDDGLIMPHDTWSDIDNGFYEKVVEFKRYGIKVSLAIGGWNDSAGDKYSKLVNDPAARARFVSHVVQFLKKYGFDGLDLDWEYPKCWQVDCSQGPDSDKEAFADLVLELSAVFKPKGLLLSAAVSPSKKVIDAGYDVPVLARHLDWIAVMTYDYHGQWDKRTGHVAPLYYHPEDSTTYFNANYTMHYWMKKGAPANKLIMGMPMYGQTFTLGSEKDSPQSLSMSSEYTGLNAPASDGGEAGEYTRAKGFLSYYEICDRIRYNGWKVVKDPYQRMGPYAYKGNQWVSFDDVEIIKKKVNFIKSLNLAGGMVWALDLDDFKNRCGQGKHPLVNAIKNGLLDSKVEEDTILSDPIIEPPNAIDDDSDDIQVRPIQKPTSRPTQMTTQVTTNWWQTTALTTTTKRPVTTVSTNLEDRYKVVCYYTNWAWYRPGPGKFTPSDIDPSLCTHIVYAFAVLDTKRLVIKPHDIWLDVENKFYEKVVSLKQKGVKVVLGLGGWDDSASDKYSRMVNSPSARRKFVIHALDFIEQYEFDGLNLDWEYPKCWQVNCEKGPSSDKQGFANLVKELRAAFRPRGLSVSAAVSASKRVIDAAYDVATLSKNLDWISLMTYDYHGQWDKKTGHLSPMYEGEDDEDKTLNTNFSVNYWISKGASPEKLILGVPFYGQSFSLEENAGSGLGAPSYAGGEAGDETRARGFLSFYEICDRIRMKGWKVSRDPGGRMGPYASFEDQWVSFDDDFMVRHKAEYVRSMGLGGSMAWALDLDDFSGKNCGCGKSPLLATINHVLRGSEAPPRCPLEELQADTPEPAEVPEPEVPEPDDIPAISVEEVDTDTNTDTDTDSNTNVDTDTDTDTDLVESSGPSLEGGACTGTVFKADENKCSKYYLCLNGHWMQLECPAPLQWHKNHCDWPEKAQCRTKAGLRIGGLEPEEVQDDKSIVGCYFTNWAFYRPGKGSFGPEQVDPSLCTHIIYAWAHLDGKSFNLVPGNSELDLENDFYGKLTQLRQTGVKVIIGAGGLTDSESRKWSTMVSSPENRKTFVNSVLKFLQRWHFDGIQIAWQYPGCRQTPCTSESFNYKERDNFADLLSELSRKLRPHNLEISAMVTASPEIAQLAYKPHILASELDWVAVAANDYYASSTGKTSYLVPLQSSHGSNNLNASLSYWTSALPPRHVVLGIPAYGRSYTLRSASNSGLGAPVTGPGEPGPYTRIPGLLAYYEVADGLASRQWKESRTSEGTFSVRGGQWASYLSVVDVGSIGAAARRAGARGAALWALDLDVRGDCSLLNAMRRGLMEPDLPLEQCAPAA

SEQ ID NO: 188 - protein, 2902 aa, Plutella xylostella
  source 1..2902: note = Chitinase_ Chi10a
MGHNIYIGCTLALVLCVHISAVPYADENEFLRSAVETIPPHHHLATPVRASVESIPPRNLRDEGDERLPLRDAVEKRPINTPDRESLYRFIEDAENLGEDIPGLRKYYGAATYLNSGNRNDERLQQGNANLQEMQQTASGPAGRKLVVCYMESWAAYRAPPLAFTAGLVPKSCTHLHYAFATLHPHTYTVLPTNEDYDIIKGGYRIATGLKRRVPGLKVLISVGGIGTGRLFSEMVKTPTRRKAFVDSAIDFLREHDFDGLDLHWKYPGEKDENEKDHLTSLLYELREKFSSYGLLLSTVLPPFRYQIEDGYDISAVSGATDYAILQGWDMTHRNRDEAPTRAHQHSPLHRDPGADYRDQKYDNIEFMVRLIIRRGMAAEKLILGVPLFGRSYTLSPDTAPAPGASVSGWGDEGKYTQTKGLLAFFEICMMLQDGKGSSSIDEDGNAYAVFGDQWVSYDSPITVVEKMKFVISSGLGGAAAYAIDMDDFRGLCGSPFSILSAISISLNGEAVQSDQSSLKIGSCEADGAYLSSDEVSCAHFHFCTGGTNYKLVCEDERLYDPSTGFCGHQDVAKCIPGQSLRISVDDATRYLTQAYDSDFEWNGQTIKEIMMNNEPGRLKDEYGELDTKKSRDNDKRLVCYMTSWSFYRRGDGKFVPEHIDSRLCTHIVYAYATLTPDELVAKEFDPWTDITNNLYERVTSQKDVKVLLGLGGWTDSAGDKYSRLVSSRANRSKFIQRAIAFLRNNNFKGLHLDWNYPVCWQSNCKKGAVSDKANFAKFVQELSKALHNANMELGVSLSGYKEVIDAAYDLPAISDAADFLSTMTYDYHGGWERTTAHHTPLTSSARDPLGYYSIEYAIKSLISGGADPKKILLGLSFYGQSYRLADSQGSKGVGAAAAGPGEPGEFTKQPGMLAYYEICYRVKNLRWKTDRQDKAGPFAYSDSQWVGYDDPQSIKEKVEWALRQGLGGVTAWAMDLDDFNNRCCNEPSPLLRAAGRALGKSVPPPPTSACERPPPPVTPAPPTTTTVAADGSMGGAAPGHDHGSHGSHGDHTSTTWPSWNPEPSTSTTPSTAMTWWPQASTTTTTTTKRPTTTTTTTTTTRRPTTTRKPTTSADTGIEGSSCRAGEYKAAPGDCESYLQCEGGQWRKSRCAPGLHWSAKETRCDWPSFAKCSESSTSQSSPATSTLAPMTSRPPPPSTTTRKPTTTTTTTTTTTTRRPFTTTTTTESAAVGSDGAAEGEPCSGQGQDYIGVANDCNAYLHCDGTWKMQKCAPGLHWSQAQKHCDWPKYAKCEGSEATSPKPIRPQRPSRPTTSPSNKPVEDGVCQSSDSHASSNACDSYLLCVSGRWKKQNCPPGLHWDDRSKRCDWVEFAMCDMKTNPTKTPLTTRRPSMRPTTSTRKPAVQADNPMKPGMSCRTGSYHAHPRCDKFLVCVNGMLVAQSCAPGLVWNTKVAQCDFPSSTSCSDKRLTGGSPGTMEQLTMCDTGSYAPFPGECTRYRHCLYGKYQEFSCSAGLHWNQEKQICDWPKNAKCRKRGNTKPSSVNQILSPPLKPQHVHETKPAVVKPIATTTSTTTAPFEDEYHNSLLKSPYKLVCYYTNWAWYRPGLGQYGPEDIDPSLCTHIVYGFAVLGDDGLITAHDPWADNDNRFYERVVEYKRYGIKVSLALGGWNDSAGDKYSKLVNNPEAREKFVTHALDFIQQYGFDGLDLDWEYPKCWQVDCSKGPDSDKEGFASLVRELSAVFKPKGLLLSSAVSPNKKVIDAGYDVPVLAKHLDWIAVMTYDFHGQWDKKTGHVAPLYYHPDDDTTYFNANYSMNYWMLKGAPASKLIMGMPMYGQTFTLDTKGRRNATGLNVPAISGGEMGEYTRAKGFLAYYEICERIKDKGWMVQKDPLGRMGPYAYKGDQWVSYDDMDILKSKVNLIKTMELGGGMVWALDLDDFRNRCGQGKHPLLNTIKQGLLDPKIYKETIIMSKPINNFLNDDLEDIEVIPSYTKKTTTTPRPEATTQLAPMHTTRKPVKPATAATEERYKVVCYYTNWAWYRPDSGKYSPSDIDPSLCTHIVYAFAVLDSSKLIIKPHDVFLDVENKFYEKVVALKRHGVKVLLGLGGWNDSAGDKYSRLVNSPSARRTFIVHALDFIEQFEFDGLDLDWEYPKCWQVECENGPSSDKQGFASLVKELKSAFTPRGLLLSAAVSPSKRVIDEGYDVPVLSKNLDWIAVMAYDYHGQWDKKTGHVSPMYVSDKDEHKTFNVNFTMHYYMQKGASRRKLIMGVPMYGQSFSLVEHAGAGLGAPAYAGGEAGDETRARGFLAFYEICERIRSRGWKVTRDPGGRMGPYATLDDQWVSFDDDSMIRHKAEYIREMGLGGSMIWSLDLDDFTGKYCGCGKAPLLQNINHVLRGKEAPPHCSLKEIEVPESEQSSMGVPASRPESSTERDEPEPEPALPAVEEVPDVPDTEENELSLDGKQCSGIRFAADATQCNKYYLCMAGHYVELTCPEGMYWNKNHCDLPSNSNCRSRAQLRLSNEENAEEIVEKPIIACYFTNWAYYREGSGRYGPEQVDATLCTHIIYTWAHLDFNSSEVIPGNPELDLENDFYGKITEQRLAGVKVIIGVGGLEDSEDNEWSMLAQMPEKRKMFVESVIRFLKRWNFDGMQLAWQYPVCKQVPCETVDLEDRDNFNMLLVELAKSMRPLGYELSAMVAASPEIAALAYDTEILMASVDWVAVAANDYYASKTGRTSYLVSLESREDSSVKSFNSTLAYWLSVMPRRQLVVGVPAYARSYTLRRPSAGAAPGDVASGPGQPGPYTDVPGFLAYSEICTNIKSHKWTETRTADGTYAQSSSQWASYLRPEEVSRIAESLHAARARGAALWALDLDDWRGHCSCRSHPLLRALRRGLLQIDTEMC

SEQ ID NO: 189 - protein, 2897 aa, Manduca sexta
  source 1..2897: note = Chitinase_ Chi10a
MGHKFATWSLWLLMLYANVLASPHTSPRTFTQSAVETVPYHEPIAPPVRASVESVPLRSIRDEGEGRVPLRDAVEKRPIEVYDGDSAFKFAEDAEVIPNNLKDYYGAATYLNNSTNRNDERLQLGNANLQEMHQTSGGSAVRRIVVCYVESWAAYRAPPLAFTASLVPRSCTHLHYAFATVHPHTYAVVPSNEDYDVVKGGYRIATGLKRRIPGLKVILSIGGEGTDRLFSEIVQEPIRRSLFIESALNFLQEHDFDGLDLHWVYPGEKDDKEREMLTSLLYEIREKFSPYGLLLSTVLPPFRYQIEDGYDLSAVSGATDYTILQAWDMTHGKRDDPPARAVQHSALHRDPGAASRDQRYDNIEFMVKYIVRHGMAPEKLVLGIPLFGRSYTLAPSTHPSPGALISGWGEEGPYTQTKGSLAYFEICMAEREGKGVTGVDDAGNSYAVFDNQWVTYDTPSTVIEKMKFVINSGLAGAAAWAIDMDDFRGLCGSPFPILGAISVTLNGEAVQTDQSSLKIGSCDSDTPYLASDEDSCAHFHFCTGGISYRMSCEEQRLYDPSTGFCGHQDVAKCLPGQSLRISVEDAAGYLSQVYESDFVWNQAPVKEIWHDVERLTFDEVDIKKNKENDKRVVCYMTSWAFYRRGDGKFVPEHIDTRLCTHVIYAYASLSADELIAKEFDPWADSTNNLYERVTSLKDVKVLLGLGGWTDSAGDKYSRLVSSAASRSKFIEKVIPFLRANNFKGLHLDWNYPVCWQSNCKKGAVSDKPNFAKFVQELGKALHSADMELGVAISGYKEVIESAYDFPSISRDADFLSAMTYDYHGGWERKTAHHTPLIPPTKDALSYYSVEYAIKAIISGGADPKKVLLGLSFYGQSYRLADSNGPKGPGAPAAGPGEPGEFTKQPGMMAYYEICYRVKNLRWKTGRQESAGPYTYSDNQWIGYDDPTSITEKVEWAMRQGLGGVTAWAIDLDDFNNHCCTEPSPLLRAAGRALGRSVPSPPTTACERPPEPVTPAPPTTTTVESDGSLGGGSHDHNHDQHTSTTWPNWTPAATTPITTQTWWPPATTTTTTTTTKRPVTTTKRPVTTTKRPVTTTKPADSGSIEGGSCGAGEYSAAPGDCEGYLQCEGGQWRKHRCAPGLHWAASVNRCDWPSFAKCTATASSAASTATSTLAPMTSRPPPRPTTRRTTTTTTTTTTTTTTTTTRKPTTKPTTVADTTGVSEGQSCTGEVYEAVPSDCNAYLHCDGNTWRLQHCAPGLHWSPLQKHCDWPKYAKCQGSTAVITTTSPRPTRPPTTTTTASGPSDKPREDGECSGTDMHASASTCDSYLLCVSGHWRKQLCPPGLHWDKRTNRCDWIEFAMCDVKPSATKSPVPVPTTSTTRKPTTTRRTTTTSKRPILDEPRKPEKGCITGTYHAHPKCEKFYVCVNNALVSQSCAPGLVWRPDRTQCDFPSAALCTDRRHGVSSPIVDATNTGSEQLQEEQPVYCENGQYAAVPTDCSRYQHCLFGKFEDFSCSAGLHWNQEKQICDWPKSAKCKVKIPSTTTTTSRPVEMDPIVPEVPEIEEPEKPIIPSKPISSAPSTGTQPEISTKPDLLKSRYKLVCYYTNWSWYRPGIGKYSPEDIDPSLCTHIIYGFAVLGADGLITPHDTWADLDHRFYDRVAEFKRHGIKVSLGLGGWNDSAGDKYSKLVNDPAARARFVTHVVSFVETHGFQGLDLDWEYPKCWQVDCSKGPDSDKEAFADLVRELSAVFKRKGLLLSAAVSPSKMVIDAGYDVPVLARHLDWIAVMTYDYHGQWDQKTGHVAPLYYHPEDDNTYFNANYTMHYWIKKGAPPSKLVMGMPMYGQTFTIDNRAVHGLNAPATGGGSPGEYTRAKGFLAYYEICERIKYNGWTVVKDQMNSMGPYAYKDNQWVSFDDPDIIKRKVNFIKSLDLAGGMVWALDLDDFRNRCGQGKHPLMNAIKNGLLEQHVEEEKPILSDELPEPPNSIDEDFDIQVKPIQPRPPKPAQTTQAPIQTTKKPSTAPSYTDERYKIVCYYTNWAWYRPGSGKFTPSDIDPSLCTHIVYAFAVLDSARLVIKPHDIWLDVENKFYEKVAALKSEGIKVVLGLGGWDDSASDKYSRLVNSASARRKFIVHAVDFVEQYGFDGLDLDWEYPKCWQVNCEKGPNSDKQGFANLVKELRAAFTPRGLLLSAAVSASKRVIDAGYDVPTLSRNLDWISLMTYDYHGQWEKKTGHLAPMYAQEDDDDMTLNTNFTIHYWMNKGAPKEKLIMGVPFYGQSFSLMENVGTDIGAPTYAGGEAGDETRARGFLAFYEICDRIRMKGWEVSRDPGGRMGPHATMQDQWVSFDDDYMVRHKAEYVRAMGLGGTMAWALDLDDFTGRYCGCGKFPLLKVMNFVLRNKEVPPQCSLEEIKAPPPPETIDENPIDSPISEPETPSKPQDVQHDTEDSGSNLDGKSCSGTVFKADETDCTKYYLCINGEFTQLTCPDPLHWNKNHCDWPGKARCNTKSQLRITGHGGQESQPERPIVACYFTNWAYYRKGNGSFGPSEVDPLLCTHIIYSWAHLDGSTYKIVPGIPELDIENDFYGKMTELRQTGIQMILGVGGLADSEDDKWREMTSSPEYRRVFIDSVFKVLQKWKFDGVQIAWQYPVCKQAPCTDVSHMADRDNFADLLSELSKTLHHYDLELSAMVSASPEIAALAYKHHVLTTELDWISIAANDYFASSSGRTAYLVPLKSDINGINSFNSSLSYWTNVVPPRKLVLGVPAYARTYTLRSASNTMVGAQVASPGEPGHFTQVPGLLAYYEICRALRSHEWKEVQSEEGTYATSGRQWASYLRPGEAHAAGAAAARARARGAALWALDLDDWRGQCACAPRPVLTALRQGLLDPALPPTLCLYK

SEQ ID NO: 190 - protein, 988 aa, Spodoptera frugiperda
  source 1..988: note = Chitinase_ Chi10b
MWPPRLLRWAFAVLLIAAIIASTSGASNVRRRLRKPSKVSTSVTSSVSRSSDQIISASVNRPKIRGRPNVASRKSSAAIDNSVTTDLHKDKDGYKIVCYYTNWSQYRTKIGKFTPEDIQPDLCTHIIFAFGWLKKGKLSSFESNDETKDGKTGLYDRINALKKANPKLKTLLAIGGWSFGTQKFKEMSATRYARQTFIYSAIPYLRDRNFDGLDIDWEYPKGGDDKKNYVLLLKELREAFEAEAQEVKKPRLLLTAAVPVGPDNIKGGYDVPAVASYLDFINVMAYDFHGKWERETGHNAPLYAPSSDSEWRKQLSVDHAAHLWVKLGAPKEKLIIGMPTYGRTFTLSNPNNFKVNSPASGGGKAGEYTKEGGFLAYYEVCDILRNGGTYVWDDEMKVPYAINGDQWVGFDDEKSIRNKMRWIKDNGFGGAMVWTVDMDDFSGSVCGGDVKYPLIGAMREELRGISRGKDAQDVDWASVASSALVEVTEKPEPIKISLSDVLKRVKKPVKNVIIKNNNNNAVIDKNRREPQILCYLTSWSSKRPSAGRFLPENVDPTLCTHIIYAFATLKDHKLTEGDEKDAEMYDKVVALREKNPNLKILLAIGGWAFGSTPFKELTSNVFRMNQFVYEAIEFLRDYQFNGLDVDWEYPRGADDRAAFVSLLKELRLAFEGEAKTSGQPRLLLSAAVPASFEAIAAGYDVPEISKYLDYINVMTYDFHGQWERQVGHNSPLFPLESATSYQKKLTVDYSAREWVRQGAPKEKLMIGMPTYGRSFTLINDTQFDIGAPASGGGAAGRFTNEAGFMSYYEICEFLREDNTTLVWDNEQMVPFAYRDDQWVGFDDERSLKTKMAWLKEEGFGGIMVWSVDMDDFRGSCGTGKYPLITAMKQELGDYKVRLEYDGPYESSNPNGQYTTKDPNEVVCEEEDGHISYHPDKADCTMYYMCEGERKHHMPCPQNLVFNYNENVCDWPENVEGCTHHTQAPAAKR

SEQ ID NO: 191 - protein, 988 aa, Plutella xylostella
  source 1..988: note = Chitinase_ Chi10b
MWPPSLLRWSAAILLVLAVLTPISDSSGAVRRRLRKPTNKVSTSVTSSVSRSSDQIISASVNRPKIRGRPSVASRKSSAAIDNSVNTEDHKDKDGYKVVCYYTNWSQYRTKIGKFVPEDISPDLCTHMIFAFGWLKKGKLSSFESNDETKDGKTGLYDRINALKKGNPKLKTLLAIGGWSFGTQKFKEMSATRYARQTFIYSSIPYLRDRNFDGLDIDWEYPKGGDDKKNYVLLLKELREAFEAEAQEVKKPRLLLTAAVPVGPDNIKSGYDVPAVASYLDFINLMAYDFHGKWERETGHNAPLYAPSTDSEWRKQLSVDHAAHLWVKLGAPKEKLIIGMPTYGRTFTLSNQNNFKVNSPASGGGKAGEYTKESGFLAYYEVCEMLRNGATYVWDDEMKVPYAISGDQWVGFDDEKSIRHKMRWIKDNGFGGAMVWTVDMDDFSGTVCGGDVKYPLIGAMREELRGISRGKDAQDVDWASVANSVIVEVTEKPEPIKISLSDVISRAKKPTKTLVIKNNNAALDKNKREPQVLCYLTSWSSKRPSSGRFTPENVDPMLCTHLIYAFATLKDHKLTEGDEKDADMYDKVVALREKNPNLKILLAIGGWAFGSTPFKELTSNVFRMNQFVYEAIEFLRDYQFNGLDVDWEYPRGADDRAAFVNLLKELRLAFEGEAKSSGQPRLLLTAAVPASFEAIAAGYDVPEISKYLDFINVMTYDFHGQWERQVGHNSPLFPLESATSYQKKLTVDYSAREWVRQGAPKEKLMIGMPTYGRSFTLINETQFDIGAPASGGGQAGRYTNEAGFMSYYEICEFLREDNTTLVWDNEQMVPFAYRDDQWVGFDDERSLKTKMAWLKEEGFGGIMVWSIDMDDFRGSCGTGKYPLITAMKQELSGYKVKLEFDGPYESSNPNGQYTTKDPNEVTCEEEDGHISYHPDKADCTMYYMCEGERKHHMPCPSNLVFNPNENVCDWPENVEGCQHHTQAPAAKR

SEQ ID NO: 192 - protein, 988 aa, Manduca sexta
  source 1..988: note = Chitinase_ Chi10b
MWPPRLLRWTFTIILIVAVIAPTSDSTTVRRRLRKPSKVSTSVSTSVSRSTDQVLSASVNRPKIRGRPNIASRKSSAAIDNSVTTDHHKDKDGYKIVCYYTNWSQYRTKIGKFMPEDIQPDLCTHIIFAFGWLKKGKLSSFESNDETKDGKTGLYDRINVLKKANPKLKTLLAIGGWSFGTQKFKEMSASRYARQTFIYSAIPYLRDRNFDGLDIDWEYPKGGDDKKNYVLLLKELREAFEAEAQEVKKPRLLLTAAVPVGPDNIKSGYDVPAVASYLDFINLMAYDFHGKWERETGHNAPLYAPSTDSEWRKQLSVDHAAHLWVKLGAPKEKLIIGMPTYGRTFTLSNVNNFKVNSPASGGGKAGEYTKESGFLAYYEVCEILRNGGTYVWDDEMKVPYAIHGDQWVGFDDEKAIRNKMRWIKDNNFGGAMVWTVDMDDFSGSVCGGDVKYPLIGAMREELRGISRGKDAKDVDWASVASSVVIEVTEKPEPIKITLKDVLSRVKKPTKNVVIKQNNIGAAIDKNRREPQVLCYLTSWSSKRPSAGRFMPENVDSSLCTHVIYAFATLKDYKLAEADEKDADMYDKVVALREKNPNLKILLAIGGWAFGSTPFKELTSNVFRMNQFVYEAIEFLRDYQFNGLDVDWEYPRGADDRAAYVSLLKELRLAFEGEAKTSGQPRLLLTAAVPASFEAIAAGYDVPEISKYLDFINVMTYDFHGQWERQVGHNSPLFPLESATSYQKKLTVDYSAREWVRQGAPKEKLMIGMPTYGRSFTLINDTQFDIGAPASGGGSAGRFTNEAGFMSYYEICEFLREDNTTLVWDNEQMVPFAYREDQWVGFDDERSLKTKMAWLKEEGFGGIMVWSIDMDDFRGSCGTGKYPLITAMKQELGDYKVKLEYDGPYESSGPTGQYTTKDPNEVTCEEEDGHISYHPDKADCTMYYMCEGERKHHMPCPQNLVFNYNENVCDWPENVEGCTHHTQAPAARR

SEQ ID NO: 193 - protein, 1015 aa, Bombyx mori
  source 1..1015: note = Chitinase_ Chi10b
MVPTGDSHYHALLSRDFPEITRPAGTQRTRPPILLRWMLTVVLVIAVIAPTSDSTTVRRRLRKPSKVSTSVSSSVSRSTDQVLSASVNRPKIRGRPSIASRKSSAALDNSVTTEHHKDKDGYKIVCYYTNWSQYRTKVGKFVPEDIQPDLCTHIIFAFGWLKKGKLSSFESNDETKDGKTGLYDKINGLKKGNPKLKTLLAIGGWSFGTQKFKDMVATRYARQTFIYSAIPYLRDRNFDGLDIDWEYPKGGDDKKNYVLLLKELREAFEAEAQEVKKPRLLLTAAVPVGPDNIKSGYDVPAVASYLDFINLMAYDFHGKWERETGHNAPLYAPSTDSEWRKQLSVDHAAHLWVKLGAPKDKLIIGMPTYGRTFTLSNPNNFKVNAPASGGGKAGEYTKESGFLAYYEVCEMLRNGGAYVWDDEMKVPYCVHGDQWVGFDDEKSIRNKMRWIKDNGFGGAMVWTVDMDDFSGNVCGGDVKYPLIGAMREELRGISRGKDAKDVDWATVASSIVIEETEKPEPIQVPLSEVLKRIKKPGKNVIIKTNGAALSDKNKREPQVLCYLTSWSAKRPSAGRFTPENVDPKLCTHIIYAFATLKDHKLAEADDKDADMYDKVVALREKNPNLKILLAIGGWAFGSTPFKELTSNVFRMNQFVYEAIEFLRDYQFNGLDVDWEYPRGADDRAAFVSLLKELRLAFEGEAKTSGQPRLLLTAAVPASFEAIAAGYDVPEISKYLDFINVMTYDFHGQWERQVGHNSPLFPLESATSYQKKLTVDYSAREWVRQGAPKEKLMIGMPTYGRSFTLINETQYDIGAPASEGGEAGRFTNEAGFMSYYEICEFLREDNTTLVWDNEQMVPFAYRGDQWVGFDDERSLKTKMAWLKEEGFGGIMVWSVDMDDFRGSCGTGKFPLITTMKQELSDYKVKLEYDGPYETVLTSGQYTTKDPTEVTCEEEDGHISYHKDQADCTMYYMCEGERKHHMPCPSNLVFNPNENVCDWPENVEGCAHHTQAPPARR

SEQ ID NO: 194 - protein, 987 aa, Spodoptera exigua
  source 1..987: note = Chitinase_ Chi10b
MWPPRLLRWAFAVLLIVAIITPTSDSTNVRRRLRKPSKVSTSVTSSVSRSSDQIISASVNRPKIRGRPNVASRKSSAAIDNSVTTDLHKDKDGYKIVCYYTNWSQYRTKIGKFTPEDIQPDLCTHIIFAFGWLKKGKLSSFESNDETKDGKTGLYDRINALKKANPKLKTLLAIGGWSFGTQKFKEMSATRYARQTFIYSAIPYLRDRNFDGLDVDWEYPKGGDDKKNYVLLLKELREAFEAEAQEVKKPRLLLTAAVPVGPDNIKGGYDVPAVASYLDFINVMAYDFHGKWERETGHNAPLYAPSSDSEWRKQLSVDHAAHLWVKLGAPKEKLIIGMPTYGRTFTLSNPNNFKVNSPASGGGKAGEYTKEGGFLAYYEVCEILRNGGTYVWDDEMKVPYAINGDQWVGFDDEKSIRNKMRWIKDNGFGGAMVWTVDMDDFSGSVCGGDVKYPLIGAMREELRGISRGKDAQDVDWASVASSALIEVTEKPEPIKISLSDVLKRVKKPVKNVIIKNNNNAAIDKNRREPQILCYLTSWSSKRPSAGRFLPENVDPTLCTHIIYAFATLKDHKLTEGDEKDAEMYDKVVALREKNPNLKILLAIGGWAFGSTPFKELTSNVFRMNQFVYEAIEFLRDYQFNGLDVDWEYPRGADDRAAFVSLLKELRLAFEGEAKTSGQPRLLLSAAVPASFEAIAAGYDVPEISKYLDFINVMTYDFHGQWERQVGHNSPLFPLESATSYQKKLTVDYSAREWVRQGAPKEKLMIGMPTYGRSFTLINDTQFDIGAPASGGGAAGRFTNEAGFMSYYEICEFLREDNTTLVWDNEQMVPFAYRDDQWVGFDDERSLKTKMAWLKEEGFGGIMVWSVDMDDFRGSCGTGKYPLITAMKQELGDYKVRLEYDGPYESSNPNGQYTTKDCNEVVCEEEDGHISYHPDKADCTMYYMCEGERKHHMPCPQNLVFNYNENVCDWPENVEGCTHHTQAPAAKR

SEQ ID NO: 195 - protein, 504 aa, Helicoverpa zea
  source 1..504: note = Chitinase_ Chi2a
MKVLSLFFVTAVVSVCGQTLGGPKHDKKVVCYIATWAVYRPDPGKFSLEDLEPSLCTHLVYSFAGLNETTNTIKSLDPWQDLETDYGKAGYKRLVGLKQKYPHLKVTLAIGGWNEGSPRYSNMASKPETRAAFIKSVLLMLNNYKFDGLDLDWEYPTKRDGKPYDRENFVLLVKELSEAFEPYGYILTAALGAGKTTMETAYDLAKLSRYLDLIHMMCYDYHGTWDRVLGANAPMKGIGENDTLSVEWTVEYLLSHGVSPYKMVLGLPMYGRTFLLTDPGTRVLEYGKTPVRENGFKGPYTGEAGFMGYNEICVQTTNKSSPWVYHWHEQTSTPYIRDGEKVIAFDNGRSIAIKVQMAIDKGLGGVMVWSIDTDDFKGNCNETDAYIDFVERHKRMVYDPVVKKAVENLHIPDVTLVGSKGPAYVIQDDKLHLRLPQPKYTNYALMQTINDAITLAVEEKRIMDEVKRLEEHNVVKTDDDKGASSFLQSSVVCVVLSVAFVLNL

SEQ ID NO: 196 - protein, 510 aa, Spodoptera frugiperda
  source 1..510: note = Chitinase_ Chi2a
MNVLCLILFVGVAASAHGQTLVEEVKGPKHGKKVVCYIATWAVYRPDPGRFSLEDLEPGLCTHLVYSFAGLNETSNTIKSLDPWQDLETDYGKAGYKRLVAMKAKYPHLKVTLAIGGWNEGSPRYSNMANKTESRAAFIKSVLLMLNNYKFDGLDLDWEYPTKRDGKPYDRENFVLLVKELSEAFEPHGYILTAALGAGKSTMETAYDLAKLSRYLDLIHMMCYDYHGTWDGVLGANAPLKGINENDTLSVEWTVEYLLSHGVSPYKMVLGLPMYGRTFLLTDPATRVLEYGSTPVKATGFKGPYTGEAGFMGYNEICAETMNKSSPWVYHWHEQSATPYLRDGEKVISYDNGRSIAIKVKMAIDKGLGGVMVWSIDTDDFRGICKETDAYIDFVERHRRMVTDPVVNKAVKNLNIPDVTQVGSKGPAYVLQDNILQLRLPQPKYINYALMQTINDAITLAVEEKRILDEVERLESDNVIRKVEDKGGNGHSIRSSIVGIVLCALVVLNL

SEQ ID NO: 197 - protein, 539 aa, Manduca sexta
  source 1..539: note = Chitinase_ Chi2a
MFEFLKSFISYLFKEVINSIVFIIRKRFYNKMVNTILLLSVALMAPILDARTLTGPTHNKVVVCYVATWANYRNATVKYEMKDFNPDLCTHLVYAFAALDPTNHTIKPHDAWLDLESPGLKGYKTAVDMKRDYPHLKVTIAIGGWNEGSEKYSNMSSSPESRKRFIDSVVAFIDHYKFDGLDLDWEFPSKRGGRPEDKANFVALVKELKDAFESKQYLLTAALGAGKDTMDTAYDVAKLSRYLDLIHMMCYDYHGTWDGVVGANAPLRGTSEDDVLSLEYTIKYMISHGVSPYKLVLGLPMYGRNFLLKDPDTTEIVFGKTPAQPEGFKGNYIQETGFMGYNEICMDLMNNTAWKKFWDEKSATPYLRDGDRVVVYDNQRSIALKVKMAVDYGLGGVMVWSIDTDDTKGVCAEELDALTDIYARFNKMLVDPLVTKAVKSLGSGNKNHNYKGQYYLIHNDKLELRLLTRGNTNYPLMEAINQAMVISLEEKRIMAEVERITNTNVIDKKDKRGDKPGAASILSSSAVTLLFCLFFIGKL

SEQ ID NO: 198 - protein, 517 aa, Plutella xylostella
  source 1..517: note = Chitinase_ Chi2a
MFGSRSVFFFSLFAMAAILDGQTLDDLSGPTHGKVVVCYVATWAAYRPELGKFELDNLDPGLCTHLVYSFAGLDEHANIIKSLDPWQDLEKDYGKNGFKRLVALKQRYPHLKVTIAIGGWNEGSLKYSLMAEDPEKRSKFVKSVMLFLDMFKFDGLDLDWEYPARRDGRPIDKVNYVSLVKELKEAFEPKGYILTAALGAGKETMDAAYELSKLSRYLDFIHMMCYDYHGTWDGVVGANAPLTGLSEEDVLSIEYTIKYMLSHGVSPYKMVLGLPMYGRSFVLTDPSTKDIIFGKTPAQSQGFKGPYTREAGFLGYNEICMEMSNSSSKWTQHWDAGSQTPYLRDGSRVISYDNARSIAIKVKMAMDYELGGLMVWSIDTDDFLGRCQPESDPYVDFTERFQRIHKDPVMTVVLERLNLPDGTRREYLNRRSAYVLDNGVLNLRLPERQKTNYALMRTINEATSLAIEEKMILDEMHKVEKENEIDVTERAKPGAGVVTSASALTVVMSCVVLARNW

SEQ ID NO: 199 - protein, 566 aa, Plutella xylostella
  source 1..566: note = Chitinase_ Chi2a
MCQTFKVIITLLCVVINYVVSSEKKIVCYYGSWSYYRHGNGKFEIENINPHLCTHLIYTFVGLNQDGSIAILDPYLDLSDNWGRGNIPKFAELKSQNPNLKTMIAVGGWNEGSGKYSTMANDPQLRQNFVNSVVENLQKFNLDGFDVDWEYPNQRDSTHGAVDIDNFTTLLKELKEQFVKYGYLLSVAAAALKHSASLSYNIAAMSQHVDFVNVMAYDFAGSWDSVIGYNAPLYAGADQESKPAEDLWTVETAIEYWLSEGCPPEKIVLGLALYGRTFSLSNPEATTAGSSASGPGLAGLYTGTSGLIGYNEFCEIQLTQPYTVKFDSQARVPYAYNGRNWITYDDPNSLTEKVEYALAKDIGGVMIWSIETDDFRGICGNAYPLLRAINEALGKHDTVTTTTTTTTTSTEAPTTGLSSTSTDAPTTTTSQVPSTSTTTKSPESSSSELPSTTDTSSSTTTETSSVSSSTDSPTTSTPETTATLTSTSTPVTTIPTSAPVTTTTVTSPALCQEAGIISHPDDCSKFIYCIDYPEGLIQPVVFQCPKNLLWDQRILTCNYVSYVTCL

SEQ ID NO: 200 - protein, 558 aa, Plutella xylostella
  source 1..558: note = Chitinase_ Chi2b
MRALLLTAFLALTCSSVQAESGRVVCYFSNWAVYRPGLGRYGLEDIPVDLCSHIIYSFIGVTENTHEVLVIDPELDVDQGGFSNFTSLKASHPKVKFTVAVGGWAEGGSKYSRMVADKKSRMAFVRSIVDFLNKYNFDGLDLDWEYPGAADRGGSFSDKDKFLYLVQELRRAFLRVGKGWELTAAVPLANFRLMEGYHVPELCQELDAIHVMAYDLRGNWAGFADVHSPLYSRPHDQYAYSKLNVNDGLKLWEEKGCPTNKLVVGIPFYGRSFTLSYGNTNYNLGTYINKEAGGGNPAPYTNATGFWAYYEICTEVNKEGSNWTIKWDEQGKCPYAYSGTQWVGYEDPRSVEIKLNWIKEKGYLGAMTWAIDMDDFQGLCGEKNALTKLLHQHMAPYQVPPPNPGSTTPTPEWARPPSTTPDPYEDVVPAGNKPATTARPQATTTTTKRPTTPAVAMTTTTTARPTEAATVSTAATTEACIDTEVPETPEVPEENEVDANDVCYAQDDYMADKKACDKYWRCVNGEPVQFSCQPGTVFNVQLNVCDWPDSANRSDCQM

SEQ ID NO: 201 - protein, 555 aa, Spodoptera frugiperda
  source 1..555: note = Chitinase_ Chi2b
MRAILATLAVLAVVTTAIEADSKARIVCYFSNWAVYRPGVGRYGIEDIPVDLCTHIIYSFIGVTEKSNEVLIIDPELDVDKNGFSNFTALRKSHPDVKFTVAVGGWAEGGSKYSHMVAQKQTRMAFVRSVVDFLKKYDFDGLDLDWEYPGAADRGGSFSDKDRFLFLVQELRRAFIREKRGWELTAAVPLANFRLMEGYHVPDLCQELDAIHVMSYDLRGNWAGFADVHSPLYKRPHDQWAYEKLNVNDGLALWEEKGCPSNKLVVGIPFYGRSFTLSAGNNNYGLGTYINKEAGGGDPAPYTNATGFWAYYEICTEVDKEGSGWTKKWDEHGKCPYAYKGTQWVGYEDPRSVEIKMNWIKEKGYLGAMTWAIDMDDFKGLCGDENPLIKLLHKHMSTYTVPPPRSGNTTPTPEWARPPSTTSDPSEGDPIVTTARPSTTTKRPMKQTTTSKPQVVIEDDEFDIAVRPEPPKAPEAPVVPESPEVPESPAENEIDDHDVCNSEEDYVPDKKKCNKYWRCVNGKGMQFTCQPGTMFNTQLNVCDWPDNAKRQDCEP

SEQ ID NO: 202 - protein, 558 aa, Manduca sexta
  source 1..558: note = Chitinase_ Chi2b
MRATLATLAVLALATAVQCMLSADSRARIVCYFSNWAVYRPGVGRYGIEDIPVEKCTHIIYSFIGVTEGNSEVLIIDPELDVDKNGFRNFTSLRSSHPSVKFMVAVGGWAEGGSKYSHMVAQKSTRMSFVRSVVSFLKKYDFDGLDLDWEYPGAADRGGSFSDKDKFLYLVQELRRAFIRVGKGWELTAAVPLANFRLMEGYHVPELCQELDAIHVMSYDLRGNWAGFADVHSPLYKRPHDQWAYEKLNVNDGLHLWEEKGCPSNKLVVGIPFYGRSFTLSAGNNNYGLGTFINKEAGGGDPAPYTNATGFWAYYEICTEVDKDDSGWTKKWDEQGKCPYAYKGTQWVGYEDPRSVEIKMNWIKQKGYLGAMTWAIDMDDFQGLCGEKNPLIKILHKHMSSYTVPPPHTENTTPTPEWARPPSTPSDPSEGDPIPTTTTAKPASTTKTTVKTTTTTTAKPPQSVIDEENDINVRPEPKPEPQPEPEVEVPPTENEVDGSEICNSDQDYIPDKKHCDKYWRCVNGEAMQFSCQHGTVFNVELNVCDWPSNATRRECQQP

SEQ ID NO: 203 - protein, 578 aa, Helicoverpa zea
  source 1..578: note = Chitinase_ Chi2b
MRVILATLAVLAVATTAIEADSKARIVCYFSNWAVYRPGVGRYGIEDIPVDLCTHIIYSFIGVTEKSNEVLIIDPELDVDKNGFRNFTALRKPHPNVKFTVAVGGWAEGGSKYSHMVAQKQTRMAFVRSVVDFLKKYDFDGLDLDWEYPGAADRGGSFSDKDRFLFLVQELRRAFIREKRGWELTAAVPLANFRLMEGYHVPDLCQELDAIHVMSYDLRGNWAGFADVHSPLYKRPHDQWAYEKLNVNDGLALWEEKGCPSNKLVVGIPFYGRSFTLSAGNNNYGLGTYINKEAGGGDPAPYTNATGFWAYYEICTEVDKEGSGWTKKWDDAGKCPYAYKGTQWVGYEDPRSVEIKMNWIKEKGYLGAMTWAIDMDDFKGLCGDENPLIKLLHKHMSTYTVPPPRSGNTTPTPEWARPPSTTSDPAEGEIVTTLKPTTAKPATTKPSTAKPTTAKPTTGKPTTAKPTTTKAPQVVTIPDDENDIAVRPEPPKKPVTPETPVVPEVPESAETPTENEIDNHDVCNSEEDYVPDKKKCDKYWRCVNGQGMLFTCQPGTVFNVKLNVCDWPDNADRSDCEP

SEQ ID NO: 204 - protein, 543 aa, Bombyx mori
  source 1..543: note = Chitinase_ Chi2b
MRAIFATLAVLASCAALVQSDSRARIVCYFSNWAVYRPGVGRYGIEDIPVDLCTHLIYSFIGVTEKSSEVLIIDPELDVDKSGFRNFTSLRSKHPDVKFMVAVGGWAEGGSKYSHMVAQKSTRMSFIRSVVDFLKKYDFDGLDLDWEYPGAADRGGSFSDKDKFLYFVQELKRAFIRAGRGWELTAAVPLANFRLMEGYHVPELCQELDAIHVMSYDLRGNWAGFADVHSPLYKRPHDQWAYEKLNVNDGLNLWEEKGCPTNKLVVGIPFYGRSFTLSAGNNNYGLGTYINKEAGGGDPAPYTNATGFWAYYEICTEVDADGSGWTKKWDEFGKCPYAYKGTQWVGYEDPRSVEIKMNWIKEKGYLGAMTWAIDMDDFKGLCGEENPLIKLLHRHMSTHTVPPARTGHTTPTPEWARPPSTPSDPSEGDPIPTTTTTTVKPTTTRTTARPTTTTTKVPHGTTEEDFDINVRPEVEELPTENEVDNADVCNSEDDYVPDKKECSKYWRCVNGEGVQFSCQPGTIFNVKLNVCDWPENTDRPECS

SEQ ID NO: 205 - protein, 557 aa, Spodoptera exigua
  source 1..557: note = Chitinase Chi2b
MRAILATLAVLAVVTTAIEADSKARIVCYFSNWAVYRPGVGRYGIEDIPVDLCTHIIYSFIGVTEKSNEVLIIDPELDVDKNGFKNFTALRKSHPDAKFTVAVGGWAEGGSKYSHMVAQKQTRMAFVRSVVDFLKKYDFDGLDLDWEYPGAADRGGSFSDKDRFLFLVQELRRAFIREKRGWELTAAVPLANFRLMEGYHVPDLCQELDAIHVMSYDLRGNWAGFADVHSPLFKRPHDQWAYEKLNVNDGLALWEEKGCPSNKLVVGIPFYGRSFTLSAGNNNYGLGTYINKEAGGGDPAPYTNATGFWAYYEICTEVDKEGSGWTKKWDEHGKCPYAYKGTQWVGYEDPRSVEIKMNWIKEKGYLGAMTWAIDMDDFKGLCGDENPLIKLLHKHMSTYTVPPPRSGNTTPTPEWARPPSTTSDPSEGEPIVTTARPSTTTKTSRPTKKPTTTKPQVIIEDDENDIAVRPEPPKAPETPVAPEAPEVPESPAENEIDDHDVCNSEEDYVPDKKKCNMYWRCVNGKGMQFTCQPGTMFNTKLNVCDWPDNADRQDCEP

SEQ ID NO: 206 - protein, 935 aa, Clostridium histolyticum; identical to SEQ ID NO: 122
  source 1..935: note = Collagenase A (ColA)

SEQ ID NO: 207 - protein, 960 aa, Bacillus pumilus
  source 1..960: note = Collagenase B (ColB); note = Bacillus pumilis
MNKNLKFTQMMIGISTVALSFGSIQTQVSAEEKVPYNILQMKPIGTETSKDEMVHATKTEEILTFEERLKVGDFSQRPTLAMKRDESQLKQSYTLAELNKMPDRELIDTLSKISWNQITDLFQFNQDTKAFYQNKERMNVIINELGQRGKSFTKEDSKGIETFVEVLRSAFYVGYYNNELSYLKERSFHDKCLPALKAIAKNLNFALGTAEQDRVVAAYGKLIGNASSDTETVQYAVNILKQYNDNLSTYVSDYAKGQAVYEIVKGIDYDIQSYLQDTNKKPYETMWYGKMDNFINEVNRIALVGNITNENSWLINNGIYYAGRLGKFHSNPYKGLEVITQAMSLYPHLSGPYFVAVEQIKTNYGGKDYSGNTVDLQKIREEGKQQYLPKTYTFDDGSIVFKTGDKVTEEKIKRLYWAAKEVKAQYHRVIGNDKALEPGNADDVLTIVIYNDPDEYQLNRQLYGYETNNGGIYIEEKGTFFTYERTPKQSIYSLEELFRHEFTHYLQGRYEVPGLFGSGEMYQNERLTWFQEGNAEFFAGSTRTNNVVPRKSMISGLSPDPASRYTAKQTLFAKYGSWDFYKYSFALQSYLYNHQFETFDKLQDLIRANDVKNYDSYREALSNNTQLNAEYQTYMQQLIDNQDKYNVPQVTNDYLIQHAPKPLAEVKNEIVDVANIKDEKITKHESQFFNTFTVEGKYTGGTSKGESEDWKTMSKKVNQTLEQLSQKGWSGYKTVTAYFVNYRVNAANQFEYDIVFHGVATEEKEKTTTIVNMNGPYSGIVNEEIRFHSDGTTSENGKVISYLWNFGDGSTSTKANPTHVYGEKGTYTVELTVKDSRGKESKEHTKVTVKQDPQTGGSLEEEKVLPFNTLVKGNLVTPDQTDVYTFNVTDPKEVDISVVNEQNIGMTWAHYHESDMQNYVACGEDEGNTIKGKFVAKPGKYYLNVYKFDDKNGEYSLLVK

SEQ ID NO: 208 - protein, 412 aa, Bacillus pumilus
  source 1..412: note = Collagenase B (ColB); note = Bacillus pumulis
IVNGKRVITKKPELLAPAGNLEKLKIAVHYGADAVFIGGQEYGLRSNADNFTIEEIAEGVEFAKKYGAKIYVTTNIFAHNENMDGLEDYLKALGDANVAGIIVADPLIIETCRRVAPNVEVHLSTQQSLSNWKAVQFWKEEGLDRVVLARETSALEIREMKEKVDIEIESFIHGAMCIAYSGRCVLSNHMTARDSNRGGCCQSCRWDYDLYQTDGANAVALYGEEDAPFAMSPKDLKLIESIPKMIEMGIDSLKIEGRMKSIHYVATVVSVYRKVIDAYCADPENFVIQKEWLEELDKCANRDTATAFFEGTPGYEEQMFGEHAKKTTYDFVGLVLNYDEDTQMVTLQQRNFFKKGDEVEFFGPEIENFTHTIETIWDEDGNELDAARHPLQIVKFKLDKKIYPSNMMRKGK

SEQ ID NO: 209 - protein, 24 aa, Bacillus thuringiensis
  source 1..24: note = Signal Peptide
MKKKVLALAAAITLVAPLQSVAFA

SEQ ID NO: 210 - protein, 28 aa, Bacillus thuringiensis
  source 1..28: note = Signal Peptide; note = Bacillus thuringiensis serovar israelensis 4Q7
MKGKLLKGVLSLGVGLGALYSGTSAQAE

SEQ ID NO: 211 - protein, 24 aa, Bacillus cereus
  source 1..24: note = Bacillus cereus ATCC 10987; note = Signal Peptide
MKKKVLALAAAITVVAPLQSVAFA

SEQ ID NO: 212 - protein, 29 aa, Clostridium perfringens
  source 1..29: note = Signal Peptide; note = Clostridium perfingens
MKRKICKALICATLATSLWAGASTKVYAW

SEQ ID NO: 213 - protein, 29 aa, Streptomyces chromofuscus
  source 1..29: note = Signal Peptide
MLAGPLAAALPARATTGTPAFLHGVASGD

SEQ ID NO: 214 - protein, 31 aa, Bacillus subtilis
  source 1..31: note = Signal Peptide; note = Bacillus subtilis subsp. subtilis str. 168
MKFVKRRIIALVTILMLSVTSLFALQPSAKA

SEQ ID NO: 215 - protein, 28 aa, Caldicellulosiruptor saccharolyticus
  source 1..28: note = Signal Peptide
MCENLEMLNLSLAKTYKDYFKIGAAVTA

SEQ ID NO: 216 - protein, 28 aa, Bacillus subtilis
  source 1..28: note = Signal Peptide; note = Bacillus subtilis subsp. subtilis str. 168
MFKFKKNFLVGLSAALMSISLFSATASA

SEQ ID NO: 217 - protein, 26 aa, Bacillus subtilis
  source 1..26: note = Signal Peptide; note = Bacillus subtilis subsp. subtilis str. 168
MRKKCSVCLWILVLLLSCLSGKSAYA

SEQ ID NO: 218 - protein, 27 aa, Geobacillus stearothermophilus
  source 1..27: note = Signal Peptide; note = Geobacillus stearothermophilus (Bacillus stearothermophilus)
MKLKKKMLTLLLTASMSFGLFGATSSA

SEQ ID NO: 219 - protein, 35 aa, Bacillus subtilis
  source 1..35: note = Signal Peptide; note = Bacillus subtilis subsp. subtilis str. 168
MKISMQKADFWKKAAISLLVFTMFFTLMMSETVFA

SEQ ID NO: 220 - protein, 29 aa, Bacillus subtilis
  source 1..29: note = Bacillus subtilis subsp. subtilis str. 168; note = Signal Peptide
MKRSISIFITCLLITLLTMGGMIASPASA

SEQ ID NO: 221 - protein, 28 aa, Bacillus subtilis
  source 1..28: note = Bacillus subtilis subsp. subtilis str. 168; note = Signal Peptide
MPYLKRVLLLLVTGLFMSLFAVTATASA

SEQ ID NO: 222 - protein, 38 aa, Bacillus circulans
  source 1..38: note = Signal Peptide
MKRSQTSEKRYRQRVLSLFLAVVMLASIGLLPTSKVQA

SEQ ID NO: 223 - protein, 38 aa, Bacillus circulans
  source 1..38: note = Signal Peptide
MKPSHFTEKRFMKKVLGLFLVVVMLASVGVLPTSKVQA

SEQ ID NO: 224 - protein, 28 aa, Bacillus subtilis
  source 1..28: note = Bacillus subtilis subsp. subtilis str. 168; note = Signal Peptide
MKKGIIRFLLVSFVLFFALSTGITGVQA

SEQ ID NO: 225 - protein, 32 aa, Bacillus subtilis
  source 1..32: note = Signal Peptide; note = Bacillus subtilis subsp. subtilis str. 168
MKKFPKKLLPIAVLSSIAFSSLASGSVPEASA

SEQ ID NO: 226 - protein, 41 aa, Bacillus subtilis
  source 1..41: note = Signal Peptide; note = Bacillus subtilis subsp. subtilis str. 168
MKKMSLFQNMKSKLLPIAAVSVLTAGIFAGAELQQTEKASA

SEQ ID NO: 227 - protein, 26 aa, Bacillus subtilis
  source 1..26: note = Bacillus subtilis subsp. subtilis str. 168; note = Signal Peptide
MKVPKTMLLSTAAGLLLSLTATSVSA

SEQ ID NO: 228 - protein, 25 aa, Bacillus subtilis
  source 1..25: note = Signal Peptide; note = Bacillus subtilis subsp. subtilis str. 168
MKKIMSAFVGMVLLTIFCFSPQASA

SEQ ID NO: 229 - protein, 46 aa, Bacillus thuringiensis
  source 1..46: note = Signal Peptide
MPLNISFILKGEMAMRSQKFTLLLLSLLLFLPLFLTNFITPNLALA

SEQ ID NO: 230 - protein, 27 aa, Bacillus thuringiensis
  source 1..27: note = Signal Peptide
MLNKFKFFCCILVMFLLLPLSPFQTQA

SEQ ID NO: 231 - protein, 43 aa, Bacillus thuringiensis
  source 1..43: note = Signal Peptide
MNKIIHLDNFLYRSVNMLNKFKFFCCILVMFLLLPLSPFQTQA

SEQ ID NO: 232 - protein, 23 aa, Bacillus pseudomycoides
  source 1..23: note = Signal Peptide
MEYKPLIMGYLHTWSKGFIGGYE

SEQ ID NO: 233 - protein, 25 aa, Bacillus thuringiensis
  source 1..25: note = Signal Peptide; note = Bacillus thuringiensis serovar israelensis 4Q7
MKKKVLALAAAITLVAPLQSVAFAH

SEQ ID NO: 234 - protein, 24 aa, Bacillus cereus
  source 1..24: note = Signal Peptide
MKKKVLALAAAITLVAPLQNVAFA

SEQ ID NO: 235 - protein, 44 aa, Burkholderia cepacia
  source 1..44: note = Signal Peptide
MARTMRSRVVAGAVACAMSIAPFAGTTAVMTLATTHAAMAATAP

SEQ ID NO: 236 - protein, 23 aa, Pseudomonas fluorescens
  source 1..23: note = Signal Peptide
MGIFDYKNLGTEGSKTLFADAMA

SEQ ID NO: 237 - protein, 40 aa, Streptomyces sp.
  source 1..40: note = Signal Peptide; note = Streptomyces species N174
MHSQHRTARIALAVVLTAIPASLATAGVGYASTQASTAVK

SEQ ID NO: 238 - protein, 32 aa, Paenibacillus sp.
  source 1..32: note = Signal Peptide; note = Paenibacillus species
MFKKWKKFGISSLALVLVAAVAFTGWSAKASA

SEQ ID NO: 239 - protein, 20 aa, Aspergillus saitoi
  source 1..20: note = Signal Peptide
MVVFSKTAALVLGLSTAVSA

SEQ ID NO: 240 - protein, 28 aa, Bacillus sp.
  source 1..28: note = Signal Peptide
MAKLQKGTILTVIAALMFVILGSAAPKA

SEQ ID NO: 241 - protein, 21 aa, Aspergillus japonicus
  source 1..21: note = Signal Peptide
MPSAKPLFCLATLAGAALAAP

SEQ ID NO: 242 - protein, 25 aa, Triticum aestivum
  source 1..25: note = Signal Peptide
MARGSMAAVLAVLAVAALRCAPAAA

SEQ ID NO: 243 - protein, 30 aa, Triticum aestivum
  source 1..30: note = Signal Peptide
MRGLGFAALSLHVLLCLANGVSSRRTSSYV

SEQ ID NO: 244 - protein, 18 aa, Triticum aestivum
  source 1..18: note = Signal Peptide
MWWGSLRLLLLLAAAVAA

SEQ ID NO: 245 - protein, 15 aa, Triticum aestivum
  source 1..15: note = Signal Peptide
MGIWRGSLPLLLLAA

SEQ ID NO: 246 - protein, 31 aa, Bacillus amyloliquefaciens
  source 1..31: note = Signal Peptide
MIQKRKRTVSFRLVLMCTLLFVSLPITKTSA

SEQ ID NO: 247 - protein, 436 aa, Bacillus subtilis
DDSAPYIGANDAWDLGYTGKGIKVAIIDTGVEYNHPDLKKNFGQYKGYDFVDNDYDPKETPTGDPRGEATDHGTHVAGTVAANGTIKGVAPDATLLAYRVLGPGGSGTTENVIAGVERAVQDGADVMNLSLGNSLNNPDWATSTALDWAMSEGVVAVTSNGNSGPNGWTVGSPGTSREAISVGATQLPLNEYAVTFGSYSSAKVMGYNKEDDVKALNNKEVELVEAGIGEAKDFEGKDLTGKVAVVKRGSIAFVDKADNAKKAGAIGMVVYNNLSGEIEANVPGMSVPTIKLSLEDGEKLVSALKAGETKTTFKLTVSKALGEQVADFSSRGPVMDTWMIKPDISAPGVNIVSTIPTHDPDHPYGYGSKQGTSMASPHIAGAVAVIKQAKPKWSVEQIKAAIMNTAVTLKDSDGEVYPHNAQGAGSARIMNAIKAD

SEQ ID NO: 248 - protein, 218 aa, Pseudomonas sp.
MTEPLILQPVKPADACVIWLHGLGADRYDFLPVAEALQEILLSTRFVLPQAPTRPVTINGGYAMPSWYDIKAMSPARAIDRDELEASADRIIELIEEQRSSGIDASRIFLAGFSQGGAVVYHTAFLKWQGPLGGVLALSTYAPTFSDELELSASQQRIPVLSLHGQFDNVVQNSMGRTAYEYLKAHGVTVTWQEYPMEHEVLPEEIRDIGTWLSERLR

SEQ ID NO: 249 - protein, 642 aa, Bacillus thuringiensis
DSPKQSQKIVGYFPSWGVYGRNYQVADIDASKLTHLNYAFADICWKGKHGNPSTHPDNPNKQTWNCKESGVPLQNKEVPNGTLVLGEPWADVTKSYPGSGTTWEDCDKYARCGNFGELKRLKAKYPHLKTIISVGGWTWSNRFSDMAADEKTRKVFAESTVAFLRAYGFDGVDLDWEYPGVETIPGGSYRPEDKQNFTLLLQDVRNALNKAGAEDGKQYLLTIASGASQRYADHTELKKISQILDWINIMTYDFHGGWEATSNHNAALYKDPNDPAANTNFYVDGAIDVYTNEGVPVDKLVLGVPFYGRGWKSCGKENNGQYQPCKPGSDGKLASKGTWDDYSTGDTGVYDYGDLTANYVNKNGFVRYWNDTAKVPYLYNATTGTFISYDDNESMKYKTDYIKTKGLSGAMFWELSGDCRTSPKYSCSGPKLLDTLVKELLGGPINQKDTEPPTNVKNIIVTNKTSSSVQLSWTASTDNVGVTEYEITAGEEKWSATTNSITIKNLKPNTEYTFSVIAKDASGNKSHPTALTVKTDEANTTPPDGNGTATFSVTSNWGSGYNFSIIIKNNGTIPIKNWKLEFDYSGNLTQVWDSKISSKTNNHYVITNAGWNGEIPPGGSITIGGAGTGNPAELLNAVISEN

SEQ ID NO: 250 - protein, 248 aa, Bacillus thuringiensis
YGFVSGVNDVLNPKASNLIKKTDVVAKEKKKTGTLQIVSLGDSLTRGVGDKEGIGYVGRMKEDLQKDYKQKIALTNLAVSGAKMPDLLKQIESNGAQYSIKQADVIVLTIGGNDLFPGWESLGKIDLETYRPDTETFQNEAKKIIEEIRKLNTDSPIFWLGLYNPFEDVEDLKGSSNIVVDWNASLEKLALNDKNVYITPTFDLFQNRGKDLLYSDHFHPNEVGYTYMAERLVQNVVSKLKLEQGGVK

SEQ ID NO: 251 - protein, 506 aa, Bacillus thuringiensis
EVVKELGKGFPDTEVFTPGEWFLGQKPANYDENKPPILFVQGRNGNADSWYGKTVYHDINDMYDYALKAGYQTVFIQLYDAAGKGSASQWDNGKLLAQKLEEIYNYFGKKVNIVAHSKGGIDTQAALVEYGANRFVGNVITLATPHHGSNLADLSYSWWAGWLASILGQKDDGTYSLQIGEMAKFRSIIDNNPAAKLNRYYTATGTSWGPTFSALSMGGLYLSSYGSNDGLVNEWSAKLSYGTHLFTDSRFDHDNIRKGSAVFARIEPYLRTTNVGLPALVASSTSSNEDLEQLNTTSNQNILGGELPQNQWIEQSISVDKKAEGIVSVLTASSDVEVQMVSPKGKIYANKDSAITTGEGESFFNRATIRTFKFDKLEVGEWKIKMMAKQSKDAYLVVSDYKDDAPFVLRMPTKVKINKPEYKLKKSPVAPEMKGDLSITVRVVNKEGKLVSEFNELQNVNTNTFTGALKDIKQPGVYNVTMDIKGMNKEGKPYNRTIVKSVYVEK

SEQ ID NO: 252 - protein, 246 aa, Geobacillus stearothermophilus
  source 1..246: note = Bacillus stearothermophilus
MKIVPPKPFFFEAGERAVLLLHGFTGNSADVRMLGRFLESKGYTCHAPIYKGHGVPPEELVHTGPDDWWQDVMNGYEFLKNKGYEKIAVAGLSLGGVFSLKLGYTVPIEGIVTMCAPMYIKSEETMYEGVLEYAREYKKREGKSEEQIEQEMEKFKQTPMKTLKALQELIADVRDHLDLIYAPTFVVQARHDEMINPDSANIIYNEIESPVKQIKWYEQSGHVITLDQEKDQLHEDIYAFLESLDW

SEQ ID NO: 253 - protein, 269 aa, Rhizopus cf. oryzae
  source 1..269: note = Rhizopus oryzae
SDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTANPTYKVIVTGHSLGGAQALLAGMDLYQREPRLSPKNLSIFTVGGPRVGNPTFAYYVESTGIPFQRTAHKRDIVPHVPPQSFGFLHPGVESWIKSGTSNVQICTSEIETKDCSNSIVPFTSILDHLSYFDINEGSCL

SEQ ID NO: 254 - protein, 181 aa, Bacillus subtilis
AEHNPVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADMIVMNYLSRLDGARNVQIHGVGHIGLLYSSQVNSLIKEGLNGGGQNTN

SEQ ID NO: 255 - protein, 280 aa, Sphingobium wenxiniae; identical to SEQ ID NO: 95

SEQ ID NO: 256 - protein, 364 aa, Burkholderia sp.
MARSMRSRVMAGAVACAMSVAPFAGTTALTTLATTQSAVAASAPADNYAATRYPIILVHGLTGTDKYAGVLEYWYGIQEDLQQHGATVYVANLSGFQSDDGPNGRGEQLLAYVKTVLAATGATKVNLVGHSQGGLTSRYVAAVAPDLVASVTTIGTPHRGSEFADFVQSVLAYDPTGLSSTVIAAFVNVFGILTSSSHNTNQDALASLKTLTTAQAATYNQNYPSAGLGAPGSCQTGAPTETVGGNTHLLYSWAGTAIQPTLSLFGVTGAQDTSTIPLVDPANALDPSTLALFGTGTVMINRGSGQNDGLVSQCSALYGKVLSTRYKWNHVDEINQLLGVRGAYAEDPVAVIRTHANRLQLAGV

SEQ ID NO: 257 - protein, 364 aa, Burkholderia cepacia
MARTMRSRVVAGAVACAMSIAPFAGTTAVMTLATTHAAMAATAPAAGYAATRYPIILVHGLSGTDKYAGVLEYWYGIQEDLQQNGATVYVANLSGFQSDDGPNGRGEQLLAYVKTVLAATGATKVNLVGHSQGGLSSRYVAAVAPDLVASVTTIGTPHRGSEFADFVQDVLAYDPTGLSSSVIAAFVNVFGILTSSSHNTNQDALAALQTLTTARAATYNQNYPSAGLGAPGSCQTGAPTETVGGNTHLLYSWAGTAIQPTLSVFGVTGATDTSTLPLVDPANVLDLSTLALFGTGTVMINRGSGQNDGLVSKCSALYGKVLSTSYKWNHLDEINQLLGVRGAYAEDPVAVIRTHANRLKLAGV

SEQ ID NO: 258 - protein, 364 aa, Burkholderia sp.
MARSMRSRVVAGAVACAMSVAPFAGTTAVMTLATTHATMAATAPADDYATTRYPIILVHGLTGTDKYAGVLEYWYGIQEDLQQHGATVYVANLSGFQSDDGPNGRGEQLLAYVKTVLAATGATKVNLVGHSQGGLTSRYVAAVAPDLVASVTTIGTPHRGSEFADFVQSVLAYDPTGLSSSVIAAFVNVFGILTSSSHNTNQDALGSLKTLTTAQAATYNQNYPSAGLGAPGSCQTGAPTETVGGNTHLLYSWAGTAIQPTLSVFGVTGATDTSTVPLVDPANALDLSTLALFGTGTVMINRGSGQNDGLVSKCSALYGQVLSTSYKWNHIDEINQLLGVRGAYAEDPVAVIRTHANRLKLAGV

SEQ ID NO: 259 - protein, 363 aa, Burkholderia ambifaria
MSRSIRAKTAAAVVAFAMSAAPAASVGTILSLAGAQAASAATTAVDDYAATRYPIILVHGLTGTDKYGGVVEYWYHIPADLQAHGAKVYVANLSGYQSDDGPNGRGEQLLAYVKQVLAATGAQKVNLIGHSQGGLTSRYVASVAPELVASVTTISTPHWGSQFADFVQQLLQADPTGLSSVVLSAFANALGTLTSSNFNTNQNAIQALSVLTTAKAAEYNQKFPSAGLGAPGSCRAGASTETVNGNTHLLYSWGGTAIQPTTTVAGVTGAVDTSVSGATDPANVLDPATLALLGSGTVMINRGSGPNDGVVSQCSALFGQVLGTYHWDHTDAINQILGVLGANVEDPVAVIRTHANRLKTQGV

SEQ ID NO: 260 - protein, 364 aa, Burkholderia oklahomensis
MARSMRSKAVAAAVAFAMSAAPAASVGTFLSLAGTQSAVAATTAVDDYAATKYPIILVHGLTGTDKYANVLEYWYGITEDLQAHGATVYVANLSGFQSDDGPNGRGEQLLAYVQQVLAATGASKVNLIGHSQGGLTSRYVAAVAPQLVASVTTIGTPHRGSEFADFVQTVLAYDPTGLSSTVIAAFVNVFGILTSSNNNTNQDALAALKTLTTSQTATYNQNYPSAGLGAPGSCQTGAPTETVGGSKHLLYSWGGTAIQPTLSILGVTGAVDTSTIPVVDPANALDPSTLAMLGSGTVMINRGSGANDGLVSRCSSLYGQVLSTSYKWNHLDEINQLLGVRGAYAEDPVAVIRTHANRLKTQGV

SEQ ID NO: 261 - protein, 344 aa, Burkholderia sp.
MTAREGRAPLARRAAVYGVVGLAAIAGVAMWSGAGWHRGAGAAGDAPDAAAVGGVPAASPLAAVPASTGLPPSLAGSSAPRLPLDAGGHLAKSRAVRDFFDYCLTAQSDLSAAALDAFVVREVAAQLDGTVAQAEALDVWHRYRAYLDALAKLRDAGAVDKADLGALQLALDQRASIAYRTLGDWSEPFFGAEQWRQRYDLARLKITEDRSLTDAQKAERLAALEQQMPAGERAAQQRIDRQRAAIDQIAQLQKSGATPDTMRAQLTQTLGPDAAARVAQMQQDDASWQRRYADYAAQRAQIESAGSSQQDRDAQIAALRQRVFTKPGEAVRAASLDRAAGSTQ

SEQ ID NO: 262 - protein, 344 aa, Burkholderia cepacia
MTARGGRAPLARRAVVYGAVGLAAIAGVAMWSGAGRHGGTGASGEPPDASAARGPAAAPPQAAVPASTSLPPSLAGSSAPRLPLDAGGHLAKARAVRDFFDYCLTAQSDLSAAGLDAFVMREIAAQLDGTVAQAEALDVWHRYRAYLDALAKLRDAGAVDKSDLGALQLALDQRASIAYRTLGDWSQPFFGAEQWRQRYDLARLKIAQDPALTDAQKAERLAALEQQMPADERAAQQRVDRQRAAIDQIAQLQKSGATPDAMRAQLTQTLGPEAAARVAQMQQDDASWQSRYADYAAQRAQIESAGLSPQDRDAQIAALRQRVFTKPGEAVRAASLDRGAGSAR

SEQ ID NO: 263 - protein, 341 aa, Burkholderia cepacia
MTVRTMLSRAALHGAVGTLAAAAVWHALDRPAASTQAVALPAARAVVPDAAVPRPVPASSDVAPPASLAGARAPRLPVGPDGRLAKRRAVRDFFDYLLLAQHDLTPAALDALVAREVAAQPIGAPAQREALDLWPRYRACLAALDAQPGTVQPGTRIDPDAIAGELERRAALASRWLGDWSTPFFADAFRQQHDDLDRIRIARDPALSDAQKRERLAALDQALPPDMRDAHEQLARQREAVAAVARLDARRATPDALRAQLGADVAARVVRQRQDDDAWQARYRDYEAERERLVAQQLAPDARDAQLAQLLQRDFPDPADALRAASLGADRADITARQRAR

SEQ ID NO: 264 - protein, 344 aa, Burkholderia sp.
MTAREGRAPLARRAAVYGVVGLAAIAGVAMWSGAGWHRGTGSAGESPDAAAVGGVAAAPPQAAVPARAGLPSSLAGSSAPRLLLDAGGHLAKVRAVRDFFDYCLTAQSDLSAAALDAFVAREIAAQLDGTAAQADALDVWRRYRAYLDALAKLRDAGAVDKSDLGALQLALDQRASIAYRTLGDWSQPFFGTEQWRQRYDLARLKIAQDRTLTDAQKAERLAALEQQMPADERMAQQRVDRQRAAIDEIAQLQKSGATPDAMRAQLTQTLGPEAAARVAQMQQDDASWQSRYADYAAQRAQIESAGLSPPDRDAQIAALRQRVFTKPGEAVRAASLDRGAGSAQ

SEQ ID NO: 265 - protein, 347 aa, Burkholderia ambifaria
MTGSKSGIARRVALYGAAGVVAAGALWYGLDARKTRDAGMSGVAPAGAVSSASAGGTAASTVPGFGASGLPPALAGSTAPRLPLDAHGHLATVVAVRGFFDYFLTAQNQLRAAELDALVARAVAAQLDGTVAQREALDVWQRYRAYFAELAKLPGGGAVDDKLDLAALQLALDRRAAIASRTLGDWSEPFVGAEQTLQRDDLARLKIVRDPSLTEEQKAAGLAALEQQLPPDRRAELERQKEQDNAIAQIDHMQKSNATPDEMRVQLAQTLGPEAAERVVKMQQADNAWRAKYNEYAAQRAQIDAQNLSTQDRDAQIAQLRQRYFTEPGEALRAASLDHGSVSTRTQ

SEQ ID NO: 266 - protein, 344 aa, Burkholderia oklahomensis
MTGEGGIARRVALYGVAGVVAAGAVWYVVGASAKHDAGASGVAPVVASSALAAAAAGADAGSGAGALPASLAGSSAPRLPLDARGHLAKARAVRDFFDYWLSAQNELSAAALDALVSREIAAQLDGTIAQGEALDVWKRYRAYLDQLAKLPDGGAPGNKFDLATLQLALDQRESIASRTLGDWSAPFFGDEQQRQRYDLARLKIMRDASLTDAQKAARLAALEQQLPLDQRAEQAREKQQNDAVARIAQLQKSGASPDEMRAQFAQSLGPQAADRVAKMQQANDAWRAKYDAYAAQRAQIDAQNLSPQDRDAQVAQLRQRYFTEPGEALRAASLDRGSAATQTQ

SEQ ID NO: 267 - protein, 26 aa, Bacillus subtilis
MRKNRILALFVLSLGLLSFMVTPVSA

SEQ ID NO: 268 - protein, 29 aa, Bacillus subtilis
MPYLKRVLLLLVTGLFMSLFAVTATASAQ

SEQ ID NO: 269 - protein, 32 aa, Bacillus thuringiensis
MRSQKFTLLLLSLLLFLPLFLTNFITPNLALA

SEQ ID NO: 270 - protein, 56 aa, Cellulosimicrobium cellulans
MDLARHRSLTPPTTPPGTSVGPRPRARRRLAGALVAALTAAAAALAVTVPATSAAA

SEQ ID NO: 271 - protein, 26 aa, Bacillus subtilis
MFKKHTISLLIIFLLASAVLAKPIEA

SEQ ID NO: 272 - protein, 20 aa, Klebsiella aerogenes
MNKKAMAAAVSMILAGGAHA

SEQ ID NO: 273 - protein, 45 aa, Bacillus subtilis
MSDEQKKPEQIHRRDILKWGAMAGAAVAIGASGLGGLAPLVQTAA

SEQ ID NO: 274 - protein, 17 aa, Bacillus thuringiensis
MKKRLFFSCCLLFLMAG

SEQ ID NO: 275 - protein, 28 aa, Bacillus subtilis
MKKVLMAFIICLSLILSVLAAPPSGAKA

SEQ ID NO: 276 - protein, 27 aa, Bacillus subtilis
MGLGKKLSVAVAASFMSLTISLPGVQA

SEQ ID NO: 277 - protein, 20 aa, Trichoderma lixii
MAPVLAIASVLAALPALTMG

SEQ ID NO: 278 - protein, 30 aa, Streptomyces coelicolor
MSRRRISAVVTALALAGAVPLLMPANEASA

SEQ ID NO: 279 - protein, 19 aa, Bacillus subtilis
MKKIAIAAITATSILALSA

SEQ ID NO: 280 - protein, 32 aa, Bacillus subtilis
MRSKKLWISLLFALTLIFTMAFSNMSAQAAGK

SEQ ID NO: 281 - protein, 32 aa, Bacillus subtilis
MIPRIKKTICVLLVCFTMLSVMLGPGATEVLA

SEQ ID NO: 282 - protein, 29 aa, Bacillus licheniformis
MKNNHLLKSILLWGAVCIIVLAGPLSAFA

SEQ ID NO: 283 - protein, 28 aa, Bacillus licheniformis
MKQQKRLYARLLTLLFALIFLLPHSAAA

SEQ ID NO: 284 - protein, 24 aa, Bacillus subtilis
MKKMLMLAFTFLLALTIHVGEASA

SEQ ID NO: 285 - protein, 42 aa, Bacillus subtilis
MAKPLSKGGILVKKVLIAGAVGTAVLFGTLSSGIPGLPAADA

SEQ ID NO: 286 - protein, 21 aa, Bacillus subtilis
MKKLIMALVILGALGTSYISA

SEQ ID NO: 287 - protein, 323 aa, Beauveria bassiana
MGLSLDKEFAAMIAPAWHILNRPKPQIHDVQGRRLAMASLTEAGTPDPLPKGTAQEILSVPAEDGFGIPLWRVSRKPGTEPMHTKPGPAVLYMHGGGFVALGVETFARKLHALVLASGVTIYAVNYRLAPEHPYPTPLEDCWTSLQYIRDHAATLGVDVARIAVMGDSAGGNLAAGVTLLGRDRGITPPIASQFLIYPMLDDRNKEEMEAGVVTLWDQDDNATGWTAYLGAKAGGDEVPVYAAPGRLTDAAGLPRLTLYVGQLDLFVKENVRYALLFLEAGIETELRVLPGLLHGFDELAKDHAASKECFALQVKFMKRLLEN

SEQ ID NO: 288 - protein, 397 aa, Pseudomonas sp.
MEQCRPLLTVFFEGAQVQIQGHYELQFEAVREAFAALFDDPQERGAGLCIQIGGETVVDLWAGTADKDGTEAWHSDTIVNLFSCTKTFTAVTALQLVAEGKLQLDAPVANYWPEFAAAGKEAITLRQLLCHQAGLPAIREMLPTEALYDWQLMVDTLAAEAPWWTPGQGHGYEAITYGWLVGELLRRADGRGPGESIVARVARPLGLDFHVGLADEEFYRVAHIARSKGNMGDEAAQRLLQVMMREPTAMTTRAFANPPSILTSTNKPEWRRMQQPAANGHGNARSLAGFYSGLLDGSLLEADMLEQLTREHSIGPDKTLLTQTRFGLGCMLDQPQLPNATFGLGPRAFGHPGAGGSVGFADPEHDVAFGFVTNTLGPYVLMDPRAQKLVGILAGCL

SEQ ID NO: 289 - protein, 589 aa, Purpureocillium lilacinum
MKLLATSSALAAMFLSRALAAPAPVPAPAPDAAPEPIPAPVAVEPHLEERIATVTVDIPTGGKVKGRSLLNVESFNAIPFADPPVGQLRLRPPKRLSTNPGTIDGTGLAPGCPQMFVSSGARDALSTILGTLLDIPFLKPITGQEDCLTVNVQRPAGTKATDKLPVLFWIYGGGFELGSTNTYDATSLLASAVGQKQPFVFVAVNYRVAGFGFMPGKEILKDGSANLGLLDQRMGLEWVADNIAAFGGDPTKVTIWGESAGAISVFDQMLLYGGNATYKGQPLFRGAIMNSGSAVPADPVDCPKGQAVYDAVVKEGGCAGASDTLKCLRDLDYETFLNAANSVPGLLSYNSVALSYLPRPDGVVLPDSPDKLGEQGRFYAVPSIIGDQEDEGTIFSIFQSNVTSVDDMTNYLSQLFFHNAPKSKLKELVNIYEPALLQGSPFRTGIFNELYPGFKRMAAILGDLTFTLTRRVVLKIATKTKPDMPIWSYLSSYDYGTPVVGTFHASDILQVFYGVLPNNAMRSCRTYYFNFLYNLDPNKGVGGFARWPQWKEKQELMWFKSAVANGILKDDFRSGAAAFIEANKGILHI

SEQ ID NO: 290 - protein, 474 aa, Archaeoglobus fulgidus
MRGLAVLVLLVFAVQVAAAEDFRPVVFVHGLAGSAGQFESQGMRFAANGYPAEYVKTFEYDTISWALVVETDMLFSGLGSEFGLNISQIIDPETLDKILSKSRERLIDETFSRLDRVIDEALAESGADKVDLVGHSMGTFFLVRYVNSSPERAAKVAHLILLDGVWGVDAPEGIPTLAVFGNPKALPALGLPEEKVVYNATNVYFNNMTHVQLCTSPETFAVMFEFINGYKPATTDIVPQDGDYVKVKGKFLAFATNGDVSGWLSIYPIDENGKRLTRLPVKFMRVKGDFEVRLRKGQLYEFQFRKDFSPIIYHYYRAPFVRDDLWARFLVSKPPLDVELLILPERLSPAAKETSGLLLIRYKEMIGEYDEEIGGVDEVYVNGVNVCTERICPIERAVNGLWVFDRGADGKSDLDREVVRYSIMPFMSAADLVVPAEGTISIAVKSRTGGEESFTIPAWSADRHSIIVQFSDYI

SEQ ID NO: 291 - protein, 487 aa, Bacillus paralicheniformis
MKKLVLLMLVLLVVYPHVSKAGGFKGGGGDPGHWFAGDPVEHPDPAKPPIVFVHGLNGSSSAWFDENDMAEQAWKNGYDAAFIDLHPDKDMQDNGAMLAAKLREIYQHFGRKVILVSYSKGGIDSQSALIHHNAYHYVERLITLGTPHHGSQLADLAYSSWAGWLADILGQKNDAVYSLQTGFMKSFRDQTDNHPNRSKTKYFTLAGNKIGGFGSVLFFGGVYLNMFGENDGAVTEKNARLPYATNLNTGKWDHFSIIKGNLTFPVFMPLLTVQAYANETAAIKENLSYPFIRGGENHGLREEEFAVEKGVKEITVHWLSNHSSGNIKLTDPHGKPLKDFSIAKTVDVFEGGFVHSAAIKNPAAGTWKIASSVKQKEAFLFIVTFDSPLNQQIKNAVTRESANLANVKASVRSIRYKNGKQAEKKSLKPASINALQNSLSFKKAGLYSVTIDLSGKTADNSPFNRTIIRSIYVNDKGEKFENSPLSD

SEQ ID NO: 292 - protein, 240 aa, Bacillus sp.
MRTPLSFDKETAILLASCCELTYEQYKQNGIFEIPDGFQYVQGFQGKAIQTTEWFGFILESEDTIIVAFRGTQTDPDWIIDSLVNQKPYPYALNGGNVHNGFLSIYESCRDSIMDMLVSLPAHKKLLATGHSLGGALATLHILDARINTAFAQYGLYTFASPKVGDIAFRNYYKLQVASSFRFVNLFDVVPLLPPRNINFNDHDWEYAHVHHNMTFTKNTKSITNNHSITTYKTCLTSHF

SEQ ID NO: 293 - protein, 223 aa, Bacillus thuringiensis
MKRFKFYLISGIVLVLLIVCNFVDKPIAKVEEVKDTVMMKLNTKESMAQIDGQTIYFKQIGEGKPPLLMLHGFGGSSDGFSDIYPELVRDHTIIAVDILGFGRSSKPVDFQYSFPAQVNLYYKLMKKLGYDQFAVLGHSMGGEMSLNLAYLYPDAVTHLILADAKGIESFQQKESYEVPPLSTDLQTVTEITKYNKNEVKNSRDDKEHYDQLTKMRERRIFVS

SEQ ID NO: 294 - protein, 496 aa, Beauveria bassiana
MQFVRALTPLLALLLCPFVLADADCDDAQPPSQDPWYTLPQGVDCSGAPGKVLRLRRAPKTARESLRGIQDAWNILFCSTDSAGEPTFSVTTVLVPHDADSRALVTYPPAYDSASVDSSPSYALSNLVGSSIEQAIFTPLLVSKRWVINMPDYEGPRAAYLAGNNSGHGLLDSIRAVRSLHDLFGLCPDFQHAVYGYSGGAFASQWAAQLQPSYAPELAIGVMAYGGLAVNATSVIPAINNGPAAGLIVLALLGTTAQSQEARMILAKNLRKTGLYNSTTFFSALKMDSQTATALFQNQDISKYFVEGFSFLNEPAFVRAFYDDTLAGRGDTPTIPMYVFHGVKDLISPIKDMDELVQKYCAAGTGIEYRRNVVGDHGGTGIAEGIRALNWLGGFFAGAHQPEKRCHVNDVTSALGSDSVALAFIVFSKQGYRIAQFLKIHALPGVPSLPPNTTNAEVFGALRLTTRASLAKIGLNDQALAQFHVSEQDLAELGIH

SEQ ID NO: 295 - protein, 301 aa, Beauveria bassiana
MQATIASTAALAGLIFATTFADGKALQTTSIGKHNDFSCQPSANHPRPVVLLHGLGATYYDDLHVLEAFLQIRGFCTFSLTYGAVKGSPFLGGFAPIDESSRQIADFIYEVHAKTVGYGGGGGGGGSSSSTRKIDIVGHSEGGFQALYVPKFRDGVAALVDHIVAIAPPTHGTPIANLLSLSPLLGHDAGILELIGCAACDQLSIGGASVVKLNDGKAIVQPGNKVTVIISNLDAIVIPPKSAFIYEDGVDNISVQDYCPLDLVGHISESYDVNIWNLVLNSLENQVGRKFACLVGGLPVK

SEQ ID NO: 296 - protein, 338 aa, Beauveria bassiana
MRVALFSLVAAAAASPIVNNQTLLDSRSVDNNFMNSFKFYAQHAAAAYCNAEGRSPGNAISCAGGECNDVMRNGATIINTFQGANTGIAGYVSVDRTRREIVFAARGSNNLRNFITNLIFTQRDCDFASGCKVHDGFAASWDEISVAATAAIRSGLQANPGYRLVITGHSLGGAIGTLAGVYLRRAGYQAAIYTFGAPRIGNEVFANFASRQRGGLYRMTHIDDPVPRLPPMIFGYRHGGTEYWLSNGEAEQINYQGNDVKVCPGIDSVGCNAGTIGFDLPAHLHYLTNTAGCAPPATKWKRDPEPSDEELEERFTNWSKQDQKLVNSCYYCYTVEVV

SEQ ID NO: 297 - protein, 377 aa, Beauveria bassiana
MWLLLFACLLHVVVAQQAPGVSVSMFASLERLARLVDITYCIGNTGIGKPFSCLSRCSDFPNVTLSRTWSTGVFLTDSCGYVAVDETPASSVAVDSHGAIIVAFRGTYSIANTVIDLSTVPQEYVPYPSQGDAPAHKCTNCTVHMGFFQTWQSARESVIPELVQLRKTYPSKPIHLVGHSLGGAVACLAALELKTSLGLDNVVVTTFGEPRVGNDGLVDFIDRVFNLNDKGDLEKRSYRRVTHANDPVPLLPPGEWGYKSHAGEIHITKPDLSPAEEDLQLCMGDQDNSCSAGQDSTSQGLARLTQFYQGVKGSQLQKRGISTRLNLWQLFFAHRDYFWRLGLCFPGGDPFDWGRRKLIEKLPEAEGLEMEAEYQEL

SEQ ID NO: 298 - protein, 454 aa, Beauveria bassiana
MLARGLLSAAIVVAHALVASAAPPMPHDPRTAPILPSQDPFYAVPDSISAAAPGTILRHRKPPSSIAVFGFDSVNLAETVQIQYRTADNLGNATAAVLTVLVPHHADMSKVLSYQVAEDAASADCAPSYTLQRESKSGPHHGTLFEQAEILLIEAVLEQGWVVVVPDYEGPNALYLAWRLAGQATLDGIRAALASEEITGIKADATVAMWGYSAGSMATAWAAEIQPQYAPELRIAGAAFGGTTPNITNVVGATNKGRFAGLLPTGIMGLSKQYPAIKAMVDRELKPEFREVVEQTKHQCLVADLAVFSDRDVTAMFTDPRFMYNDPDAVKVLAEIDLGQAVPEIPIYWYKSVHDEVSPVADSDAVYAKYCAGGATVEYLRDEASEHASAMATGAPRAVAWLKNIMNGGKPKPGCSKETTLTSLWDLSTIEVLPGFILDALLDLAGKPVGPWLA

SEQ ID NO: 299 - protein, 487 aa, Beauveria bassiana
MKFLSLVAAVLPLVSALTIPEQSITTPANDPLSVPPSGLELAEDASALTVPEKRITTPANDPFYVPPSGFESAKPGTVLRERPIVASFFGLIPAPIDSRQLLYRTTAIDGSAIATVVTIFKPLFAKRDRFISFNTAYDSSASICNPSYNYRLGALQTDVISSAEFLIIQAYLLSGYTVASADYEGPDVAFSVGRLSGMGVLDGIRAVVNYGPKIGLDKNPMVVNAGYSGGAIASGWAASLHPTYAPDLNLKGFIAGGTPANLTEVLLYVDGTLFAGFVPGALAGQMMPSAYGARLKPVLDKVIGPRGKEALALGTSQCAPVNLIAFAGKSIFDTSFQTLGKDLLYDKDVAWVLAESTLGLKKNETPTVPVMLYHSPDDEVIPFAGADGLRKRWCDNGANVRFVNYAAGGHVTAEVVAVIDAIKFASDAFSGSVPGGCASRTVLDDKLNPLALGLSLEPVLAGLVNILLTLGKKDANFVSGLSQGKHI

SEQ ID NO: 300 - protein, 546 aa, Beauveria bassiana
MMLLMQLMCVAGALGAAVALSADIDGAPLIHLDYATYRGSRVAGAGVDQFLGMRFAKPPMGDLRFRAPEEPFHEDQVQDASKLGALCVGTVDGSSVGPNDVFGEDCLFANLFKPANATTASKLPVWVFIQGGGYAHNSNGNYNGTQIVQQSGGNVVFVSFNYRVGVLGFLASEEVRKDGALNAGLLDQRLFLKWVQKYIGQFGGDPNHVVIHGDSAGGGSVSHHLTAYGGNDKENLFVGGIPESPFWPTQRKVDQMEFQYTRLLRDTKCDSLDCLRKLDAGALGNASQPSPFPEAHTEDPVPLWYWLPVIDGDLVQDNMYKQFLTGRFKRVPLLVGADTDEGTLFAVNASTETEVSSFLRANYPRLQKWQLDIVSTIYPKMKPLPKHAAYFPSAAAVYGDSTFVCAGNTMSDAISAFVGPDKLWNYRYNVADPEQVAEGFGVPHVSELGAIFGPDNIRGQEAKSLRTTNTPIVPVTMAYFTSFVRFLDPNTSRQPGSPLWQNWGAFVSEQGQRMRLETNNTAMETVPFDLVAKCALWKLLAESMDV

SEQ ID NO: 301 - protein, 259 aa, Neobacillus niacini
MKKVILTIICLLLLIISYSYFEKNDETKQNESEEKTEKTSAPNWIDKQTNESFYHLVLGDSLAKGYGSTQGGFAELASKQIEAQIHKPITVENLGVNGLTTDRLAKKVQSEDVKEKIRAANIITINIGGNNLFRLNRDVGVIDGIKMLNKEKAHFEADIKSIVKTVRDQNPDALLILSELYNPLQLDDSIASYADMFLDGWNESVYSISKANQPSIVLPIRKLISNDKKELLFDQVHPNDNGYAIIANTFTKQVLSYKY

SEQ ID NO: 302 - protein, 445 aa, Neobacillus niacini
MKSKIFKALGISIVVVLLLSMMTGFSKRDSTYDMRPIVFVHGFFGSANQFESQAMRFSSNGYPDNYLAAFEYDSSTASVTPAESRFTRLDALINQLLKETGADKIDLMGHSLGTTEMTNYLNSSPERAKKVAHYVNIDGVAGKALPGGVPTLAIWAELGTPGRTFVGAENVTLAGQSHVQAATSPEAFAHIFKFFTGKEPKTTQIVAEPRGQVRLSGKALIFPQNTAPEKATLEIYKVNGKTGEREKKKPEAVFTIAADGSWGPFKAKGGDNYEFVIIRDGVANHHLFFEEFIRSDHFVRLLTSPPEGGVTASMDTSDHHTNLSINRSKEFFGDQGANNDILKINGTNIVNAENSAANLRTINYFVYDEGADKVNHLNVPTKAFAGLPFLKGVDMYIPGATPPNSTVTVELQSRGSNGKKQVINLPNWASSKLRSVTVQFKDYVQ

SEQ ID NO: 303 - protein, 311 aa, Pseudomonas aeruginosa
MKKKSLLPLGLAIGLASLAASPLIQASTYTQTKYPIVLAHGMLGFDNILGVDYWFGIPSALRRDGAQVYVTEVSQLDTSEVRGEQLLQQVEEIVALSGQPKVNLIGHSHGGPTIRYVAAVRPDLIASATSVGAPHKGSDTADFLRQIPPGSGGEAVLSGLVNSLGALISFLSSGSTGTQNSLGSLESLNSEGAARFNAKYPQGIPTSACGEGAYKVNGVSYYSWSGSSPLTNFLDPSDAFLGASSLTFKNGTANDGLVGTCSSHLGMVIRDNYRMNHLDEVNQVFGLTSLFETSPVSVYRQHANRLKNASL

SEQ ID NO: 304 - protein, 296 aa, Pseudomonas sp.
MSQGTATRYPLVLVPGMLGFIRLLLYPYWYGIIKALRRGGATVIAVQVSPLNSTEVRGEQLLARIDEILRETGAARVNLFGHSQGSLTARYAAAKRPDLVASVTSVAGPNHGSELADYLQQHYPANTAKGRLLEALLRVVGWLMALLETGYHGPKLPVDIHASHQSLTREGVALFNQRYPQGLPETWGGQGPEEVNGVRYYSWSGTLQPGKTDRGGNLFDGTNRSCRLFAKTFVREPGQCDGMVGRYSSHLGTVIGDDYPLDHFDIVNQSLGLVGKGADPVRLFVEHAARLKAAGL

SEQ ID NO: 305 - protein, 289 aa, Pseudomonas sp.
MQRNATTRYPILLVHGLFGFDRIGHFELFHDVKQALREAGGQVFVPQLSATHDNEVRGQQLMFQIERVLRGTGAGKVNLIGHSQGALACRYAAALAPEKVASVTSVSGPNHGSELADALRLALVPGGLPEQVAEHAATQFARFLSLLSGQPGLPQAAVAALNALTTADVGQFNKKYPQGLPSTWGGQGAERVNDVHYYSWSGTLQDNLLQALDPVHSVCHALSSHFVTEAGQNDGFVGRYSSHLGRVIRSDYPLNHLGSLRHPGGLRIPGAYPVDLYVEHAMRLKAAQL

SEQ ID NO: 306 - protein, 476 aa, Pseudomonas sp.
MGIFDYKNLGTEGSKALFADAMAITLYTYHNLDNGFAVGYQHNGLGLGLPATLVGALLGSTNSQGVIPGIPWNPDSEKAALEAVQKAGWTPISASTLGYGGKVDARGTYFGEKAGYTTAQVEVLGKYDDAGKLLEIGIGFRGTSGPRETLISDSIGDLVSDLLAALGPKDYAKNYAGEAFGGLLKNVADYAAAHGLTGKDVVVSGHSLGGLAVNSMADLSTNKWSGFYTDANYVAYASPTQSAGDKVLNIGYENDPVFRALDGSSFNLSSLGVHDKPHESTTDNIVSFNDHYASTLWNVLPFSIVNLPTWVSHLPTAYGDGMTRILDSGFYDQMTRDSTVIVANLSDPARATTWVQDLNRNAEPHKGNTFIIGSDGNDLIQGGKGADFIEGGKGNDTIRDNSGHNTFLFSGQFGNDRVIGYQTTDKLVFQDVQGSTDLRDHAKVVGADTVLTFGADSVTLVGVGHGGLWADGVSIG

SEQ ID NO: 307 - protein, 457 aa, Purpureocillium lilacinum
MVAFGLRLVASLAVSAGVAFAAPPSARLLVDRAQGVLPSKDPWYAVPDGLDAVKPGTILKHRTPPKPIAAFGLLPVNLEAAYQIQYRTTDSLGNATATALTVMVPHSADYTKVLSYQVAEDSATIDCAPSYAFQFDHAKGPDMGTMVTEAELILVEAALEQGWVVIAPDFLGHDGAFLANALAGHATLDGIRAAINSNSFTGISTNPTVTMWGYSGGSLASLWAAELQPTYAPELKIAGAAVGGTVPNITTVVTSINGKGSAGLIPTGVVGLMNQYPELVPVVEQHLLPQYRDEFFKVRGQCLAANSKQFENKDIVGMFDDRNLVYTNPTAVRIIAENAQGITGTPRIPLFIYKSVNDEISPISETDALVDKWCADGTRIEYQRDKESDHEKLAILAAPKALSWLIDTMNGRNRNTECVTRTVFSSLLDPAAIGVLPKILIDALLDLLGKPVGPLIG

SEQ ID NO: 308 - protein, 567 aa, Pseudomonas chlororaphis
MSMSIFDYKTALGGDGKALYSEAITLALYASTPTGEALPGTAWRPISVSQLGYQGNVSAQGTISGEQAIVSDAQSRCWANTTRPGSCCPSASSFRGTRQPQGRYQRLAGGLRVGLLPTTTSRLAFDNLLGKVAAFAAAQGLSGSDVLVTGHSLGGLGGQPRGGHEQRSLGRLLPGRQLLGFASPTQSANSSQVLNIGYETTRCSAPWTALISTALSLGTHWQAPGVATNNIVSFTDHYSSFLGEVDSPEHPQSAVLVGPQCGGLRRGLNRLINSDFYDLTSRDSTVVISNLSEGKRDQVWVKDLNLYAEKHTGSTFIIGTQSNDLLHGGKGNDYLDGGAGDDRFRDDGGYNIIHGGQGHNVLELQQPLKNFSIANDGDGTLYIRDAYGGISMTRDVGALVSHETGSWWQLFGKDVSHSVTADGLQNGNQWTAYNHSLNGDAYGNALVASVDGDWLFGHGGDDLLSSDKANVTFVGGTGNDVMHSSGGGGNTFLFSGNFGFDLIHGYQNTDKLVFMGVPGVDAHYDYSQHLSQNGNDTLVQVFGEFLRVNPWWGSAWTSLSGSGLVFA

SEQ ID NO: 309 - protein, 614 aa, Serratia marcescens
MGIFSYKDLDENASKALFSDALAISTYAYHNIDNGFDEGYHQTGFGLGLPLTLITALIGSTQSQGGLPGLPWNPDSEQAAQEAVNNAGWSVISATQLGYAGKTDARGTYYGETAGYTTAQAEVLGKYDSEGNLTAIGISFRGTSGPRESLIGDTIGDVINDLLAGFGPKGYADGYTLKAFGNLLGDVAKFAQAHGLSGEDVVVSGHSLGGLAVNSMAAQSDANWGGFYAQSNYVAFASPTQYEAGGKVINIGYENDPVFRALDGTSLTLPSLGVHDAPHTSATNNIVNFNDHYASDAWNLLPFSILNIPTWLSHLPFFYQDGLIRVLNSEFYSLTDKDSTIIVSNLSNVTRGSTWVEDLNRNAETHSGPTFIIGSDGNDLIKGGKGNDYLEGRDGDDIFRDAGGYNLIAGGKGHNIFDTQQALKNTEVAYDGNTLYLRDAKGGITLADDISTLRSKETSWLIFNKEVDHQVTAAGLKSDSGLKAYAAAATGGDGDDVLQARSHDAWLFGNAGNDTLIGHAGGNLTFVGGSGDDILKGVGNGNTFLFSGDFGRDQLYGFNASDKLVFIGTEGASGNIRDYATQQNDDLVLAFGHSQVTLIGVSLDHISTDQVVLA

SEQ ID NO: 310 - protein, 538 aa, Bacillus sp.
MRNLTKTSLLLAGLCTAAQMVFVTHASADQSIKYDHTYQTPSYIIEKSSQKPVQNTTQKESLFSYLDKHQTQFKLKGNANSHFRISKTIKDPKTKQTFFKLTEVYKGIPIYGFEQAVAMKENKQVKSFFGKVHPQIKDVSITPSISEKKAIHTARRELEASIGKIEYLDGEPKGELYIYPHDGEYDLAYLVRLSTSEPDPGYWHYFIDAKNGKVIESFNAIHEAAGTGIGVSGDEKSFDVTEQNGRFYLADETRGKGINTFDAKNLNETLFTLLSQLIGYTGKEIVSGTSVFNEPAAVDAHANAQAVYDYYSKTFGRDSFDQNGARITSTVHVGKQWNNAAWNGVQMVYGDGDGSKFKPLSGSLDIVAHEITHAVTQYSAGLLYQGEPGALNESISDIMGAMADRDDWEIGEDVYTPGIAGDSLRSLEDPSKQGNPDHYSNRYTGTEDYGGVHINSSIHNKAAYLLAEGGVHHGVQVEGIGREASEQIYYRALTYYVTASTDFSMMKQAAIEAANDLYGEGSKQSASVEKAYEAVGIL

SEQ ID NO: 311 - protein, 313 aa, Bacillus subtilis
MKLVPRFRKQWFAYLTVLCLALAAVVSFGVPAKAAENPQTSVSNTGKEADATKNQTSKADQVSAPYEGTGKTSKSLYGGQTELEKNIQTLQPSSIIGTDERTRISSTTSFPYRATVQLSIKYPNTSSTYGCTGFLVNPNTVVTAGHCVYSQDHGWASTITAAPGRNGSSYPYGTYSGTMFYSVKGWTESKDTNYDYGAIKLNGSPGNTVGWYGYRTTNSSSPVGLSSSVTGFPCDKTFGTMWSDTKPIRSAETYKLTYTTDTYGCQSGSPVYRNYSDTGQTAIAIHTNGGSSYNLGTRVTNDVFNNIQYWANQ

SEQ ID NO: 312 - protein, 645 aa, Bacillus subtilis
MKNMSCKLVVSVTLFFSFLAIGPLAHAQNSSEKEVIVVYKNKAGKETILDSDADVEQQYKHLPAVAVTADQETVKELKQDPDILYIENNVSFTAADNTDFKVLSDGTDTSDNFEQWNLEPIQVRQAWKAGLTGKNIKIAVIDSGISPHDDLSIAGGYSAVSYTSSYKDDNGHGTHVAGIIGAKHNGYGIDGIAPEAQIYAVKALDQNGSGDLQSLLQGIDWSIANGMDIVNMSLGTTSDSKILHDAVNKAYEQGVLLVAASGNDGNGKPVNYPAAYSSVVAVSATNEKNQLASFSTTGDEVEFSAPGTNITSTYLNQYYATGSGTSQATPHAAAMFALLKQRDPAETNVQLREEMRRNIVDLGTAGRDQQFGYGLIQYKAQATDSAYAAAEQAVKKAEQTKAQTDINKARELVSQLPGSDAKTALHKRLDKVQSYRNVKDAKDKVAKAEKYKTQQTVDTAQTAINKLPNGTDKKNLQKRLDQVKRYIASKQAKDKVAKAEKSKKKTDVDSAQSAIGKLPASSEKTSLQKRLNKVKSTNLKTAQQSVSAAEKKSTDANAAKAQSAVNQLQAGKDKTALQKRLDKVKKKVAAAEAKKVETAKAKVKKAEKDKTKKSKTSAQSAVNQLKASNEKTKLQKRLNAVKPKK

SEQ ID NO: 313 - protein, 442 aa, Bacillus subtilis
MFGYSMVQMVRANAHKLDWPLRETLLQLYKPFKWTPCFLHKFFETKLQNRKKMSVIIEFEEGCHETGLQIAGEVLQKEKRSKLKSRFNKINCCSAEVTPSALHSLLSECSNIRKVYLNREVKALLDTATEASHAKEVVRNGQTLTGKGVTVAVVDTGIYPHPDLEGRIIGFADMVNQKTEPYDDNGHGTHCAGDVASSGASSSGQYRGPAPEANLIGVKVLNKQGSGTLADIIEGVEWCIQYNEDNLDEPIDIISMSLGGDALRYDHEQEDPLVRAVEEAWNAGIVVCVAAGNSGPDSQTIASPGVSEKVITVGALDDNNTASSDDDTVASFSSRGPTVYGKEKPDILAPGVNIISLRSPNSYIDKLQKSSRVGSQYFTMSGTSMATPICAGIAALILQQNPDLTPDEVKELLKNGTDKWKDEDPNVYGAGAVNAENSVPGQ

SEQ ID NO: 314 - protein, 566 aa, Bacillus thuringiensis
MKKKSLALVLATGMAVTTFGGTGSAFADSKNVLSTKKYNETVQSPEFISGDLTEATGKKAESVVFDYLNAAKGDYKLGEKSAQDSFKVKQVKKDAVTDSTVVRMQQVYEGVPVWGSTQVAHVSKDGSLKVLSGTVAPDLDKKEKLKNKNKIEGAKAIEIAQQDLGVTPKYEVEPKADLYVYQNGEETTYAYVVNLNFLDPSPGNYYYFIEADSGKVLNKFNTIDHVTNDDKSPVKQEAPKQDAKAVVKPVTGTNKVGTGKGVLGDTKSLNTTLSGSSYYLQDNTRGATIFTYDAKNRSTLPGTLWADADNVFNAAYDAAAVDAHYYAGITYDYYKNTFNRNSINDAGAPLKSTVHYGSNYNNAFWNGSQMVYGDGDGVTFTSLSGGIDVIGHELTHAVTENSSNLIYQNESGALNEAISDIFGTLVEFYDNRNPDWEIGEDIYTPGKAGDALRSMSDPTKYGDPDHYSKRYTGSSDNGGVHTNSGIINKQAYLLANGGTHYGVTVNGIGKDKLGAIYYRANTQYFTQSTTFSQARAGAVQAAADLYGANSAEVAAVKQSFSAVGIN

SEQ ID NO: 315 - protein, 799 aa, Bacillus thuringiensis
MRRKAPFKVLSSLAIAAIIGCTSVMSAPLAYAETAAKKKDNVSTTPIDYNLIQEDRLAEALKERGTINPSSSKEETKKAVEQYIEKKQGDQANKEILPDAHSQETSDFTKKVKEKKMEEKENVKKAEKNVSLEQKPEPNKNQLNGQVPTSKAKQAPYKGSVRSDKVLVLLVEFSDYKHNNIDQTPGYMYSKDFSREHYQKMLFGNEPYTLFDGSKVKTFKQYYEEQSGGSYTTDGYVTEWLTVPGKASDYGADGSSGHDNKGPKGARDLVKEALHAAAEKGLDLSQFDQFDRYDTNGDGNQNEPDGVIDHLMVIHAGVGQEAGGGKLGDDAIWSHRSKLAVDPVAIEGTKSKVDYFGGKVAAHDYTIEPEDGAVGVFAHEFGHDLGLPDEYDTKYTGNGSPVEAWSLMSGGSWTGKIAGTEPTSFSPQNKDFLQKNMGGNWAKILEVDYDKIKSGVGVPTYIDQSVTKSNRPGVVRVNLPGKSVETIKPEFGKHAYYSTRGDDMHTTLETPFFDLTKGTNAKFDYKANYELEAECDFVEVHAVTEDGTKTLIDRLGEKVVQGDKDTTDGKWIDKSYDLSQFKGKKVKLQFDYITDPAVTYKGFAMDNVNVTVDGQVVFSDDAEGQSKMNLNGFVVSDGTEKKAHYYYLEWRNYAGSDNGLKAGKGPVYNTGLVVWYADDSFKDNWVGMHPGEGFLGVVDSHPEALVGNLNGKPAYGNTGMQIADAAFSFDKTPAWSVNSLTRGQFNYSGLQGVTTFDDSKVYSNKQIADAGRKVPNLGLKFQVVGQAEDKSAGAVWIKR

SEQ ID NO: 316 - protein, 965 aa, Bacillus thuringiensis
MNKKSKINKVMLSISTMALSLGALQTHAAAEEKVPYNVLKTKPVGIEKSVDEVGHISKVDETLSFQERLKVGDFSQRPASITKKTAVKQVKESYSMADLNKMNDRELVETLGSIKWHQITDLFQFNEDAKAFYKDKGKMQVIIDELAHRGSTFTKDDSKGIQTFTEVLRSAFYLAFYNSELSDLNERSFQDKCLPALKAIANNPNFKLGTVEQDTVVSAYGKLISNASSDVETVQYASNILKQYNDNYTTYVNDRMKGQAIYDIMQGIDYDIQSYLTEARKEANETMWYGKVDGFINEINRIALLNEVTPENKWLVNNGIYFASRLGKFHSNPNKGLEVVTQAMHMYPRLSEPYFVAVEQITTNYNGKDYSGNTVDLEKIRKEGKEQYLPKTYTFDDGSIVFKTGDKVSEEKIKRLYWAAKEVKAQYHRVIGNDKALEPGNADDVLTIVIYNSPDEYQLNRQLYGYETNNGGIYIEETGTFFTYERTPEQSIYSLEELFRHEFTHYLQGRYEVPGLFGRGDMYQNERLTWFQEGNAEFFAGSTRTNNVVPRKSIISGLSSDPASRYTAERTLFAKYGSWDFYNYSFALQSYLYTHQFETFDKIQDLIRANDVKNYDAYHENLSKDPKLNKEYQEYMQQLIDNQDKYNVPEVADDYLAEHAPKSLTEVKKEISDTLPMKDTKMTKHNSQFFNTFTLEGTYTGSVTKGESEDWKAMSKRVNESLEQLAQKEWSGYKTVTAYFVNYRVNSSNEFEYDVVFHGIAKDDGENKAPTVNINGPYSGLVKEGIQFKSDGSNDEDGKIVSYLWEFGDGSTSAEVNPVHVYEREGSYKVSLRVKDDKGKESRSETTVTIKDGSLTESEPNNRLEEANRIGLNSTIKGSLIGGDHTDVYTFNVASAKDIDISVLNEYGIGMTWVLHHESDMQNYAAYGQANGNHIEAKFNAKPGKYYLYVYKYDNGDGTYKLSVK

SEQ ID NO: 317 - protein, 426 aa, Bacillus velezensis
MKFSFMSAAVAASVIWGAAAGPLSETHAYSAVTEPVFDADEALDTMRDLSETIGPRETGTAKEKEAAEYISNRLRAQGASVKVQSFPLKKYYSGSVTAGDHEFPFRSADQSAVEKELKIEGGIFDGGCGSRNDVTGAKGKIAVIREGKIPVSQKIKHAEQAGAKAVIIYNQTTKPLAVRPHTDKGITIPAAGMKKEDGEALLQEKQAVIQFKNYKNRTSQNIIGTRKAAQANPSDADIIYVTAHYDSVKGAPGADDNASGTSAVLEISKQLTHIPKDKEIRFILFGAEELWSAGSSYYVSTLTNNERKHSQANFNADMIATDWEEASQMCVTTPDGRENPSVRYAEKAAEKSRLVKPNLIQLGYSDHVSFYEAGIPSANWIWLSPDTQEPGPWYHTENDNMKHISKERLEKAGALVSAAVNEAMKE

SEQ ID NO: 318 - protein, 382 aa, Bacillus velezensis
MRGKKVWISLLFALALIFTMAFGSTSPAQAAGKSNGEKKYIVGFKQTMSTMSAAKKKDVISEKGGKVQKQFKYVDAASATLNEKAVKELKKDPSVAYVEEDHVAQAYAQSVPYGVSQIKAPALHSQGFTGSNVKVAVIDSGIDSSHPDLKVAGGASMVPSETNPFQDNNSHGTHVAGTVAALNNSVGVLGVAPSASLYAVKVLGADGSGQYSWIINGIEWAIANNMDVINMSLGGPSGSAALKAAVDKAVASGIVVVAAAGNEGTSGSSSTVGYPGKYPSVIAVGAVNSSNQRASFSSVGSELDVMAPGVSIQSTLPGNKYGAYNGTSMASPHVAGAAALILSKHPNWTNTQVRSSLENTTTKLGDAFYYGKGLINVQAAAQ

SEQ ID NO: 319 - protein, 388 aa, Metarhizium robertsii
MHLSALLTLLPAVLAAPATIGRRAEPAPLFTPQAESIIADKYIVKFKDDIARIATDDTVSALTSKADFVYEHAFHGFAGSLTKEELKMLREHPGVDFIEKDAVMRISGITEQSGAPWGLGRISHRSKGSTTYRYDDSAGQGTCVYIIDTGIEASHPEFEGRATFLKSFISGQNTDGHGHGTHCAGTIGSKTYGVAKKAKLYGVKVLDNQGSGSYSGIISGMDYVAQDSKTRGCPNGAIASMSLGGGYSASVNQGAAALVNSGVFLAVAAGNDNRDAQNTSPASEPSACTVGASAENDSRSSFSNYGRVVDIFAPGSNVLSTWIGGRTNTISGTSMATPHIAGLAAYLSALQGKTTPAALCKKIQDTATKNVLTGVPSGTVNYLAYNGA

SEQ ID NO: 320 - protein, 874 aa, Mythimna unipuncta granulovirus
MACVESRNSDLLLLTVPVPSLRVPPYLRETDGWMALRHGRVPAGIMVYASATVSCRLAENSPAVPTTVRFLNNNRATEHSLAISTFSWTEYTVLHDSVVFVDRTLNSVATRVEFKVDGVVCEVPVYVHGATNESQFKSEYSASECPYALIRFPVVDILVPPADKANVLQLDLQALQSFYSLIVGLYDDLAGLRSDARSNHGIDDNFDKRFFAKADATGAGAAYYSATWTANSRDTLGNYLQARPGNWLVFHEFGHAYDFMFVVRTGLNEVWNNVYSDRMQFQLMDKAQRQTMASVYENGNRARVETDVMGLIESSVSYFEWSHFQKLTIFTFIMNTQYGPDTWRVINAQFRLLRSIEAQPVYPNIALWLLDACPADLAALFGLFSIHVPQYSVTSARGIARSFPYLVNVAHAKHALYPIKYLVPDFDVLTANDYNFKRYLESNLDLVTPHELIQTDLQINILVIQCVIGDVAQVAGEPFAVYDGLTMVFKGEVNSSGIVLITMLGPGVYTMRAPRGRDKRYHVSFNEPRNPADHLIIDGKHNVYRLHYTEYTHNSLLPDVGHVLGINDVYSLEFRVRYDLQVVEIKVYFKAINSNFGTRLYFSITIEEPSGLFELSGSGDGVEDGGVYRLAFTPERSIMNVYHAQANMHRVWFLGRSLTALTVQYRLLADSVVPVNTNIAIPSRAERLLAEIEHHARWLDSHPVMLAIENEARDFIFVGVQTFEGDEQHDVLTERYNRYFPDYYRIRHYQIDMNGFSDLPRFTFVCDLLLGRADLTRYTAPSGPNVHFGTSDYIGFELINDTNVSVLKQLFQGSQSFAPAQFSNLPIGDGYRVELFHAEPRRIQMSNNDRLITDVTFTENTVLVVRGQRFHMLI

SEQ ID NO: 321 - protein, 241 aa, Paenibacillus larvae
MPFIDTPYGDQNAEVEYNIEGKQIPLPIYQPCGNEMEFFQQWDNEQAGFALVQGPSFQLLVPKKDKEFLRNLKDFKSIDELIQYYEEIFHLYNDMIGLEDTDTGTNRMSKNRYFLKADVNGCGGACYYDWCTVNSEDTVDMWLKKNNWGPLHEIGHGYQAAFDDKGMYTGEVSNNLFGVQHQYSKNGKDADKIGWLFNYGKKESVEKNLYQAIIKEGKGYTEVDDLRFQLILLTMLKQKAG

SEQ ID NO: 322 - protein, 855 aa, Serratia marcescens
MPTITKTQSLYTLTRPVWLDAAGMSKGIDHDRQHLGIILAAGQVLRVRQTNAAFTGKLTLRLLNDDSKTEAECSVGASWVEAQVNAVSVPFIDTPYGQGEPVLEFEYPDTAKALPVYRRGENEAAFFARWDDRDAEFALVDAEYAVLLVPKISKPALKSLGDAKNIDGLIAYYDRIFTFYNALTGVSFEAEHESDRNIRNRYFMKADKHGAGAAYYGGRWTAETSNSVSSFWLTPKDSNWGSLHEIAHGYQGHFMGDKYFSTGEMWNNIYAACYQDVMLGERKYQEGWLYNYGNAAGVEKKIIDLIAQGTPLNQWDLRSKLFFAMLMVDKAGKNAFTHFNQQYRQSCNTPGFVPSEHALLDLLSASFAAAGEQIDVTPFVELTGGVITQAQRDRNAFSHAKAVYPLYQLVDAQSLPAVQAQLQLDSSLRLVDSCQLKASGLKGDVTLSLDIDDFAQIYGEDLILMEGARYAHKVRIDTPTLALTALPVGVYTLRLPTGKDRKYRPTAEYLTVKQGKNAVTLRFERKAASPIVSQEINLLGLSDAVFATVVVDRAKGAVVVDVTSTTPHAYFPGETYARVTLRDRQGAGRFSKEIQGTQATLSHDELPFAEGDDIEIYHVEPSRVRLNPAATGVIDNKNKTNVLRIEKSGLKNPALSGDPEQALLARLEQAAQRLRHLLPAYYAPSSSFKDDIYLAIGAFAEPQRESLLATYRDCLPADNTPPGANVGNAFTVAAKGIGDRQFLTGAIDLVKKTVTVTLAAGIAHHYFADSYAFLSVTDADGNVLLSYDVIGSQNQPAKTWVLPLSGYGGEVIQLRHEEPDNRLAIVNEMQHLRLTEKGKQQTYCITPVGLKRIND

SEQ ID NO: 323 - protein, 487 aa, Serratia sp.
MQSTKKAIEVTESSLAAATTGYDAVDDLLHYHERGNGIQVNGKDSFSTEQAGLFITRENQTWNGHNVFGQPAKLTFSFPDYKFSSTNVAGDTGLSKFSAEQQQQAKLSLQSWSDVANITFTEVAANQKANITFGNYSQDRPGHYDYDTQAYAFLPNTIYQGQNLGGQTWYNVNQSNVEHPASEDYGRQTFTHEIGHALGLSHPGDYNAGEGNPTYRDASYAEDTREFSLMSYWSETNTGGDNGGHYAAAPLLDDISAIQHLYGANLSTRTGDTVYGFNSNTDRDFLSTASNSQKVIFAAWDAGGNDTFDFSGYTANQRINLNEKSFSDVGGLKGNVSIAAGVTIENAIGGSGDDVIVGNAANNVLKGGAGNDVLFGGGGADELWGGAGKDTFVFSAVSDSAPGASDWIRDFQKGTDKIDLSYFNKGAQAGDQIHFVDHFSGAVGEALLSYDASSNVSDLALNIGGHQAPDFLVKIVGQVDVATDFIV

SEQ ID NO: 324 - protein, 409 aa, Trichoderma atroviride
MTSIRRLALYLGALLPAVLAAPAALHKKPEAVPNKFIVTLKEGASIDTDSHLAWVTDIHTRSLTKRSTAGVEKTYNIHTWNAYAGEFDEETIEQIKSNPDVASVEPDYIMHLSDIVEDKRALTTQSGAPWGLGTVSHRTSGSTSYIYDSSAGAGTFAYVVDSGINTSHQQFGGRASLGYNAAGGQHVDTLGHGTHVSGTIGGSTYGVAKQASLISVKVFAGESASTSVILDGYNWAVNDIVSKSRASKSAINMSLGGPASSTWTTAINAAFNQGVLTIVAAGNGDSLGNPQPVSGTSPANVPNAITVAALDINWRTASFTNYGAGVDVFAPGVNILSSWIGSNTATNTISGTSMATPHVVGLALYLQSLEGLTSPTAVTNRIKALATSGRVTGSLNGSPNVIIFNGNSS

SEQ ID NO: 325 - protein, 857 aa, Trichoplusia ni granulovirus
MSLVVSVQAPVVQRPAWIKSDQLSYLGIRHKRTPLPYIITPQTLLRYRTTTEGSVSLRLLNFNNNNEKTYNVTASWQISINHAEGVLFVDSVEFGTHTVEVELGGAWTHLPMYILGETNENAFKTLLTTARFAWLDLRVVSIFVPPNSINVLRNTSVSALYSFYKTIISFYDAFTGLVHDPTMFSINSNTNKQYFIKADSSGRDTAYYSSNWLAMSQSDLGKFLEPSFTNWLVLQTLGDAYNYGFTRYDTYWLGVWSGMLSDRLQYYYQDISERQTLSKIYAGERNRIEEEINVTIQDDLNLDLWTELHKIVFFTWILNSTGDGLVSEMNQSFRYRNTLNVKQPHHWVWLASLCKYDLVPLLYLCHATPALYIMLTDAKSLIEAGTAPPDHVLPHHLPLVQFISFKKCLYPINRLVGNFNINSNKIKEYVETNFTLFSPLQTRQLKISVSFTLTFQIHDMKQIAGDTVTIYDGLDVVAKKVVPDSGLVQLDDITPGVYMVHHPRGRNYFYSVCYDHFTTNDPYLLVHQNTTNVYVRYTRRVSSLFFDEEVILMGLHQVVMGVMYVNGPERRINVYLNNIQCDPFQPQTLFHHITLVYGNNTETQIRMTGATSTLTHGWHKFSNVTRLIINSKTLSCIFLETMLPAQPVRFALTDTGVMLDTDKALKIPDAEQRLRLRVNRHCKWLDSNPMALIIENDVRDNIYLATQDMETARYDRYYPDRVRRVAANYDFMFAGSAYKNYIKLSIRLLENIGTLVVEYGTLHDNFDTTYAGVQIKNSVGAVVLRSVFTGDEILESSHEDFPLCDEYIIQLYHREPNKLSVYKNFKLVPNFNDGKQNVFLKVQNNLLILTQGPNLID

SEQ ID NO: 326 - protein, 828 aa, Trichoplusia ni granulovirus
MSDRQCETRDITTLTVQVVNIPYTTSNQGRSPVGVFIKAGAYIDIRLNAGSPVYPIVFELMNNNRNTEFTITVMRHGMNETRLIQNDSALFVRWIKRPGRYSAILNIRGDYLKLPMYSGNLSKFRSEYASSECEYAYLNLGRVAMLVPPTDKVNTLALDLRVLAKYYSQITQHYDTTTGTNTYACVPFFIKADAGGPGDHYYSDSYIANSANTLGPFLLSTITNWPALHQIGHGYDHHFTNNTSLKEVWSSVLADSMQYQWMNTTERQNLPMIYDGDRREHAEAEIVKELAESVAFHDLDTNWRTILLAFVLKSNFGTATWRNMYSEENQIYDTFLRMVMQCSADLLAYFSLISLSIPIYVNVEEHIYARAYPFSAALAYHKPSVYPVRFLIDDYDTVSNNYDLKMFLDSNLSLVSVNDLVQAKIVQEKVTVKCVIDDPTQIIDEVYSLYDGSKLMQQGIITESLTIEFSQLYHGVYTLRPPRGLDKRYKIKLDTMYTHLIVDGAEKEHTLVYTPYNHSDLCYHTGHILSPQNGYCATFYIDFVNKKIVVDVHLADPVPTSPNIIFVRILIDTPSNKLDIMGANTPLGRHVLNFVPGAVLRILTDPQHLSLLVIFDNDVLFENSFKLLEDHIEERPDSDFIIENINQAGRWLDVHRNMLFVENEVSDDIYLAVQHFKDDEIYEELVTVFQHYFPQKLRPDYNYLFTFNNSVGIPVIVLTCNTLNNVANLIIYTGRVDFDNDNSDVYVGVEIITDKEILYSRYLTSETEVANLININDLLIKNGSIIKLIHAQPENLYLLKSGQIQDVSFKTDNTLIVVNNQLVLQDEV

SEQ ID NO: 327 - protein, 901 aa, Trichoplusia ni granulovirus
MSYKVIVPATVLPPWLRVGENWIFARHRRTEVGVVLPANTKFRVRADFSRAGFTRPVIVRLLNNNRSTEREINLNNDQWMEVEHAHESVPFVDWLVGEKNTMAEVYFEIDGPHIPLPVYVFNTRPVEHFKSEYRQSSSGYCFLYLDLVCMLVPPASKNALLDVNIFELHQFYNEIINYYDDLCGLVEDPYADTVDSNLPNKAAFVKADAGGPGGAYYGPFWTAPASSNLGDYLRISPTNWMVIHELGHAYDFVFTVNTILIEIWNNSLCDRIQYKWMNKIKRQQLARVYENRRPQKEATIQALIDNNSPFDNWGFFERLIIFTWLYNPQRGLDTLRNINHSYRVHATRNSSIPYPQIWSWLTTSAYDNFWLYFNLVGVYPADFYVNEHNKVVHFNLHLRALALGQSVRYPIKYIITDFDLVSKNYDIKQYLESNFDLVIPEELRQTDLLADVRVVCVIDDPSQIVGEPFSVYDGNERVFESTVATDGNMYLVGVGPGVYTLRAPRGKNKRYKLHLAHSPREPVHPANDHMYLLVTYPYYNQTLTYTPYVNSDLAVDMAHLFGSNDRRYVATIYFNPFEQTVTVHLNNIRAGRENNTTLYFEMVISNPFNGQSQTFTILEDNPTLRQGYYKFDVVTYSSIRLNMSVAGRLLFGDTFLPEGTTTLTMFPNQVLEPNLFPDGSALNRTLARLREQAAFLDNYSQLMYIENELRDSIYLASQLVDPASDEFVKYYPDYFRDPHTYVYLFRFRGLGDFVLLDLQIVPLLNLATVRIANIQNGPHSYFDTLYFKVELRDTNGAIVFSYSRRGNEPMTPEHHKFEVYSGYTVELFMREPGNRLQLIVNKMLDTALPSTQNIFARITDTQLVVGDTSIEDNLVTSINVDCGDDDNQKIRVVETLKMIAF

SEQ ID NO: 328 - protein, 1118 aa, Clostridium histolyticum
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK